(19) United States
(12) Patent Application Publication
(10) Pub. No.: US 2026/0109709 A1
(43) Pub. Date: Apr. 23, 2026

(54) 2-AMINO IMIDAZOLE DERIVATIVES AS PRMT5 INHIBITORS

(71) Applicant: MIRATI THERAPEUTICS, INC., San Diego, CA (US)

(72) Inventors: Svitlana KULYK, Redwood City, CA (US); Christopher Ronald SMITH, San Diego, CA (US); Anthony IVETAC, San Diego, CA (US); John David LAWSON, Carlsbad, CA (US)

(21) Appl. No.: 18/707,295

(22) PCT Filed: Nov. 4, 2022
(86) PCT No.: PCT/US2022/048973

§ 371 Date: May 3, 2024

Related U.S. Application Data
(60) Provisional application No. 63/276,479, filed on Nov. 5, 2021.
(60) Provisional application No. 63/356,861, filed on Jun. 29, 2022.

(51) Int. Cl. C07D 498/04; A61K 31/4184; A61K 31/4192; A61K 31/4196; A61K 31/422; A61K 31/427; A61K 31/433; A61K 31/437; A61K 31/4439; A61K 31/444; A61K 31/454; A61K 31/4709; A61K 31/4725; A61K 31/497; A61K 31/4985; A61K 31/501; A61K 31/506; A61K 31/5377; A61K 31/5383; C07D 235/30; C07D 401/04; C07D 401/06; C07D 401/12; C07D 401/14; C07D 403/04; C07D 403/12; C07D 403/14; C07D 405/04; C07D 405/12; C07D 405/14; C07D 409/12; C07D 413/04; C07D 413/06; C07D 413/12; C07D 417/12; C07D 471/04; C07D 487/04

(52) U.S. Cl. CPC C07D 498/04; A61K 31/4184; A61K 31/4192; A61K 31/4196; A61K 31/422; A61K 31/427; A61K 31/433; A61K 31/437; A61K 31/4439; A61K 31/444; A61K 31/454; A61K 31/4709; A61K 31/4725; A61K 31/497; A61K 31/4985; A61K 31/501; A61K 31/506; A61K 31/5377; A61K 31/5383; C07D 235/30; C07D 401/04; C07D 401/06; C07D 401/12; C07D 401/14; C07D 403/04; C07D 403/12; C07D 403/14; C07D 405/04; C07D 405/12; C07D 405/14; C07D 409/12; C07D 413/04; C07D 413/06; C07D 413/12; C07D 417/12; C07D 471/04; C07D 487/04

(57) ABSTRACT

Disclosed herein are 2-amino imidazole derivatives. These compounds inhibit Protein Arginine N-Methyl Transferase 5 (PRMT5) activity. In addition to the compounds, disclosed herein are pharmaceutical compositions containing the compounds, and methods of use, such as methods of treating cancer using the compounds and pharmaceutical compositions of the present invention.

### CROSS-REFERENCE TO RELATED APPLICATIONS

This application claims priority from U.S. Provisional Application No. 63/276,479, filed Nov. 5, 2021, and U.S. Provisional Application No. 63/356,861, filed Jun. 29, 2022, the disclosure of each of which is hereby incorporated by reference in its entirety.

### FIELD OF THE INVENTION

The present invention relates to compounds that are MTA-cooperative inhibitors of Protein Arginine N-Methyl Transferase 5 (PRMT5). In particular, the present invention relates to compounds, pharmaceutical compositions comprising the compounds and methods for use therefor.

### BACKGROUND OF THE INVENTION

Protein Arginine N-Methyl Transferase (PRMT5) is a type II arginine methyltransferase that catalyzes the transfer of a methyl group from S-adenosyl-L-methionine (SAM) to an omega-nitrogen of the guanidino function of protein L-arginine residues (omega-monomethylation) and the transfer of a second methyl group to the other omega-nitrogen, yielding symmetric dimethylarginine (sDMA). PRMT5 forms a complex with MEP50 (methylosome protein 50), which is required for substrate recoginition and orientation and is also required for PRMT5-catalyzed histone 2A and histone 4 methyltransferase activity (e.g., see Ho et al., (2013) PLOS ONE 8 (8): 10.1371/annotation/e6b5348e-9052-44ab-8f06-90d01dc88fc2).

Homozygous deletions of p16/CDKN2a are prevalent in cancer and these mutations commonly involve the co-deletion of adjacent genes, including the gene encoding methylthioadenosine phosphorylase (MTAP). It is estimated that approximately 15% of all human cancers have a homozygous deletion of the MTAP gene (e.g., see Firestone & Schramm (2017) J. Am. Chem Soc. 139 (39): 13754-13760. doi: 10.1021/jacs.7b05803. Epub 2017 Sep. 20).

Cells lacking MTAP activity have elevated levels of the MTAP substrate, methylthioadenosine (MTA), which is a potent inhibitor of PRMT5. Inhibition of PRMT5 activity results in reduced methylation activity and increased sensitivity of cellular proliferation to PRMT5 depletion or loss of activity. Hence, the loss of MTAP activity reduces methylation activity of PRMT5 making the cells selectively dependent on PRMT5 activity.

### SUMMARY OF THE INVENTION

There is a need to develop new MTA-cooperative PRMT5 inhibitors that are capable of inhibiting PRMT5 activity in the presence of elevated MTA concentrations, particularly in MTAP-deficient cells.

The inventors recognized that MTA-cooperative inhibition of PRMT5 activity in MTAP deleted cancers will provide therapeutic benefit for a wide range of cancers. The compounds of the present invention provide this therapeutic benefit as MTA-cooperative inhibitors of PRMT5 that negatively modulate the activity of MTA-bound PRMT5 in a cell, particularly an MTAP-deficient cell, or for treating various forms of MTAP-associated cancer.

In one aspect of the invention, compounds are provided that are represented by Formula I:

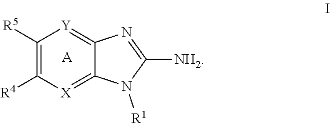

wherein:
X is CR² or N;
Y is CR³ or N;
R¹ is —C1-C2 alkyl or —CH₂-phenyl;
R² is hydrogen, cyano, —C1-C2 haloalkyl, or halogen;
R³ is hydrogen, halogen, or -L-R³⁰, wherein
L is absent or is selected from the group consisting of —(CH₂)₀₋₂—NH—(CH₂)₀₋₂—, —N(CH₃)—(CH₂)₀₋₂—, —N(C(O)CH₃)—(CH₂)₀₋₂—, (—CH₂—)₁₋₂, —C(halo)₂—, —C(CH₃)H—, —O—, —S—, —C(O)—, —NH—(CH₂)₀₋₁—CH(CH₃)—(CH₂)₀₋₁—, —NH—C(O)—(CH₂)₀₋₁— and —NH—CH(phenyl)-(CH₂)₀₋₁—;
R³⁰ is phenyl, isoxazole, oxazole, pyrrolidinone, morpholine, imidazopyridine, piperidinone, pyridinone, naphthalene, pyrazine, pyrazolidinone, —N(C₁-C₂ alkyl)₂, thiazole, thiadiazole, pyrimidine, imidazole optionally substituted with naphthyl where said naphthyl is further optionally substituted with one or more substituents selected from of cyano, halogen and —C(O)NH₂, pyrrolidine optionally fused to cyclopropane, pyrazole optionally substituted with —CH₂—CH₂-pyrrolidinone or naphthalene, pyridine optionally fused to cyclohexane, triazole optionally fused to a ring that forms pyrrolidine or morpholine, or tetrahydrofuran optionally spiro-bound to cyclobutane, wherein each R³⁰ is optionally substituted, or is optionally further substituted, with one or more substituents independently selected from —C₁-C₃ alkyl, —C₁-C₂ haloalkyl, halogen, hydroxy, —C(O)NH₂, —C₁-C₂ alkoxy, phenyl, cyano-phenyl and cyano;
R⁴ is hydrogen, —C₁-C₂ alkyl, —CH₂—O—CH₃ or halogen;
R⁵ is
hydrogen,
-L-R⁵⁰, wherein L is defined as above and
   R50 is
      —CH(OH)—CH₃, pyridine, pyrazine, isoxazole, pyridazine, pyrimidine, pyrazole, triazole optionally fused to a ring that forms pyrrolidine or morpholine, tetrahydro-thiopyran-dioxide, thiazole, furan, isoquinoline, naphthalene, pyrrolidine, phenyl where L is not absent, imidazole optionally fused with piperidine, tetrahydrofuran optionally substituted with imidazole, tetrahydropyran optionally spiro-bound to cyclobutane, or cyclobutane fused to tetrahydropyran,
      wherein each R⁵⁰ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl;
—(C₀-C₃)alkyl-(CO)N(R¹⁰)(R¹¹) where
   R¹⁰ is pyridyl(C₁-C₆ alkyl) where the pyridyl is optionally substituted with halogen (preferably bromo) or trifluoromethyl and
   R¹¹ is pyridyl(C₁-C₆ alkyl), pyrimidinyl(C₁-C₆ alkyl), naphthyl optionally substituted in the 1, 3 or 4 position, where position 2 is the point of attachment to N, independently with one, two or three of cyano, C₁-C₃ alkyl, cyclopropylmethyl, cyclopropyl, halogen or C₁-C₂ alkoxy, or 5,6,7,8-tetrahydroquinoxalinyl, or
—(C₀-C₃)alkyl-NH—C(O)(C₁-C₂alkyl)(R¹²) where R¹² is hydrogen, C₁-C₂ alkyl, or naphthyl optionally substituted with one or more independently selected cyano, chloro, fluoro, cyclopropyloxy, cyclopropylmethyl, or C₁-C₂ alkyl;
wherein at least one of R³, R⁴ and R⁵ are present and are not (i) hydrogen, (ii) C1 alkyl, (iii) C1 alkoxy where R² is not cyano, (iv) halogen, (v) cyano or (vi) unsubstituted phenyl at R⁵, wherein Y is nitrogen, or wherein R² is cyano and R¹ is C₁ alkyl; and wherein R¹ is not C₁ alkyl if Y is nitrogen, or a pharmaceutically acceptable salt thereof.

In another aspect of the invention, compounds and salts of Formula I are provided wherein ring A is selected from:

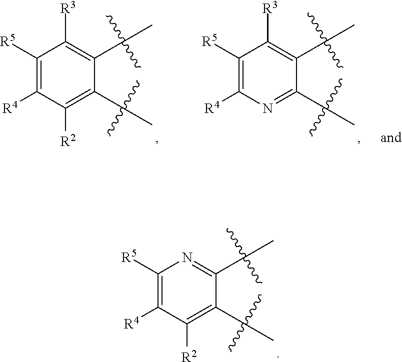

In another aspect of the invention, intermediates are provided that are useful for the preparation of compounds of Formula I.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting PRMT5 activity in a in a cell, comprising contacting the cell with a compound of Formula I. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula I or a pharmaceutically acceptable salt thereof, or a pharmaceutical composition thereof as defined herein. In one embodiment, the cell is an MTAP-deficient cell.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method for treating cancer in a patient in need thereof, the method comprising (a) determining that the cancer is associated with MTAP double deletion (e.g., an MTAP-associated cancer); and (b) administering to the patient a therapeutically effective amount of a compound of Formula I or a pharmaceutically acceptable salt thereof, or a pharmaceutical composition thereof.

### DETAILED DESCRIPTION OF THE INVENTION

The present invention relates to MTA-cooperative PRMT5 inhibitors. In particular, the present invention relates to compounds that inhibit PRMT5 activity in the presence of bound MTA, pharmaceutical compositions comprising a therapeutically effective amount of the compounds, and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference to the extent they are consistent with the present disclosure. Terms and ranges have their generally defined definition unless expressly defined otherwise.

For simplicity, chemical moieties are defined and referred to throughout primarily as univalent chemical moieties (e.g., alkyl, aryl, etc.). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for N, 2 for O, and 2, 4, or 6 for S, depending on the oxidation state of the S).

As used herein, “PRMT5” refers to a mammalian Protein Arginine N-Methyl Transferase 5 (PRMT5) enzyme.

As used herein, a “PRMT5 inhibitor” or “MTA-cooperative PRMT5 inhibitor” refers to compounds of the present invention that are represented by Formula (I) as described herein. These compounds are capable of negatively modulating or inhibiting all or a portion of the enzymatic activity of the PRMT5 in the presence of bound MTA in vitro or in vivo, or in cells expressing elevated levels of MTA.

As used herein, “MTAP” refers to a mammalian methylthioadenosine phosphorylase (MTAP) enzyme.

An “MTAP-associated disease or disorder” as used herein refers to diseases or disorders associated with or mediated by or having a loss of MTAP activity resulting in sensitizing the disorder to selective inhibition of PRMT5 activity. A non-limiting example of an MTAP-associated disease or disorder is an MTAP-associated cancer.

The term “amino” refers to —NH₂.

The term “acetyl” refers to —C(O)CH₃.

As herein employed, the term “acyl” refers to an alkylcarbonyl or arylcarbonyl substituent wherein the alkyl and aryl portions are as defined herein.

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups. Examples of alkyl groups include, without limitation, methyl, ethyl, propyl, isopropyl, butyl, isobutyl, sec-butyl, tert-butyl, pentyl, and hexyl.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups. Examples of alkenyl groups include, without limitation, ethenyl, propenyl, butenyl, pentenyl, and hexenyl.

The term “alkynyl” as used herein means an unsaturated straight or branched chain aliphatic group with one or more carbon-carbon triple bonds, having from 2 to 12 carbon atoms. As such, “alkynyl” encompasses C₂, C₃, C₄, C₅, C₆, C₇, C₈, C₉, C₁₀, C₁₁ and C₁₂ groups. Examples of alkynyl groups include, without limitation, ethynyl, propynyl, butynyl, pentynyl, and hexynyl.

An “alkylene,” “alkenylene,” or “alkynylene” group is an alkyl, alkenyl, or alkynyl group, as defined hereinabove, that is positioned between and serves to connect two other chemical groups. Examples of alkylene groups include, without limitation, methylene, ethylene, propylene, and butylene. Representative alkenylene groups include, without limitation, ethenylene, propenylene, and butenylene. Representative alkynylene groups include, without limitation, ethynylene, propynylene, and butynylene.

The term “alkoxy” refers to —OC₁-C₆ alkyl.

The term “cycloalkyl” as employed herein is a saturated and partially unsaturated cyclic hydrocarbon group having 3 to 12 carbons. As such, “cycloalkyl” includes C₃, C₄, C₅, C₆, C₇, C₈, C₉, C₁₀, C₁₁ and C₁₂ cyclic hydrocarbon groups. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are independently replaced O, S, or NRX, wherein Rx is hydrogen or C₁-C₃ alkyl. Examples of heteroalkyl groups include methoxymethyl, methoxyethyl and methoxypropyl.

An “aryl” group is a C₆-C₁₄ aromatic moiety comprising one to three aromatic rings. As such, “aryl” includes C₆, C₁₀, C₁₃, and C₁₄ cyclic hydrocarbon groups. A representative aryl group is a C₆-C₁₀ aryl group. Particular aryl groups include, without limitation, phenyl, naphthyl, anthracenyl, and fluorenyl. An “aryl” group also includes fused multicyclic (e.g., bicyclic) ring systems in which one or more of the fused rings is non-aromatic, provided that at least one ring is aromatic, such as indenyl.

An “aralkyl” or “arylalkyl” group comprises an aryl group covalently linked to an alkyl group wherein the moiety is linked to another group via the alkyl moiety. A representative aralkyl group is —(C₁-C₆)alkyl(C₆-C₁₀) aryl, including, without limitation, benzyl, phenethyl, and naphthylmethyl. For example, an arC₁-C₃alkyl is an aryl group covalently linked to a C₁-C₃ alkyl.

A “heterocyclyl” or “heterocyclic” group is a mono- or bicyclic (fused or spiro) ring structure having from 3 to 12 atoms, (3, 4, 5, 6, 7, 8, 9, 10, 11 or 12 atoms), for example 4 to 8 atoms, wherein one or more ring atoms are independently —C(O)—, N, NR⁴, O, or S, and the remainder of the ring atoms are quaternary or carbonyl carbons. Examples of heterocyclic groups include, without limitation, epoxy, oxiranyl, oxetanyl, azetidinyl, aziridinyl, THFyl, tetrahydropyranyl, tetrahydrothiophenyl, pyrrolidinyl, piperidinyl, piperazinyl, imidazolidinyl, thiazolidinyl, thiatanyl, dithianyl, trithianyl, azathianyl, oxathianyl, dioxolanyl, oxazolidinyl, oxazolidinonyl, decahydroquinolinyl, piperidonyl, 4-piperidonyl, thiomorpholinyl, dimethyl-morpholinyl, and morpholinyl. Specifically excluded from the scope of this term are compounds having adjacent ring O and/or S atoms.

As used herein, “L-heterocyclyl” refers to a heterocyclyl group covalently linked to another group via a linker (e.g., an alkylene linker).

As used herein, the term “heteroaryl” refers to a group having 5 to 14 ring atoms, preferably 5, 6, 10, 13 or 14 ring atoms; having 6, 10, or 14 π electrons shared in a cyclic array; and having, in addition to carbon atoms, from one to three heteroatoms that are each independently N, O, or S. “Heteroaryl” also includes fused multicyclic (e.g., bicyclic) ring systems in which one or more of the fused rings is non-aromatic, provided that at least one ring is aromatic and at least one ring contains an N, O, or S ring atom.

Examples of heteroaryl groups include acridinyl, azocinyl, benzimidazolyl, benzofuranyl, benzo[d]oxazol-2 (3H)-one, 2H-benzo[b][1,4]oxazin-3 (4H)-one, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “L-heteroaryl” group comprises a heteroaryl group covalently linked to another group via a linker (e.g., an alkylene linker).

An “arylene,” “heteroarylene,” or “heterocyclylene” group is a bivalent aryl, heteroaryl, or heterocyclyl group, respectively, as defined hereinabove, that is positioned between and serves to connect two other chemical groups.

As employed herein, when a moiety (e.g., cycloalkyl, aryl, heteroaryl, heterocyclyl, urea, etc.) is described as “optionally substituted” without expressly stating the substituents it is meant that the group optionally has from one to four, preferably from one to three, more preferably one or two, non-hydrogen substituents.

The term “halogen” or “halo” as employed herein refers to chlorine, bromine, fluorine, or iodine.

The term “haloalkyl” refers to an alkyl chain in which one or more hydrogens have been replaced by a halogen. Representative haloalkyls are trifluoromethyl, difluoromethyl, flurochloromethyl, chloromethyl, and fluoromethyl.

The term “hydroxyalkyl” refers to -alkylene-OH.

As used herein, “an effective amount” of a compound is an amount that is sufficient to negatively modulate or inhibit the activity of PRMT5 enzyme.

As used herein, a “therapeutically effective amount” of a compound is an amount that is sufficient to ameliorate or in some manner reduce a symptom or stop or reverse progression of a condition, or negatively modulate or inhibit the activity of PRMT5. Such amount may be administered as a single dosage or may be administered according to a regimen, whereby it is effective.

As used herein, “treatment” means any manner in which the symptoms or pathology of a condition, disorder or disease in a patient are ameliorated or otherwise beneficially altered.

As used herein, “amelioration of the symptoms of a particular disorder by administration of a particular compound or pharmaceutical composition” refers to any lessening, whether permanent or temporary, lasting or transient, that can be attributed to or associated with administration of the composition.

### Compounds

In one aspect of the invention, compounds are provided that are represented by Formula I:

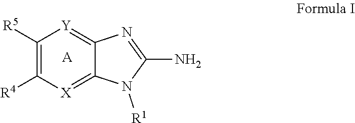

wherein:
X is CR² or N
Y is CR³ or N;
R¹ is —C₁-C₂ alkyl or —CH₂-phenyl;
R² is hydrogen, cyano, —C₁-C₂ haloalkyl, or halogen;
R³ is hydrogen, halogen, or -L-R³⁰, wherein
L is absent or is selected from the group consisting of —(CH₂)₀₋₂—NH—(CH₂)₀₋₂—, —N(CH₃)—(CH₂)₀₋₂—, —N(C(O)CH₃)—(CH₂)₀₋₂—, (—CH₂—)₁₋₂, —C(halo)₂—, —C(CH₃) H—, —O—, —S—, —C(O)—, —NH—(CH₂)₀₋₁—CH(CH₃)—(CH₂)₀₋₁—, —NH—C(O)—(CH₂)₀₋₁— and —NH—CH(phenyl)-(CH₂)₀₋₁—;
R³⁰ is phenyl, isoxazole, oxazole, pyrrolidinone, morpholine, imidazopyridine, piperidinone, pyridinone, naphthalene, pyrazine, pyrazolidinone, —N(C₁-C₂ alkyl)₂, thiazole, thiadiazole, pyrimidine, imidazole optionally substituted with naphthyl where said naphthyl is further optionally substituted with one or more substituents selected from of cyano, halogen and —C(O)NH₂, pyrrolidine optionally fused to cyclopropane, pyrazole optionally substituted with —CH₂—CH₂-pyrrolidinone or naphthalene, pyridine optionally fused to cyclohexane, triazole optionally fused to a ring that forms pyrrolidine or morpholine, or tetrahydrofuran optionally spiro-bound to cyclobutane,
   wherein each R³⁰ is optionally substituted, or is optionally further substituted, with one or more substituents independently selected from —C₁-C₃ alkyl, —C₁-C₂ haloalkyl, halogen, hydroxy, —C(O)NH₂, —C₁-C₂ alkoxy, phenyl, cyano-phenyl and cyano;
R⁴ is hydrogen, —C₁-C₂ alkyl, —CH₂—O—CH₃ or halogen;
R⁵ is
hydrogen,
   -L-R⁵⁰, wherein L is defined as above and R⁵⁰ is
      —CH(OH)—CH₃, pyridine, pyrazine, isoxazole, pyridazine, pyrimidine, pyrazole, triazole optionally fused to a ring that forms pyrrolidine or morpholine, tetrahydro-thiopyran-dioxide, thiazole, furan, isoquinoline, naphthalene, pyrrolidine, phenyl where L is not absent, imidazole optionally fused with piperidine, tetrahydrofuran optionally substituted with imidazole, tetrahydropyran optionally spiro-bound to cyclobutane, or cyclobutane fused to tetrahydropyran,
   wherein each R⁵⁰ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl;
(C₀-C₃)alkyl-(CO)N(R¹⁰)(R¹¹) where
   R¹⁰ is pyridyl(C₁-C₆ alkyl) where the pyridyl is optionally substituted with halogen (preferably bromo) or trifluoromethyl and
   R¹¹ is pyridyl(C₁-C₆ alkyl), pyrimidinyl(C₁-C₆ alkyl), naphthyl optionally substituted in the 1, 3 or 4 position, where position 2 is the point of attachment to N, independently with one, two or three of cyano, C₁-C₃ alkyl, cyclopropylmethyl, cyclopropyl, halogen or C₁-C₂ alkoxy, or 5,6,7,8-tetrahydroquinoxalinyl, or
—(C₀-C₃)alkyl-NH—C(O)(C₁-C₂ alkyl)(R¹²) where R¹² is hydrogen, C₁-C₂ alkyl, or naphthyl optionally substituted with one or more independently selected cyano, chloro, fluoro, cyclopropyloxy, cyclopropylmethyl, or C₁-C₂ alkyl;
wherein at least one of R³, R⁴ and R⁵ are present and are not (i) hydrogen, (ii) C₁ alkyl, (iii) C1 alkoxy where R² is not cyano, (iv) halogen, (v) cyano or (vi) unsubstituted phenyl at R⁵, wherein Y is nitrogen, or wherein R² is cyano and R¹ is C₁ alkyl; and wherein R¹ is not C₁ alkyl if Y is nitrogen,
or a pharmaceutically acceptable salt thereof.

In certain embodiments of formula I:
at least one of R³ (when present), R⁴ and R⁵ is not hydrogen, C₁ alkyl, or C₁ alkoxy;
R² is not cyano or halogen when R³ (when present), R⁴ and R⁵ are each hydrogen;
R⁵ is not unsubstituted phenyl when R¹ is C₁ alkyl, and R² (when present), R³ (when present), and R⁴ are each hydrogen;
when Y is nitrogen or R² is cyano, R¹ is not C₁ alkyl; and
R¹ is not C₁ alkyl if Y is nitrogen.

In another aspect of the invention, compounds and salts of Formula I are provided wherein ring A is selected from:

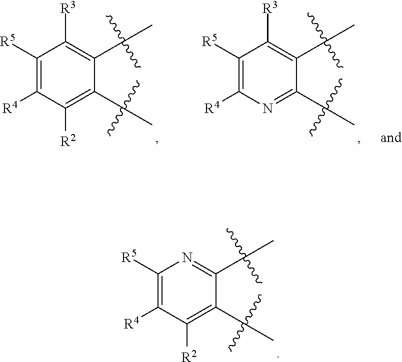

In one embodiment, the compound or salt of Formula I is provided wherein A is:

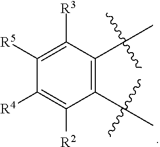

In another aspect of the invention, compounds and salts of Formula I are provided wherein ring A is:

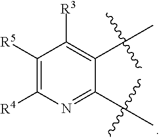

In another aspect of the invention, compounds and salts of Formula I are provided wherein ring A is:

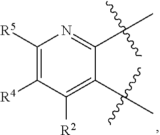

wherein R¹ is not C1 alkyl.

In one aspect of the invention, compounds are provided that are represented by Formula IA:

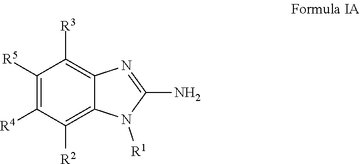

wherein:
R¹ is —C₁-C₂ alkyl or —CH₂-phenyl;
R² is hydrogen, cyano, —C₁-C₂ haloalkyl, or halogen;
R³ is hydrogen, halogen, -L-phenyl, -L-isoxazole, -L-oxazole, -L-pyrrolidinone, -L-morpholine, -L-imidazopyridine, -L-piperidinone, -L-pyridinone, -L-naphthalene, -L-pyrazine, -L-pyrazolidinone, -L-N(C₁-C₂ alkyl)₂, -L-thiazole, -L-thiadiazole, -L-pyrimidine, -L-imidazole optionally substituted with naphthyl where said naphthyl is further optionally substituted with one or more substituents selected from of cyano, halogen and —C(O)NH₂, -L-pyrrolidine optionally fused to cyclopropane, -L-pyrazole optionally substituted with —CH₂—CH₂-pyrrolidinone or naphthalene, -L-pyridine optionally fused to cyclohexane, -L-triazole optionally fused to a ring that forms pyrrolidine or morpholine, and -L-tetrahydrofuran optionally spiro-bound to cyclobutane, wherein R³ is optionally substituted, or is optionally further substituted, with one or more substituents independently selected from —C₁-C₃ alkyl, —C₁-C₂ haloalkyl, halogen, hydroxy, —C(O)NH₂, —C₁-C₂ alkoxy, phenyl, cyano-phenyl and cyano;
R⁴ is hydrogen, —C₁-C₂ alkyl, —CH₂—O—CH₃ or halogen;
R⁵ is hydrogen, -L-CH(OH)—CH₃, -L-pyridine, -L-pyrazine, -L-isoxazole, -L-pyridazine, -L-pyrimidine, -L-pyrazole, -L-triazole optionally fused to a ring that forms pyrrolidine or morpholine, -L-tetrahydro-thiopyran-dioxide, -L-thiazole, -L-furan, -L-isoquinoline, -L-naphthalene, -L-pyrrolidine, -L-phenyl where L is not absent, -L-imidazole optionally fused with piperidine, -L-tetrahydrofuran optionally substituted with imidazole, -L-tetrahydropyran optionally spiro-bound to cyclobutane, and -L-cyclobutane fused to tetrahydropyran, wherein R⁵ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl;
L is absent or is selected from the group consisting of —(CH₂)₀₋₂—NH—(CH₂)₀₋₂—, —N(CH₃)—(CH₂)₀₋₂—, —N(C(O)CH₃)—(CH₂)₀₋₂—, (—CH₂—)₁₋₂, —C(halo)₂-, —C(CH₃) H—, —O—, —S—, —C(O)—, —NH—(CH₂)₀₋₁—CH(CH₃)—(CH₂)₀₋₁—, —NH—C(O)—(CH₂)₀₋₁— and —NH—CH(phenyl)-(CH₂)₀₋₁—;
wherein at least one of R³, R⁴ and R⁵ are present and are not (i) hydrogen, (ii) C₁ alkyl, (iii) C₁ alkoxy where R² is not cyano, (iv) halogen, (v) cyano or (vi) unsubstituted phenyl at R⁵, or wherein R² is cyano and R¹ is C₁ alkyl, or a pharmaceutically acceptable salt thereof.

In one embodiment, the compound of Formula I or IA is provided wherein L is absent.

In one embodiment, the compound of Formula I or IA is provided wherein L is —NH—.

In one embodiment, the compound of Formula I or IA is provided wherein L is —(CH₂)₀₋₂—NH—(CH₂)₀₋₂.

In one embodiment, the compound of Formula I or IA is provided wherein R³ comprises a heteroaryl. In this embodiment, for example, R³ is -L-isoxazole, -L-oxazole, -L-imidazopyridine, -L-pyrazine, -L-pyrazolidinone, -L-thiazole, -L-thiadiazole, -L-pyrimidine, -L-imidazole optionally substituted with naphthyl where said naphthyl is further optionally substituted with one or more substituents selected from of cyano, halogen and —C(O)NH₂, -L-pyrazole optionally substituted with —CH₂—CH₂-pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted.

In one embodiment, the compound of Formula I or IA is provided wherein R³ comprises a heterocycle. In this embodiment, for example, R³ is -L-pyrrolidinone, -L-morpholine, -L-piperidinone, -L-pyridinone, -L-pyrrolidine optionally fused to cyclopropane, or -L-tetrahydrofuran optionally spiro-bound to cyclobutane, wherein R³ is optionally substituted, or is optionally further substituted.

In one embodiment, the compound of Formula I or IA is provided wherein R⁵ comprises a heteroaryl. In this embodiment, for example, R⁵ is -L-pyridine, -L-pyrazine, -L-isoxazole, -L-pyridazine, -L-pyrimidine, -L-pyrazole, -L-triazole optionally fused to a ring that forms pyrrolidine or morpholine, -L-thiazole, -L-furan, -L-isoquinoline, or -L-imidazole optionally fused with piperidine, wherein R⁵ is optionally substituted, or is optionally further substituted.

In one embodiment, the compound of Formula I or IA is provided wherein R⁵ comprises a heterocycle. In this embodiment, for example, R⁵ is -L-tetrahydro-thiopyran-dioxide, -L-pyrrolidine, -L-tetrahydrofuran optionally substituted with imidazole, -L-tetrahydropyran optionally spiro-bound to cyclobutane, wherein R⁵ is optionally substituted, or is optionally further substituted.

In one embodiment, the compound of Formula I or IA is provided wherein R³ or R⁵, but not both, comprise a heteroaryl. In this embodiment, for example,
(a) R³ is -L-isoxazole, -L-oxazole, -L-imidazopyridine, -L-pyrazine, -L-pyrazolidinone, -L-thiazole, -L-thiadiazole, -L-pyrimidine, -L-imidazole optionally substituted with naphthyl where said naphthyl is further optionally substituted with one or more substituents selected from of cyano, halogen and —C(O)NH₂, -L-pyrazole optionally substituted with —CH₂—CH₂-pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted, and
R⁵ is hydrogen, -L-CH(OH)—CH₃, -L-tetrahydro-thiopyran-dioxide, -L-naphthalene, -L-pyrrolidine, -L-phenyl where L is not absent, -L-tetrahydrofuran optionally substituted with imidazole, -L-tetrahydropyran optionally spiro-bound to cyclobutane, or -L-cyclobutane fused to tetrahydropyran, wherein R⁵ is optionally substituted, or is optionally further substituted;
or
(b) R³ is hydrogen, halogen, -L-phenyl, -L-pyrrolidinone, -L-morpholine, -L-piperidinone, -L-pyridinone, -L-naphthalene, -L-N(C₁-C₂ alkyl)₂, -L-pyrrolidine optionally fused to cyclopropane, or -L-tetrahydrofuran optionally spiro-bound to cyclobutane, wherein R³ is optionally substituted, or is optionally further substituted, with one or more substituents, and
   R⁵ is -L-pyridine, -L-pyrazine, -L-isoxazole, -L-pyridazine, -L-pyrimidine, -L-pyrazole, -L-triazole optionally fused to a ring that forms pyrrolidine or morpholine, -L-thiazole, -L-furan, -L-isoquinoline, or -L-imidazole optionally fused with piperidine, wherein R⁵ is optionally substituted, or is optionally further substituted.

In one embodiment, the compound of Formula I or IA is provided wherein R³ or R⁵, but not both, comprise a heterocycle. In this embodiment, for example,
(a) R³ is -L-pyrrolidinone, -L-morpholine, -L-piperidinone, -L-pyridinone, -L-pyrrolidine optionally fused to cyclopropane, or -L-tetrahydrofuran optionally spiro-bound to cyclobutane, wherein R³ is optionally substituted, or is optionally further substituted, and R⁵ is hydrogen, -L-CH(OH)—CH₃, -L-pyridine, -L-pyrazine, -L-isoxazole, -L-pyridazine, -L-pyrimidine, -L-pyrazole, -L-triazole optionally fused to a ring that forms pyrrolidine or morpholine, -L-thiazole, -L-furan, -L-isoquinoline, -L-naphthalene, -L-phenyl where L is not absent, -L-imidazole optionally fused with piperidine, or -L-cyclobutane fused to tetrahydropyran, wherein R⁵ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl; or
(b) R³ is hydrogen, halogen, -L-phenyl, -L-isoxazole, -L-oxazole, -L-imidazopyridine, -L-naphthalene, -L-pyrazine, -L-pyrazolidinone, -L-N(C₁-C₂ alkyl)₂, -L-thiazole, -L-thiadiazole, -L-pyrimidine, -L-imidazole optionally substituted with naphthyl where said naphthyl is further optionally substituted with one or more substituents selected from of cyano, halogen and —C(O)NH₂, -L-pyrazole optionally substituted with —CH₂—CH₂-pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted, and R⁵ is -L-tetrahydro-thiopyran-dioxide, -L-pyrrolidine, -L-tetrahydrofuran optionally substituted with imidazole, -L-tetrahydropyran optionally spiro-bound to cyclobutane, wherein R⁵ is optionally substituted, or is optionally further substituted.

In one embodiment, R⁵ is triazolylmethylamino substituted on the triazole with fluoromethyl, difluoromethyl or trifluoromethyl.

In one embodiment, R⁵ is triazolylmethylamino substituted on the triazole with methyol and fluoromethyl, difluoromethyl or trifluoromethyl.

In one embodiment, R⁵ is ((5-(difluoromethyl)-1-methyl-1H-1,2,3-triazol-4-yl)methyl)amino.

In one embodiment, R⁵ is triazolylmethylamino substituted on the triazole with C₁-C₆ alkyl, preferably methyl or ethyl.

In one embodiment, R⁵ is ((4-ethyl-4H-1,2,4-triazol-3-yl)methyl)amino.

In one embodiment, R⁵ is —(C₁-C₂ alkyl)-NH—C(O)(C₁-C₂ alkyl) (R¹²) where R¹² is C₁-C₂ alkyl or naphthyl optionally substituted independently with one or more cyano, chloro, fluoro, cyclopropyloxy, cyclopropylmethyl, or C₁-C₂ alkyl.

In one embodiment, R⁵ is —(C₁-C₂ alkyl)-NH—C(O)(C₁-C₂ alkyl)(R¹²) where R¹² is naphthyl substituted with one or two independently selected cyano, chloro, fluoro, cyclopropyloxy, cyclopropylmethyl, or C₁-C₂ alkyl.

In one embodiment, R⁵ is —(C₁-C₂ alkyl)-NH—C(O)(C₁-C₂ alkyl) (R¹²) where R¹² is naphthyl substituted with cyano.

In one embodiment, R⁵ is (N-(1-cyanonaphthalen-2-yl) acetamido)methyl.

In one embodiment, the compound of Formula I is selected from:

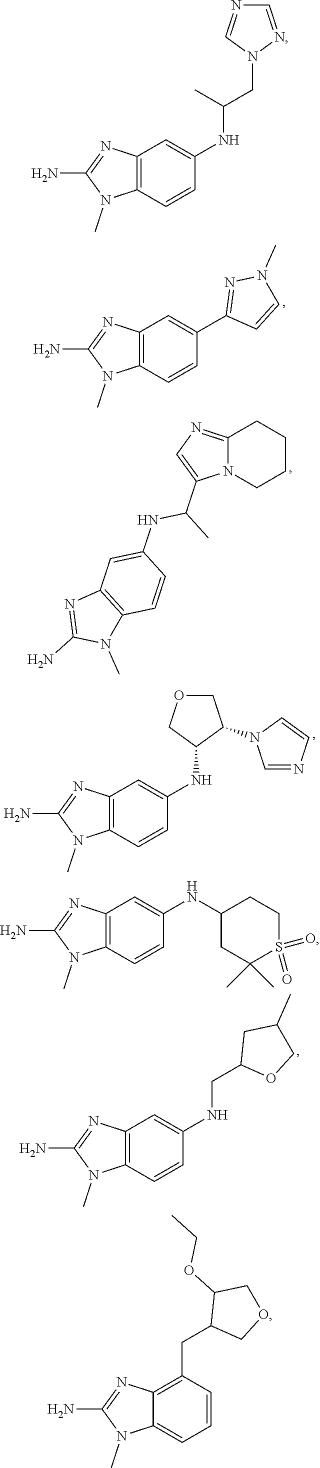

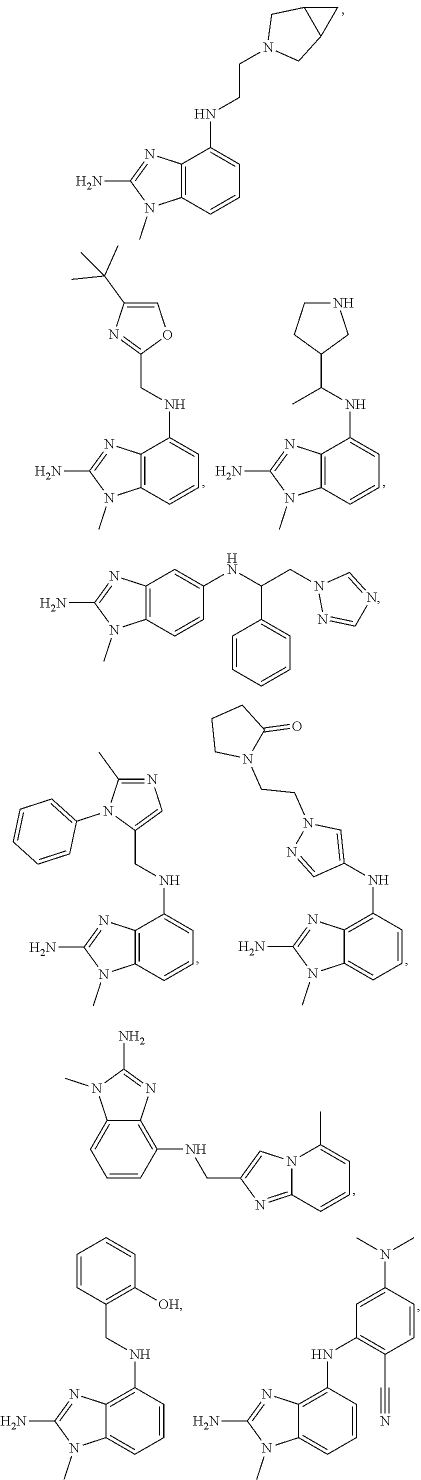

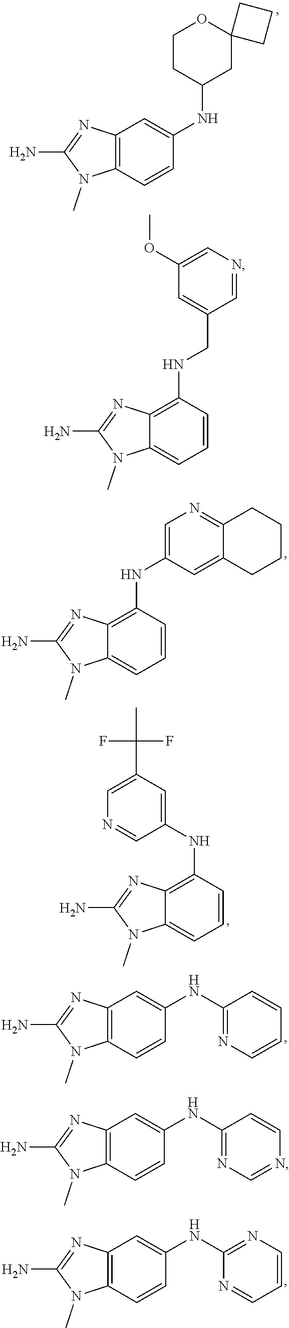

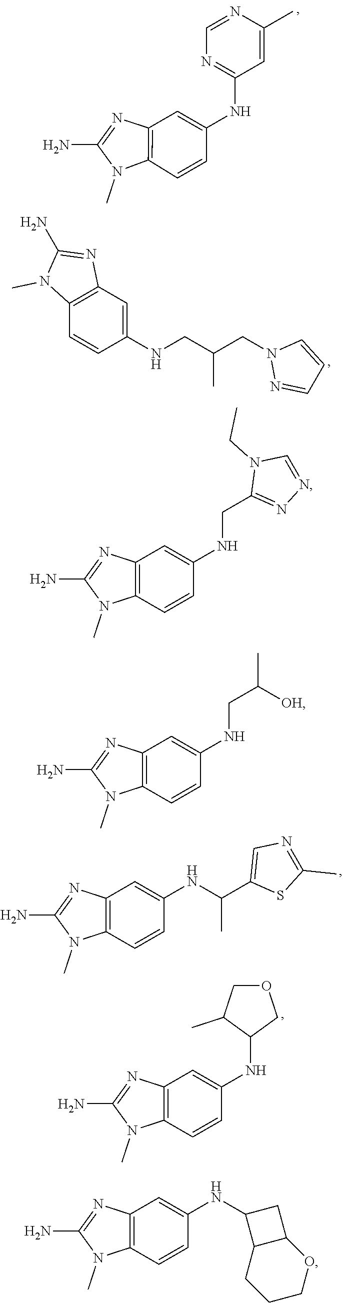

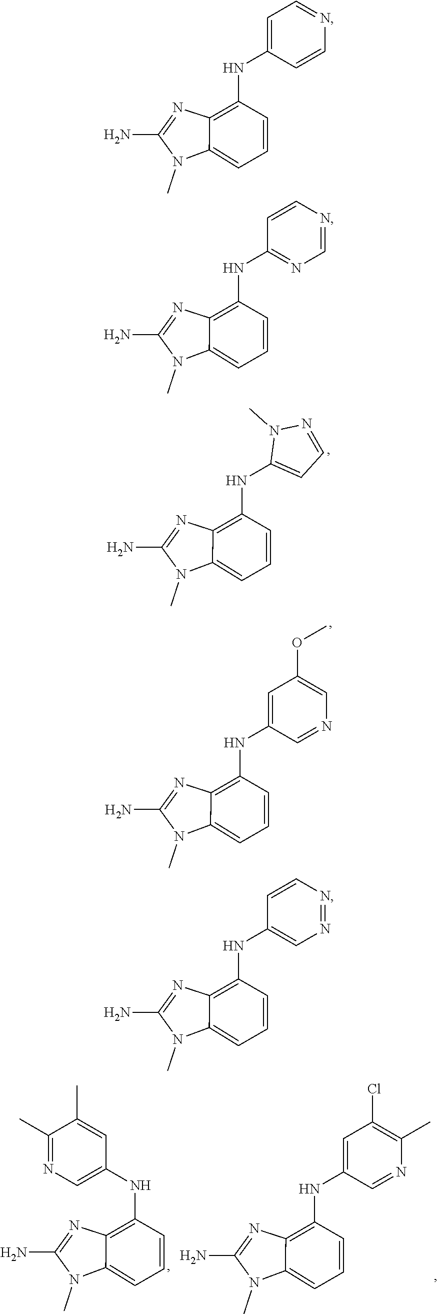

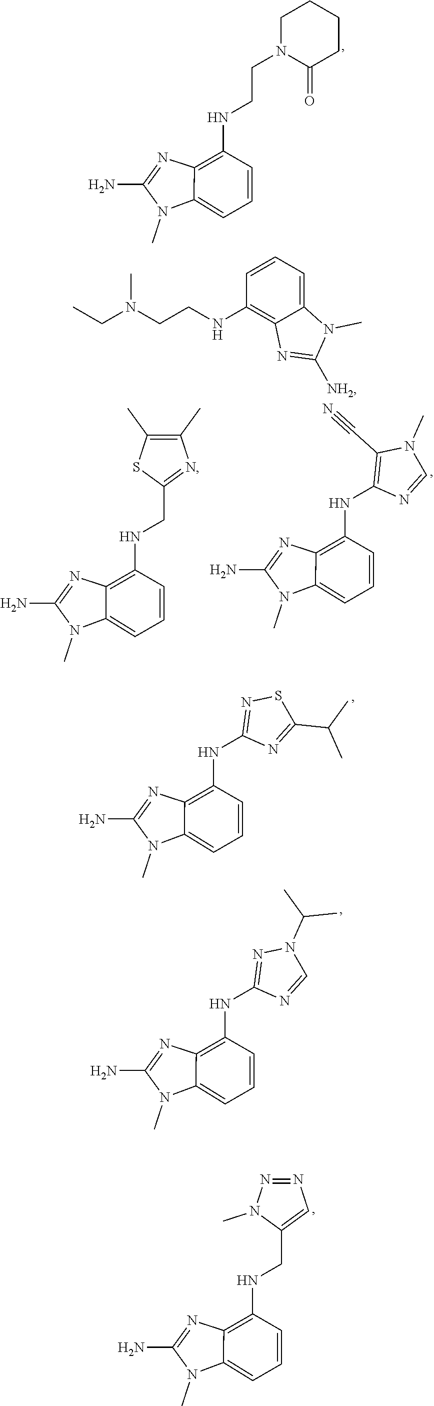

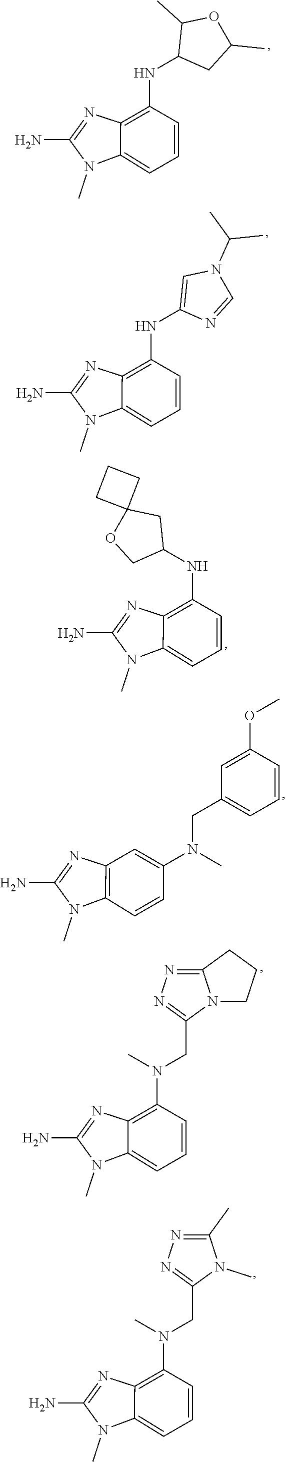

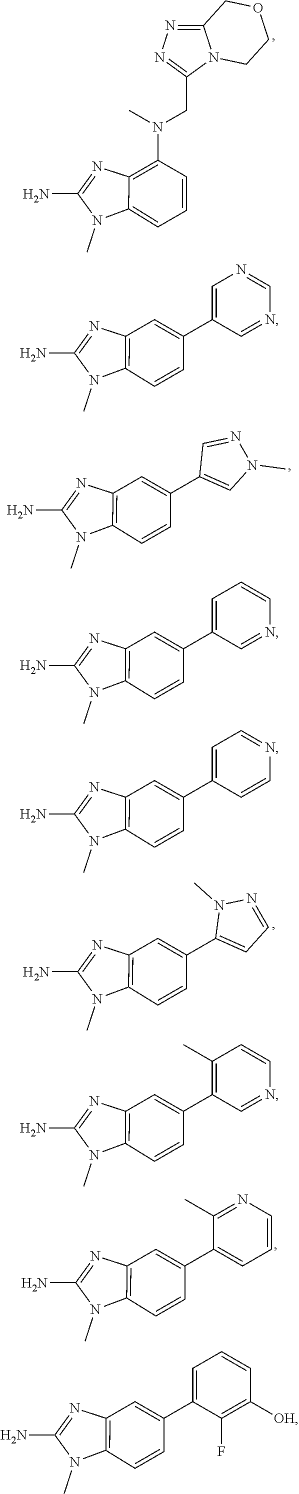

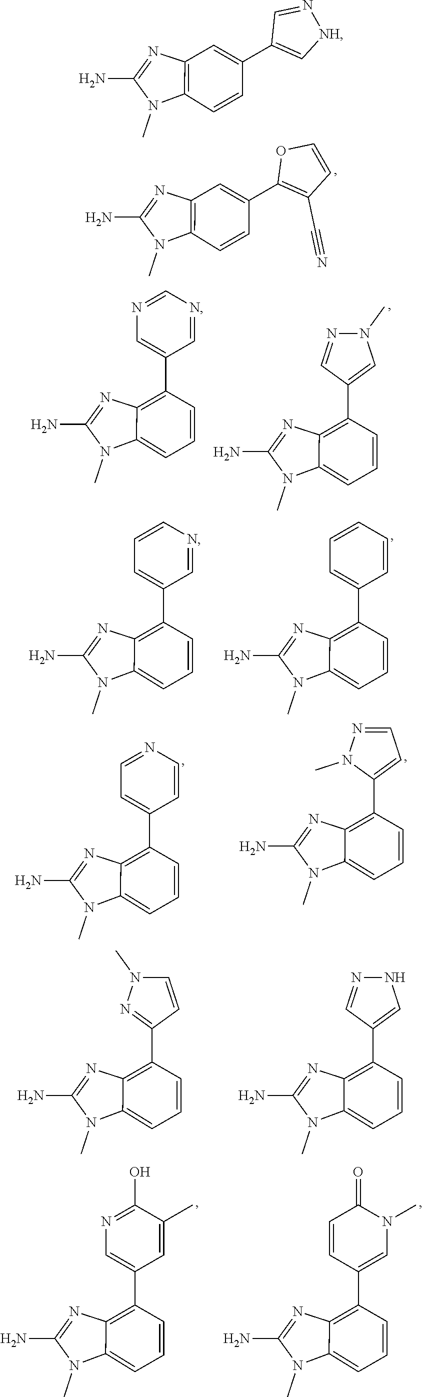

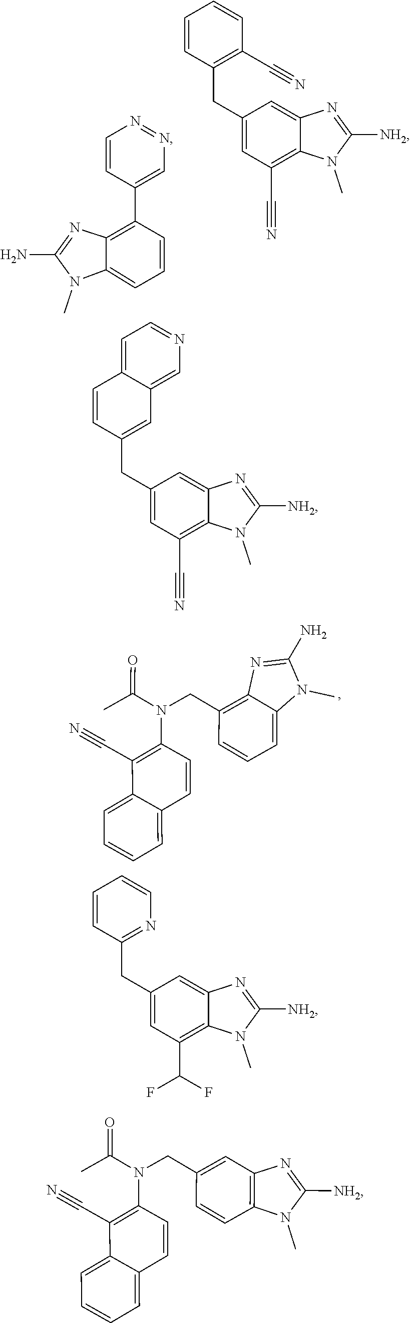

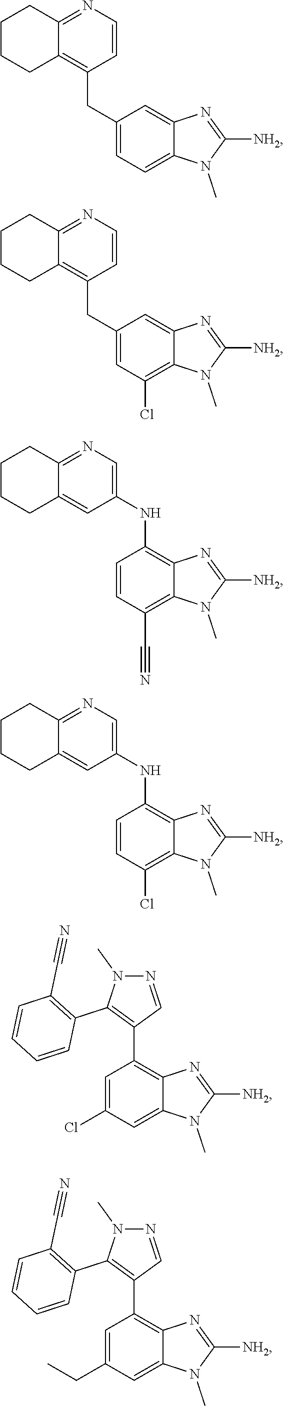 [SMILES: Cn1c(N)nc2cc(Cc3ccnc4c3CCCC4)ccc21 ; Cn1c(N)nc2cc(Cc3ccnc4c3CCCC4)cc(Cl)c21 ; Cn1c(N)nc2c(Nc3cnc4c(c3)CCCC4)ccc(C#N)c21 ; Cn1c(N)nc2c(Nc3cnc4c(c3)CCCC4)ccc(Cl)c21 ; Cn1ncc(-c2cc(Cl)cc3c2nc(N)n3C)c1-c1ccccc1C#N ; CCc1cc(-c2cnn(C)c2-c2ccccc2C#N)c2nc(N)n(C)c2c1]

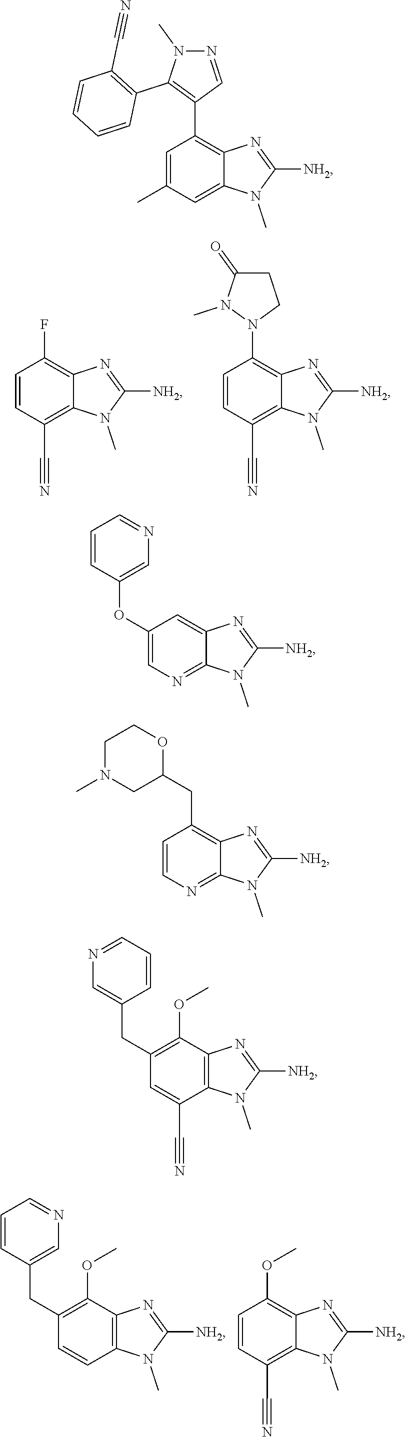

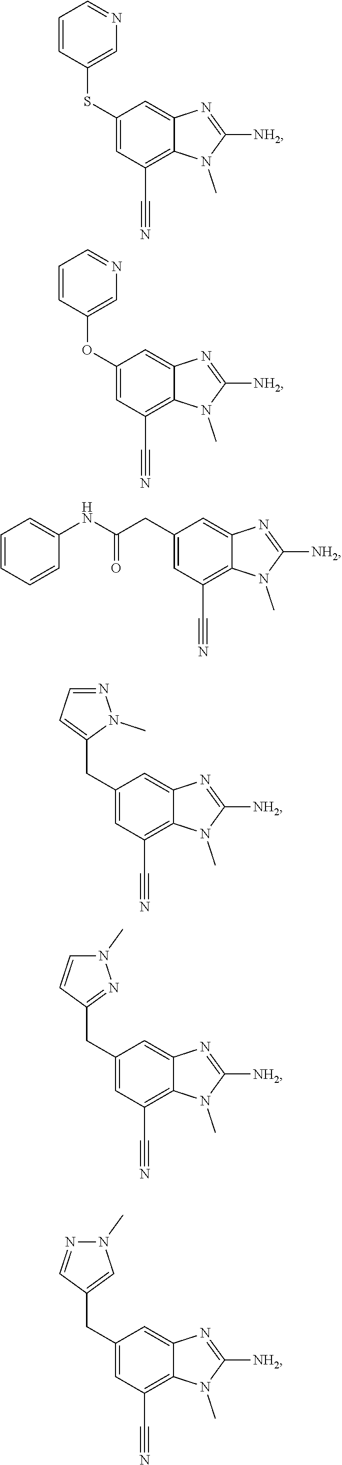

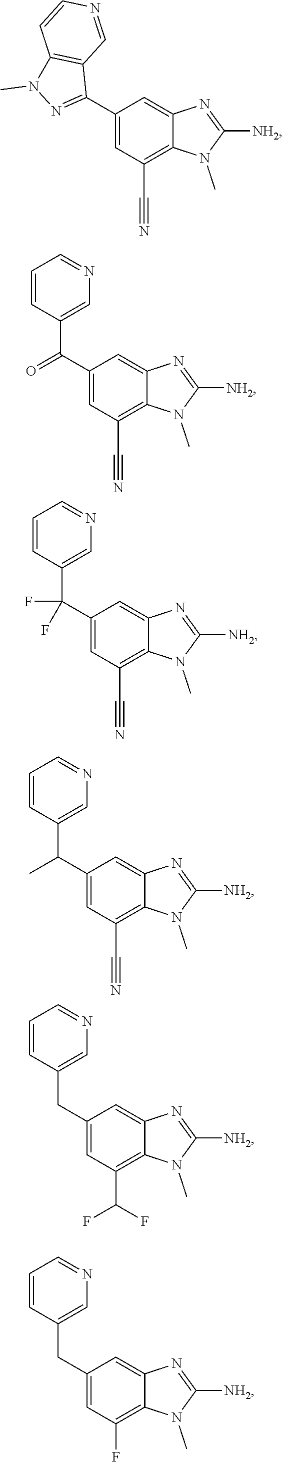

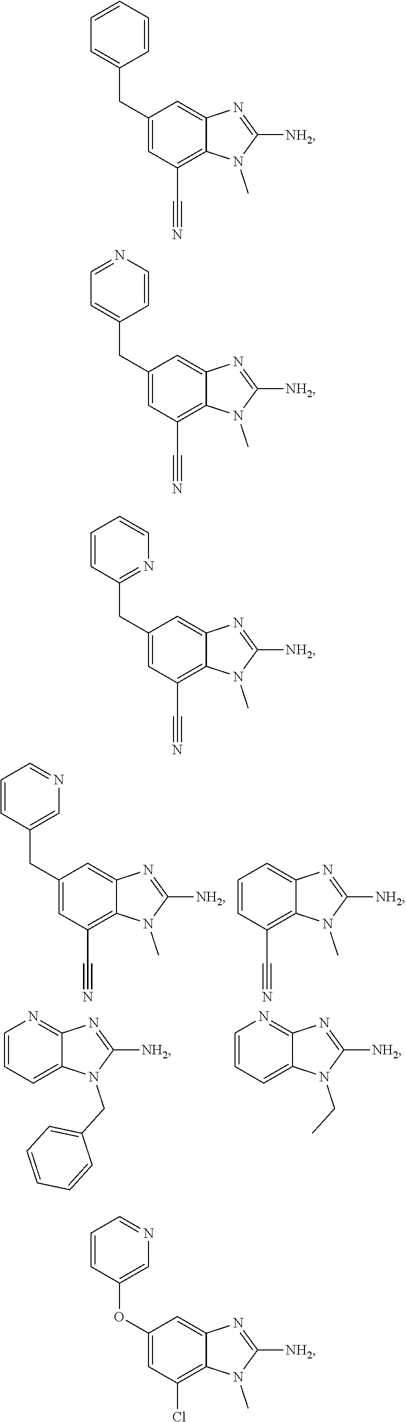

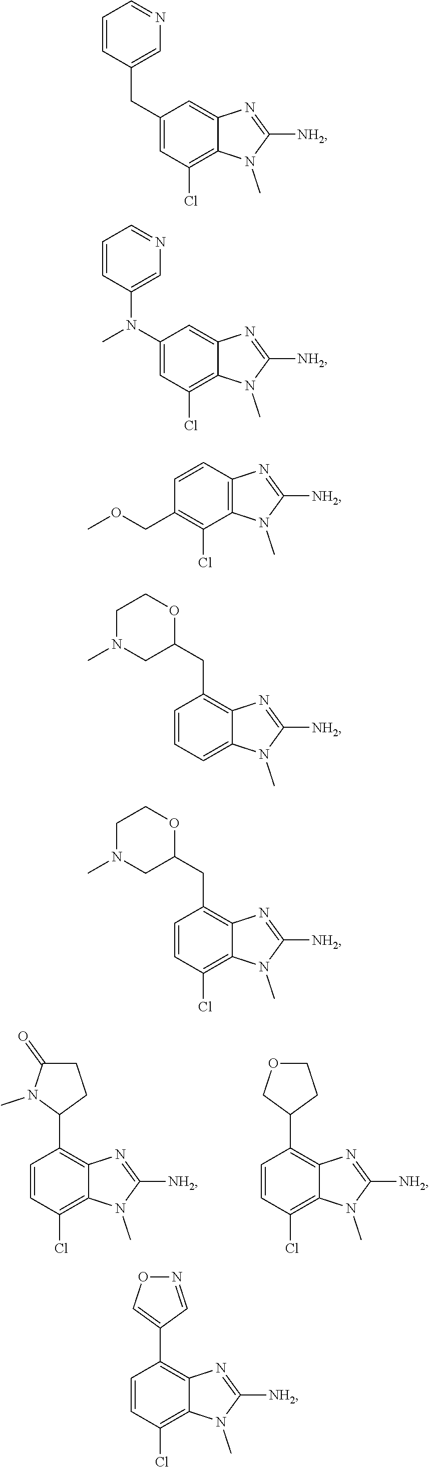

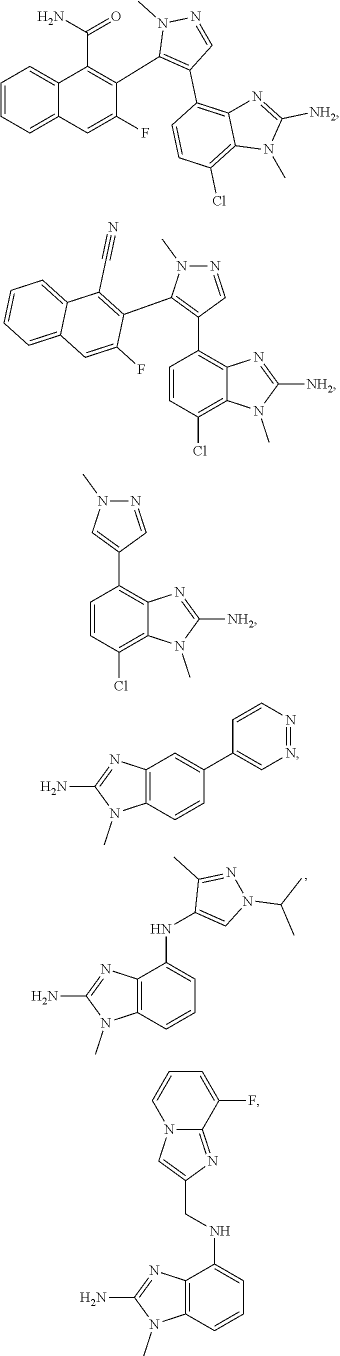

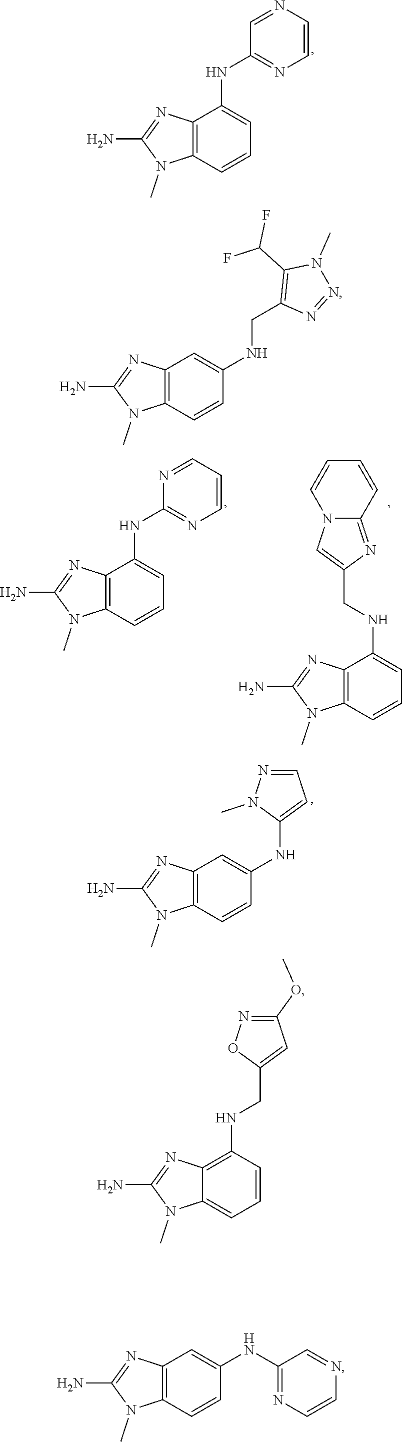

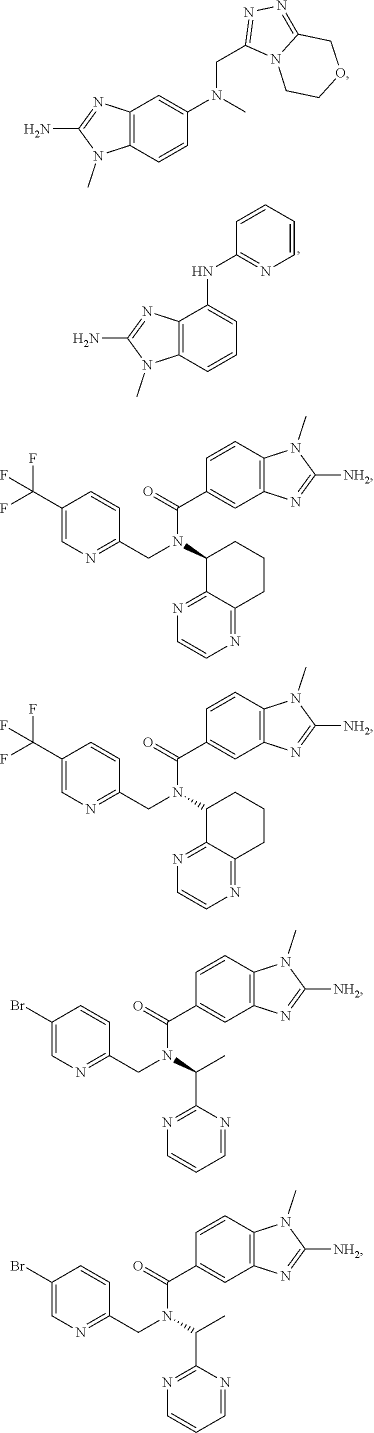

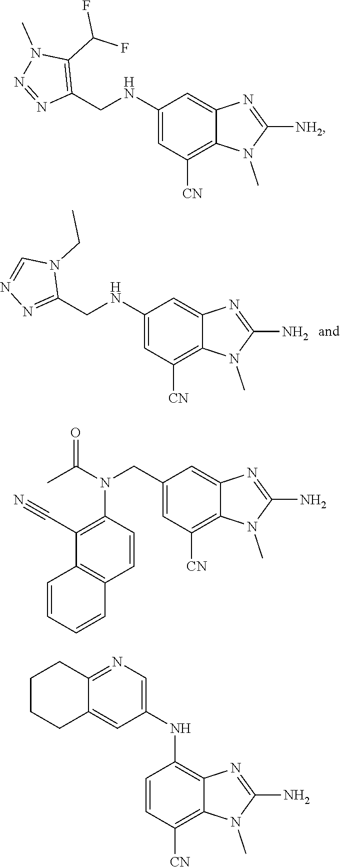

or a pharmaceutically acceptable salt of one of the foregoing compounds.

### Pharmaceutical Compositions

The compounds of Formula I may be formulated into pharmaceutical compositions.

In another aspect, the invention provides pharmaceutical compositions comprising a PRMT5 inhibitor according to the invention and a pharmaceutically acceptable carrier, excipient, or diluent. Compounds of the invention may be formulated by any method well known in the art and may be prepared for administration by any route, including, without limitation, parenteral, oral, sublingual, transdermal, topical, intranasal, intratracheal, or intrarectal. In certain embodiments, compounds of the invention are administered intravenously in a hospital setting. In certain other embodiments, administration may preferably be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term “pharmaceutically acceptable salts” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. A dose of the active compound for all of the above-mentioned conditions is in the range from about 0.01 to 300 mg/kg, preferably 0.1 to 100 mg/kg per day, more generally 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods described herein.

### Methods of Use

In yet another aspect, the invention provides for methods for inhibiting PRMT5 activity in a cell, comprising contacting the cell in which inhibition of PRMT5 activity is desired in vitro with an effective amount of a compound of Formula I, pharmaceutically acceptable salts thereof or pharmaceutical compositions containing the compound or pharmaceutically acceptable salt thereof. In one embodiment, the cell is an MTAP-deficient cell.

The compositions and methods provided herein are particularly deemed useful for inhibiting PRMT5 activity in a cell in vivo. In one embodiment, a cell in which inhibition of PRMT5 activity is desired is contacted in vivo with a therapeutically effective amount of a compound of Formula I, or a pharmaceutically acceptable salt thereof, to negatively modulate the activity of PRMT5. In other embodiments, a therapeutically effective amount of pharmaceutically acceptable salt or pharmaceutical compositions containing the compound of Formula I may be used. In one embodiment, the cell is an MTAP-deficient cell. In one embodiment, the negatively modulating the activity of PRMT5 occurs in the presence of bound MTA.

By negatively modulating the activity of PRMT5, particularly in cases for cells that lack MTAP activity, the methods are designed to inhibit PRMT5 activity to block cellular proliferation. The cells may be contacted in a single dose or multiple doses in accordance with a particular treatment regimen to affect the desired negative modulation of PRMT5. The degree PRMT5 inhibition may be monitored in vitro against the enzyme in the presence and absence of MTA and in the cell using well known methods, including those described in Example B below, to assess the effectiveness of treatment and dosages.

In another aspect, methods of treating cancer comprising administering to a patient having cancer a therapeutically effective amount of a compound of Formula I, pharmaceutically acceptable salts thereof or pharmaceutical compositions comprising the compound or pharmaceutically acceptable salts thereof are provided. In one embodiment, the cancer is an MTAP-associated cancer.

The compositions and methods provided herein may be used for the treatment of a wide variety of cancer including tumors such as prostate, breast, brain, skin, cervical carcinomas, testicular carcinomas, etc More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is diffuse large B-cell lymphoma (DLBCL).

In one embodiment, the cancer is an MTAP-associated cancer selected from hepatocellular carcinoma, breast cancer, skin cancer, bladder cancer, liver cancer, pancreatic cancer, and head and neck cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

### Reaction Schemes, Intermediates and Examples

The compounds of the present invention may be prepared using commercially available reagents and intermediates in the synthetic methods and reaction schemes described herein, or may be prepared using other reagents and conventional methods well known to those skilled in the art.

### INTERMEDIATE A

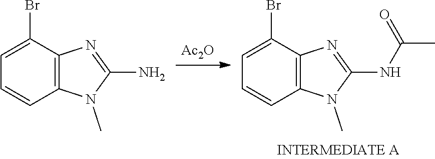

4-bromo-1-methyl-1H-1,3-benzodiazol-2-amine (20 g, 88.5 mol, 1 eq.) and acetic anhydride (9.93 g, 0.097 mol, 1.1 eq.) were combined in CHCl₃/MeCN (1:1 v/v, 500 mL) at room temperature. The mixture was stirred for 16 h at 50° C., cooled to room temperature and concentrated in vacuum. The residue was treated with sat. sodium bicarbonate solution (200 mL), and the product was extracted with ethyl acetate (250 mL×3). Combined organic layers were washed with brine (300 mL), dried over sodium sulfate, filtered and evaporated under reduced pressure to give N-(4-bromo-1-methyl-1H-1,3-benzodiazol-2-yl) acetamide, Intermediate A (23.5 g, 87.68 mol, 99% yield) as a brown solid. LCMS [ESI, M+1]⁺: 268.0/270.0. ¹H NMR (400 MHZ, DMSO-d₆) δ 10.81 (s, 1H), 7.53 (dd, J=8.0, 0.9 Hz, 1H), 7.42 (dd, J=7.8, 0.9 Hz, 1H), 7.22-7.06 (m, 1H), 3.61 (s, 3H), 2.16 (s, 3H).

### INTERMEDIATE B

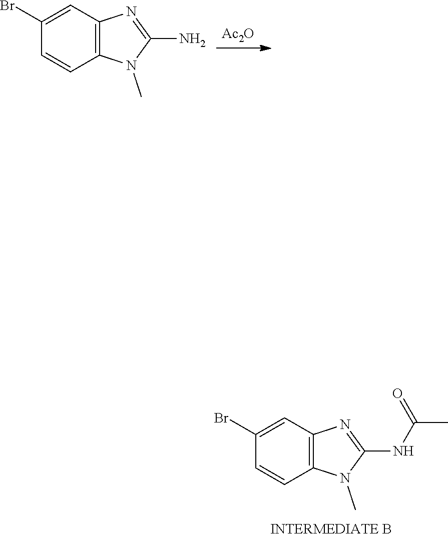

Intermediate B was synthesized from 5-bromo-1-methyl-1H-1,3-benzodiazol-2-amine following the same procedure as for intermediate A to afford N-(5-bromo-1-methyl-1H-1,3-benzodiazol-2-yl) acetamide, Intermediate B as a brown solid. LCMS [ESI, M+1]⁺: 268.2/270.2. ¹H NMR (400 MHZ, DMSO-d₆) δ 10.69 (s, 1H), 7.73 (d, J=1.9 Hz, 1H), 7.49 (d, J=8.7 Hz, 1H), 7.37 (dd, J=8.4, 1.9 Hz, 1H), 3.59 (s, 3H), 2.15 (s, 3H).

### INTERMEDIATE C

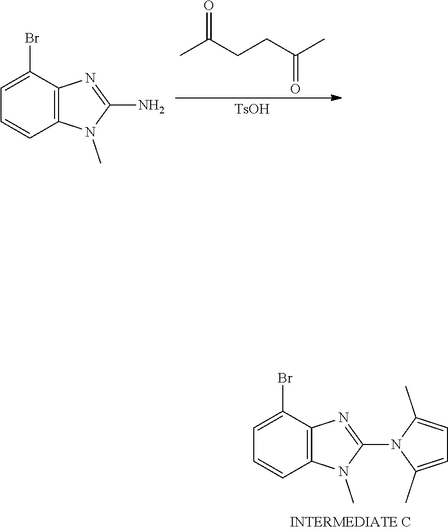

To a solution of 4-bromo-1-methyl-1H-benzo[d]imidazol-2-amine (2.00 g, 8.85 mmol, 1.00 eq.) in toluene (100 mL) and dimethyl formamide (20.0 mL) was added 4-methylbenzenesulfonic acid hydrate (101 mg, 531 umol, 0.06 eq.) and hexane-2,5-dione (5.05 g, 44.2 mmol, 5.19 mL, 5.00 eq.). The mixture was stirred at 145° C. for 48 hours beforebeing concentrated. The residue was diluted with water (200 mL) and extracted with ethyl acetate (50.0 mL×3). The organic layer was washed with brine (100 mL), dried over sodium sulfate, filtered and concentrated. The residue was purified by silica gel chromatography (ethyl acetate in petroleum ether 5-20%) to afford 4-bromo-2-(2,5-dimethyl-1H-pyrrol-1-yl)-1-methyl-1H-benzo[d]imidazole, intermediate C (1.60 g, 5.26 mmol, 60% yield) as a light yellow solid. LCMS [ESI, M+1]⁺=306.0. ¹H NMR (400 MHZ, CDCl₃) δ=7.59 (d, J=7.6 Hz, 1H), 7.39 (d, J=8.0 Hz, 1H), 7.32-7.28 (m, 1H), 5.97 (s, 2H), 3.52 (s, 3H), 2.09 (s, 6H).

### INTERMEDIATES D and E and E-1

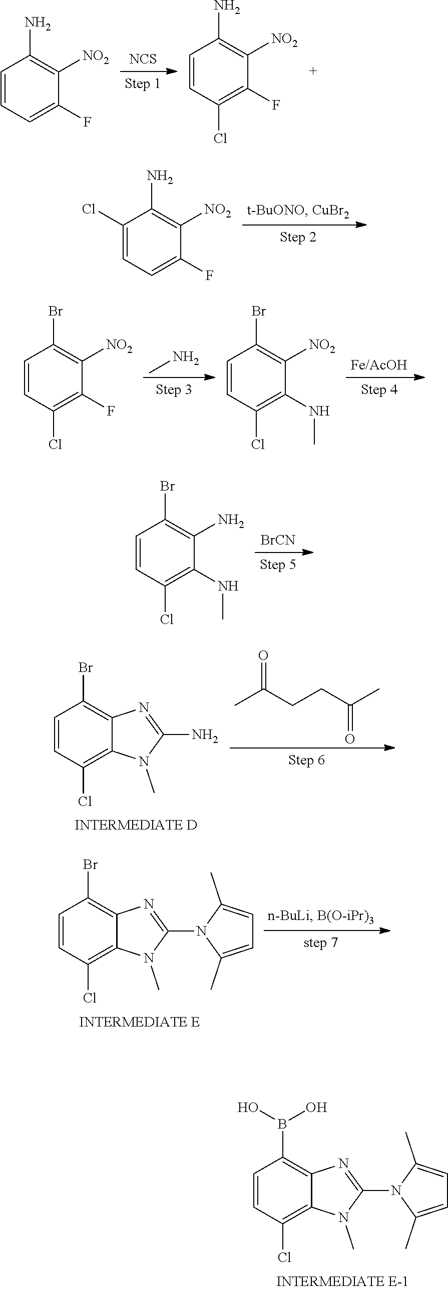

Step 1: To a solution of 3-fluoro-2-nitro-aniline (2.00 g, 12.8 mmol, 1.00 eq.) in N,N-dimethylformamide (30.0 mL) was added N-chlorosuccinimide (1.71 g, 12.8 mmol, 1.00 eq.) in N,N-dimethylformamide (20.0 mL) at 0° C. The mixture was stirred at 0° C. for 0.5 hour before being was warmed to 10° C. and stirred for 16 hours. The mixture was poured into water (200 mL) and extracted with ethyl acetate (50.0 mL×3). The combined organic layers were washed with water (50.0 mL) and brine (50.0 mL), dried over anhydrous sodium sulfate, filtered and concentrated in vacuum. The residue was purified by flash silica gel chromatography (Ethyl acetate in Petroleum ether 15-30%). The product was further purified by Prep-HPLC (methanol/water (0.05% formic acid) 0-40%) to give two fractions that were concentrated in vacuum to remove acetonitrile. The resulting aqueos mixture was extracted with ethyl acetate (20.0 mL×3). The combined organic layers were dried over sodium sulfate, filtered and concentrated in vacuum to afford 4-chloro-3-fluoro-2-nitro-aniline (500 mg, 2.62 mmol, 21% yield) as a yellow solid. GCMS [ESI, M]+: 189.9. ¹H NMR (400 MHZ, CDCl₃) δ=7.30-7.11 (m, 1H), 6.50 (dd, J=2.0, 9.2 Hz, 1H), 5.55 (br s, 2H); FNMR (400 MHZ, CDCl₃)>=−117.65 (s, 1F) and 6-chloro-3-fluoro-2-nitroaniline (400 mg, 2.10 mmol, 16.4% yield) as an orange solid.

¹H NMR (400 MHZ, CDCl₃) δ=7.41 (dd, J=4.8, 8.8 Hz, 1H), 6.50 (dd, J=8.8, 10.8 Hz, 1H), 6.06 (br s, 2H); FNMR (400 MHZ, CDCl₃) δ=−117.10 (s, 1F);

Step 2: To a solution of 4-chloro-3-fluoro-2-nitro-aniline (500 mg, 2.62 mmol, 1.00 eq.) in acetonitrile (10.0 mL) was added copper (II) bromide (879 mg, 3.94 mmol, 184 μL, 1.50 eq.) followed by tert-butyl nitrite (468 μL, 3.94 mmol, 1.50 eq.) dropwise at 10° C. under nitrogen atmosphere. The mixture was stirred at 10° C. for 16 hours. The mixture was poured into water (30.0 mL) and extracted with ethyl acetate (15.0 mL×3). The combined organic layers were washed with water (15.0 mL×3), brine (15.0 mL×2), dried over anhydrous sodium sulfate, filtered and concentrated in vacuum to afford 1-bromo-4-chloro-3-fluoro-2-nitrobenzene (500 mg, 1.97 mmol, 75% yield) as an orange solid. GCMS [ESI, M]*: 254.8.

Step 3: To a solution of 1-bromo-4-chloro-3-fluoro-2-nitrobenzene (400 mg, 1.57 mmol, 1.00 eq.) in tetrahydrofuran (1.00 mL) was added methylamine (2 M in tetrahydrofuran, 3.14 mL, 4.00 eq.) at 10° C. The mixture was stirred at 60° C. for 16 hours. The mixture was poured into saturated sodium bicarbonate solution (15.0 mL) and extracted with ethyl acetate (10.0 mL×3). The combined organic layers were washed with water (10.0 mL×2) and brine (10.0 mL), dried over anhydrous sodium sulfate, filtered and concentrated in vacuum to afford 3-bromo-6-chloro-N-methyl-2-nitroaniline (400 mg, 1.45 mmol, 92% yield) as an orange solid. LCMS [ESI, M+1]⁻: 264.8/266.8. ¹H NMR (400 MHZ, CDCl₃) δ=7.22 (d, J=8.4 Hz, 1H), 6.93 (d, J=8.4 Hz, 1H), 2.89 (s, 3H).

Step 4: To a solution of 3-bromo-6-chloro-N-methyl-2-nitroaniline (90.0 mg, 339 μmol, 1.00 eq.) in ethyl acetate (1.20 mL) and water (0.04 mL) was added acetic acid (420 mg, 6.99 mmol, 0.40 mL, 20.6 eq.). The mixture was warmed to 50° C. Iron powder (75.7 mg, 1.36 mmol, 4.00 eq.) was added to the mixture at 50° C. The resulting mixture was stirred at 80° C. for 2 hours. The mixture was poured into water (10.0 mL) and extracted with ethyl acetate (10.0 mL×3). The combined organic layers were washed with water (10.0 mL×3) and brine (10.0 mL). The organic layer was separated, dried over anhydrous sodium sulfate, filtered and concentrated in vacuum to afford 3-bromo-6-chloro-NI-methyl-benzene-1,2-diamine (54.0 mg, 224 μmol, 66% yield) as a light yellow solid. LCMS [ESI, M+1]⁻: 234.9/236.9

Step 5: To a solution of 3-bromo-6-chloro-NI-methyl-benzene-1,2-diamine (150 mg, 637 μmol, 1.00 eq.) in water (3.00 mL) and tetrahydrofuran (0.30 mL) was added cyanic bromide (81.0 mg, 764 μmol, 56.2 μL, 1.20 eq.). The resulting mixture was stirred at 50° C. for 6 hours. The mixture was diluted with water (10.0 mL) and extracted with a mixture of chloroform/isopropanol 3:1 (10 mL×5). The combined organic layers were washed with brine, dried over anhydrous sodium sulfate, filtered and concentrated to afford 4-bromo-7-chloro-1-methyl-benzimidazol-2-amine, Intermediate D (140 mg, 521 μmol, 82% yield) as a yellow solid. LCMS [ESI, M+1]⁻: 262.0. ¹H NMR (400 MHZ, CD₃OD) δ-7.38 (d, J=8.4 Hz, 1H), 7.15 (d, J=8.4 Hz, 1H), 3.94 (s, 3H).

Step 6: A mixture of 4-bromo-7-chloro-1-methyl-benzimidazol-2-amine (2.00 g, 7.68 mmol, 1.00 eq.), hexane-2,5-dione (4.38 g, 38.4 mmol, 4.50 mL, 5.00 eq.) and 4-methylbenzenesulfonic acid (79.3 mg, 461 μmol, 0.06 eq.) in toluene (20.0 mL) was stirred at 130° C. for 16 hours. Then hexane-2,5-dione (2.63 g, 23.0 mmol, 2.70 mL, 3.00 eq.) and 4-methylbenzenesulfonic acid (39.7 mg, 230 μmol, 0.03 eq.) were added and the resulting solution was stirred at 130° C. for 20 hours. The reaction mixture was concentrated. Then the crude was diluted with water (60.0 mL) and extracted with ethyl acetate (20.0 mL×3). The combined organic layers were dried over sodium sulfate, filtered and concentrated. The residue was purified by silica gel chromatography (ethyl acetate in petroleum ether 0-30%) to afford 4-bromo-7-chloro-2-(2,5-dimethylpyrrol-1-yl)-1-methyl-benzimidazole, intermediate E (330 mg, 870 μmol, 11% yield) as a black oil; LCMS [ESI, M+1]⁻: 338.1/340.1. ¹H NMR (400 MHZ, CDCl₃) δ=7.44 (d, J=8.4 Hz, 1H), 7.19 (d, J=8.4 Hz, 1H), 5.93 (s, 2H), 3.71 (s, 3H), 2.05 (s, 6H).

Step 7: To a solution of 4-bromo-7-chloro-2-(2,5-dimethylpyrrol-1-yl)-1-methyl-benzimidazole (330 mg, 975 μmol, 1.00 eq.) in tetrahydrofuran (5.00 mL) was added n-butyllithium (2.5 M, 585 μL, 1.50 eq.) at −70° C. under nitrogen atmosphere. The mixture was stirred at −70° C. for 1 hour under nitrogen atmosphere. Triisopropyl borate (403 μL, 1.75 mmol, 1.80 eq.) in tetrahydrofuran (1.00 mL) was added to the mixture at −70° C. under nitrogen atmosphere. The mixture was stirred at −70° C. for 0.5 hour before being slowly warmed up to 10° C. and stirred at 10° C. for 16 hours. The mixture was quenched with saturated ammonium chloride solution (20.0 mL) and extracted with ethyl acetate (20.0 mL×3). The combined organic layers were washed with water (20.0 mL), dried over anhydrous sodium sulfate, filtered and concentrated in vacuum. The residue was purified by flash silica gel chromatography (Ethyl acetate in Petroleum ether 30%) to afford [7-chloro-2-(2,5-dimethylpyrrol-1-yl)-1-methyl-benzimidazol-4-yl]boronic acid, Intermediate E-1 (58.0 mg, 173 μmol, 18% yield) as a yellow solid. LCMS [ESI, M+1]⁻: 304.1.

### INTERMEDIATES F and F-1

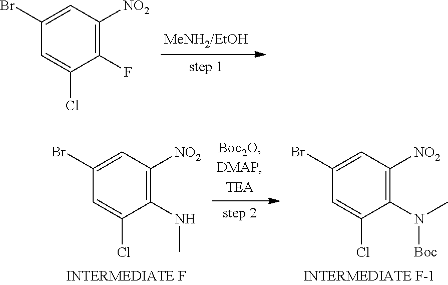 [SMILES: O=[N+]([O-])c1cc(Br)cc(Cl)c1F ; "MeNH2/EtOH" ; CNc1c(Cl)cc(Br)cc1[N+](=O)[O-] ; "Boc2O,DMAP,TEA" ; CN(C(=O)OC(C)(C)C)c1c(Cl)cc(Br)cc1[N+](=O)[O-]]

Step 1: To a solution of 5-bromo-1-chloro-2-fluoro-3-nitrobenzene (2.00 g, 7.86 mmol, 1.00 eq.) in ethanol (20.0 mL) was added methylamine/ethanol (10.0 g, 96.6 mmol, 30% of methylamine, 12.3 eq.). The mixture was stirred at 20° C. for 12 hours. The mixture was concentrated under vacuum. The crude product was triturated with water (20.0 mL) at 15° C. for 15 min to produce 4-bromo-2-chloro-N-methyl-6-nitroaniline, Intermediate F (2.20 g, crude) as a red solid. ¹H NMR (400 MHZ, CDCl₃-d) δ=8.05 (d, J=2.4 Hz, 1H), 7.60 (d, J=2.4 Hz, 1H), 6.91-6.60 (m, 1H), 3.11 (s, 3H).

Step 2: To a solution of 4-bromo-2-chloro-N-methyl-6-nitroaniline (1.10 g, 4.14 mmol, 1.00 eq.) in tetrahydrofuran (20.0 mL) was added 4-dimethylaminopyridine (50.6 mg, 414 μmol, 0.10 eq.), triethylamine (1.73 mL, 12.4 mmol, 3.00 eq.) and di-tert-butyldicarbonate (1.90 mL, 8.29 mmol, 2.00 eq.). The mixture was stirred at 50° C. for 12 hours. After being cooled to room temperature, the mixture was concentrated under vacuum. The residue was diluted with ethyl acetate (30.0 mL) and water (30.0 mL). The layers were separated, and the aqueous phase was extracted with ethyl acetate (3× 50.0 mL). The organic layer was dried over sodium sulfate, filtered and concentrated under vacuum. The residue was purified by silica gel chromatography (ethyl acetate in petroleum ether 0-10%) to afford tert-butyl N-(4-bromo-2-chloro-6-nitro-phenyl)-N-methyl-carbamate, Intermediate F-1 (1.50 g, 4.02 mmol, 97% yield) as a yellow oil. ¹H NMR (400 MHZ, CDCl₃-d) δ=7.93 (d, J=2.4 Hz, 1H), 7.85 (d, J=2.4 Hz, 1H), 3.16 (s, 3H), 1.31 (s, 9H).

### INTERMEDIATES G, H, G-1 and H-1

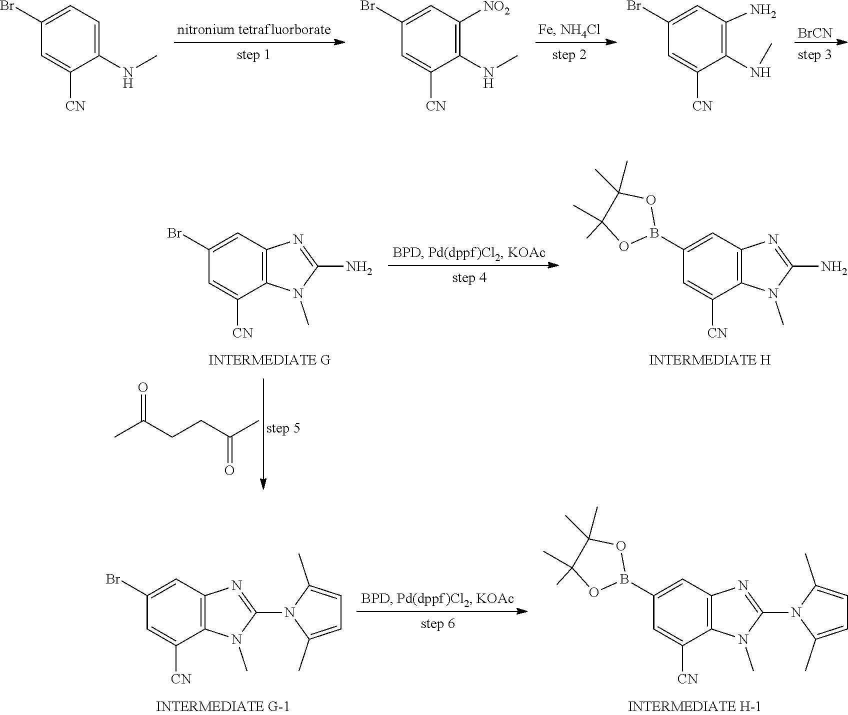

Step 1: To a solution of 5-bromo-2-(methylamino)benzonitrile (15.0 g, 71.1 mmol, 1.00 eq.) in acetonitrile (300 mL) was added nitronium tetrafluoroborate (8.78 mL, 85.3 mmol, 1.20 eq.) at 0° C. The reaction was stirred at 20° C. for 14 hours. The reaction mixture was diluted with water (100 mL) and stirred for 5 min. The aqueous phase was extracted with ethyl acetate (150 mL×3). The combined organic phase was dried with anhydrous sodium sulfate, filtered and concentrated in vacuum to give a residue. The residue was purified by column chromatography (SiO₂, ethyl acetate in petroleum ether/1-33%) to produce 5-bromo-2-(methylamino)-3-nitro-benzonitrile (6.00 g, crude) as a yellow solid. ¹H NMR (400 MHZ, DMSO-d₆) δ=8.90-8.88 (m, 1H), 8.87-8.85 (m, 1H), 3.71 (s, 3H).

Step 2: To a solution of 5-bromo-2-(methylamino)-3-nitrobenzonitrile (5.00 g, 19.5 mmol, 1.00 eq.) in ethyl acetate (50.0 mL) and water (1.50 mL) was added acetic acid (15.0 mL) and iron powder (10.9 g, 195 mmol, 10.0 eq.). The reaction was stirred at 60° C. for 1 hour. The reaction mixture was diluted with water (100 mL) and stirred for 5 min. The aqueous phase was extracted with ethyl acetate (200 mL×3). The combined organic phase was dried over anhydrous sodium sulfate, filtered and concentrated in vacuum to afford 3-amino-5-bromo-2-(methylamino)benzonitrile (4.10 g, 18.1 mmol, 93% yield) as a black brown gum. LCMS [M+1]⁺=227.9. ¹H NMR (400 MHZ, DMSO-d₆) δ=6.84 (m, 2H), 5.31 (br s, 2H), 4.48 (m, 1H), 3.06 (br d, J=4.8 Hz, 3H).

Step 3: To a solution of 3-amino-5-bromo-2-(methylamino)benzonitrile (4.10 g, 18.1 mmol, 1.00 eq.) in ethyl alcohol (45.0 mL) was added cyanogen bromide (3.84 g, 36.3 mmol, 2.67 mL, 2.00 eq.). The reaction was stirred at 20° C. for 2 hours. The reaction mixture was then diluted with water (50.0 mL) and stirred for 5 min. The aqueous phase was extracted with ethyl acetate (40.0 mL×3). The combined organic phase was dried over anhydrous sodium sulfate, filtered and concentrated in vacuum to give a residue. The residue was purified by column chromatography (SiO₂, ethyl acetate in petroleum ether 30-50%, then methanol in ethyl acetate 10%) to get 2-amino-6-bromo-3-methyl-benzimidazole-4-carbonitrile, Intermediate G (1.80 g, 6.77 mmol, 37% yield) as a dark brown solid. LCMS [M+1]⁺=252.8. ¹H NMR (400 MHZ, DMSO-d₆) δ=7.54 (d, J=1.6 Hz, 1H), 7.44 (d, J=2.0 Hz, 1H), 7.10 (s, 2H), 3.74 (s, 3H).

Step 4: A mixture of Intermediate G (270 mg, 1.08 mmol, 1.00 eq.), 4,4,4′,4′,5,5,5′,5′-octamethyl-2,2′-bi (1,3,2-dioxaborolane) (1.09 g, 4.30 mmol, 4.00 eq.), potassium acetate (211 mg, 2.15 mmol, 2.00 eq.), Pd(dppf)C₁2 (70.1 mg, 108 μmol, 0.10 eq.) in dioxane (5.00 mL) was degassed and stirred at 90° C. for 6 hours under nitrogen atmosphere. After being cooled to room temperature, the mixture was concentrated under vacuum. The residue was diluted with ethyl acetate (20.0 mL) and water (20.0 mL). The layers were separated and the aqueous phase was extracted with ethyl acetate (3×20.0 mL). The organic layer was dried over anhydrous sodium sulfate, filtered and concentrated under vacuum. The crude product was purified by reversed-phase HPLC (0.1% FA condition) to produce 2-amino-3-methyl-6-(4,4,5,5-tetramethyl-1,3,2-dioxaborolan-2-yl)benzimidazole-4-carbonitrile, Intermediate H (140 mg, 470 μmol, 44% yield) as a dark brown solid. ¹H NMR (400 MHZ, DMSO-d₆) δ=7.93 (s, 1H), 7.83 (s, 1H), 3.90 (s, 3H), 1.35 (s, 12H).

Step 5: To a solution of 2-amino-6-bromo-3-methyl-benzimidazole-4-carbonitrile (300 mg, 1.19 mmol, 1.00 eq.) in toluene (18.0 mL) and N,N-dimethylformamide (6.00 mL) was added 4-methylbenzenesulfonic acid hydrate (13.6 mg, 71.7 μmol, 0.06 eq.) and hexane-2,5-dione (682 mg, 5.97 mmol, 701 μL, 5.00 eq.) at 10° C. The mixture was stirred at 140° C. (reflux) for 16 hours with dean-Stark trap. The mixture was poured into water (30.0 mL) and extracted with ethyl acetate (15.0 mL×3). The combined organic layers were washed with water (10.0 mL) and brine (10.0 mL), dried over anhydrous sodium sulfate, filtered and concentrated in vacuum. The residue was purified by silica gel chromatography (ethyl acetate in petroleum ether 20%) to afford 6-bromo-2-(2,5-dimethylpyrrol-1-yl)-3-methyl-benzimidazole-4-carbonitrile, Intermediate G-1 (150 mg, 454 μmol, 38% yield) as a white solid. LCMS [ESI, M+1]⁺: 329.0/331.0. ¹H NMR (400 MHZ, CDCl₃) δ=8.35 (d, J=2.0 Hz, 1H), 8.13 (d, J=2.0 Hz, 1H), 5.96 (s, 2H), 3.66 (s, 3H), 1.98 (s, 6H).

Step 6: To a solution of 6-bromo-2-(2,5-dimethylpyrrol-1-yl)-3-methyl-benzimidazole-4-carbonitrile (600 mg, 1.82 mmol, 1.00 eq.) and 4,4,5,5-tetramethyl-2-(4,4,5,5-tetramethyl-1,3,2-dioxaborolan-2-yl)-1,3,2-dioxaborolane (1.39 g, 5.47 mmol, 3.00 eq.) in dioxane (9.00 mL) was added potassium acetate (537 mg, 5.47 mmol, 3.00 eq.), followed by [1,1′-bis(diphenylphosphino) ferrocene]dichloropalladium (II) (200 mg, 273 μmol, 0.15 eq.) at 15° C. under nitrogen atmosphere. The mixture was stirred at 80° C. for 16 hours. The mixture was poured into water (20 ml) and extracted with ethyl acetate (20.0 mL×3). The combined organic layers were dried over anhydrous sodium sulfate, filtered and concentrated in vacuum. The residue was purified by silica gel chromatography (ethyl acetate in petroleum ether 25%) to afford 2-(2,5-dimethylpyrrol-1-yl)-3-methyl-6-(4,4,5,5-tetramethyl-1,3,2-dioxaborolan-2-yl)benzimidazole-4-carbonitrile, Intermediate H-1 (710 mg, crude) as a white solid that was used in the next steps without further purification. LCMS [ESI, M+1]⁺: 377.2/295.1. ¹H NMR (400 MHZ, CDCl₃) δ=8.40 (s, 1H), 8.06 (s, 1H), 5.90 (s, 2H), 3.67 (s, 3H), 1.97 (s, 6H), 1.31 (s, 12H).

### INTERMEDIATES I, I-1, J and J-1

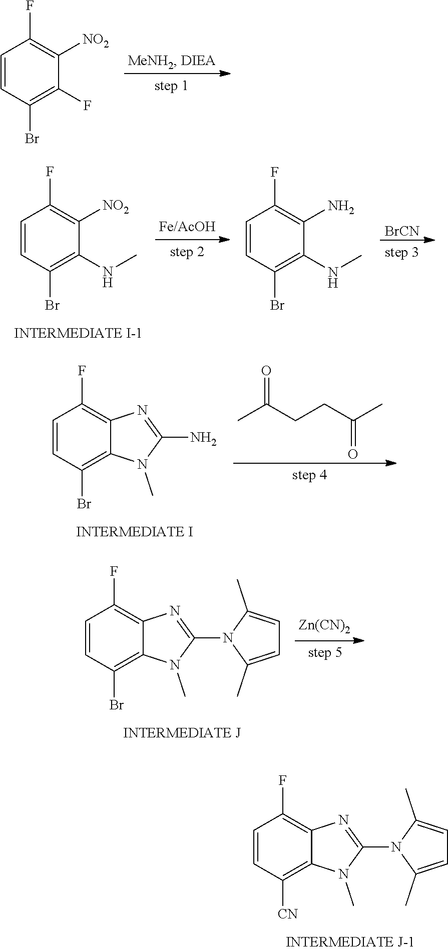

Step 1: To a mixture of 1-bromo-2,4-difluoro-3-nitrobenzene (7.00 g, 29.4 mmol, 1.00 eq.) in dimethyl formamide (105 mL) was added diisopropylethylamine (11.4 g, 88.2 mmol, 15.4 mL, 3.00 eq.) and methanamine hydrochloride (2.18 g, 32.3 mmol, 1.10 eq.). The reaction mixture was stirred at 15° C. for 16 hours. The reaction mixture was poured into water (500 mL), and extracted with ethyl acetate (200 mL×3). The combined organic layers were washed with brine (300 mL), dried over anhydrous sodium sulfate, filtered and concentrated. The residue was purified by silica gel chromatography (ethyl acetate in petroleum 0-30%) to afford 6-bromo-3-fluoro-N-methyl-2-nitroaniline, Intermediate I-1 (5.00 g, 20.1 mmol, 68% yield) as a yellow solid. ¹H NMR (400 MHZ, CDCl₃) δ=7.48 (dd, J=5.6, 8.8 Hz, 1H), 6.46-6.41 (m, 1H), 5.80 (s br, 1H), 2.87 (s, 3H).

Step 2: To a mixture of 6-bromo-3-fluoro-N-methyl-2-nitroaniline (5.00 g, 20.1 mmol, 1.00 eq.) in ethyl acetate (50.0 mL) and water (1.80 mL) was added acetic acid (24.1 g, 401 mmol, 23.0 mL, 20.0 eq.) at 25° C. The mixture was warmed to 50° C. and iron powder (4.48 g, 80.3 mmol, 4.00 eq.) was added. Then the reaction mixture was heated to 80° C. and stirred at 80° C. for 2 hours. The reaction mixture was washed with water (200 mL), extracted with ethyl acetate (100 mL×3). The combined organic layers were adjusted to pH=8 with saturated sodium bicarbonate and the organic layer was dried over anhydrous sodium sulfate, filtered and concentrated to afford 3-bromo-6-fluoro-N2-methyl-benzene-1,2-diamine (4.00 g, 18.3 mmol, 91% yield) as a black oil. ¹H NMR (400 MHZ, CDCl₃) δ=6.84 (dd, J=5.6, 8.8 Hz, 1H), 6.67-6.62 (m, 1H), 3.96 (s br, 2H), 2.70 (s, 3H).

Step 3: To a mixture of 3-bromo-6-fluoro-N2-methyl-benzene-1,2-diamine (2.00 g, 9.13 mmol, 1.00 eq.) in ethyl alcohol (30.0 mL) and water (30.0 mL) was added cyanogen bromide (3.87 g, 36.5 mmol, 4.00 eq.). The reaction mixture was stirred at 50° C. for 2 hours. The reaction mixture was adjusted to pH=12 by 2 M using sodium hydroxide solution and the solid was generated. Then the precipitate was filtered and concentrated to afford 7-bromo-4-fluoro-1-methyl-benzimidazol-2-amine, Intermediate I (900 mg, 3.49 mmol, 38% yield) as a brown solid. LCMS [ESI, M+1]: 244.1. ¹H NMR (400 MHz, DMSO-d₆) δ=6.97 (dd, J=4.4, 8.8 Hz, 1H), 6.85-6.68 (m, 3H), 3.76 (s, 3H)

Step 4: A mixture of 7-bromo-4-fluoro-1-methyl-benzimidazol-2-amine (600 mg, 2.46 mmol, 1.00 eq.), hexane-2,5-dione (1.40 g, 12.3 mmol, 1.44 mL, 5.00 eq.) and p-toluenesulfonic acid monohydrate (93.5 mg, 492 μmol, 0.20 eq.) in toluene (30.0 mL) was stirred at 130° C. for 16 hours with a Dean-Stark trap. The mixture was washed with water (30.0 mL), extracted with ethyl acetate (15 mL×3). The combined organic layers were dried over anhydrous sodium sulfate, filtered and concentrated. The residue was purified by silica gel chromatography (ethyl acetate in petroleum ether 0-10%) to afford 7-bromo-2-(2,5-dimethylpyrrol-1-yl)-4-fluoro-1-methyl-benzimidazole, Intermediate J (400 mg, 1.19 mmol, 48% yield) as a yellow oil. LCMS [ESI, M+1]: 323.9. ¹H NMR (400 MHZ, CDCl₃) δ=7.40 (dd, J=4.4, 8.4 Hz, 1H), 6.93 (dd, J=8.8, 9.6 Hz, 1H), 5.95 (s, 2H), 3.72 (s, 3H), 2.05 (s, 6H).

Step 5: To a mixture of Intermediate J (300 mg, 931 μmol, 1.0 eq.) in dimethyl formamide (5 mL) at 20° C. under nitrogen atmosphere was added tris(dibenzylideneacetone) dipalladium (0) (85 mg, 93 μmol, 0.1 eq.), zinc cyanide (328 mg, 2.79 mmol, 3.0 eq.), 1,1′-bis(diphenylphosphino) ferrocene (103 mg, 186 μmol, 0.2 eq.) and zinc powder (61 mg, 931 μmol, 1.0 eq.). The reaction mixture was stirred at 100° C. for 16 hours under nitrogen atmosphere. The reaction mixture was then washed with water (30 mL), extracted with ethyl acetate (15 mL×4). The combined organic layers were dried over anhydrous sodium sulfate, filtered and concentrated. The residue was purified by flash silica gel chromatography (40 g silica flash column, eluent of 0 ˜ 30% ethyl acetate/petroleum ether gradient) to afford 2-(2,5-dimethylpyrrol-1-yl)-7-fluoro-3-methyl-benzimidazole-4-carbonitrile, Intermediate J-1 (290 mg, 999 μmol, 99% yield) as yellow solid. LCMS [ESI, M+1]: 269.2. ¹H NMR (400 MHz, CDCl₃) δ=7.67 (dd, J=4.4, 8.4 Hz, 1H), 7.13-7.09 (m, 1H), 6.00-5.94 (m, 2H), 3.75 (s, 3H), 2.07-2.04 (m, 6H).

### INTERMEDIATE K

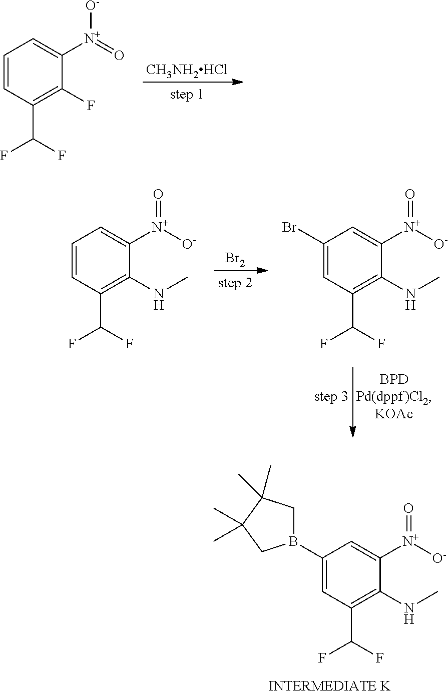

Step 1: To a solution of 1-(difluoromethyl)-2-fluoro-3-nitro-benzene (300 mg, 1.57 mmol, 1.00 eq.) and triethylamine (318 mg, 3.14 mmol, 437 μL, 2.00 eq.) in ethanol (5.00 mL) at 25° C. was added methylamine hydrochloride (212 mg, 3.14 mmol, 2.00 eq.) slowly. Then reaction mixture was stirred at 25° C. for 5 hours. The reaction mixture was quenched by addition of water (50.0 mL). The aqueous phase was extracted with dichloromethane (20.0 mL×3) and the combined organic extracts were dried with sodium sulfate, filtered and concentrated under reduced pressure to give 2-(difluoromethyl)-N-methyl-6-nitroaniline (280 mg, 1.39 mmol, 88% yield) as a yellow solid. ¹H NMR (400 MHZ, CDCl₃) δ=8.23 (d, J=8.4 Hz, 1H), 7.81 (d, J=7.2 Hz, 1H), 7.15-6.85 (m, 2H), 3.15 (s, 3H).

Step 2: To a solution of 2-(difluoromethyl)-N-methyl-6-nitroaniline (140 mg, 693 μmol, 1.00 eq.) in acetic acid (1.50 mL) at 25° C. was slowly added bromine (43.0 μL, 831 μmol, 1.20 eq.). Then reaction mixture was stirred at 25° C. for 2 hours before being quenched by addition of conc. aq.sodium thiosulfate (10.0 mL). The aqueous phase was extracted with ethyl acetate (20.0 mL×3) and the combined organic extracts were dried with sodium sulfate, filtered and concentrated under reduced pressure to give 4-bromo-2-(difluoromethyl)-N-methyl-6-nitroaniline (176 mg, 595 μmol, 86% yield) as a red solid. ¹H NMR (400 MHZ, CDCl₃) δ=8.36 (d, J=2.4 Hz, 1H), 7.88 (d, J=2.4 Hz, 1H), 7.76-7.49 (s br, 1H), 6.96 (t, J=54.4 Hz, 1H), 3.15 (s, 3H).

Step 3: To a solution of 4-bromo-2-(difluoromethyl)-N-methyl-6-nitroaniline (176 mg, 626 μmol, 1.00 eq.) and potassium acetate (92.0 mg, 939 μmol, 1.50 eq.) in dioxane (2.00 mL) were added 4,4,5,5-tetramethyl-2-(4,4,5,5-tetramethyl-1,3,2-dioxaborolan-2-yl)-1,3,2-dioxaborolane (191 mg, 752 μmol, 1.20 eq.) and Pd(dppf)C₁2 (46.0 mg, 63.0 μmol, 0.10 eq.). The mixture was stirred at 90° C. for 12 hours under nitrogen. The reaction mixture was cooled down and filtered, then diluted with ice water (10.0 mL) and extracted with ethyl acetate (10.0 mL×3). The combined organic layers were washed with brine (10.0 mL×2), dried over sodium sulfate, filtered and concentrated under reduced pressure. The residue was purified by prep-TLC (SiO2, petroleum ether/ethyl acetate 10:1) to give 2-(difluoromethyl)-N-methyl-6-nitro-4-(4,4,5,5-tetramethyl-1,3,2-dioxaborolan-2-yl) aniline, Intermediate K (180 mg, 549 μmol, 88% yield) as a yellow oil. ¹H NMR (400 MHZ, CDCl₃) δ=8.64 (s, 1H), 8.11 (s, 1H), 6.96 (t, J=54.4 Hz, 1H), 3.19 (s, 3H), 1.35 (s, 12H).

### INTERMEDIATE L

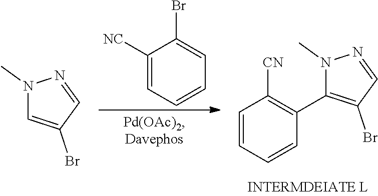

A solution of 4-bromo-1-methyl-pyrazole (500 mg, 3.11 mmol, 1.00 eq.) and 2-bromobenzonitrile (565 mg, 3.11 mmol, 1.00 eq.) in N-methylpyrrolidone (7 mL) was degassed with nitrogen. Then, palladium acetate (7.0 mg, 31.1 μmol, 0.01 eq.) and 2-(2-dicyclohexylphosphanylphenyl)-N,N-dimethyl-aniline (DavePhos) (24.0 mg, 62.1 μmol, 0.02 eq.) was added. To the resulting dark brown solution, tetrabutylammonium acetate (1.87 g, 6.21 mmol, 2 mL, 2.00 eq.) and pivalic acid (82.1 mg, 932 μmol, 86.4 μL, 0.30 eq.) were added and the resulting solution stirred at 100° C. for 10 hours. After the reaction was completed, the mixture was cooled. Ethyl acetate (100 mL) was added and the resulting mixture was washed with brine (3×100 mL). The organic phase was dried over anhydrous sodium sulfate and concentrated in vacuum to give a crude oil. The crude oil was purified by silica gel chromatography (ethyl acetate in petroleum ether, 30%) to give 2-(4-bromo-2-methyl-pyrazol-3-yl)benzonitrile, Intermediate L (400 mg, 1.53 mmol, 49% yield) as a white solid. LCMS [M+1]=261.9. ¹H NMR (400 MHZ, CDCl₃-d) δ=7.86 (dd, J=0.8, 7.6 Hz, 1H), 7.76 (dt, J=1.2, 7.6 Hz, 1H), 7.63 (dt, J=1.2, 7.6 Hz, 1H), 7.60 (s, 1H), 7.49 (dd, J=0.8, 7.6 Hz, 1H), 3.81 (s, 3H).

### INTERMEDIATE M

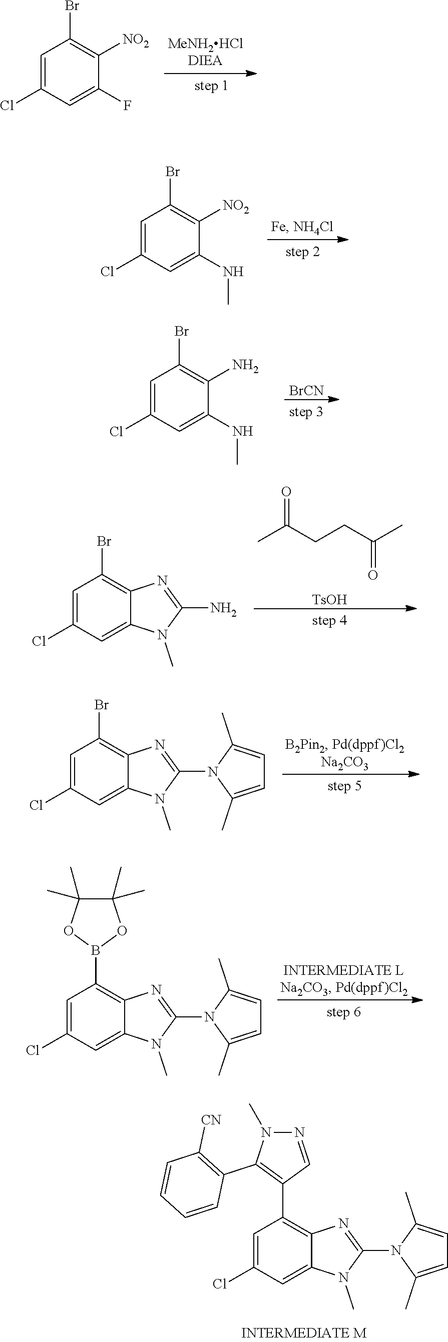

Step 1: To a solution of 1-bromo-5-chloro-3-fluoro-2-nitro-benzene (2.30 g, 9.04 mmol, 1.00 eq.) and methylamine hydrochloride (671 mg, 9.94 mmol, 1.10 eq.) in N,N-dimethylformamide (45.0 mL) was added diisopropylethylamine (3.50 g, 27.1 mmol, 4.72 mL, 3.00 eq.) and the resulting mixture was stirred at 25° C. for 12 hours. The mixture was diluted with water (200 mL) and extracted with ethyl acetate (50.0 mL×3). The combined organic layers were washed with brine, dried over anhydrous sodium sulfate, filtered and concentrated. The residue was purified by silica gel chromatography (ethyl acetate in petroleum ether 5-10%) to afford 3-bromo-5-chloro-N-methyl-2-nitro-aniline (1.10 g, 3.84 mmol, 42% yield) as a yellow solid. LCMS [ESI, M+1]⁺: 265.0/267.0. ¹H NMR (400 MHZ, CDCl₃) δ-6.97 (d, J=2.0 Hz, 1H), 6.73 (d, J=2.0 Hz, 1H), 6.20-5.98 (m, 1H), 2.93 (s, 3H).

Step 2: A mixture of 3-bromo-5-chloro-N-methyl-2-nitro-aniline (1.10 g, 4.14 mmol, 1.00 eq.), iron powder (1.16 g, 20.7 mmol, 5.00 eq.) and saturated ammonium chloride (1.11 g, 20.7 mmol, 5.00 eq.) in ethanol (10.0 mL) and water (2.00 mL) was stirred at 80° C. for 1 hour. The mixture was filtered and the filtrate was concentrated. The residue was purified by fish silica gel chromatography (ethyl acetate in petroleum ether 10-20%) to afford 3-bromo-5-chloro-N1-methyl-benzene-1,2-diamine (460 mg, 1.89 mmol, 46% yield) as a yellow solid. LCMS [ESI, M+1]⁻: 237.0/235.0. ¹H NMR (400 MHZ, CDCl₃) δ=7.03 (br s, 1H), 6.70 (d, J=2.0 Hz, 1H), 2.89 (br s, 3H).

Step 3: To a mixture of 3-bromo-5-chloro-N1-methyl-benzene-1,2-diamine (570 mg, 2.42 mmol, 1.00 eq.) in ethanol (6.00 mL) and water (6.00 mL) was added cyanogen bromide (513 mg, 4.84 mmol, 356 μL, 2.00 eq.). The resulting mixture was stirred at 50° C. for 12 hours. The mixture was adjusted to pH 8-9 with sat. sodium bicarbonate and extracted with ethyl acetate (30.0 mL×5). The combined organic layers were washed with brine, dried over anhydrous sodium sulfate, filtered and concentrated to afford 4-bromo-6-chloro-1-methyl-benzimidazol-2-amine (400 mg, 1.53 mmol, 63% yield) as an off-white solid. LCMS [ESI, M+1]⁻: 262.0/260.0. ¹H NMR (400 MHZ, DMSO-d₆) δ=7.29 (d, J=2.0 Hz, 1H), 7.17 (d, J=2.0 Hz, 1H), 6.86 (s, 2H), 3.49 (s, 3H).

Step 4: A mixture of 4-bromo-6-chloro-1-methyl-benzimidazol-2-amine (400 mg, 1.54 mmol, 1.00 eq.), hexane-2,5-dione (876 mg, 7.68 mmol, 901 μL, 5.00 eq.) and 4-methylbenzenesulfonic acid hydrate (29.2 mg, 154 μmol, 0.10 eq.) in toluene (10.0 mL) in N,N-dimethylformamide (2.00 mL) was stirred at 145° C. for 24 hours with a Dean-Stark trap. The mixture was concentrated to remove toluene and the residue was diluted with water (30.0 mL). The mixture was extracted with ethyl acetate (15.0 mL×3). The combined organic layers were washed with brine, dried over anhydrous sodium sulfate, filtered and concentrated. The residue was purified by silica gel chromatography (ethyl acetate in petroleum ether 20%) to afford 4-bromo-6-chloro-2-(2,5-dimethylpyrrol-1-yl)-1-methyl-benzimidazole (400 mg, 1.18 mmol, 77% yield) as a white solid. LCMS [ESI, M+1]⁻: 338.0/340.0. ¹H NMR (400 MHZ, CDCl₃) δ=7.57 (d, J=1.6 Hz, 1H), 7.37 (d, J=1.6 Hz, 1H), 5.94 (s, 2H), 3.46 (s, 3H), 2.05 (s, 6H).

Step 5: To a mixture of 4-bromo-6-chloro-2-(2,5-dimethylpyrrol-1-yl)-1-methyl-benzimidazole (100 mg, 295 μmol, 1.00 eq.) and 4,4,5,5-tetramethyl-2-(4,4,5,5-tetramethyl-1,3,2-dioxaborolan-2-yl)-1,3,2-dioxaborolane (75.0 mg, 295 μmol, 1.00 eq.) in dioxane (3.00 mL) was added [1,1′-bis(diphenylphosphino) ferrocene]dichloropalladium (II) (21.6 mg, 29.5 μmol, 0.10 eq.) and potassium acetate (72.5 mg, 738 μmol, 2.50 eq.). The mixture was degassed stirred at 80° C. for 12 hours under nitrogen atmosphere. The mixture was diluted with water (15.0 mL) and extracted with ethyl acetate (10.0 mL×3). The combined organic layers were washed with brine, dried over anhydrous sodium sulfate, filtered and concentrated. The residue was purified by silica gel chromatography (ethyl acetate in petroleum ether 20-30%) to afford 6-chloro-2-(2,5-dimethylpyrrol-1-yl)-1-methyl-4-(4,4,5,5-tetramethyl-1,3,2-dioxaborolan-2-yl)benzimidazole (108 mg, 264 μmol, 89% yield) as an off-white solid. LCMS [ESI, M-82]+: 304.1. ¹H NMR (400 MHZ, DMSO-d₆) δ=7.97-7.95 (m, 1H), 7.54 (d, J=2.0 Hz, 1H), 5.95 (s, 2H), 3.47 (s, 3H), 1.95 (s, 6H), 1.32 (s, 12H).

Step 6: To a mixture of 6-chloro-2-(2,5-dimethylpyrrol-1-yl)-1-methyl-4-(4,4,5,5-tetramethyl-1,3,2-dioxaborolan-2-yl)benzimidazole (108 mg, 280 μmol, 1.00 eq.) and 2-(4-iodo-2-methyl-pyrazol-3-yl)benzonitrile (86.6 mg, 280 μmol, 1.00 eq.) in dioxane (2.00 mL) and water (0.40 mL) was added [1,1′-bis(diphenylphosphino) ferrocene]dichloropalladium (II) (20.5 mg, 28.0 μmol, 0.10 eq.) and sodium carbonate (59.4 mg, 560 μmol, 2.00 eq.). The mixture was degassed with nitrogen and stirred at 80° C. for 12 hours under nitrogen atmosphere. The mixture was filtered and the filtrate was concentrated. The residue was purified by silica gel chromatography (ethyl acetate in petroleum ether 20-30%) to afford 2-[4-[6-chloro-2-(2,5-dimethylpyrrol-1-yl)-1-methyl-benzimidazol-4-yl]-2-methyl-pyrazol-3-yl]benzonitrile, Intermediate M (50.0 mg, 108 μmol, 39% yield) as an off-white solid. LCMS [ESI, M+1]⁺: 441.2.

### INTERMEDIATE N

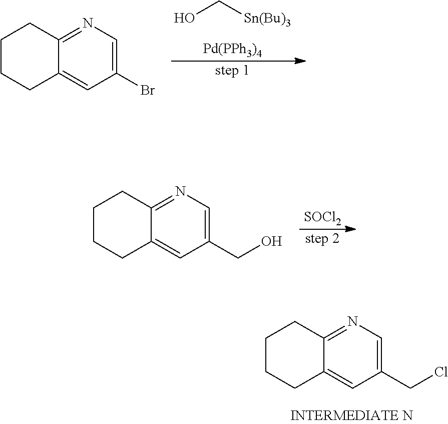

Step 1: A mixture of 3-bromo-5,6,7,8-tetrahydroquinoline (140 mg, 660 μmol, 1.00 eq.), tributylstannylmethanol (424 mg, 1.32 mmol, 2.00 eq.) and Pd (PPh3)+ (76.3 mg, 66.0 μmol, 0.10 eq.) in dioxane (2.00 mL) was degassed and stirred at 100° C. for 16 hours under nitrogen atmosphere. The reaction mixture was concentrated under reduced pressure to give a residue. The residue was purified by prep-TLC (SiO₂, Petroleum ether/Ethyl acetate 1:100) to give 5,6,7,8-tetrahydroquinolin-3-ylmethanol (65.0 mg, 339 μmol, 51% yield) as a colorless oil.

¹H NMR (400 MHZ, CDCl₃) δ=8.10 (d, J=1.6 Hz, 1H), 7.32 (s, 1H), 4.53 (s br, 3H), 2.78-2.74 (m, 2H), 2.65 (t, J=6.4 Hz, 2H), 1.79-1.73 (m, 2H), 1.66 (br s, 2H).

Step 2: To a solution of 5,6,7,8-tetrahydroquinolin-3-ylmethanol (40.0 mg, 246 μmol, 1.00 eq.) in dichloromethane (0.50 mL) was added thionyl chloride (58.3 mg, 490 μmol, 35.6 μL, 2.00 eq.) and dimethylformamide (0.05 mL). The mixture was stirred at 0° C. for 2 hours followed by stirring at 20° C. for 1 hour. The reaction mixture was concentrated under reduced pressure to give 3-(chloromethyl)-5,6,7,8-tetrahydroquinoline, Intermediate N (30.0 mg, 165 μmol, 67% yield) as a yellow oil. LC-MS [M+1]⁺=182.2.

### INTERMEDIATE O

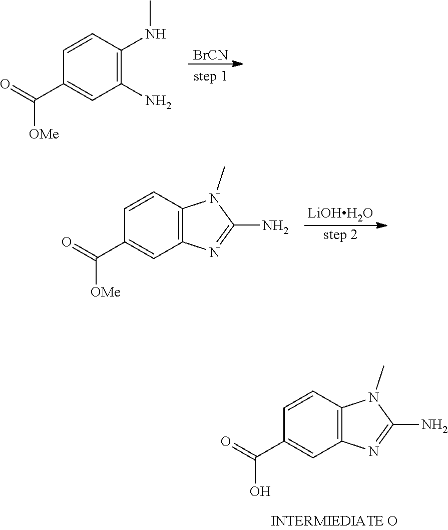

Step 1: To a solution of methyl 3-amino-4-(methylamino)benzoate (500 mg, 2.77 mmol, 1 eq) in MeOH (10 mL) was slowly added BrCN (588 mg, 5.55 mmol, 408 μL, 2.00 eq.). The mixture was stirred at 20° C. for 12 hours. Then the reaction mixture concentrated under reduced pressure to give a residue. The residue was triturated with ethyl acetate (10 mL). The solid was collected through filtration and dried in vacuum to produce methyl 2-amino-1-methyl-1H-benzo[d]imidazole-5-carboxylate (870 mg, crude) as a brown solid. LCMS [M+1]+=206.0. ¹H NMR (400 MHZ, DMSO-d₆) δ=12.74 (br s, 1H), 8.91 (s, 2H), 7.93-7.85 (m, 2H), 7.61 (d, J=8.4 Hz, 1H), 3.86 (s, 3H), 3.66 (s, 3H).

Step 2: To a solution of methyl 2-amino-1-methyl-1H-benzo[d]imidazole-5-carboxylate (400 mg, 1.95 mmol, 1 eq) in MeOH (8 mL) and H₂O (2 mL) was added LiOH·H₂O (409 mg, 9.75 mmol, 5.00 eq). The mixture was stirred at 70° C. for 12 hours. The reaction mixture was concentrated under reduced pressure to give a residue. The residue was diluted with was water (10 mL) and pH was adjusted to 3 with 1 N HCl aqueous solution, the precipitate was collected through filtration and dried in vacuum to produce 2-amino-1-methyl-1H-benzo[d]imidazole-5-carboxylic acid, Intermediate O (150 mg, 785 μmol, 40.3% yield) as a red solid. LCMS [M+1]⁺=192.0. ¹H NMR (400 MHZ, DMSO-d₆) δ-7.72 (s, 1H), 7.62 (dd, J=1.2, 8.4 Hz, 1H), 7.23 (d, J=8.0 Hz, 1H), 6.87 (br s, 2H), 3.55 (s, 3H).

### INTERMEDIATES P-1 and P-2

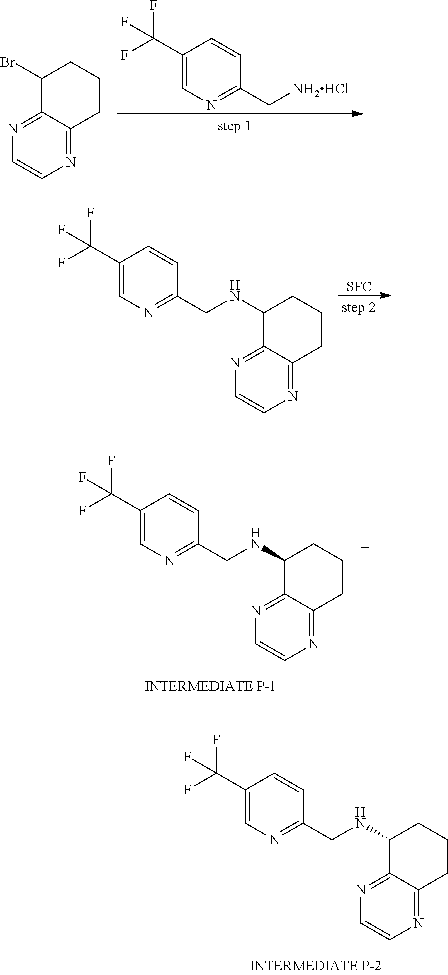

Step 1: A mixture of 5-bromo-5,6,7,8-tetrahydroquinoxaline (625 mg, 1.00 eq.), [5-(trifluoromethyl)-2-pyridyl]methanamine hydrochloride (748 mg, 3.00 mmol, 1.02 eq.) and potassium carbonate (1.62 g, 11.7 mmol, 4.00 eq.) in dimethyl formamide (15.0 mL) was degassed and stirred at 40° C. for 2 hours under nitrogen atmosphere. The mixture was extracted with ethyl acetate (30.0 mL×3), washed with brine, dried over sodium sulfate, filtered and concentrated under reduced pressure to give a residue. The residue was purified by prep-HPLC (neutral condition) give to N-[[5-(trifluoromethyl)-2-pyridyl]methyl]-5,6,7,8-tetrahydroquinoxalin-5-amine (150 mg, 48.0% yield) as a yellow oil. LCMS (ESI, M+1): m/z=309.0. ¹H NMR (400 MHZ, CDCl₃) δ=8.85 (s, 1H), 8.39 (s, 2H), 7.90 (dd, J=2.0, 8.4 Hz, 1H), 7.57 (d, J=8.4 Hz, 1H), 4.19 (s, 2H), 3.96 (br t, J=6.4 Hz, 1H), 3.04-2.97 (m, 2H), 2.26-2.11 (m, 2H), 1.90-1.81 (m, 2H)

Step 2 Racemic N-[[5-(trifluoromethyl)-2-pyridyl]methyl]-5,6,7,8-tetrahydroquinoxalin-5-amine (150 mg) was separated by SFC (condition: column: DAICEL CHIRALPAK IC (250 mm*30 mm, 5 μm); mobile phase: [ACN/IPA (0.1% NH₃H₂O)]; B %: 20%-20%, 6 min) to give peak 1 (5S)—N-[[5-(trifluoromethyl)-2-pyridyl]methyl]-5,6,7,8-tetrahydroquinoxalin-5-amine, Intermediate P-1 (50.0 g, 28.2% yield, 84.6% purity) as a white solid. LCMS (ESI, M+1): m/z=309.0. ¹H NMR (400 MHZ, CDCl;) 8=8.85 (s, 1H), 8.39 (s, 2H), 7.90 (dd, J=1.8, 8.2 Hz, 1H), 7.57 (d, J=8.2 Hz, 1H), 4.19 (s, 2H), 3.99-3.92 (m, 1H), 3.04-2.95 (m, 2H), 2.25-2.12 (m, 2H), 1.93-1.79 (m, 2H) and peak 2 (5R)—N-[[5-(trifluoromethyl)-2-pyridyl]methyl]-5,6,7,8-tetrahydroquinoxalin-5-amine, Intermediate P-2 ((50 mg, 26.7% yield) as a white solid. LCMS (ESI, M+1): m/z=309.0. ¹H NMR (400 MHZ, CDCl₃) δ=8.85 (s, 1H), 8.39 (s, 2H), 7.90 (dd, J=1.7, 8.1 Hz, 1H), 7.58 (d, J=8.3 Hz, 1H), 4.20 (s, 2H), 3.96 (br t, J=6.4 Hz, 1H), 3.06-2.97 (m, 2H), 2.26-2.11 (m, 2H), 1.93-1.80 (m, 2H).

### INTERMEDIATES R-1 and R-2

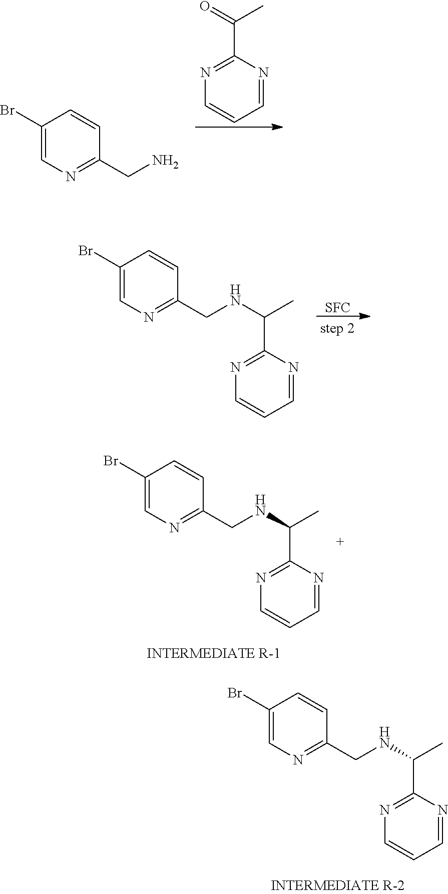

Step 1: To a solution of (5-bromo-2-pyridyl) methanamine (1.60 g, 8.55 mmol, 1.0 equiv) and 1-pyrimidin-2-ylethanone (1.25 g, 10.3 mmol, 1.2 equiv) in dichloromethane (20 mL) was added potassium acetate (1.26 g, 12.8 mmol, 1.5 equiv). The mixture was stirred at 25° C. for 0.5 hour. Then NaBH (OAc) 3 (2.72 g, 12.8 mmol, 1.5 equiv) was added and the mixture was stirred at 25° C. for 1.5 hours. The mixture was diluted with water (50 mL) and the pH was adjusted to ˜4 with HCl (aq., 1.0 M,). The mixture was washed with dichloromethane (50 mL×2). Then the aqueous phase was basified with sodium hydroxide (aq., 10%) to pH 9 and exacted with dichloromethane (100 mL×3). The combined organic layers were washed with brine (30 mL×3), dried over anhydrous sodium sulfate, filtered and concentrated under reduced pressure to give N-[(5-bromo-2-pyridyl)methyl]-1-pyrimidin-2-yl-ethanamine (2.12 g, 7.23 mmol, 84.5% yield) as a yellow oil.

Step 2: Racemic N-[(5-bromo-2-pyridyl)methyl]-1-pyrimidin-2-yl-ethanamine was separated by prep-SFC (column: DAICEL CHIRALPAK AD (250 mm*30 mm, 10 μm);mobile phase: [0.1% NH₃H2O ETOH];B %: 40%-40%,4 min) to give peak 1 (S)—N-((5-bromopyridin-2-yl)methyl)-1-(pyrimidin-2-yl) ethan-1-amine, Intermediate R-1, LCMS (ESI, M+1): m/z=293.1 and peak 2 (R)-N-((5-bromopyridin-2-yl)methyl)-1-(pyrimidin-2-yl) ethan-1-amine, Intermediate R-2, LCMS (ESI, M+1): m/z=293.1

### General Coupling Methods for the Preparation of Library Examples 1-1 to 1-84

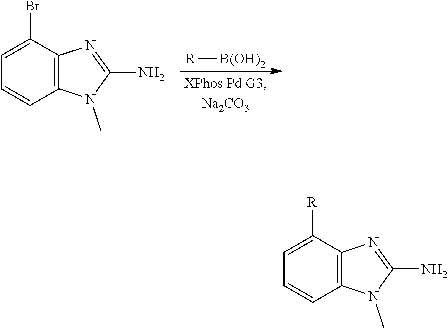

Coupling Method 1-A (CM1-A): To a mixture of boronate or boronic acid (1.5 eq.), 4-bromo-1-methyl-1H-1,3-benzodiazol-2-amine (1 eq.) in dry dioxane (˜0.7 mL per 100 mg of product) was added XPhos Pd G3 (0.05 eq.) (as 0.2M solution in dioxane) and sodium carbonate (2.5 eq.) (as 1M stock solution in Water) under inert atmosphere. The reaction mixture was sealed and heated for 16 hours at 100° C. The mixture was cooled to the ambient temperature and treated with TFA dropwise until pH ˜7. The mixture was evaporated under reduced pressure and the residue was dissolved in the DMSO (˜1 mL per 100 mg of product). DMSO solution was treated with SiliaMetS DMT scavenger, filtered and purified by prep-HPLC (DI water/methanol (+0.05% NH₄OH)) to give a product.

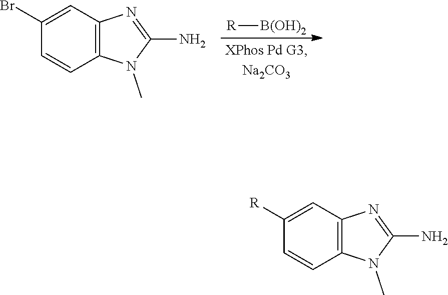

Coupling Method 1-B (CM1-B): To a mixture of boronate or boronic acid (1.5 eq.), 5-bromo-1-methyl-1/-1,3-benzodiazol-2-amine (1 eq.) in dry dioxane (appr. 0.7 mL per 100 mg of product) was added XPhos Pd G3 (0.05 eq.) (as 0.2M solution in dioxane) and sodium carbonate (2.5 eq.) (as 1M stock solution in Water) under inert atmosphere. The reaction mixture was sealed and heated for 16 hours at 100° C. The mixture was cooled to the ambient temperature and treated with TFA dropwise until pH ˜7. The mixture was evaporated under reduced pressure and the residue was dissolved in the DMSO (˜1 mL per 100 mg of product). DMSO solution was treated with SiliaMetS DMT scavenger, filtered and purified by prep-HPLC (DI water/methanol (+0.05% NH₄OH)) to give a product.

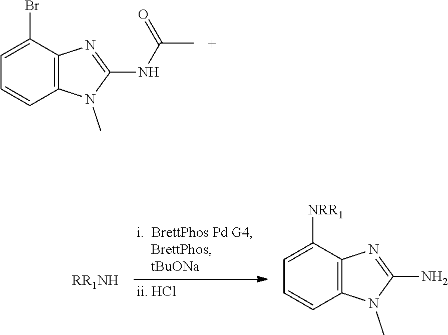

Coupling Method 1-C(CM1-C): To a mixture of Intermediate A (1 eq.) and amine (1.3 eq.) in dry dioxane (appr. 0.5 mL per 100 mg of product) was added BrettPhos Pd G4 (0.1 eq. as a stock solution in dioxane ˜0.05 mL per 100 mg of product), BrettPhos (0.1 eq. as a stock solution in dioxane ˜0.05 mL per 100 mg of product) and t-BuONa (2.5 eq. as a stock solution in THF, ˜0.15 mL per 100 mg of product) under inert atmosphere. The reaction mixture was sealed and heated with shaking for 15 hours at 100° C. The mixture was cooled to the ambient temperature and concentrated to dryness in vacuum The residue was treated with conc. aq. HCl-MeOH mixture (1:6, 4 mL), heated at 50° C. until reaction completion (LCMS control, 16-48 h), cooled to room temperature, evaporated to dryness under reduced pressure The residue was dissolved in the DMSO (˜1 mL per 100 mg of product) and the resulting solution was treated with SiliaMetS DMT scavenger, filtered and purified by prep-HPLC (DI water/methanol (+0.05% NH₄OH)) to give a product.

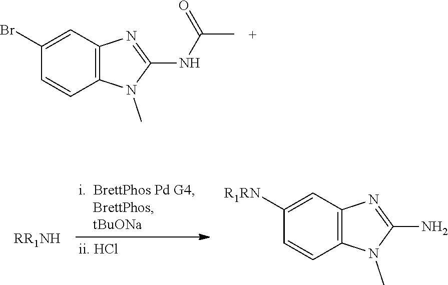

Coupling Method 1-D (CM1-D): To a mixture of Intermediate B (1 eq.) and amine (1.3 eq.) in dry dioxane (appr. 0.5 mL per 100 mg of product) was added BrettPhos Pd G4 (0.1 eq. as a stock solution in dioxane ˜0.05 mL per 100 mg of product), BrettPhos (0.1 eq. as a stock solution in dioxane ˜0.05 mL per 100 mg of product) and t-BuONa (2.5 eq. as a stock solution in THF, ˜0.15 mL per 100 mg of product) under inert atmosphere. The reaction mixture was sealed and heated with shaking for 15 hours at 100° C. The mixture was cooled to the ambient temperature and concentrated to dryness in vacuum. The residue was treated with conc aq. HCl-MeOH mixture (1:6, 4 mL), heated at 50° C. until reaction completion (LCMS control, 16-48 h), cooled to room temperature, evaporated to dryness under reduced pressure. The residue was dissolved in the DMSO (˜1 mL per 100 mg of product) and the resulting solution was treated with SiliaMetS DMT scavenger, filtered and purified by prep-HPLC (DI water/methanol (+0.05% NH₄OH)) to give a product.

Following the teachings of the above-provided reaction schemes, the coupling methods CM1-A, CM1-B, CM1-C and CM1-D and the Intermediates disclosed herein, Examples 1-1 to 1-84 were prepared as shown in Table 1, or using similar methods:

TABLE 1

| | | Coupling | Yield, | Compound Name and |
|---|---|---|---|---|
| Example | Structure | Method | % | Characterization |
| 1-1 | 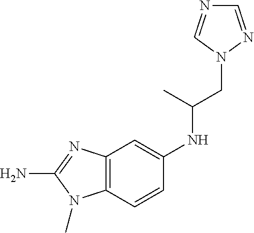 [SMILES: CC(Cn1cncn1)Nc1ccc2c(c1)nc(N)n2C] | CM1-D | 5 | rac-N5-(1-(1H-1,2,4-triazol- 1-yl)propan-2-yl)-1-methyl- 1H-benzo[d]imidazole-2,5- diamine. LCMS [M + 1]⁺ = 272.2 |
| 1-2 | 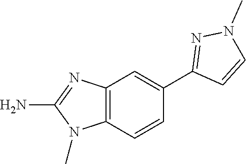 [SMILES: Cn1ccc(-c2ccc3c(c2)nc(N)n3C)n1] | CM1-B | 59 | 1-methyl-5-(1-methyl-1H- pyrazol-3-yl)-1H- benzo[d]imidazol-2-amine. LCMS [M + 1]⁺ = 228.1 ¹H NMR (400 MHz, DMSO-d₆) δ 8.98 (s, 2H), 7.74 (d, J = 1.5 Hz, 1H), 7.66 (dd, J = 8.4, 1.6 Hz, 1H), 7.57 (d, J = 2.2 Hz, 1H), 7.39 (d, J = 8.3 Hz, 1H), 6.59 (d, J = 2.3 Hz, 1H), 3.92 (s, 3H), 3.67 (s, 3H). |
| 1-3 | 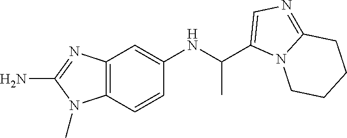 [SMILES: CC(Nc1ccc2c(c1)nc(N)n2C)c1cnc2n1CCCC2] | CM1-D | 2 | rac-1-methyl-N5-(1-(5,6,7,8- tetrahydroimidazo[1,2- a]pyridin-3-yl)ethyl)-1H- benzo[d]imidazole-2,5- diamine. LCMS [M + 1]⁺ = 311.2 |
| 1-4 | 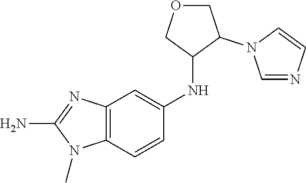 [SMILES: Cn1c(N)nc2cc(NC3COCC3n3ccnc3)ccc21] | CM1-D | 4 | rac-N5-((3R,4S)-4-(1H- imidazol-1- yl)tetrahydrofuran-3-yl)-1- methyl-1H- benzo[d]imidazole-2,5- diamine. LCMS [M + 1]⁺ = 299.2 |
| 1-4a | 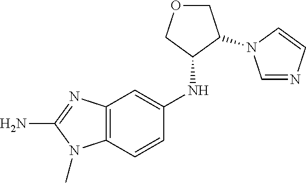 | | | N5-((3S,4R)-4-(1H- imidazol-1- yl)tetrahydrofuran-3-yl)-1- methyl-1H- benzo[d]imidazole-2,5- diamine |
| 1-4b | 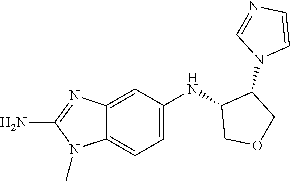 [SMILES: Cn1c(N)nc2cc(N[C@H]3COC[C@H]3n3ccnc3)ccc21] | | | N5-((3R,4S)-4-(1H- imidazol-1- yl)tetrahydrofuran-3-yl)-1- methyl-1H- benzo[d]imidazole-2,5- diamine |
| 1-5 | 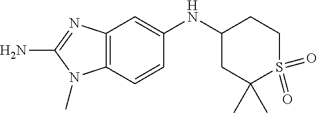 [SMILES: Cn1c(N)nc2cc(NC3CCS(=O)(=O)C(C)(C)C3)ccc21] | CM1-D | 2 | rac-4-((2-amino-1-methyl- 1H-benzo[d]imidazol-5- yl)amino)-2,2- dimethyltetrahydro-2H- thiopyran 1,1-dioxide. LCMS [M + 1]⁺ = 323.3 |
| 1-6 | 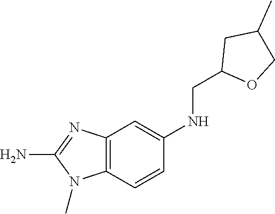 [SMILES: CC1COC(CNc2ccc3c(c2)nc(N)n3C)C1] | CM1-D | 13 | 1-methyl-N5-((4- methyltetrahydrofuran-2- yl)methyl)-1H- benzo[d]imidazole-2,5- diamine. LCMS [M + 1]⁺ = 261.2 |
| 1-7 | 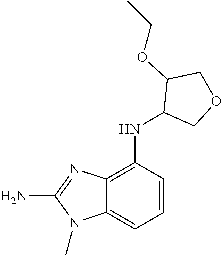 [SMILES: CCOC1COCC1Nc1cccc2c1nc(N)n2C] | CM1-C | 14 | rac-N4-((3R,4S)-4- ethoxytetrahydrofuran-3-yl)- 1-methyl-1H- benzo[d]imidazole-2,4- diamine. LCMS [M + 1]⁺ = 277.2 |
| 1-8 | 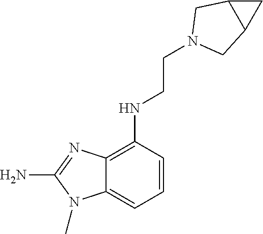 [SMILES: Cn1c(N)nc2c(NCCN3CC4CC4C3)cccc21] | CM1-C | 14 | N4-(2-(3- azabicyclo[3.1.0]hexan-3- yl)ethyl)-1-methyl-1H- benzo[d]imidazole-2,4- diamine LCMS [M + 1]⁺ = 272.2 |
| 1-9 | 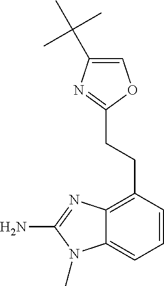 [SMILES: Cn1c(N)nc2c(CCc3nc(C(C)(C)C)co3)cccc21] | CM1-C | 6 | N4-((4-(tert-butyl)oxazol-2- yl)methyl)-1-methyl-1H- benzo[d]imidazole-2,4- diamine. LCMS [M + 1]⁺ = 300.2 |
| 1-10 | 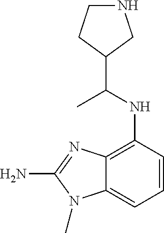 | CM1-C | 2 | rac-1-methyl-N4-(1- (pyrrolidin-3-yl)ethyl)-1H- benzo[d]imidazole-2,4- diamine LCMS [M + 1]⁺ = 206.2 |
| 1-11 | 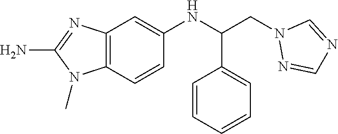 [SMILES: Cn1c(N)nc2cc(NC(Cn3cncn3)c3ccccc3)ccc21] | CM1-D | 1 | rac-(R)-1-methyl-N5-(1- phenyl-2-(1H-1,2,4-triazol- 1-yl)ethyl)-1H- benzo[d]imidazole-2,5- diamine LCMS [M + 1]⁺ = 334.2 |
| 1-12 | 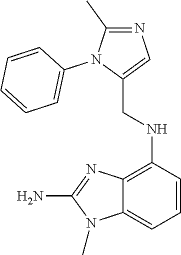 [SMILES: Cc1ncc(CNc2cccc3c2nc(N)n3C)n1-c1ccccc1] | CM1-C | 14 | 1-methyl-N4-((2-methyl-1- phenyl-1H-imidazol-5- yl)methyl)-1H- benzo[d]imidazole-2,4- diamine LCMS [M + 1]⁺ = 333.2 |
| 1-13 | 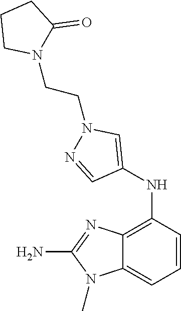 [SMILES: Cn1c(N)nc2c(Nc3cnn(CCN4CCCC4=O)c3)cccc21] | CM1-C | 11 | 1-(2-(4-((2-amino-1-methyl- 1H-benzo[d]imidazol-4- yl)amino)-1H-pyrazol-1- yl)ethyl)pyrrolidin-2-one. LCMS [M + 1]⁺ = 340.2 |
| 1-14 | 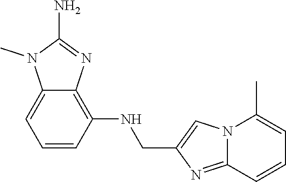 [SMILES: Cc1cccc2nc(CNc3cccc4c3nc(N)n4C)cn12] | CM1-C | 8 | 1-methyl-N4-((5- methylimidazo[1,2- a]pyridin-2-yl)methyl)-1H- benzo[d]imidazole-2,4- diamine LCMS [M + 1]⁺ = 307.2 |
| 1-15 | 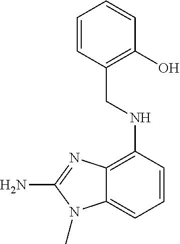 [SMILES: Cn1c(N)nc2c(NCc3ccccc3O)cccc21] | CM1-C | 7 | 2-(((2-amino-1-methyl-1H- benzo[d]imidazol-4- yl)amino)methyl)phenol. LCMS [M + 1]⁺ = 269.2 |
| 1-16 | 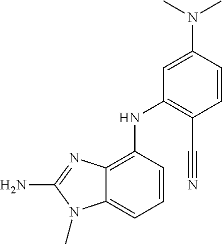 [SMILES: CN(C)c1ccc(C#N)c(Nc2cccc3c2nc(N)n3C)c1] | CM1-C | 11 | 2-((2-amino-1-methyl-1H- benzo[d]imidazol-4- yl)amino)-4- (dimethylamino)benzonitrile. LCMS [M + 1]⁺ = 307.4 |
| 1-17 | 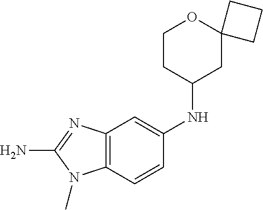 [SMILES: Cn1c(N)nc2cc(NC3CCOC4(CCC4)C3)ccc21] | CM1-D | 9 | rac-1-methyl-N5-(5- oxaspiro[3.5]nonan-8-yl)- 1H-benzo[d]imidazole-2,5- diamine. LCMS [M + 1]⁺ = 287.2 |
| 1-18 | 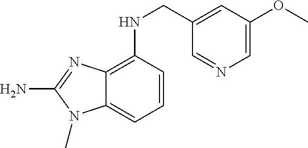 [SMILES: COc1cncc(CNc2cccc3c2nc(N)n3C)c1] | CM1-C | 4 | N4-((5-methoxypyridin-3- yl)methyl)-1-methyl-1H- benzo[d]imidazole-2,4- diamine. LCMS [M + 1]⁺ = 284.1 |
| 1-19 | 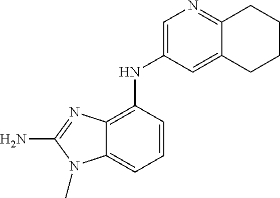 [SMILES: Cn1c(N)nc2c(Nc3cnc4c(c3)CCCC4)cccc21] | CM1-C | 46 | 1-methyl-N4-(5,6,7,8- tetrahydroquinolin-3-yl)-1H- benzo[d]imidazole-2,4- diamine. LCMS [M + 1]⁺ = 294.2 |
| 1-20 | 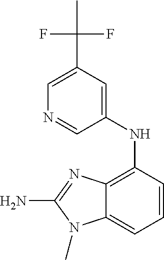 [SMILES: Cn1c(N)nc2c(Nc3cncc(C(C)(F)F)c3)cccc21] | CM1-C | 21 | N4-(5-(1,1- difluoroethyl)pyridin-3-yl)- 1-methyl-1H- benzo[d]imidazole-2,4- diamine. LCMS [M + 1]⁺ = 304.2 |
| 1-21 | 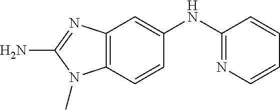 [SMILES: Cn1c(N)nc2cc(Nc3ccccn3)ccc21] | CM1-D | 29 | 1-methyl-N5-(pyridin-2-yl)- 1H-benzo[d]imidazole-2,5- diamine. LCMS [M + 1]⁺ = 240.2 |
| 1-22 | 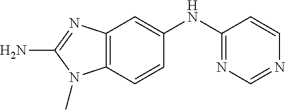 [SMILES: Cn1c(N)nc2cc(Nc3ccncn3)ccc21] | CM1-D | 18 | 1-methyl-N5-(pyrimidin-4- yl)-1H-benzo[d]imidazole- 2,5-diamine. LCMS [M + 1]⁺ = 241.2 |
| 1-23 | 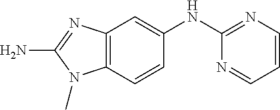 [SMILES: Cn1c(N)nc2cc(Nc3ncccn3)ccc21] | CM1-D | 19 | 1-methyl-N5-(pyrimidin-2- yl)-1H-benzo[d]imidazole- 2,5-diamine. LCMS [M + 1]⁺ = 241.2 |
| 1-24 | 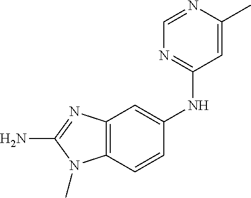 [SMILES: Cc1cc(Nc2ccc3c(c2)nc(N)n3C)ncn1] | CM1-D | 2 | 1-methyl-N5-(6- methylpyrimidin-4-yl)-1H- benzo[d]imidazole-2,5- diamine. LCMS [M + 1]⁺ = 255.2 |
| 1-25 | 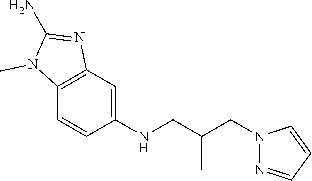 [SMILES: CC(CNc1ccc2c(c1)nc(N)n2C)Cn1cccn1] | CM1-D | 8 | rac-(1-methyl-N5-(2- methyl-3-(1H-pyrazol-1- yl)propyl)-1H- benzo[d]imidazole-2,5- diamine LCMS [M + 1]⁺ = 285.2 |
| 1-26 | 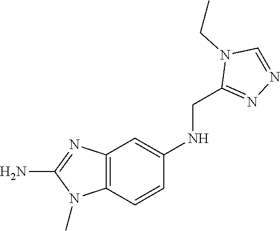 [SMILES: CCn1cnnc1CNc1ccc2c(c1)nc(N)n2C] | CM1-D | 3 | N5-((4-ethyl-4H-1,2,4- triazol-3-yl)methyl)-1- methyl-1H- benzo[d]imidazole-2,5- diamine. LCMS [M + 1]⁺ = 272.2 |
| 1-27 | 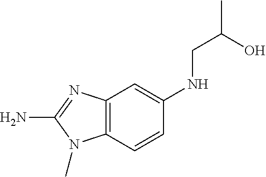 [SMILES: CC(O)CNc1ccc2c(c1)nc(N)n2C] | CM1-D | 19 | rac-1-((2-amino-1-methyl- 1H-benzo[d]imidazol-5- yl)amino)propan-2-ol. LCMS [M + 1]⁺ = 221.2 |
| 1-28 | 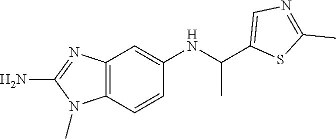 [SMILES: Cc1ncc(C(C)Nc2ccc3c(c2)nc(N)n3C)s1] | CM1-D | 4 | rac-1-methyl-N5-(1-(2- methylthiazol-5-yl)ethyl)- 1H-benzo[d]imidazole-2,5- diamine. LCMS [M + 1]⁺ = 288.2 |
| 1-29 | 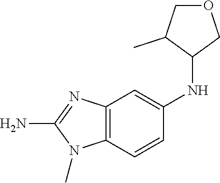 [SMILES: CC1COCC1Nc1ccc2c(c1)nc(N)n2C] | CM1-D | 2 | 1-methyl-N5-(4- methyltetrahydrofuran-3-yl)- 1H-benzo[d]imiazole-2,5- diamine. LCMS [M + 1]⁺ = 247.2 |
| 1-30 | 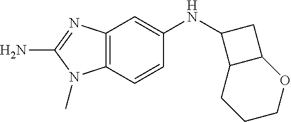 [SMILES: Cn1c(N)nc2cc(NC3CC4OCCCC34)ccc21] | CM1-D | 3 | N5-(2- oxabicyclo[4.2.0]octan-7- yl)-1-methyl-1H- benzo[d]imidazole-2,5- diamine. LCMS [M + 1]⁺ = 273.4 |
| 1-31 | 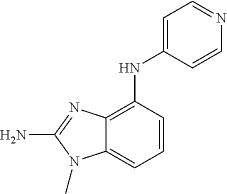 [SMILES: Cn1c(N)nc2c(Nc3ccncc3)cccc21] | CM1-C | 37 | 1-methyl-N4-(pyridin-4-yl)- 1H-benzo[d]imidazole-2,4- diamine. LCMS [M + 1]⁺ = 240.0 |
| 1-32 | 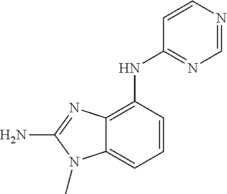 [SMILES: Cn1c(N)nc2c(Nc3ccncn3)cccc21] | CM1-C | 27 | 1-methyl-N4-(pyrimidin-4- yl)-1H-benzo[d]imidazole- 2,4-diamine LCMS [M + 1]⁺ = 241.1 |
| 1-33 | 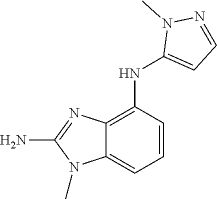 [SMILES: Cn1nccc1Nc1cccc2c1nc(N)n2C] | CM1-C | 27 | 1-methyl-N4-(1-methyl-1H- pyrazol-5-yl)-1H- benzo[d]imidazole-2,4- diamine. LCMS [M + 1]⁺ = 243.2 |
| 1-34 | 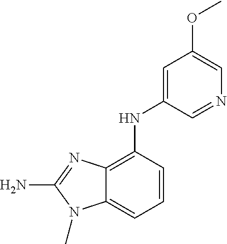 [SMILES: COc1cncc(Nc2cccc3c2nc(N)n3C)c1] | CM1-C | 9 | N4-(5-methoxypyridin-3-yl)- 1-methyl-1H- benzo[d]imidazole-2,4- diamine. LCMS [M + 1]⁺ = 270.1 |
| 1-35 | 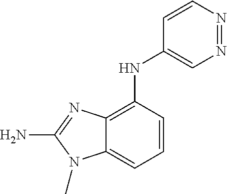 [SMILES: Cn1c(N)nc2c(Nc3ccnnc3)cccc21] | CM1-C | 12 | 1-methyl-N4-(pyridazin-4- yl)-1H-benzo[d]imidazole- 2,4-diamine. LCMS [M + 1]⁺ = 241.0 |
| 1-36 | 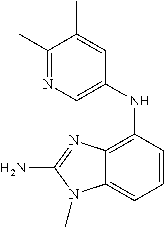 [SMILES: Cc1cc(Nc2cccc3c2nc(N)n3C)cnc1C] | CM1-C | 31 | N4-(5,6-dimethylpyridin-3- yl)-1-methyl-1H- benzo[d]imidazole-2,4- diamine. LCMS [M + 1]⁺ = 268.2. ¹H NMR (600 MHz, DMSO-d₆) δ 8.03 (d, J = 2.7 Hz, 1H), 7.45 (s, 1H), 7.15 (s, 1H), 6.81-6.77 (m, 1H), 6.77- 6.72 (m, 2H), 6.25 (s, 2H), 3.47 (s, 3H), 2.28 (s, 3H), 2.13 (s, 3H). |
| 1-37 | 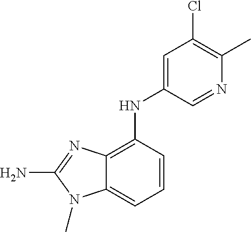 [SMILES: Cc1ncc(Nc2cccc3c2nc(N)n3C)cc1Cl] | CM1-C | 24 | N4-(5-chloro-6- methylpyridin-3-yl)-1- methyl-1H- benzo[d]imidazole-2,4- diamine. LCMS [M + 1]⁺ = 288.2 |
| 1-38 | 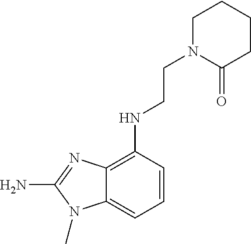 [SMILES: Cn1c(N)nc2c(NCCN3CCCCC3=O)cccc21] | CM1-C | 5 | 1-(2-((2-amino-1-methyl- 1H-benzo[d]imidazol-4- yl)amino)ethyl)piperidin-2- one. LCMS [M + 1]⁺ = 288.1 |
| 1-39 | 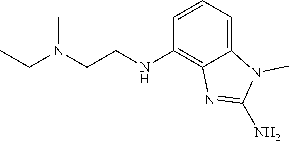 [SMILES: CCN(C)CCNc1cccc2c1nc(N)n2C] | CM1-C | 13 | N4-(2- (ethyl(methyl)amino)ethyl)- 1-methyl-1H- benzo[d]imidazole-2,4- diamine. LCMS [M + 1]⁺ = 248.4 |
| 1-40 | 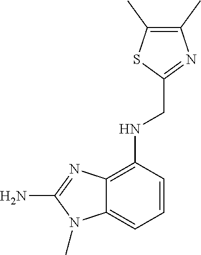 [SMILES: Cc1nc(CNc2cccc3c2nc(N)n3C)sc1C] | CM1-C | 21 | N4-((4,5-dimethylthiazol-2- yl)methyl)-1-methyl-1H- benzo[d]imidazole-2,4- diamine. LCMS [M + 1]⁺ = 288.2 |
| 1-41 | 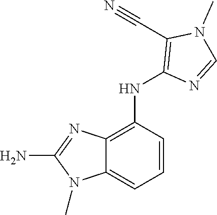 [SMILES: Cn1cnc(Nc2cccc3c2nc(N)n3C)c1C#N] | CM1-C | 8 | 4-((2-amino-1-methyl-1H- benzo[d]imidazol-4- yl)amino)-1-methyl-1H- imidazole-5-carbonitrile. LCMS [M + 1]⁺ = 268.0 |
| 1-42 | 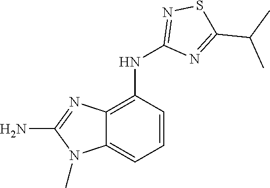 [SMILES: CC(C)c1nc(Nc2cccc3c2nc(N)n3C)ns1] | CM1-C | 26 | N4-(5-isopropyl-1,2,4- thiadiazol-3-yl)-1-methyl- 1H-benzo[d]imidazole-2,4- diamine. LCMS [M + 1]⁺ = 289.0 |
| 1-43 | 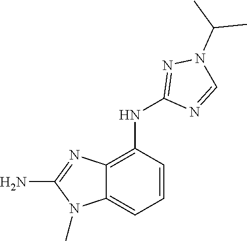 [SMILES: CC(C)n1cnc(Nc2cccc3c2nc(N)n3C)n1] | CM1-C | 8 | N4-(1-isopropyl-1H-1,2,4- triazol-3-yl)-1-methyl-1H- benzo[d]imidazole-2,4- diamine. LCMS [M + 1]⁺ = 272.2 |
| 1-44 | 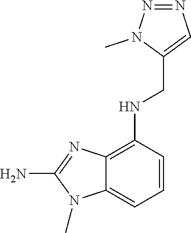 [SMILES: Cn1nncc1CNc1cccc2c1nc(N)n2C] | CM1-C | 7 | 1-methyl-N4-((1-methyl-1H- 1,2,3-triazol-5-yl)methyl)- 1H-benzo[d]imidazole-2,4- diamine. LCMS [M + 1]⁺ = 258.2 |
| 1-45 | 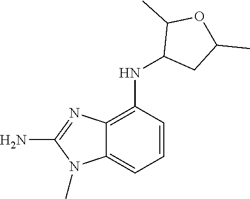 [SMILES: CC1CC(Nc2cccc3c2nc(N)n3C)C(C)O1] | CM1-C | 2 | N4-(2,5- dimethyltetrahydrofuran-3- yl)-1-methyl-1H- benzo[d]imidazole-2,4- diamine. LCMS [M + 1]⁺ = 261.2 |
| 1-46 | 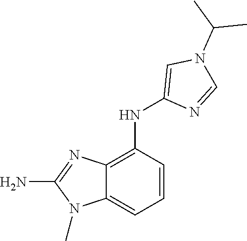 [SMILES: CC(C)n1cnc(Nc2cccc3c2nc(N)n3C)c1] | CM1-C | 8 | N4-(1-isopropyl-1H- imidazol-4-yl)-1-methyl-1H- benzo[d]imidazole-2,4- diamine. LCMS [M + 1]⁺ = 271.2 |
| 1-47 | 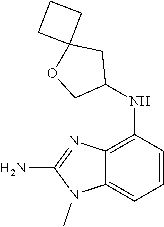 [SMILES: Cn1c(N)nc2c(NC3COC4(CCC4)C3)cccc21] | CM1-C | 5 | rac-(1-methyl-N4-(5- oxaspiro[3.4]octan-7-yl)-1H- benzo[d]imidazole-2,4- diamine. LCMS [M + 1]⁺ = 273.2 |
| 1-48 | 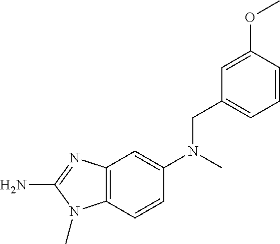 [SMILES: COc1cccc(CN(C)c2ccc3c(c2)nc(N)n3C)c1] | CM1-C | 33 | N5-(3-methoxybenzyl)-N5,1- dimethyl-1H- benzo[d]imidazole-2,5- diamine. LCMS [M + 1]⁺ = 297.1 |
| 1-49 | 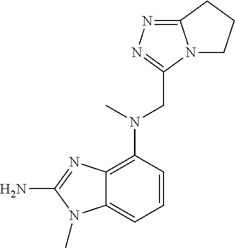 [SMILES: CN(Cc1nnc2n1CCC2)c1cccc2c1nc(N)n2C] | CM1-C | 4 | N4-((6,7-dihydro-5H- pyrrolo[2,1-c][1,2,4]triazol- 3-yl)methyl)-N4,1-dimethyl- 1H-benzo[d]imidazole-2,4- diamine. LCMS [M + 1]⁺ = 298.2 |
| 1-50 | 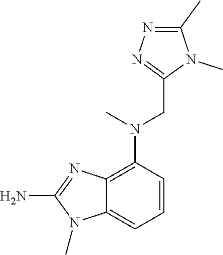 [SMILES: Cc1nnc(CN(C)c2cccc3c2nc(N)n3C)n1C] | CM1-C | 32 | N4-((4,5-dimethyl-4H-1,2,4- triazol-3-yl)methyl)-N4,1- dimethyl-1H- benzo[d]imidazole-2,4- diamine. LCMS [M + 1]⁺ = 286.0 |
| 1-51 | 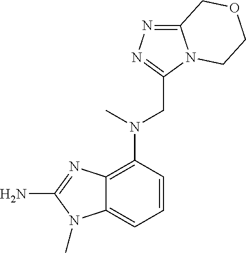 [SMILES: CN(Cc1nnc2n1CCOC2)c1cccc2c1nc(N)n2C] | CM1-C | 14 | N4-((5,6-dihydro-8H- [1,2,4]triazolo[3,4- c][1,4]oxazin-3-yl)methyl)- N4,1-dimethyl-1H- benzo[d]imidazole-2,4- diamine LCMS [M + 1]⁺ = 314.2 |
| 1-52 | 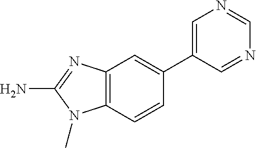 [SMILES: Cn1c(N)nc2cc(-c3cncnc3)ccc21] | CM1-B | 26 | 1-methyl-5-(pyrimidin-5-yl)- 1H-benzo[d]imidazol-2- amine. LCMS [M + 1]⁺ = 226.2. ¹H NMR (400 MHz, DMSO-d₆) δ 9.08 (s, 3H), 7.55 (s, 1H), 7.37-7.20 (m, 2H), 6.49 (s, 2H), 3.54 (s, 3H). |
| 1-53 | 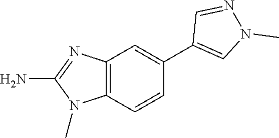 [SMILES: Cn1cc(-c2ccc3c(c2)nc(N)n3C)cn1] | CM1-B | 24 | 1-methyl-5-(1-methyl-1H- pyrazol-4-yl)-1H- benzo[d]imidazol-2-amine. LCMS [M + 1]⁺ = 228.1 |
| 1-54 | 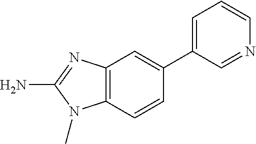 [SMILES: Cn1c(N)nc2cc(-c3cccnc3)ccc21] | CM1-B | 34 | 1-methyl-5-(pyridin-3-yl)- 1H-benzo[d]imidazol-2- amine. LCMS [M + 1]⁺ = 225.0 |
| 1-55 | 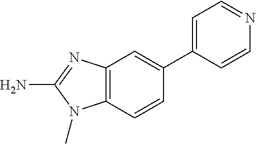 [SMILES: Cn1c(N)nc2cc(-c3ccncc3)ccc21] | CM1-B | 2 | 1-methyl-5-(pyridin-4-yl)- 1H-benzo[d]imidazol-2- amine. LCMS [M + 1]⁺ = 225.2 |
| 1-56 | 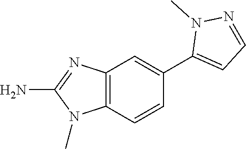 [SMILES: Cn1nccc1-c1ccc2c(c1)nc(N)n2C] | CM1-B | 54 | 1-methyl-5-(1-methyl-1H- pyrazol-5-yl)-1H- benzo[d]imidazol-2-amine. LCMS [M + 1]⁺ = 228.1. ¹H NMR (600 MHz, DMSO-d₆) δ 7.40 (d, J = 1.8 Hz, 1H), 7.21-7.18 (m, 2H), 6.99 (dd, J = 8.0, 1.6 Hz, 1H), 6.49 (s, 2H), 6.27 (d, J = 1.8 Hz, 1H), 3.80 (s, 3H), 3.51 (s, 3H). |
| 1-57 | 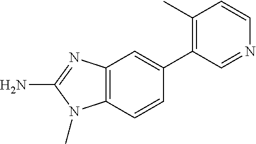 [SMILES: Cc1ccncc1-c1ccc2c(c1)nc(N)n2C] | CM1-B | 42 | 1-methyl-5-(4- methylpyridin-3-yl)-1H- benzo[d]imidazol-2-amine. LCMS [M + 1]⁺ = 239.1. 1H NMR (600 MHz, DMSO- d6) δ 8.39-8.26 (m, 2H), 7.27 (d, J = 5.1 Hz, 1H), 7.18 (d, J = 8.1 Hz, 1H), 7.07 (s, 1H), 6.86 (d, J = 8.1 Hz, 1H), |
| | | | | 6.44 (s, 2H), 3.52 (s, 3H), |
| | | | | 2.25 (s, 3H). |
| 1-58 | 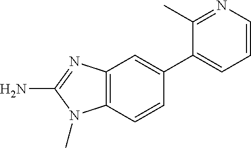 [SMILES: Cc1ncccc1-c1ccc2c(c1)nc(N)n2C] | CM1-B | 32 | 1-methyl-5-(2- methylpyridin-3-yl)-1H- benzo[d]imidazol-2-amine. LCMS [M + 1]⁺ = 239.2 |
| 1-59 | 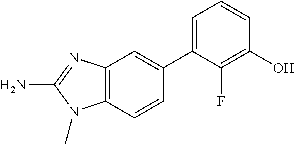 [SMILES: Cn1c(N)nc2cc(-c3cccc(O)c3F)ccc21] | CM1-B | 26 | 3-(2-amino-1-methyl-1H- benzo[d]imidazol-5-yl)-2- fluorophenol. LCMS [M + 1]⁺ = 258.2 |
| 1-60 | 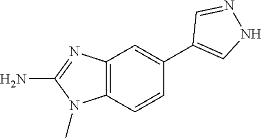 [SMILES: Cn1c(N)nc2cc(-c3cn[nH]c3)ccc21] | CM1-B | 39 | 1-methyl-5-(1H-pyrazol-4- yl)-1H-benzo[d]imidazol-2- amine. LCMS [M + 1]⁺ = 214.0 |
| 1-61 | 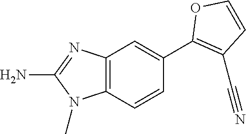 [SMILES: Cn1c(N)nc2cc(-c3occc3C#N)ccc21] | CM-1B | 19 | 2-(2-amino-1-methyl-1H- benzo[d]imidazol-5- yl)furan-3-carbonitrile. LCMS [M + 1]⁺ = 239.2 |
| 1-62 | 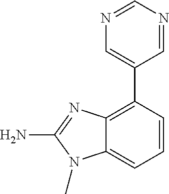 [SMILES: Cn1c(N)nc2c(-c3cncnc3)cccc21] | CM-1A | 14 | 1-methyl-4-(pyrimidin-5-yl)- 1H-benzo[d]imidazol-2- amine. LCMS [M + 1]⁺ = 226.2 |
| 1-63 | 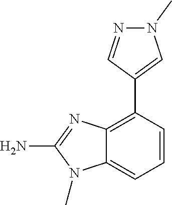 [SMILES: Cn1cc(-c2cccc3c2nc(N)n3C)cn1] | CM-1A | 28 | 1-methyl-4-(1-methyl-1H- pyrazol-4-yl)-1H- benzo[d]imidazol-2-amine. LCMS [M + 1]⁺ = 228.2. ¹H NMR (600 MHz, DMSO- d₆) δ 8.38 (s, 1H), 8.09 (s, 1H), 7.21 (d, J = 7.6 Hz, 1H), 6.94 (d, J = 7.7 Hz, 1H), 6.92- 6.81 (m, 1H), 6.42 (s, 2H), 3.86 (s, 3H), 3.48 (s, 3H). |
| 1-64 | 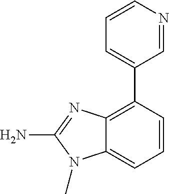 [SMILES: Cn1c(N)nc2c(-c3cccnc3)cccc21] | CM-1A | 46 | 1-methyl-4-(pyridin-3-yl)- 1H-benzo[d]imidazol-2- amine LCMS [M + 1]⁺ = 225.1. ¹H NMR (600 MHz, DMSO-d₆) δ 9.22 (d, J = 2.3 Hz, 1H), 8.46 (dt, J = 4.7, 2.3 Hz, 1H), 8.41 (dt, J = 7.9, 2.0 Hz, 1H), 7.45-7.39 (m, 1H), 7.26 (d, J = 7.7 Hz, 1H), 7.16 (d, J = 7.8 Hz, 1H), 7.07-6.96 (m, 1H), 6.59 (s, 2H), 3.52 (s, 3H). |
| 1-65 | 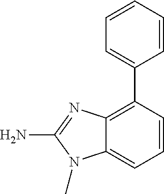 [SMILES: Cn1c(N)nc2c(-c3ccccc3)cccc21] | CM1-A | 50 | 1-methyl-4-phenyl-1H- benzo[d]imidazol-2-amine. LCMS [M + 1]⁺ = 224.2. |
| 1-66 | 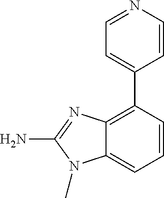 [SMILES: Cn1c(N)nc2c(-c3ccncc3)cccc21] | CM1-A | 46 | 1-methyl-4-(pyridin-4-yl)- 1H-benzo[d]imidazol-2- amine LCMS [M + 1]⁺ = 225.1 |
| 1-67 | 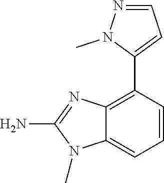 [SMILES: Cn1nccc1-c1cccc2c1nc(N)n2C] | CM1-A | 43 | 1-methyl-4-(1-methyl-1H- pyrazol-5-yl)-1H- benzo[d]imidazol-2-amine LCMS [M + 1]⁺ = 228.2 |
| 1-68 | 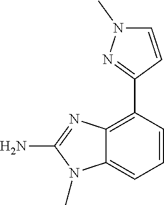 [SMILES: Cn1ccc(-c2cccc3c2nc(N)n3C)n1] | CM1-A | 40 | 1-methyl-4-(1-methyl-1H- pyrazol-3-yl)-1H- benzo[d]imidazol-2-amine LCMS [M + 1]⁺ = 228.1 |
| 1-69 | 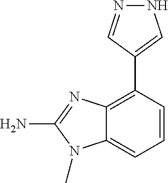 [SMILES: Cn1c(N)nc2c(-c3cn[nH]c3)cccc21] | CM1-A | 41 | 1-methyl-4-(1H-pyrazol-4- yl)-1H-benzo[d]imidazol-2- amine LCMS [M + 1]⁺ = 214.2 |
| 1-70 | 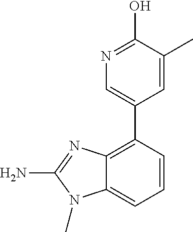 [SMILES: Cc1cc(-c2cccc3c2nc(N)n3C)cnc1O] | CM1-A | 33 | 5-(2-amino-1-methyl-1H- benzo[d]imidazol-4-yl)-3- methylpyridin-2-ol LCMS [M + 1]⁺ = 255.1 |
| 1-71 | 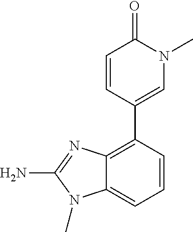 [SMILES: Cn1cc(-c2cccc3c2nc(N)n3C)ccc1=O] | CM1-A | 36 | 5-(2-amino-1-methyl-1H- benzo[d]imidazol-4-yl)-1- methylpyridin-2(1H)-one LCMS [M + 1]⁺ = 255.1 |
| 1-72 | 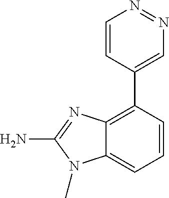 [SMILES: Cn1c(N)nc2c(-c3ccnnc3)cccc21] | CM1-A | 18 | N4-(2-(3- azabicyclo[3.1.0]hexan-3- yl)ethyl)-1-methyl-1H- benzo[d]imidazole-2,4- diamine LCMS [M + 1]⁺ = 226.2 |
| 1-73 | 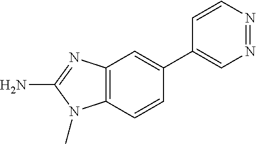 [SMILES: Cn1c(N)nc2cc(-c3ccnnc3)ccc21] | CM1-B | 40 | 1-methyl-5-(pyridazin-4-yl)- 1H-benzo[d]imidazol-2- amine LCMS [M + 1]⁺ = 226.0 |
| 1-74 | 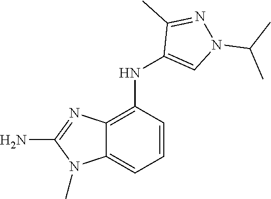 [SMILES: Cc1nn(C(C)C)cc1Nc1cccc2c1nc(N)n2C] | CM1-C | 10 | N4-(1-isopropyl-3-methyl- 1H-pyrazol-4-yl)-1-methyl- 1H-benzo[d]imidazole-2,4- diamine LCMS [M + 1]⁺ = 285.4. ¹H NMR (400 MHz, DMSO- d₆) δ 12.75 (br s, 1H), 8.58 (s, 2H), 7.72 (s, 1H), 7.04 (t, J = 8.1 Hz, 1H), 6.76 (d, J = 7.9 Hz, 1H), 6.35 (d, J = 8.1 Hz, 1H), 4.44-4.34 (m, 1H), 3.58 (s, 3H), 2.03 (s, 3H), 1.40 (d, J = 6.7 Hz, 6H) |
| 1-75 | 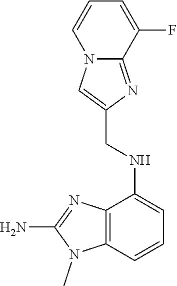 [SMILES: Cn1c(N)nc2c(NCc3cn4cccc(F)c4n3)cccc21] | CM1-C | 11 | N4-((8-fluoroimidazo[1,2- a]pyridin-2-yl)methyl)-1- methyl-1H- benzo[d]imidazole-2,4- diamine LCMS [M + 1]⁺ = 311.2 |
| 1-76 | 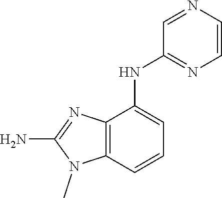 | CM1-C | 21 | 1-methyl-N4-(pyrazin-2-yl)- 1H-benzo[d]imidazole-2,4- diamine LCMS [M + 1]⁺ = 341.2 |
| 1-77 | 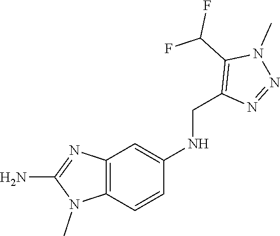 [SMILES: Cn1nnc(CNc2ccc3c(c2)nc(N)n3C)c1C(F)F] | CM1-D | 19 | N5-((5-(difluoromethyl)-1- methyl-1H-1,2,3-triazol-4- yl)methyl)-1-methyl-1H- benzo[d]imidazole-2,5- diamine LCMS [M + 1]⁺ = 308.1 |
| 1-78 | 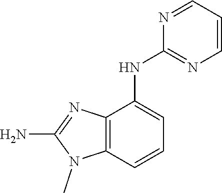 [SMILES: Cn1c(N)nc2c(Nc3ncccn3)cccc21] | CM1-C | 13 | 1-methyl-N4-(pyrimidin-2- yl)-1H-benzo[d]imidazole- 2,4-diamine LCMS [M + 1]⁺ = 241.2 |
| 1-79 | 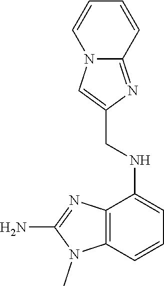 [SMILES: Cn1c(N)nc2c(NCc3cn4ccccc4n3)cccc21] | CM1-C | 7 | N4-(imidazo[1,2-a]pyridin- 2-ylmethyl)-1-methyl-1H- benzo[d]imidazole-2,4- diamine LCMS [M + 1]⁺ = 293.1 |
| 1-80 | 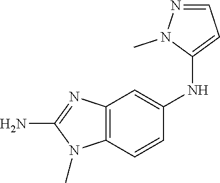 [SMILES: Cn1nccc1Nc1ccc2c(c1)nc(N)n2C] | CM1-D | 17 | 1-methyl-N5-(1-methyl-1H- pyrazol-5-yl)-1H- benzo[d]imidazole-2,5- diamine LCMS [M + 1]⁺ = 243.2. ¹H NMR (400 MHz, DMSO-d₆) δ 12.34 (br s, 1H), 8.53 (s, 2H), 7.42 (d, J = 1.8 Hz, 1H), 7.33 (d, J = 8.6 Hz, 1H), 6.90- 6.87 (m, 1H), 6.82 (dd, J = 2.1, 8.6 Hz, 1H), 5.98 (d, J = 2.0 Hz, 1H), 3.66 (s, 3H), 3.57 (s, 3H) |
| 1-81 | 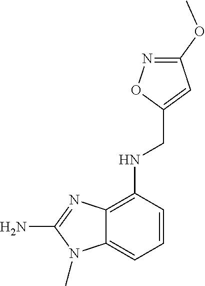 [SMILES: COc1cc(CNc2cccc3c2nc(N)n3C)on1] | CM1-C | 4 | N4-((3-methoxyisoxazol-5- yl)methyl)-1-methyl-1H- benzo[d]imidazole-2,4- diamine LCMS [M + 1]⁺ = 274.2 |
| 1-82 | 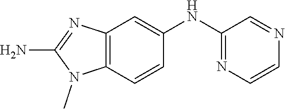 [SMILES: Cn1c(N)nc2cc(Nc3cnccn3)ccc21] | CM1-D | 7 | 1-methyl-N5-(pyrazin-2-yl)- 1H-benzo[d]imidazole-2,5- diamine LCMS [M + H]⁺ = 241.2. H NMR (400 MHz, DMSO- d₆) δ = 8.78 (s, 1H), 8.33 (d, J = 1.4 Hz, 1H), 8.06 (dd, J = |
| | | | | 1.4, 2.7 Hz, 1H), 7.84 (d, J = |
| | | | | 2.7 Hz, 1H), 7.70-7.63 (m, |
| | | | | 1H), 6.86 (d, J = 4.2 Hz, 2H), |
| | | | | 6.32 (s, 2H), 3.32 (s, 3H). |
| 1-83 | 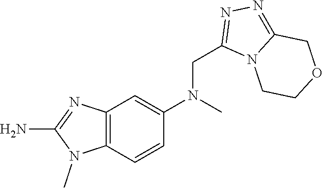 [SMILES: CN(Cc1nnc2n1CCOC2)c1ccc2c(c1)nc(N)n2C] | CM1-D | 5 | N5-((5,6-dihydro-8H- [1,2,4]triazolo[3,4- c][1,4]oxazin-3-yl)methyl)- N5,1-dimethyl-1H- benzo[d]imidazole-2,5- diamine LCMS [M + 1]⁺ = 314.2 |
| 1-84 | 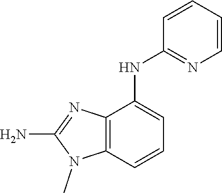 [SMILES: Cn1c(N)nc2c(Nc3ccccn3)cccc21] | CM1-C | 7 | 1-methyl-N4-(pyridin-2-yl)- 1H-benzo[d]imidazole-2,4- diamine LCMS [M + 1]⁺ = 240.2 |

### EXAMPLE 1-85

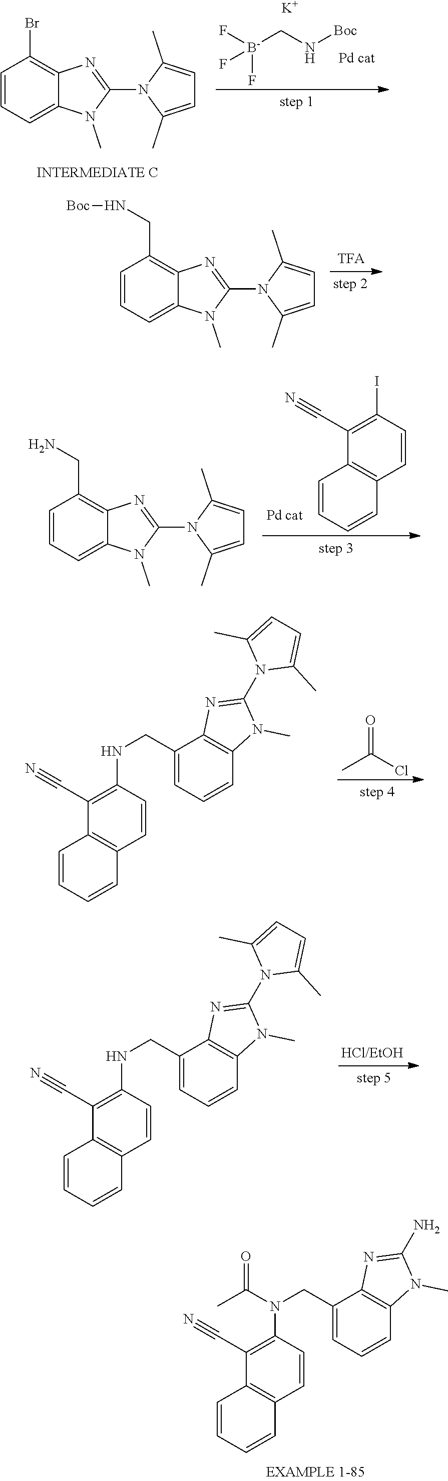

Step 1: A mixture of Intermediate C (250 mg, 821 μmol, 1.00 eq.), potassium (((tert-butoxycarbonyl)amino)methyl)trifluoroborate (487 mg, 2.05 mmol, 2.50 eq.), sodium carbonate (261 mg, 2.47 mmol, 3.00 eq.) and CataCXium A Pd G3 (59.9 mg, 82.2 μmol, 0.10 eq.) in dioxane (5.00 mL) and water (1.00 mL) was degassed and stirred at 80° C. for 2 hours under nitrogen atmosphere. The mixture was concentrated under reduced pressure to give a residue. The residue was purified by silica gel chromatography (ethyl acetate in petroleum ether 0-50%) to give tert-butyl N-[[2-(2,5-dimethylpyrrol-1-yl)-1-methyl-benzimidazol-4-yl]methyl]carbamate (100 mg, 264 μmol, 32% yield) as a yellow solid. LCMS [M+1]⁺=355.2. 1H NMR (400 MHZ, CDCl₃-d) 8-7.38-7.28 (m, 3H), 5.97 (s, 2H), 4.76 (br d, J=6.0 Hz, 2H), 3.49 (s, 3H), 2.06 (s, 6H), 1.44 (s, 9H).

Step 2: A mixture of tert-butyl N-[[2-(2,5-dimethylpyrrol-1-yl)-1-methyl-benzimidazol-4-yl]methyl]carbamate (90.0 mg, 254 μmol, 1.00 eq.) in trifluoroacetic acid (0.50 mL) and dichloromethane (1.50 mL) was stirred at 25° C. for 1 hour under nitrogen atmosphere. The mixture was concentrated under reduced pressure to give a residue. The crude product was purified by reversed-phase HPLC (0.1% Formic acid condition) to give [2-(2,5-dimethylpyrrol-1-yl)-1-methyl-benzimidazol-4-yl]methanamine (40.0 mg, 146 μmol, 58% yield) as a white solid. LCMS [M-16]+=238.0

Step 3: A mixture of 2-iodonaphthalene-1-carbonitrile (32.9 mg, 118 μmol, 1.20 eq.), [2-(2,5-dimethylpyrrol-1-yl)-1-methyl-benzimidazol-4-yl]methanamine (25.0 mg, 98.3 μmol, 1.00 eq.), Pd(dppf)C₁2 (7.19 mg, 9.83 μmol, 0.10 eq.), sodium tert-butoxide (28.3 mg, 295 μmol, 3.00 eq.) and DPPF (16.35 mg, 29.49 μmol, 0.3 eq) in dioxane (2.00 mL) was degassed and stirred at 100° C. for 2 hours under nitrogen atmosphere. The mixture was concentrated under reduced pressure to give a residue. The residue was purified by prep-TLC (SiO₂, petroleum ether/ethyl acetate 5:1) to give -[[2-(2,5-dimethylpyrrol-1-yl)-1-methyl-benzimidazol-4-yl]methylamino]naphthalene-1-carbonitrile (25.0 mg, 55.1 μmol, 56% yield) as a white solid. LCMS [M+1]⁺=406.2.

Step 4: To a solution of 2-[[2-(2,5-dimethylpyrrol-1-yl)-1-methyl-benzimidazol-4-yl]methylamino]naphthalene-1-carbonitrile (25.0 mg, 61.7 μmol, 1.00 eq.) in dimethyl formamide (1.00 mL) at 0° C. was added sodium hydride (7.40 mg, 185 μmol, 60.0% in mineral oil, 3.00 eq.) followed by acetyl chloride (14.5 mg, 185 μmol, 13.2 μL, 3.00 eq.) and the mixture was stirred at 25° C. for 2 hours. The mixture was quenched by water (0.50 mL), stirred for 10 minutes, then filtered and concentrated under reduced pressure. The residue was purified by prep-HPLC (0.1% Formic acid condition) to give N-(1-cyano-2-naphthyl)-N-[[2-(2,5-dimethylpyrrol-1-yl)-1-methyl-benzimidazol-4-yl]methyl]acetamide (15.0 mg, 32.2 μmol, 65% yield) as a white solid. LCMS [M+1]⁺=448.3.

Step 5: To a solution of N-(1-cyano-2-naphthyl)-N-[[2-(2,5-dimethylpyrrol-1-yl)-1-methyl-benzimidazol-4-yl]methyl]acetamide (10.0 mg, 22.3 μmol, 1.00 eq.) in ethyl alcohol (2.00 mL) was added conc. hydrochloric acid (0.25 mL). The mixture was stirred at 120° C. in a microwave reactor for 3 hours and then concentrated under reduced pressure to give a residue. The residue was purified by prep-HPLC (0.1% Formic acid condition) to give N-[(2-amino-1-methyl-benzimidazol-4-yl)methyl]-N-(1-cyano-2-naphthyl) acetamide, Example 1-85 (2.71 mg, 7.28 μmol, 33% yield) as a white solid. LCMS [M+1]⁺=370.1. ¹H NMR (400 MHZ, MeOD-d₄) δ=8.56-8.43 (m, 1H), 8.20 (d, J=8.8 Hz, 1H), 8.07-8.02 (m, 2H), 7.79-7.69 (m, 2H), 7.42 (d, J=8.8 Hz, 1H), 7.24 (d, J=8.0 Hz, 1H), 7.04 (t, J=7.6 Hz, 1H), 6.94 (d, J=7.6 Hz, 1H), 5.38-5.33 (m, 1H), 5.25-5.19 (m, 1H), 3.59 (s, 3H), 1.96 (s, 3H).

### EXAMPLE 1-86

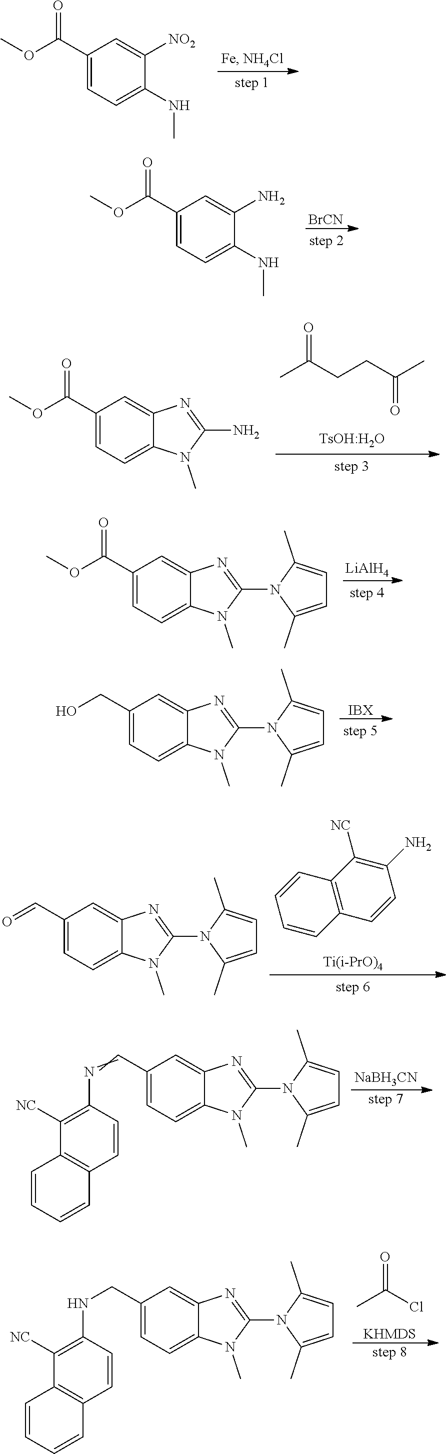

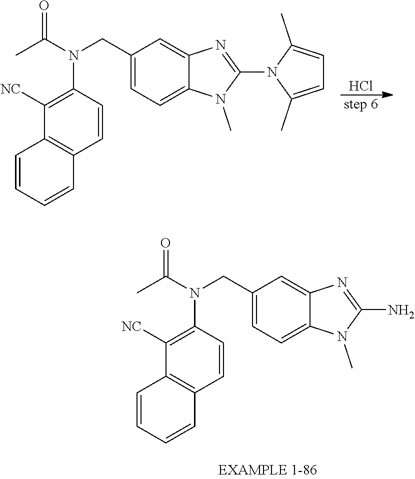

Step 1: To a mixture of methyl 4-(methylamino)-3-nitro-benzoate (5.00 g, 23.8 mmol, 1.00 eq.) in ethanol (60.0 mL) and water (12.0 mL) was added iron powder (6.64 g, 119 mmol, 5.00 eq.) and ammonium chloride (6.36 g, 119 mmol, 5.00 eq.). The reaction mixture was stirred at 80° C. for 1 hour. The reaction mixture was filtered and the filtrate was concentrated to remove ethyl alcohol. The aqueous remaining mixture was extracted with ethyl acetate (30.0 mL×3). The combined organic layers were dried over anhydrous sodium sulfate, filtered and concentrated to afford methyl 3-amino-4-(methylamino)benzoate (4.10 g, 20.2 mmol, 85% yield) as a black solid. LCMS [ESI, M+1]: 181.1. ¹H NMR (400 MHZ, CDCl₃) δ=7.61 (dd, J=1.2, 8.4 Hz, 1H), 7.40 (d, J=1.2 Hz, 1H), 6.58 (d, J=8.4 Hz, 1H), 3.84 (s, 3H), 3.34-3.06 (m, 3H), 2.91 (s, 3H).

Step 2: To a mixture of methyl 3-amino-4-(methylamino)benzoate (2.00 g, 11.1 mmol, 1.00 eq.) in ethyl alcohol (20.0 mL) and water (20.0 mL) was added cyanogen bromide (4.70 g, 44.4 mmol, 4.00 eq.). The reaction mixture was stirred at 50° C. for 16 hours. The reaction mixture was adjusted to pH=8 with saturated aq.sodium bicarbonate and concentrated to remove ethyl alcohol. The aqueous residual mixture was lyophilizated. The crude was purified by reversed-phase HPLC (0.1% ammonium hydroxide condition) to afford methyl 2-amino-1-methyl-benzimidazole-5-carboxylate (1.90 g, 9.12 mmol, 82% yield) as a white solid. LCMS [ESI, M+1]: 206.1. ¹H NMR (400 MHZ, MeOD-d₄) δ=7.88 (d, J=1.2 Hz, 1H), 7.75 (dd, J=1.6, 8.4 Hz, 1H), 7.21 (d, J=8.4 Hz, 1H), 3.89 (s, 3H), 3.59 (s, 3H).

Step 3: A mixture of methyl 2-amino-1-methyl-benzimidazole-5-carboxylate (1.40 g, 6.82 mmol, 1.00 eq.), hexane-2,5-dione (3.89 g, 34.1 mmol, 4.00 mL, 5.00 eq.) and p-toluenesulfonic acid monohydrate (259 mg, 1.36 mmol, 0.20 eq.) in toluene (90.0 mL) and dimethyl formamide (30.0 mL) was stirred at 130° C. for 16 hours with a Dean-Stark trap. The reaction mixture was washed with water (200 mL), extracted with ethyl acetate (40.0 mL×4). The combined organic layers were dried over anhydrous sodium sulfate, filtered and concentrated. The residue was purified by silica gel chromatography (ethyl acetate in petroleum ether gradient 0-40%) to afford methyl 2-(2,5-dimethylpyrrol-1-yl)-1-methyl-benzimidazole-5-carboxylate (1.90 g, 6.69 mmol, 98% yield) as a yellow oil. LCMS [ESI, M+1]: 284.2. ¹H NMR (400 MHZ, DMSO) d=8.31 (d, J=1.2 Hz, 1H), 8.00 (dd, J=1.6, 8.4 Hz, 1H), 7.77 (d, J=8.4 Hz, 1H), 5.95 (s, 2H), 3.89 (s, 3H), 3.52 (s, 3H), 1.97 (s, 6H).

Step 4: To a mixture of methyl 2-(2,5-dimethylpyrrol-1-yl)-1-methyl-benzimidazole-5-carboxylate (1.90 g, 6.71 mmol, 1.00 eq.) in tetrahydrofuran (30.0 mL) was added dropwise lithium aluminum hydride (509 mg, 13.4 mmol, 2.00 eq.) at 0° C. After the addition was completed, the mixture was stirred at 0° C. for 1 hour. The reaction mixture was quenched by sodium sulfate decahydrate (500 mg), diluted by ethyl acetate (20.0 mL) and filtered. The filtrate was concentrated to afford [2-(2,5-dimethylpyrrol-1-yl)-1-methyl-benzimidazol-5-yl]methanol (1.40 g, 5.31 mmol, 80% yield) as a colorless oil. LCMS [ESI, M+1]: 256.2. 1H NMR (400 MHZ, CDCl₃) δ=7.80 (d, J=0.8 Hz, 1H), 7.44-7.35 (m, 2H), 5.94 (s, 2H), 4.81 (d, J=3.6 Hz, 2H), 3.46 (s, 3H), 2.03 (s, 6H).

Step 5: To a mixture of [2-(2,5-dimethylpyrrol-1-yl)-1-methyl-benzimidazol-5-yl]methanol (600 mg, 2.35 mmol, 1.00 eq.) in ethyl acetate (10.0 mL) was added 1-hydroxy-1,2-benziodoxol-3 (1H)-one1-oxide (1.32 g, 4.70 mmol, 2.00 eq.). The mixture was stirred at 80° C. for 2 hours. The reaction mixture was filtered and the filtrate was concentrated. The residue was purified by silica gel chromatography (ethyl acetate in petroleum ether 0-60%) to afford 2-(2,5-dimethylpyrrol-1-yl)-1-methyl-benzimidazole-5-carbaldehyde (300 mg, 1.18 mmol, 50% yield) as a white solid. LCMS [ESI, M+1]: 254.0. ¹H NMR (400 MHZ, CDCl₃-d) δ=10.11 (s, 1H), 8.32 (d, J=1.2 Hz, 1H), 7.98-7.96 (m, 1H), 7.50 (d, J=8.4 Hz, 1H), 5.97 (s, 2H), 3.52 (s, 3H), 2.05 (s, 6H).

Step 6: To a mixture of 2-(2,5-dimethylpyrrol-1-yl)-1-methyl-benzimidazole-5-carbaldehyde (300 mg, 1.18 mmol, 1.00 eq.) and 2-aminonaphthalene-1-carbonitrile (199 mg, 1.18 mmol, 1.00 eq.) in toluene (9.00 mL) was added titanium (IV) isopropoxide (673 mg, 2.37 mmol, 699 μL, 2.00 eq.). The reaction mixture was stirred at 110° C. for 16 hours before being concentrated to afford 2-[[2-(2,5-dimethylpyrrol-1-yl)-1-methyl-benzimidazol-5-yl]methyleneamino]naphthalene-1-carbonitrile (460 mg, crude) as a yellow oil.

Step 7: To a mixture of 2-[[2-(2,5-dimethylpyrrol-1-yl)-1-methyl-benzimidazol-5-yl]methyleneamino]naphthalene-1-carbonitrile (460 mg, 1.14 mmol, 1.00 eq.) in tetrahydrofuran (10.0 mL) at 0° C. was added sodium cyanoborohydride (107 mg, 1.71 mmol, 1.50 eq.). The mixture was stirred at 50° C. for 2 hours. The reaction mixture was quenched by saturated aq. ammonium chloride (15.0 mL), extracted with ethyl acetate (10.0 mL×4). The combined organic layers were dried over anhydrous sodium sulfate, filtered and concentrated. The residue was purified by silica gel chromatography (ethyl acetate in petroleum ether 0-60%) and further purified by prep-HPLC (0.225% formic acid condition) to afford 2-[[2-(2,5-dimethylpyrrol-1-yl)-1-methyl-benzimidazol-5-yl]methylamino]naphthalene-1-carbonitrile (90.0 mg, 203 μmol, 18% yield) as a yellow oil. LCMS [ESI, M+1]: 406.3. ¹H NMR (400 MHz, MeOD-d₄) δ=7.84-7.78 (m, 2H), 7.74-7.69 (m, 2H), 7.59-7.48 (m, 3H), 7.31-7.23 (m, 1H), 7.10-6.99 (m, 1H), 6.00-5.90 (m, 2H), 4.82 (s, 2H), 3.50 (s, 3H), 2.01 (d, J=5.6 Hz, 6H).

Step 8: To a mixture of 2-[[2-(2,5-dimethylpyrrol-1-yl)-1-methyl-benzimidazol-5-yl]methylamino]naphthalene-1-carbonitrile (50.0 mg, 123 μmol, 1.00 eq.) in tetrahydrofuran (1.50 mL) at 0° C. under nitrogen atmosphere was added KHMDS (1.00 M, 493 μL, 4.00 eq.). The reaction mixture was stirred at 0° C. for 0.5 hour. Then acetyl chloride (48.4 mg, 616 μmol, 44.0 μL, 5.00 eq.) was added and the mixture was warmed to 25° C. and stirred at 25° C. for 2 hours. The reaction mixture was quenched by water (10.0 mL), extracted with ethyl acetate (5.00 mL×3). The combined organic layers were dried over anhydrous sodium sulfate, filtered and concentrated. The residue was purified by silica gel chromatography (ethyl acetate in petroleum ether 0-80%) to afford N-(1-cyano-2-naphthyl)-N-[[2-(2,5-dimethylpyrrol-1-yl)-1-methyl-benzimidazol-5-yl]methyl]acetamide (20.0 mg, 44.7 μmol, 36% yield) as a yellow oil. LCMS [ESI, M+1]: 448.3.

Step 9: To a mixture of N-(1-cyano-2-naphthyl)-N-[[2-(2,5-dimethylpyrrol-1-yl)-1-methyl-benzimidazol-5-yl]methyl]acetamide (20.0 mg, 44.7 μmol, 1.00 eq.) in ethyl alcohol (1.00 mL) was added 12M hydrochloric acid (0.10 mL). The mixture was stirred at 120° C. for 0.5 hour in a microwave reactor. The reaction mixture was adjusted to pH=8 with ammonium hydroxide and concentrated. The residue was purified by Prep-HPLC (ammonium bicarbonate condition to afford N-[(2-amino-1-methyl-benzimidazol-5-yl)methyl]-N-(1-cyano-2-naphthyl) acetamide, Example 1-86 (1.90 mg, 4.78 μmol, 11% yield) as an off-white solid. LCMS [ESI, M+1]: 370.1. ¹H NMR (400 MHZ, MeOD) δ=8.12 (dd, J=8.4, 13.2 Hz, 2H), 8.02 (d, J=8.0 Hz, 1H), 7.81-7.74 (m, 1H), 7.73-7.65 (m, 1H), 7.24 (d, J=8.8 Hz, 1H), 7.02 (d, J=8.0 Hz, 1H), 6.96 (s, 1H), 6.93-6.84 (m, 1H), 5.28 (d, J=14.0 Hz, 2H), 3.50 (s, 3H), 1.92 (s, 3H).

### EXAMPLE 1-87

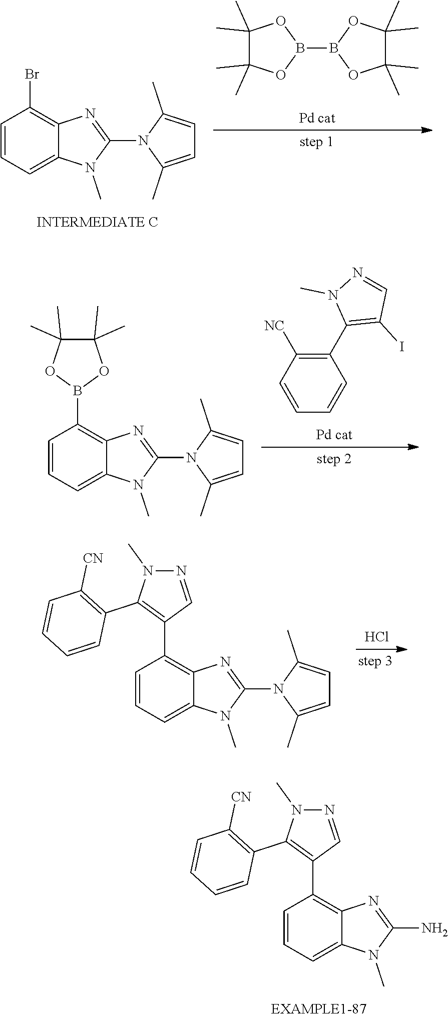

Step 1: To a solution of Intermediate C (100 mg, 329 μmol, 1.00 eq.) and 4,4,4′,4′,5,5,5′,5′-octamethyl-2,2′-bi (1,3,2-dioxaborolane) (125 mg, 493 μmol, 1.50 eq.) in dioxane (2.50 mL) was added potassium acetate (80.7 mg, 822 μmol, 2.50 eq.) and [1,1′-bis(diphenylphosphino) ferrocene]dichloropalladium (II) (12.0 mg, 16.4 μmol, 0.05 eq.). The mixture was stirred at 80° C. for 16 hours under nitrogen atmosphere. The mixture was concentrated. The residue was purified by silica gel chromatography (ethyl acetate in petroleum ether 5-20%) to afford 2-(2,5-dimethyl-1H-pyrrol-1-yl)-1-methyl-4-(4,4,5,5-tetramethyl-1,3,2-dioxaborolan-2-yl)-1H-benzo[d]imidazole (90.0 mg, crude) as a light yellow oil. LCMS [ESI, M+1]⁺=270.2. ¹HNMR (400 MHZ, CDCl₃) δ=7.92-7.84 (m, 1H), 7.59-7.48 (m, 2H), 6.00 (s, 1H), 5.93 (s, 1H), 3.54 (s, 3H), 2.08-2.06 (m, 6H), 1.42 (s, 3H).

Step 2: To a solution of Intermediate L (50.0 mg, 162 μmol, 1.00 eq.) and 2-(2,5-dimethyl-1H-pyrrol-1-yl)-1-methyl-4-(4,4,5,5-tetramethyl-1,3,2-dioxaborolan-2-yl)-1H-benzo[d]imidazole (68.2 mg, 194 μmol, 1.20 eq.) in dioxane (1.50 mL) and water (0.30 mL) was added sodium carbonate (42.9 mg, 404 μmol, 2.50 eq.) and [1,1′-bis(diphenylphosphino) ferrocene]dichloropalladium (II) (11.8 mg, 16.2 μmol, 0.10 eq.). The mixture was stirred at 80° C. for 3 hours under nitrogen atmosphere before being diluted with water (30.0 mL) and extracted with ethyl acetate (10.0 mL×2). The organic layer was washed with brine (10.0 mL), dried over sodium sulfate, filtered and concentrated. The residue was purified by silica gel chromatography (ethyl acetate in petroleum ether 5-20%) to afford 2-(4-(2-(2,5-dimethyl-1H-pyrrol-1-yl)-1-methyl-1H-benzo[d]imidazol-4-yl)-1-methyl-1H-pyrazol-5-yl)benzonitrile (50.0 mg, 114 μmol, 71% yield) as a light yellow oil. LCMS [ESI, M+1]⁺=407.2.

Step 3: To a solution of 2-(4-(2-(2,5-dimethyl-1H-pyrrol-1-yl)-1-methyl-1H-benzo[d]imidazol-4-yl)-1-methyl-1H-pyrazol-5-yl)benzonitrile (50.0 mg, 123 μmol, 1.00 eq.) in ethanol (2.00 mL) was added concentrated hydrochloric acid (12.0 M, 0.20 mL, 19.5 eq.). The mixture was stirred at 120° C. for 1 hour in a microwave reactor. The mixture was concentrated. The residue was purified by prep-HPLC (HCl condition) to afford 2-(4-(2-amino-1-methyl-1H-benzo[d]imidazol-4-yl)-1-methyl-1H-pyrazol-5-yl)benzonitrile hydrochloride, Example 1-87 (28.3 mg, 77.4 μmol, 63% yield) as a white solid. LCMS [ESI, M+1]⁺=329.2. 1HNMR (400 MHZ, DMSO-d₆) δ=12.80 (s, 1H), 8.33 (s, 2H), 7.95-7.83 (m, 3H), 7.83-7.78 (m, 1H), 7.68 (dt, J=1.2, 7.6 Hz, 1H), 7.36 (d, J=8.0 Hz, 1H), 7.08 (t, J=8.0 Hz, 1H), 6.56 (dd, J=0.8, 7.6 Hz, 1H), 3.78 (s, 3H), 3.64 (s, 3H).

### EXAMPLE 1-88

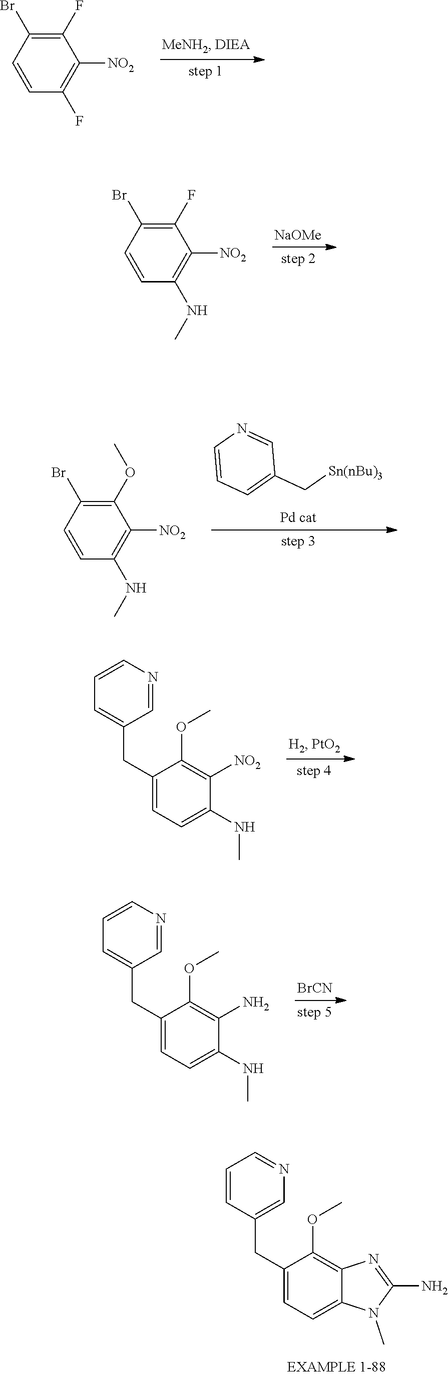

Step 1: To a solution of 1-bromo-2,4-difluoro-3-nitro-benzene (6.70 g, 28.2 mmol, 1.00 eq.) in dimethylformamide (80.0 mL) were added to diisopropyl ethyl amine (10.9 g, 84.5 mmol, 14.7 mL, 3.00 eq.) and methanamine (3.80 g, 56.3 mmol, 2.00 eq., hydrochloride) at 20° C. Then the mixture was stirred at 20° C. for 16 hours. The residue was diluted with ethyl acetate (30.0 mL) and water (30.0 mL). The layers were separated and the aqueous phase was extracted with ethyl acetate (80.0 mL×3). The combined organic extracts were washed brine (60.0 mL×3) and then dried over anhydrous sodium sulfate, filtered and concentrated under vacuum to give a residue. The residue was purified by silica gel chromatography (ethyl acetate in petroleum ether 0-5%). The residue was further purified by prep-TLC (SiO₂, petroleum ether/ethyl acetate 5:1) to give compound of 4-bromo-3-fluoro-N-methyl-2-nitro-aniline (778 mg, 3.12 mmol, 11% yield) as a yellow solid. ¹H NMR (400 MHZ, CDCl₃-d) δ=7.54-7.45 (m, 1H), 7.20 (s br, 1H), 6.53 (d, J=9.2 Hz, 1H), 2.98 (br, J=4.8 Hz, 3H).

Step 2: To a solution of 4-bromo-3-fluoro-N-methyl-2-nitro-aniline (778 mg, 3.12 mmol, 1.00 eq.) in methanol (8.00 mL) was added to sodium methoxide (5.40 M, 5.79 mL, 10.0 eq.) at 20° C., then the mixture was stirred at 20° C. for 16 hours. The residue was diluted with ethyl acetate (20.0 mL) and water (20.0 mL). The layers were separated and the aqueous phase was extracted with ethyl acetate (3×20 mL). The combined organic extracts were washed brine (30.0 mL) and then dried over anhydrous sodium sulfate, filtered and concentrated under vacuum. The residue was purified by silica gel chromatography (ethyl acetate in petroleum ether 0-50%) to give 4-bromo-3-methoxy-N-methyl-2-nitro-aniline (760 mg, 2.91 mmol, 93% yield) as an orange solid. LCMS [M+1]⁺: 263.0. ¹H NMR (400 MHZ, CDCl₃-d) δ=7.48 (d, J=9.2 Hz, 1H), 6.46 (d, J=9.2 Hz, 1H), 6.05 (s, br 1H), 3.98 (s, 3H), 2.91 (d, J=5.2 Hz, 3H).

Step 3: To a solution of 4-bromo-3-methoxy-N-methyl-2-nitro-aniline (100 mg, 383 μmol, 1.00 eq.), tributyl(3-pyridylmethyl) stannane (190 mg, 498 μmol, 1.30 eq.) and bis(triphenylphosphine) palladium (II) chloride (53.8 mg, 76.6 μmol, 0.20 eq.) in dimethylformamide (2.00 mL) was degassed and heated to 110° C. for 6 hours. After being cooled to room temperature the mixture was diluted with ethyl acetate (10.0 mL) and water (10.0 mL). The layers were separated and the aqueous phase was extracted with ethyl acetate (20.0 mL×3). The combined organic extracts were washed brine (20.0 mL) and dried over anhydrous sodium sulfate, filtered and concentrated under vacuum. The residue was purified by prep-TLC (SiO₂, petroleum ether/ethyl acetate 1:1) to give 3-methoxy-N-methyl-2-nitro-4-(3-pyridylmethyl) aniline (150 mg, crude) as a yellow solid. LCMS [M+1]⁺: 273.8. ¹H NMR (400 MHZ, CDCl₃-d) δ=8.49 (d, J=2.0 Hz, 1H), 8.45 (dd, J=1.4, 4.8 Hz, 1H), 7.70-7.67 (m, 1H), 7.20 (dd, J=4.8, 8.0 Hz, 1H), 7.11 (d, J=8.8 Hz, 1H), 6.49 (d, J=8.8 Hz, 1H), 3.91 (s, 2H), 3.75 (s, 3H), 2.90 (d, J=5.2 Hz, 3H).

Step 4: A solution of 3-methoxy-N-methyl-2-nitro-4-(3-pyridylmethyl) aniline (75.0 mg, 274 μmol, 1.00 eq.), platinum dioxide (75.0 mg, 330 μmol, 1.20 eq.) in methanol (2.00 mL) was degassed and charged with hydrogen for three times and then stirred at 20° C. for 2 hours. The mixture was used into next step without a work-up. LCMS [M+1]⁺: 244.0

Step 5: To a solution of 3-methoxy-N1-methyl-4-(3-pyridylmethyl)benzene-1,2-diamine (66.0 mg, 272 μmol, 1.00 eq.) in methanol (2.00 mL) was added cyanogen bromide (57.5 mg, 543 μmol, 39.9 μL, 2.00 eq.) at 20° C. and the mixture stirred at 20° C. for 16 hours under nitrogen atmosphere. The reaction mixture was filtered and then concentrated under reduced pressure to give a residue. The residue was purified by prep-HPLC (HCl condition) to give 4-methoxy-1-methyl-5-(3-pyridylmethyl)benzimidazol-2-amine hydrochloride, Example 1-88 (6.54 mg, 23.9 μmol, 9% yield) as an off-white solid. LCMS [M+1]⁺: 268.9. ¹H NMR (400 MHZ, MeOD-d₄) δ=8.77 (s, 1H), 8.71 (d, J=5.6 Hz, 1H), 8.48 (d, J=8.0 Hz, 1H), 8.00 (dd, J=6.0, 8.0 Hz, 1H), 7.33-7.28 (m, 1H), 7.26-7.21 (m, 1H), 4.34 (s, 2H), 3.95 (s, 3H), 3.67 (s, 3H).

### EXAMPLE 1-89

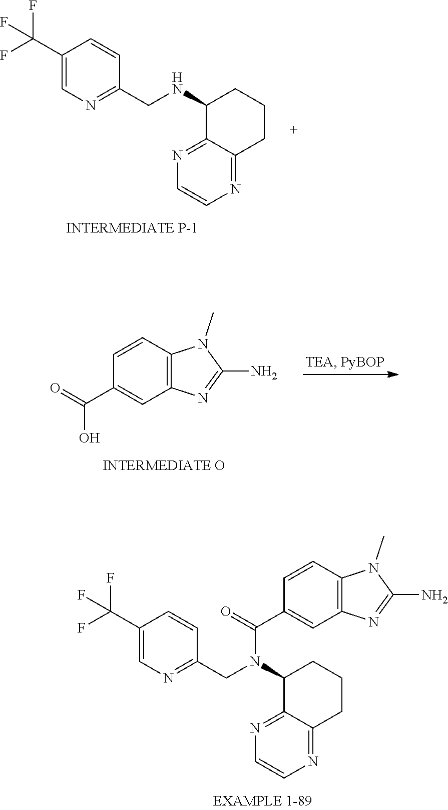 [SMILES: FC(F)(F)c1ccc(CN[C@H]2CCCc3nccnc32)nc1 ; Cn1c(N)nc2cc(C(=O)O)ccc21 ; Cn1c(N)nc2cc(C(=O)N(Cc3ccc(C(F)(F)F)cn3)[C@H]3CCCc4nccnc43)ccc21]

To a solution of Intermediate O (30.0 mg, 1.00 eq.) in N,N-dimethyl acetamide (0.60 mL) was added Intermediate P-1 (53.2 mg, 1.10 eq.), triethylamine (39.7 mg, 2.50 eq.) and PyBOP (98.0 mg, 1.20 eq.). The mixture was stirred at 25° C. for 48 hours. The reaction mixture was diluted with water (10.0 mL) and extracted with ethyl acetate (3× 10.0 mL). The combined organic layers were dried over anhydrous sodium sulfate, filtered and concentrated under reduced pressure to give a residue. The residue was purified by prep-HPLC (HCl condition) to afford(S)-2-amino-1-methyl-N-(5,6,7,8-tetrahydroquinoxalin-5-yl)-N-((5-(trifluoromethyl)pyridin-2-yl)methyl)-1H-benzo[d]imidazole-5-carboxamide, Example 1-89 (5.00 mg, 7% yield) as a yellow solid. LCMS [M+1]⁺=482.0. ¹H NMR (400 MHZ, DMSO-d₆) δ=8.82 (s, 1H), 8.67 (br s, 2H), 8.49-8.41 (m, 2H), 8.12 (dd, J=2.0, 8.4 Hz, 1H), 7.72 (d, J=8.0 Hz, 1H), 7.65-7.45 (m, 3H), 5.28-5.09 (m, 1H), 4.81 (br d, J=17.0 Hz, 1H), 3.65 (s, 3H), 3.01-2.91 (m, 1H), 2.87-2.76 (m, 1H), 2.23 (br d, J=2.0 Hz, 1H), 2.16-2.05 (m, 1H), 2.03-1.94 (m, 1H), 1.77-1.60 (m, 1H).

### EXAMPLE 1-90

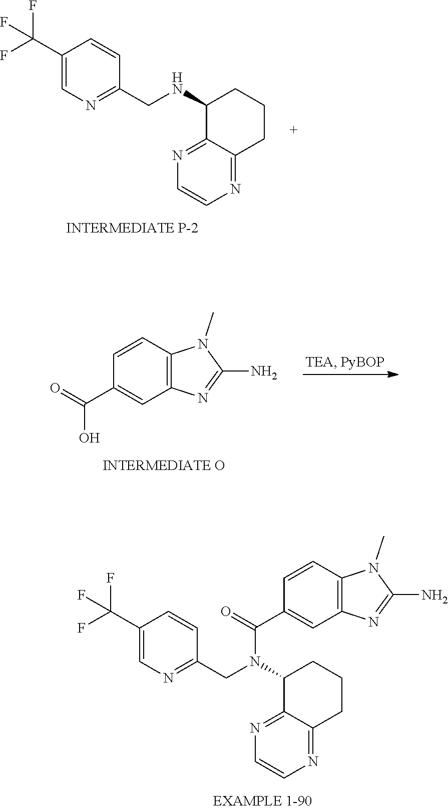 [SMILES: FC(F)(F)c1ccc(CN[C@H]2CCCc3nccnc32)nc1 ; Cn1c(N)nc2cc(C(=O)O)ccc21 ; Cn1c(N)nc2cc(C(=O)N(Cc3ccc(C(F)(F)F)cn3)[C@@H]3CCCc4nccnc43)ccc21]

To a solution of Intermediate O (55.0 mg, 1.00 eq.) in N,N-dimethyl acetamide (1.10 mL) was added Intermediate P-2 (97.6 mg, 1.10 eq.), triethylamine (72.8 mg, 2.50 eq.) and PyBOP (180 mg, 1.20 eq.). The mixture was stirred at 40° C. for 16 hours. The mixture was diluted with water (10.0 mL) and extracted with ethyl acetate (3×10.0 mL). The combined organic layers were dried over anhydrous sodium sulfate, filtered and concentrated under reduced pressure to give a residue. The residue was purified by prep-HPLC (TFA condition). The desired fractions were collected and neutralized with solid sodium bicarbonate and concentrated under vacuum to remove acetonitrile. The aqueous layer was extracted with ethyl acetate (3×20.0 mL). The combined organic layers were dried over anhydrous sodium sulfate, filtered and concentrated under vacuum to afford (R)-2-amino-1-methyl-N-(5,6,7,8-tetrahydroquinoxalin-5-yl)-N-((5-(trifluoromethyl)pyridin-2-yl)methyl)-1H-benzo[d]imidazole-5-carboxamide, Example 1-90 (5.90 mg, 4% yield) as a yellow oil. LCMS [M+1]⁺=482.1. ¹H NMR (400 MHZ, MeOD-d₄) δ=8.91-8.69 (m, 1H), 8.49 (br s, 1H), 8.42-8.32 (m, 1H), 8.10 (dd, J=2.3, 8.4 Hz, 1H), 7.97-7.74 (m, 1H), 7.63-7.15 (m, 3H), 5.41-5.05 (m, 1H), 4.94 (br s, 1H), 4.16 (br d, J=16.6 Hz, 1H), 3.63-3.53 (m, 3H), 3.04-2.80 (m, 2H), 2.36-2.28 (m, 1H), 2.13-2.00 (m, 2H), 1.65-1.56 (m, 1H).

### EXAMPLE 1-91

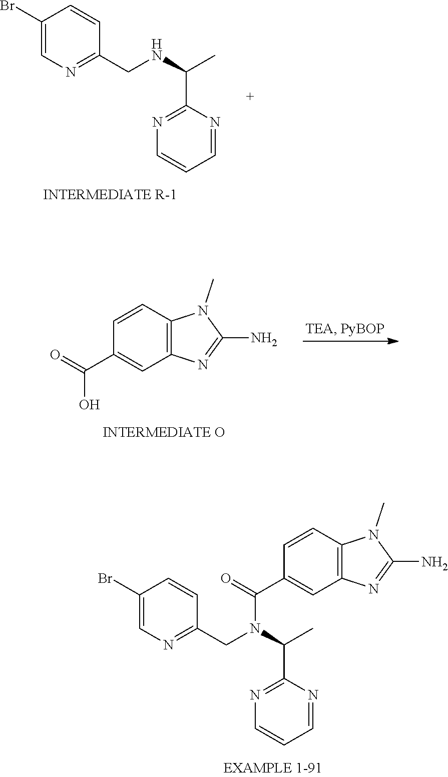 [SMILES: C[C@H](NCc1ccc(Br)cn1)c1ncccn1 ; Cn1c(N)nc2cc(C(=O)O)ccc21 ; C[C@@H](c1ncccn1)N(Cc1ccc(Br)cn1)C(=O)c1ccc2c(c1)nc(N)n2C]

To a solution of Intermediate O (30.0 mg, 157 μmol, 1.00 eq.) in N,N-dimethyl acetamide (0.60 mL) was added Intermediate R-1 (50.6 mg, 173 μmol, 1.10 eq.), triethylamine (39.7 mg, 392 μmol, 54.6 μL, 2.50 eq.) and PyBOP (98.0 mg, 188 μmol, 1.20 eq.). The mixture was stirred at 25° C. for 12 hours. The reaction mixture was quenched by addition of water (10.0 mL) and extracted with ethyl acetate (10.0 mL). The organic layer was dried over anhydrous sodium sulfate, filtered and concentrated under reduced pressure to give a residue. The residue was purified by prep-HPLC (ammonium hydroxide condition) to afford(S)-2-amino-N-((5-bromopyridin-2-yl)methyl)-1-methyl-N-(1-(pyrimidin-2-yl)ethyl)-1H-benzo[d]imidazole-5-carboxamide, Example 1-91 (6.30 mg, 8.50% yield, 98.0% purity) as a yellow solid. LCMS [M+1]⁺=466.0. ¹H NMR (400 MHZ, MeOD-d₄) δ=8.72 (br d, J=4.8 Hz, 2H), 8.50 (br s, 1H), 7.93-7.86 (m, 1H), 7.46 (br s, 1H), 7.38 (d, J=8.4 Hz, 1H), 7.31 (t, J=4.8 Hz, 1H), 7.25 (br d, J=4.0 Hz, 2H), 5.70-5.44 (m, 1H), 4.94 (br d, J=16.8 Hz, 1H), 4.64-4.49 (m, 1H), 3.57 (br s, 3H), 1.64 (br d, J=6.5 Hz, 3H).

### EXAMPLE 1-92

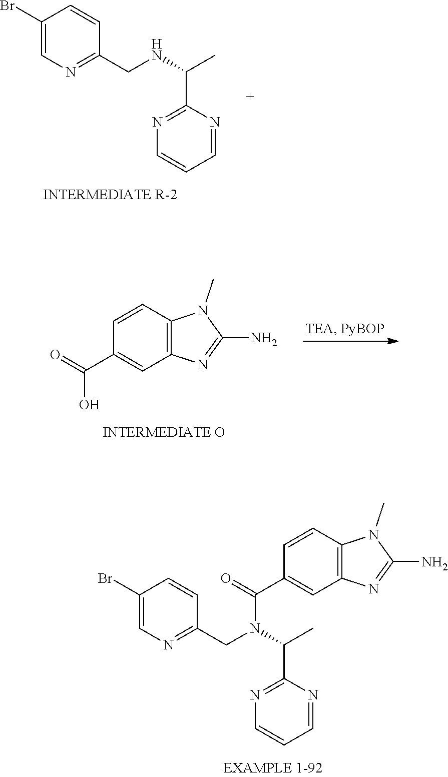 [SMILES: C[C@@H](NCc1ccc(Br)cn1)c1ncccn1 ; Cn1c(N)nc2cc(C(=O)O)ccc21 ; C[C@H](c1ncccn1)N(Cc1ccc(Br)cn1)C(=O)c1ccc2c(c1)nc(N)n2C]

To a solution of Intermediate O (30.0 mg, 157 μmol, 1.00 eq) in DMA (0.60 mL) was added Intermediate R-2 (50.6 mg, 172 μmol, 1.10 eq), Et3N (39.7 mg, 392 μmol, 54.6 μL, 2.50 eq.) and PyBop (97.9 mg, 188 μmol, 1.20 eq). The mixture was stirred at 25° C. for 12 hours. The reaction mixture was diluted with water (10 mL) and ethyl acetate (10 mL) and filtered. The filtrate was extracted with ethyl acetate (10 mL×2). The combined organic layers were dried over Na₂SO₄, filtered and concentrated under reduced pressure to give a residue. The residue was purified by prep-HPLC (ammonium hydroxide condition) to afford (R)-2-amino-N-((5-bromopyridin-2-yl)methyl)-1-methyl-N-(1-(pyrimidin-2-yl)ethyl)-1H-benzo[d]imidazole-5-carboxamide, Example 1-92 (8.00 mg, 16.8 μmol, 10.7% yield) as a white solid. LCMS [M+1]⁺=466.0. ¹H NMR (400 MHZ, METHANOL-d₄) δ=8.75 (br d, J=4.8 Hz, 2H), 8.52 (br s, 1H), 7.92 (br d, J=7.6 Hz, 1H), 7.54-7.37 (m, 2H), 7.33 (t, J=4.8 Hz, 1H), 7.31-7.09 (m, 2H), 5.77-5.41 (m, 1H), 4.96 (br d, J=16.4 Hz, 1H), 4.69-4.45 (m, 1H), 3.59 (br s, 3H), 1.66 (br d, J=6.4 Hz, 3H)

### EXAMPLE 2-1

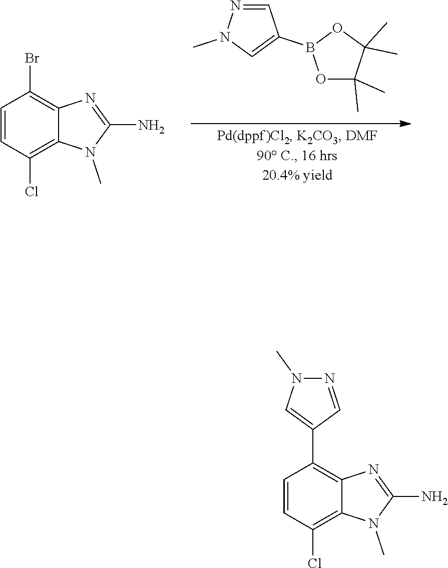

To a solution of Intermediate D (50.0 mg, 192 μmol, 1.00 eq.) and 1-methyl-4-(4,4,5,5-tetramethyl-1,3,2-dioxaborolan-2-yl) pyrazole (59.9 mg, 288 μmol, 1.50 eq.) in N,N-dimethylformamide (2.00 mL) was added potassium carbonate (79.6 mg, 576 μmol, 3.00 eq.), followed by [1,1′-bis(diphenylphosphino) ferrocene]dichloropalladium (II) (14.0 mg, 19.2 μmol, 0.10 eq.) at 10° C. under nitrogen atmosphere. The mixture was stirred at 90° C. for 16 hours. The mixture was filtered and the filtrate was purified by prep-HPLC (HCl condition) then further purified by prep-HPLC (ammonium bicarbonate condition) to afford 7-chloro-1-methyl-4-(1-methylpyrazol-4-yl)benzimidazol-2-amine hydrochloride, Example 2-1 (10.4 mg, 39.2 μmol, 20% yield) as a white solid. LCMS [ESI, M+1]⁺: 262.1. ¹H NMR (400 MHZ, MeOD) δ=7.53 (d, J=2.0 Hz, 1H), 7.08-7.02 (m, 1H), 7.02-6.97 (m, 1H), 6.38 (d, J=2.0 Hz, 1H), 3.92 (s, 3H), 3.77 (s, 3H).

### EXAMPLE 2-2

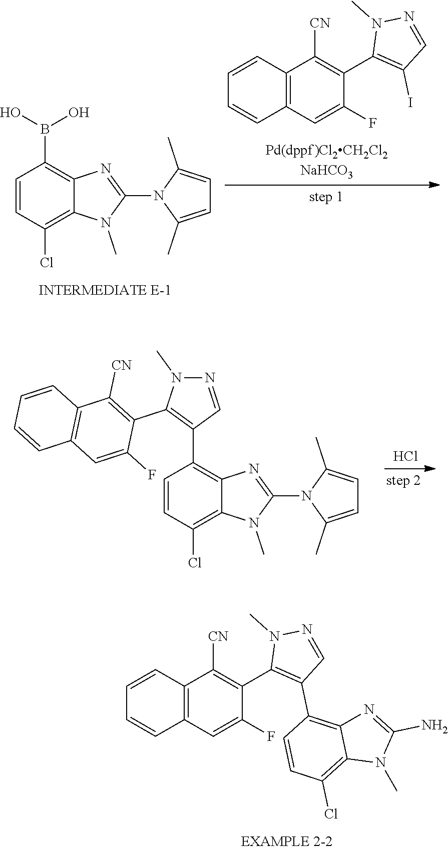 [SMILES: Cc1ccc(C)n1-c1nc2c(B(O)O)ccc(Cl)c2n1C ; Cn1ncc(I)c1-c1c(F)cc2ccccc2c1C#N ; Cc1ccc(C)n1-c1nc2c(-c3cnn(C)c3-c3c(F)cc4ccccc4c3C#N)ccc(Cl)c2n1C ; Cl ; Cn1ncc(-c2ccc(Cl)c3c2nc(N)n3C)c1-c1c(F)cc2ccccc2c1C#N]

Step 1: To a solution of Intermediate E-1 (58.0 mg, 191 μmol, 1.00 eq.) and 3-fluoro-2-(4-iodo-2-methyl-pyrazol-3-yl) naphthalene-1-carbonitrile (see WO2021050915) (108 mg, 287 μmol, 1.50 eq.) in dioxane (0.90 mL) and water (0.18 mL) was added sodium bicarbonate (64.2 mg, 764 μmol, 29.7 μL, 4.00 eq.), followed by 1,1′-bis(diphenylphosphino) ferrocene-palladium (II) dichloride dichloromethane (31.2 mg, 38.2 μmol, 0.20 eq.) at 10° C. under nitrogen atmosphere. The mixture was stirred at 80° C. for 16 hours. The mixture was concentrated in vacuum. The residue was purified by flash silica gel chromatography (Ethyl acetate in Petroleum ether 30%) to afford 2-[4-[7-chloro-2-(2,5-dimethylpyrrol-1-yl)-1-methyl-benzimidazol-4-yl]-2-methyl-pyrazol-3-yl]-3-fluoro-naphthalene-1-carbonitrile (52.0 mg, 87.8 μmol, 46% yield) as a yellow oil. LCMS [ESI, M+1]⁺: 509.3.

Step 2: A mixture of 2-[4-[7-chloro-2-(2,5-dimethylpyrrol-1-yl)-1-methyl-benzimidazol-4-yl]-2-methyl-pyrazol-3-yl]-3-fluoro-naphthalene-1-carbonitrile (24.0 mg, 47.2 μmol, 1.00 eq.), hydrochloric acid (12 M, 480 μL, 122 eq.) and ethanol (9.40 mL) was heated at 120° C. for 30 min in a microwave reactor. The mixture was concentrated in vacuum. The residue was purified by prep-HPLC (HCl condition) to afford 2-[4-(2-amino-7-chloro-1-methyl-benzimidazol-4-yl)-2-methyl-pyrazol-3-yl]-3-fluoro-naphthalene-1-carbonitrile hydrochloride, Example 2-2 (23.4 mg, 49.9 μmol, 53% yield) as an off-white solid. LCMS [ESI, M+1]⁺: 430.9. ¹H NMR (400 MHZ, MeOD) δ=8.20 (d, J=10.0 Hz, 1H), 8.14-8.06 (m, 2H), 7.98 (s, 1H), 7.81-7.72 (m, 2H), 7.03 (d, J=8.4 Hz, 1H), 6.62 (d, J=8.0 Hz, 1H), 3.98 (s, 3H), 3.90 (d, J=0.4 Hz, 3H).

### EXAMPLE 2-3

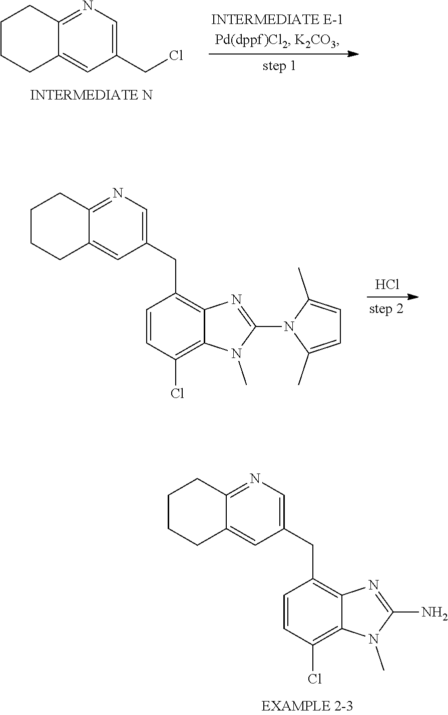 [SMILES: ClCc1cnc2c(c1)CCCC2 ; Cc1ccc(C)n1-c1nc2c(Cc3cnc4c(c3)CCCC4)ccc(Cl)c2n1C ; Cl ; Cn1c(N)nc2c(Cc3cnc4c(c3)CCCC4)ccc(Cl)c21]

Step 1: A mixture of Intermediate N (30.0 mg, 165 μmol, 1 eq.), Intermediate E-1 (65.2 mg, 215 μmol, 1.30 eq.), potassium carbonate (45.7 mg, 332 μmol, 2.00 eq.) and Pd(dppf)Cl₂ (12.1 mg, 16.5 μmol, 0.10 eq.) in dimethylformamide (0.50 mL) was degassed and stirred at 100° C. for 4 hours under nitrogen atmosphere. The reaction mixture was concentrated under reduced pressure to give a residue. The residue was purified by prep-TLC (SiO₂, Petroleum ether/Ethyl acetate 1:1) to give 3-[[7-chloro-2-(2,5-dimethylpyrrol-1-yl)-1-methyl-benzimidazol-4-yl]methyl]-5,6,7,8-tetrahydroquinoline (45.0 mg, 108 μmol, 66% yield) as a yellow oil. LC-MS [M+1]⁺=405.3. ¹H NMR (400 MHZ, CHLOROFORM-d) 8=8.34 (d, J=2.0 Hz, 1H), 7.38 (s, 1H), 7.21 (d, J=8.0 Hz, 1H), 6.98 (d, J=8.0 Hz, 1H), 5.97 (s, 2H), 4.31 (s, 2H), 3.72 (s, 3H), 2.89-2.85 (m, 2H), 2.68 (t, J=6.3 Hz, 2H), 2.08 (s, 6H), 1.89-1.83 (m, 2H), 1.80-1.74 (m, 2H).

Step 2: To a solution of 3-[[7-chloro-2-(2,5-dimethylpyrrol-1-yl)-1-methyl-benzimidazol-4-yl]methyl]-5,6,7,8-tetrahydroquinoline (22.0 mg, 54.3 μmol, 1.00 eq.) in ethyl alcohol (0.50 mL) was added hydrochloric acid (12 M, 91 μL, 1.09 mmol, 20.0 eq.). The mixture was stirred at 120° C. for 1 hour in a microwave reactor. The reaction mixture was concentrated under reduced pressure. The residue was purified by prep-HPLC (HCl condition) to give 7-chloro-1-methyl-4-(5,6,7,8-tetrahydroquinolin-3-ylmethyl)benzimidazol-2-amine hydrochloride, Example 2-3 (10.0 mg, 30.0 μmol, 55% yield) as a white solid. LC-MS [M+1]+=327.1. ¹H NMR (400 MHZ, MeOH-d₄) δ=8.46 (s, 1H), 8.12 (s, 1H), 7.30 (d, J=8.4 Hz, 1H), 7.13 (d, J=8.4 Hz, 1H), 4.39 (s, 2H), 3.98 (s, 3H), 3.08 (t, J=6.4 Hz, 2H), 2.92 (t, J=6.4 Hz, 2H), 2.03-1.94 (m, 2H), 1.93-1.85 (m, 2H).

### EXAMPLE 2-4

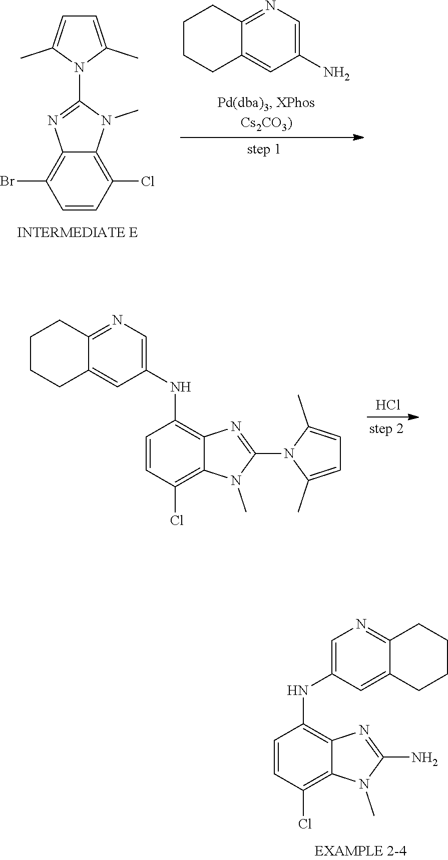

Step 1: To a solution of Intermediate E (300 mg, 886 μmol, 1.00 eq.) and 5,6,7,8-tetrahydroquinolin-3-amine (158 mg, 1.06 mmol, 1.20 eq.) in tert-butanol (6.00 mL) was added 2-(dicyclohexylphosphino)-2,4,6-triisopropylbiphenyl (84.5 mg, 177 μmol, 0.20 eq.), cesium carbonate (866 mg, 2.66 mmol, 3.00 eq.) and tris(dibenzylideneacetone) dipalladium (0) (81.1 mg, 88.6 μmol, 0.10 eq.). The mixture was stirred at 100° C. for 3 hours under nitrogen atmosphere. The mixture was diluted with water (30.0 mL) and extracted with ethyl acetate (10.0 mL×3). The organic layer was washed with brine (20.0 mL), dried over anhydrous sodium sulfate, filtered and concentrated. The residue was purified by flash silica gel chromatography (ethyl acetate in petroleum ether 5-30%) to afford N-(7-chloro-2-(2,5-dimethyl-1H-pyrrol-1-yl)-1-methyl-1H-benzo[d]imidazol-4-yl)-5,6,7,8-tetrahydroquinolin-3-amine (300 mg, 698 μmol, 79% yield) as a yellow oil. LCMS [ESI, M+1]: 406.3. ¹H NMR (400 MHZ, CDCl₃) δ=8.37 (d, J=2.0 Hz, 1H), 7.32 (d, J=1.6 Hz, 1H), 7.15 (d, J=8.4 Hz, 1H), 6.95 (d, J=8.4 Hz, 1H), 6.78 (s, 1H), 5.98 (s, 2H), 3.72 (s, 3H), 2.91 (t, J=6.4 Hz, 2H), 2.78 (t, J=6.0 Hz, 2H), 2.08 (s, 6H), 1.92 (quin, J=6.0 Hz, 2H), 1.87-1.79 (m, 2H).

Step 2: To a solution of N-(7-chloro-2-(2,5-dimethyl-1H-pyrrol-1-yl)-1-methyl-1H-benzo[d]imidazol-4-yl)-5,6,7,8-tetrahydroquinolin-3-amine (60.0 mg, 148 μmol, 1.00 eq.) in ethyl alcohol (2.00 mL) was added hydrochloric acid (12.0 M, 0.20 mL, 16.2 eq.). The mixture was stirred at 120° C. for 1 hour in a microwave reactor. The mixture was concentrated. The residue was purified by prep-HPLC (HCl condition) and lyophilized to afford 7-chloro-1-methyl-N⁴-(5,6,7,8-tetrahydroquinolin-3-yl)-1H-benzo[d]imidazole-2,4-diamine hydrochloride, Example 2-4 (18.5 mg, 50.9 μmol, 34% yield) as a yellow solid. LCMS [ESI, M+1]: 328.0. ¹H NMR (400 MHZ, DMSO-d₆) δ=9.37 (br s, 1H), 8.89-8.26 (m, 2H), 8.06 (d, J=2.4 Hz, 1H), 7.65 (s, 1H), 7.25 (d, J=8.8 Hz, 1H), 7.14 (d, J=8.8 Hz, 1H), 3.87 (s, 3H), 2.93 (br t, J=6.0 Hz, 2H), 2.81 (br t, J=6.0 Hz, 2H), 1.88-1.79 (m, 2H), 1.79-1.70 (m, 2H).

### EXAMPLE 2-5

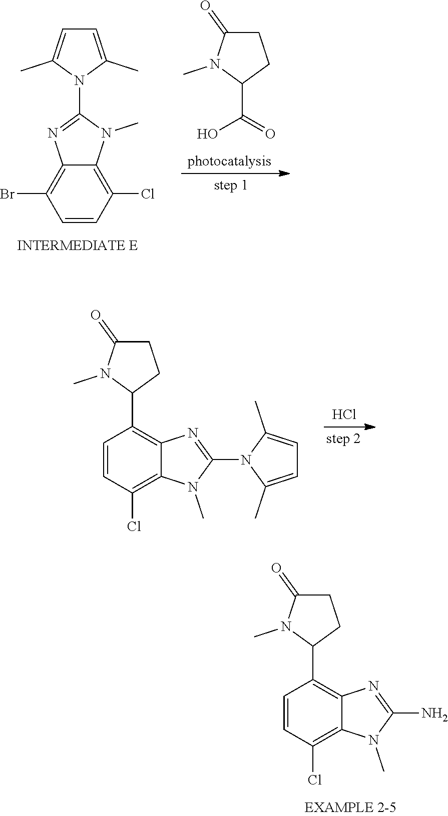

Step 1: A mixture of Intermediate E-1 (200 mg, 591 μmol, 1.00 eq.), 1-methyl-5-oxo-pyrrolidine-2-carboxylic acid (254 mg, 1.77 mmol, 3.00 eq.), Ir [dF (CF3) ppy]2 (dtbpy) (PF6) (6.63 mg, 5.91 μmol, 0.01 eq.), NiClz.dtbbpy (11.8 mg, 29.5 μmol, 0.05 eq.) and cesium carbonate (288 mg, 886 μmol, 1.50 eq.) in N,N-dimethylacetamide (5.00 mL) was degassed irradiated with two 34 W blue LED lamps at approximately 7 cm away from the light source and with cooling fan to keep the reaction temperature at 25° C. for 16 hours under nitrogen atmosphere. The reaction mixture was poured into water (50.0 mL), extracted with trichloromethane/isopropyl alcohol=3/1 (10.0 mL×4). The combined organic layers were dried over anhydrous sodium sulfate, filtered and concentrated. The residue was purified by flash silica gel chromatography (ethyl acetate in petroleum ether 0-80%) to afford 5-[7-chloro-2-(2,5-dimethylpyrrol-1-yl)-1-methyl-benzimidazol-4-yl]-1-methyl-pyrrolidin-2-one (60.0 mg, 155 μmol, 26% yield) as a yellow oil. LCMS [ESI, M+1]: 357.2. ¹H NMR (400 MHZ, CDCl₃) δ=7.26 (s, 1H), 6.95 (d, J=8.0 Hz, 1H), 5.92 (s, 2H), 5.35-5.33 (m, 1H), 3.70 (s, 3H), 2.73 (s, 3H), 2.64-2.49 (m, 2H), 2.47-2.35 (m, 1H), 2.34-2.14 (m, 1H), 2.02 (d, J=6.4 Hz, 6H).

Step 2: To a solution of 5-[7-chloro-2-(2,5-dimethylpyrrol-1-yl)-1-methyl-benzimidazol-4-yl]-1-methyl-pyrrolidin-2-one (30.0 mg, 84.1 μmol, 1.00 eq.) in ethyl alcohol (2.00 mL) was added concentrated hydrochloric acid (12.0 M, 0.20 mL, 28.5 eq.). The mixture was stirred at 120° C. for 0.5 hour in a microwave reactor. The reaction mixture was concentrated. The crude was purified by prep-HPLC (HCl condition) and lyophilized to afford 5-(2-amino-7-chloro-1-methyl-benzimidazol-4-yl)-1-methyl-pyrrolidin-2-one hydrochloride, Example 2-5 (5.35 mg, 18.9 μmol, 23% yield) as a white solid. LCMS [ESI, M+1]: 279.1. ¹H NMR (400 MHZ, MeOD) δ=7.36 (d, J=8.4 Hz, 1H), 7.09 (d, J=8.4 Hz, 1H), 5.06 (t, J=6.8 Hz, 1H), 4.00 (s, 3H), 2.70 (s, 3H), 2.67-2.54 (m, 2H), 2.54-2.43 (m, 1H), 2.01-1.86 (m, 1H).

### EXAMPLE 2-6

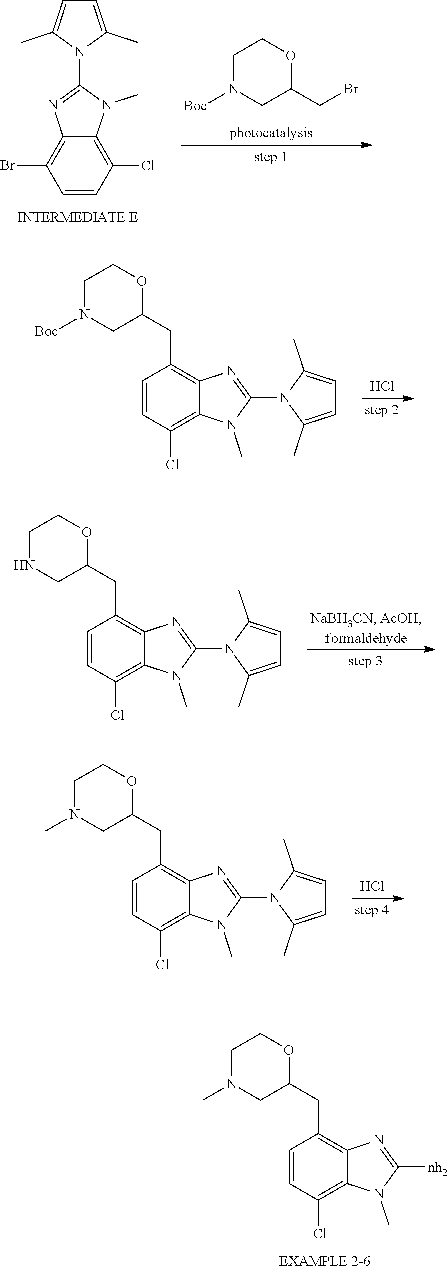

Step 1: To an 40 mL vial equipped with a stir bar was added tert-butyl 2-(bromomethyl) morpholine-4-carboxylate (827 mg, 2.95 mmol, 2.00 eq.), Intermediate E (500 mg, 1.48 mmol, 1.00 eq.), (4,4-Di-tert-butyl-2,2-bipyridine)bis[3,5-difluoro-2-[5-trifluoromethyl-2-pyridinyl-kappaN)phenyl-kappaC]iridium (III) Hexafluorophosphate (16.6 mg, 14.8 μmol, 0.01 eq.), [4,4′-Bis(1,1-dimethylethyl)-2,2′-bipyridine]nickel (II) dichloride (2.94 mg, 7.38 μmol, 0.005 eq), 1,1,1,3,3,3-hexamethyl-2-(trimethylsilyl)trisilane (367 mg, 1.48 mmol, 456 μL, 1.00 eq.), sodium carbonate (313 mg, 2.95 mmol, 2.00 eq.) and 1,2-dimethoxyethane (2.00 mL). The vial was sealed under nitrogen atmosphere. The reaction was stirred and irradiated with a 34 W blue LED lamp (7 cm away), with cooling fan to keep the reaction temperature at 25° C. for 14 hours. The mixture was concentrated in vacuo. The residue was purified by flash silica gel chromatography (Ethyl acetate in Petroleum ether 22%) to afford tert-butyl 2-[[7-chloro-2-(2,5-dimethylpyrrol-1-yl)-1-methyl-benzimidazol-4-yl]methyl]morpholine-4-carboxylate (500 mg, 796 μmol, 54% yield) as a yellow oil. LCMS [ESI, M+1]⁻: 459.2.

Step 2: To a solution of tert-butyl 2-[[7-chloro-2-(2,5-dimethylpyrrol-1-yl)-1-methyl-benzimidazol-4-yl]methyl]morpholine-4-carboxylate (60.0 mg, 95.5 μmol, 73.0% purity, 1.00 eq.) in methanol (0.50 mL) was added HCl/methanol (4 M, 0.50 mL, 21.0 eq.) at 10° C. The mixture was stirred at 10° C. for 1 hour before being concentrated in vacuo. The residue was diluted with methanol (1.00 mL) and basified with saturated sodium bicarbonate solid until pH 8. The mixture was filtered to give filtrate. The filtrate was concentrated in vacuo to afford 2-[[7-chloro-2-(2,5-dimethylpyrrol-1-yl)-1-methyl-benzimidazol-4-yl]methyl]morpholine (40.0 mg, 78.6 μmol, 82% yield) as a yellow oil. LCMS [ESI, M+1]⁺: 359.2.

Step 3: To a solution of 2-[[7-chloro-2-(2,5-dimethylpyrrol-1-yl)-1-methyl-benzimidazol-4-yl]methyl]morpholine (20.0 mg, 39.3 μmol, 70.5% purity, 1.00 eq.) in methanol (0.60 mL) was added sodium cyanoborohydride (3.70 mg, 58.9 μmol, 1.50 eq), acetic acid (5.90 mg, 98.2 μmol, 5.62 μL, 2.50 eq.) and formaldehyde (37% purity, 14.6 μL, 196 μmol, 5.00 eq.) at 10° C. The mixture was stirred at 10° C. for 16 hours. The mixture was concentrated in vacuum. The residue was purified by flash silica gel chromatography (methanol/ethyl acetate 8:1) to afford 2-[[7-chloro-2-(2,5-dimethylpyrrol-1-yl)-1-methyl-benzimidazol-4-yl]methyl]-4-methyl-morpholine (30.0 mg, 60.5 μmol, 77% yield) as a white solid. LCMS [ESI, M+1]⁻: 373.2.

Step 4: To a solution of 2-[[7-chloro-2-(2,5-dimethylpyrrol-1-yl)-1-methyl-benzimidazol-4-yl]methyl]-4-methyl-morpholine (30.0 mg, 80.5 μmol, 1.00 eq.) in ethyl alcohol (1.50 mL) was added hydrochloric acid (12 M, 0.15 mL, 22.4 eq.) at 10° C. The sealed tube was heated at 120° C. for 2 hours in a microwave reactor. The mixture was basified with ammonium hydroxide until pH 8. The resulting mixture was purified by Prep-HPLC (ammonium bicarbonate condition) to afford 7-chloro-1-methyl-4-[(4-methylmorpholin-2-yl)methyl]benzimidazol-2-amine, Example 2-6 (7.48 mg, 25.2 μmol, 42% yield) as an off-white solid. LCMS [ESI, M+1]⁺: 295.1. ¹H NMR (400 MHZ, MeOD-d₄) δ=6.91-6.82 (m, 2H), 3.93-3.82 (m, 4H), 3.60 (dt, J=2.0, 11.6 Hz, 1H), 3.04 (dd, J=6.8, 14.0 Hz, 1H), 2.94-2.85 (m, 1H), 2.72 (br dd, J=5.2, 11.2 Hz, 2H), 2.27 (s, 3H), 2.21 (dt, J=3.2, 11.6 Hz, 1H), 1.98 (t, J=10.8 Hz, 1H).

### EXAMPLE 2-7

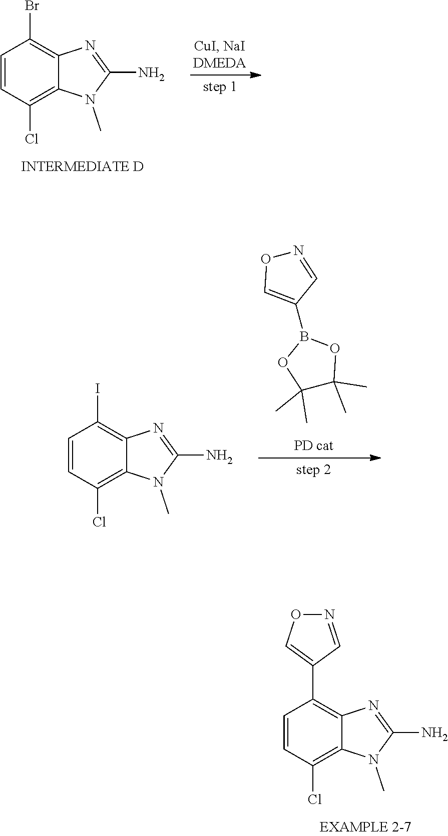 [SMILES: Cn1c(N)nc2c(Br)ccc(Cl)c21 ; Cn1c(N)nc2c(I)ccc(Cl)c21 ; CC1(C)OB(c2cnoc2)OC1(C)C ; Cn1c(N)nc2c(-c3cnoc3)ccc(Cl)c21]

Step 1: To a solution of 4-bromo-7-chloro-1-methyl-benzimidazol-2-amine (200 mg, 768 μmol, 1.00 eq.) and N,N-dimethylethane-1,2-diamine (40.6 mg, 460 μmol, 49.6 μL, 0.60 eq.) in dioxane (4.00 mL) was added copper iodide (43.9 mg, 230 μmol, 0.30 eq.) and sodium iodide (575 mg, 3.84 mmol, 5.00 eq.). The mixture was stirred at 120° C. for 16 hours. The reaction was poured into water (10.0 mL) and extracted with ethyl acetate (20.0 mL×3), the combined organic layers were concentrated in vacuum. . . . The residue was purified by silica gel chromatography (ethyl acetate in petroleum ether 0-100%) to give 7-chloro-4-iodo-1-methyl-benzimidazol-2-amine (180 mg, 585 μmol, 76% yield) as a yellow solid. ¹H NMR (400 MHZ, DMSO-d₆) δ=7.29 (d, J=8.4 Hz, 1H), 6.93 (s, 2H), 6.69 (d, J=8.4 Hz, 1H), 3.75 (s, 3H).

Step 2: To a solution of 7-chloro-4-iodo-1-methyl-benzimidazol-2-amine (80.0 mg, 260 μmol, 1.00 eq.) and 4-(4,4,5,5-tetramethyl-1,3,2-dioxaborolan-2-yl) isoxazole (254 mg, 1.30 mmol, 5.00 eq.) in dioxane (1.00 mL) was added Pd(dppf)C₁2 (25.4 mg, 39.0 μmol, 0.15 eq.) and potassium phosphate (166 mg, 780 μmol, 3.00 eq.). The mixture was stirred at 40° C. for 2 hours before being filted through celite and concentrated in vacuum. The residue was purified by silica gel chromatography (methanol in ethyl acetate 0-100%) then further re-purified by prep-TLC (100% ethyl acetate) to give 7-chloro-4-isoxazol-4-yl-1-methyl-benzimidazol-2-amine, Example 2-7 (4.38 mg, 16.8 μmol, 6% yield) as a yellow gum. LCMS [ESI, M+1]⁺=249. ¹H NMR (400 MHZ, MeOD-d₄) δ=9.19 (s, 1H), 8.87 (s, 1H), 7.36 (s, 1H), 7.34-7.30 (m, 1H), 3.99 (s, 3H).

### EXAMPLE 2-8

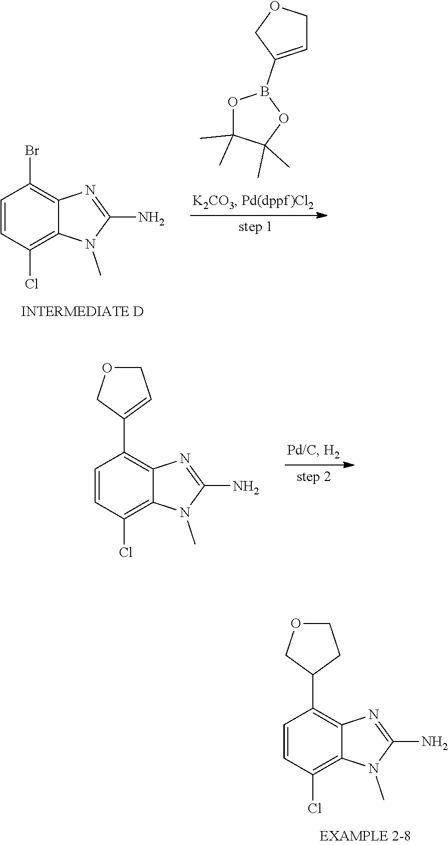 [SMILES: Cn1c(N)nc2c(Br)ccc(Cl)c21 ; CC1(C)OB(C2=CCOC2)OC1(C)C ; Cn1c(N)nc2c(C3=CCOC3)ccc(Cl)c21 ; Cn1c(N)nc2c(C3CCOC3)ccc(Cl)c21]

Step 1: A mixture of Intermediate D (180 mg, 690. μmol, 1.00 eq.), 2-(2,5-dihydrofuran-3-yl)-4,4,5,5-tetramethyl-1,3,2-dioxaborolane (203 mg, 1.04 mmol, 1.50 eq.), [1,1′-bis(diphenylphosphino) ferrocene]dichloropalladium (II) dichloromethane (75.8 mg, 103 μmol, 0.15 eq.), potassium carbonate (286 mg, 2.07 mmol, 3.00 eq.) in dioxane (0.50 mL) and water (0.10 mL) was degassed and stirred at 90° C. for 3 hours under nitrogen atmosphere. The mixture was poured into water (10.0 mL) and extracted with dichloromethane (50.0 mL×3). The combined organic layer was concentrated under vacuum. The crude product was purified by Prep-HPLC (TFA condition) to give 7-chloro-4-(2,5-dihydrofuran-3-yl)-1-methyl-benzimidazol-2-amine trifluoroacetate (60.0 mg, 240 μmol, 35% yield) as a yellow solid. LCMS [ESI, M+1]: 250.0.

Step 2: To a solution of 7-chloro-4-(2,5-dihydrofuran-3-yl)-1-methyl-benzimidazol-2-amine (40.0 mg, 160.2 μmol, 1.00 eq.) in ethyl acetate (1.00 mL) was added palladium on carbon (10.0%, 10.0 mg) and the resulting mixture was stirred under hydrogen atmosphere (1 atm) for 10 min before being filtered. The filtrate was concentrated under vacuum to give a residue. The crude product was purified by prep-HPLC (HCl condition) and lyophilized to afford 7-chloro-1-methyl-4-tetrahydrofuran-3-yl-benzimidazol-2-amine hydrochloride, Example 2-8 (8.01 mg, 30.6 μmol, 19% yield) as an off-white solid. LCMS [ESI, M+1]: 252.0. 1H NMR (400 MHZ, MeOD) δ=7.35-7.08 (m, 2H), 5.06-4.92 (m, 1H), 4.17-4.05 (m, 2H), 4.01-3.96 (m, 3H), 3.93-3.87 (m, 1H), 3.80-3.67 (m, 1H), 2.57-2.38 (m, 1H), 2.11-1.91 (m, 1H).

### EXAMPLE 2-9

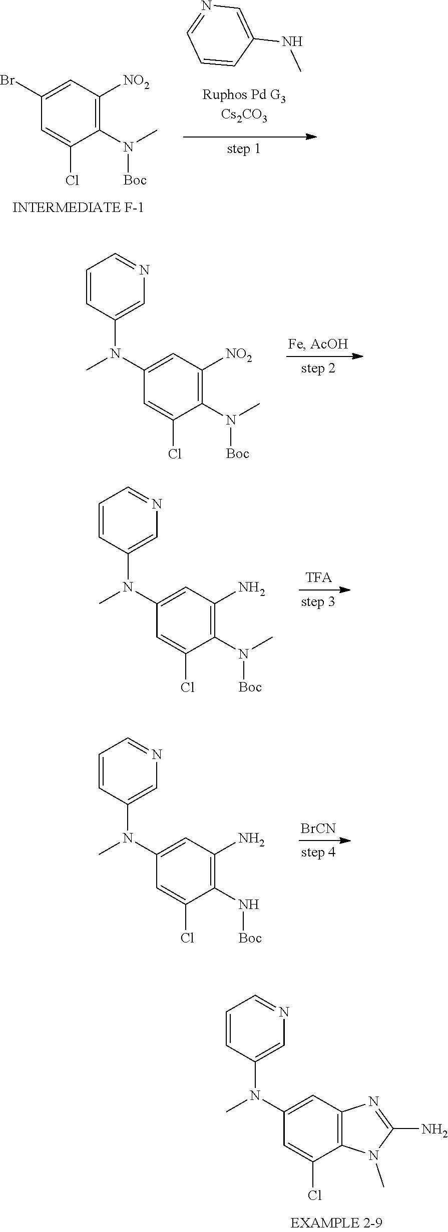

Step 1: A mixture of Intermediate F-1 (400 mg, 1.09 mmol, 1.00 eq.), N-methylpyridin-3-amine (118 mg, 1.09 mmol, 1.00 eq.), methanesulfonato (2-dicyclohexylphosphino-2,6-di-i-propoxy-1,1-biphenyl) (2-amino-1,1-biphenyl-2-yl) palladium (II) (45.8 mg, 54.7 μmol, 0.05 eq.) and cesium carbonate (1.07 g, 3.28 mmol, 3.00 eq.) in toluene (5.00 mL) was degassed and stirred at 90° C. for 5 hours under nitrogen atmosphere. After being cooled to room temperature, the mixture was concentrated under vacuum. The residue was diluted with ethyl acetate (20.0 mL) and water (20.0 mL). The layers were separated and the aqueous phase was extracted with ethyl acetate (3×20.0 mL). The combined organic layers were dried over anhydrous sodium sulfate, filtered and concentrated under vacuum. The residue was purified by silica gel chromatography (ethyl acetate in petroleum ether 0-50%) to give tert-butyl N-[2-chloro-4-[methyl (3-pyridyl)amino]-6-nitro-phenyl]-N-methyl-carbamate (300 mg, 725 μmol, 66% yield) as a yellow oil. ¹H NMR (400 MHz, CDCl₃-d) δ=8.54-8.49 (m, 2H), 7.57-7.51 (m, 1H), 7.42-7.36 (m, 1H), 7.15 (d, J=2.8 Hz, 1H), 7.03 (d, J=2.8 Hz, 1H), 3.39 (s, 3H), 3.15 (s, 3H), 1.31 (s, 9H).

Step 2: To a solution of tert-butyl N-[2-chloro-4-[methyl (3-pyridyl)amino]-6-nitro-phenyl]-N-methyl-carbamate (150 mg, 381 μmol, 1.00 eq.) in ethyl acetate (5.00 mL) was added iron powder (128 mg, 2.29 mmol, 6.00 eq.), acetic acid (1.00 mL) and water (0.10 mL).

The mixture was stirred at 60° C. for 3 hours. The resulting mixture was filtered over celite, and the filtrate was concentrated under reduced pressure. The residue was partitioned between ethyl acetate (30.0 mL) and 1 N aqueous sodium hydroxide (30. 0 mL). The organic phase was separated and the aqueus layer extracted with ethyl acetate (3×20.0 mL). The combined organic layers were dried over anhydrous sodium sulfate, filtered and concentrated under vacuum to give tert-butyl N-[2-amino-6-chloro-4-[methyl (3-pyridyl)amino]phenyl]-N-methyl-carbamate (150 mg, crude) as a yellow oil. ¹H NMR (400 MHZ, CDCl₃-d) δ=8.40-8.35 (m, 1H), 8.27-8.19 (m, 1H), 7.39-7.33 (m, 1H), 7.25-7.18 (m, 1H), 6.42 (d, J=2.4 Hz, 1H), 6.23 (d, J=2.4 Hz, 1H), 3.83 (s, 2H), 3.29 (s, 3H), 3.09 (s, 3H), 1.38 (s, 9H).

Step 3: To a solution of tert-butyl N-[2-amino-6-chloro-4-[methyl (3-pyridyl)amino]phenyl]-N-methyl-carbamate (150 mg, 413 μmol, 1.00 eq.) in dichloromethane (10.0 mL) was added trifluoroacetic acid (1.54 g, 13.5 mmol, 1.00 mL, 32.7 eq.). The mixture was stirred at 15° C. for 1 hour. The reaction mixture was diluted with dichloromethane (10.0 mL) and very slowly added to a saturated aqueous sodium bicarbonate (20.0 mL). The mixture was extracted with dichloromethane (3×20.0 mL). The organic layer was dried over anhydrous sodium sulfate, filtered and concentrated under vacuum to give 6-chloro-N1,N4-dimethyl-N4-(3-pyridyl)benzene-1,2,4-triamine (150 mg, crude) as a yellow oil. ¹H NMR (400 MHZ, CDCl₃-d) 8=8.19 (d, J=2.4 Hz, 1H), 8.04 (d, J=4.6 Hz, 1H), 7.16-7.00 (m, 2H), 6.44 (d, J=2.4 Hz, 1H), 6.29 (d, J=2.4 Hz, 1H), 4.12-3.77 (m, 2H), 3.25 (s, 3H), 2.62 (s, 3H).

Step 4: To a solution of 6-chloro-N1, N4-dimethyl-N4-(3-pyridyl)benzene-1, 2, 4-triamine (150 mg, 571 μmol, 1.00 eq.) in ethanol (5.00 mL) was added cyanogen bromide (121 mg, 1.14 mmol, 2.00 eq.). The mixture was stirred at 15° C. for 3 hours before being concentrated under vacuum. The residue was purified by prep-HPLC (HCl condition) and then further re-purified by prep-HPLC (ammonium bicarbonate condition) to give 7-chloro-N5-1-dimethyl-N5-(3-pyridyl)benzimidazole-2,5-diamine hydrochloride, Example 2-9 (6.29 mg, 21.64 μmol, 4% yield) as a white solid. LCMS [M+1]⁺=287.9. ¹H NMR (400 MHZ, MeOD-d₄) δ=7.97 (d, J=2.4 Hz, 1H), 7.89 (dd, J=1.6, 4.0 Hz, 1H), 7.24-7.15 (m, 2H), 6.98 (d, J=2.0 Hz, 1H), 6.80 (d, J=2.0 Hz, 1H), 3.87 (s, 3H), 3.30 (s, 3H).

### EXAMPLE 2-10

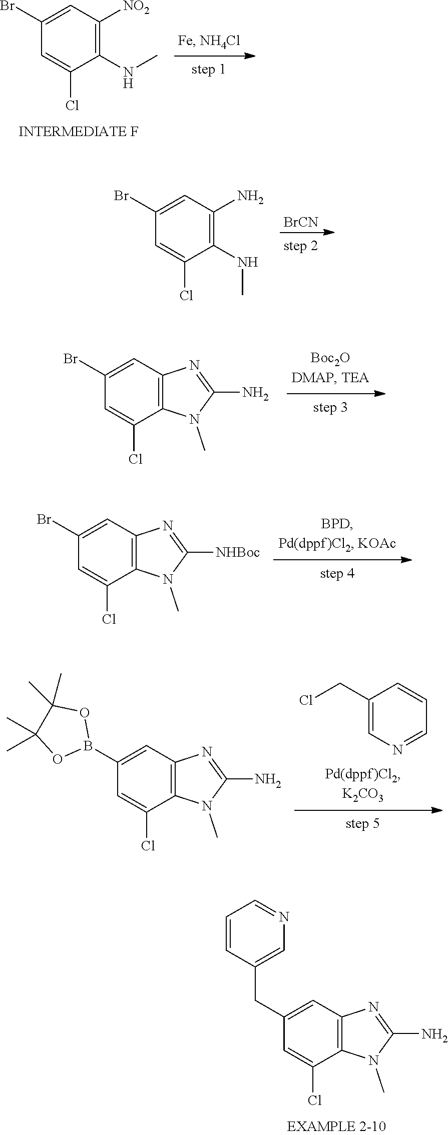

Step 1: A mixture of Intermediate F (1.00 g, 3.77 mmol, 1.00 eq.), iron powder (1.26 g, 22.6 mmol, 6.00 eq.), acetic acid (5.00 mL) and water (0.50 mL) in ethyl acetate (15.0 mL) was stirred at 70° C. for 2 hours. The resulting mixture was filtered over celite, and the filtrate was concentrated under reduced pressure. The residue was partitioned between ethyl acetate (30.0 mL) and 1 N aqueous sodium hydroxide (30.0 mL). The organic phase was separated and the aqueous phase was extracted with ethyl acetate (3×20.0 mL). The combined organic layers were dried over sodium sulfate, filtered and concentrated under vacuum to get 5-bromo-3-chloro-N2-methyl-benzene-1,2-diamine (1 g, crude) as a yellow oil. LCMS [M+3]⁺: 237.0. ¹H NMR (400 MHZ, CDCl₃-d) δ=6.74 (d, J=2.0, 1H), 6.70 (d, J=2.0, 1H), 5.32 (s, 2H), 3.88 (q, J=5.6 Hz, 1H), 2.58 (d, J=5.6 Hz, 3H).

Step 2: To a solution of 5-bromo-3-chloro-N2-methyl-benzene-1,2-diamine (1.20 g, 5.10 mmol, 1.00 eq.) in ethanol (15.0 mL) was added cyanogen bromide (811 mg, 7.66 mmol, 1.50 eq.). The mixture was stirred at 15° C. for 6 hours. The mixture was concentrated under vacuum to get 5-bromo-7-chloro-1-methyl-benzimidazol-2-amine (1.5 g, crude) as a yellow solid. LCMS [M+3]⁺: 261.9. ¹H NMR (400 MHZ, CDCl₃-d) δ=8.70 (s, 2H), 7.50 (s, 2H), 3.82 (s, 3H).

Step 3: To a solution of 5-bromo-7-chloro-1-methyl-benzimidazol-2-amine (500 mg, 1.92 mmol, 1.00 eq.) in tetrahydrofuran (20.0 mL) was added N,N-dimethylpyridin-4-amine (46.9 mg, 384 μmol, 0.20 eq.), triethylamine (971 mg, 9.60 mmol, 1.34 mL, 5 eq.) and di-tert-butyl dicarbonate (1.26 g, 5.76 mmol, 1.32 mL, 3.00 eq.). The mixture was stirred at 40° C. for 12 hours. The residue was diluted with ethyl acetate (10.0 mL) and water (10.0 mL). The layers were separated and the aqueous phase was extracted with ethyl acetate (3×10.0 mL). The combined organic layers were dried over anhydrous sodium sulfate, filtered and concentrated under vacuum. The residue was purified by silica gel column chromatography (ethyl acetate in petroleum ether 10-20%) to to get tert-butyl N-(5-bromo-7-chloro-1-methyl-benzimidazol-2-yl) carbamate (530 mg, 1.47 mmol, 77% yield) as a white solid. LCMS [M+3]⁺: 362.0. ¹H NMR (400 MHZ, DMSO-d₆) δ=12.34-9.94 (m, 1H), 7.75-7.35 (m, 2H), 3.86-3.64 (m, 3H), 1.48-1.46 (m, 9H).

Step 4: To a solution of tert-butyl N-(5-bromo-7-chloro-1-methyl-benzimidazol-2-yl) carbamate (300 mg, 832 μmol, 1.00 eq.) and 4,4,4′,4′,5,5,5′,5′-octamethyl-2,2′-bi (1,3,2-dioxaborolane) (253 mg, 998 μmol, 1.20 eq.) in dioxane (5.00 mL) were added Pd (dppf) C₁₂ (60.9 mg, 83.2 μmol, 0.10 eq.) and potassium acetate (245 mg, 2.50 mmol, 3.00 eq.). The mixture was degassed and stirred at 90° C. for 14 hours. The mixture was quenched with saturated aqueous ammonium chloride (5.00 mL) and then extracted with ethyl acetate (20.0 mL). The organic layer was dried over anhydrous sodium sulfate, filtered and concentrated under vacuum to produce 7-chloro-1-ethyl-5-(4,4,5,5-tetramethyl-1,3,2-dioxaborolan-2-yl)benzimidazol-2-amine (350 mg, crude) as a brown gum. LCMS [M+1]⁺: 308.3.

Step 5: To a mixture of 3-(chloromethyl)pyridine (70.5 mg, 553 μmol, 1.00 eq.), 7-chloro-1-methyl-5-(4,4,5,5-tetramethyl-1,3,2-dioxaborolan-2-yl)benzimidazol-2-amine (170 mg, 553 μmol, 1.00 eq.), Pd(dppf)Cl₂ (40.4 mg, 55.3 μmol, 0.10 eq.) and potassium carbonate (229 mg, 1.66 mmol, 3.00 eq.) in dioxane (5.00 mL) and water (1.00 mL) was degassed and attired at 100° C. for 4 hours. The reaction mixture was diluted with ethyl acetate (30.0 mL) and water (30.0 mL). The organic layer was separated, dried over sodium sulfate and then concentrated under vacuum. The residue was purified by silica gel column chromatography (methanol in dichloromethane 3-10%) to get the crude product (28 mg, 63% purity) that was further re-purified by prep-HPLC (HCl condition) to afford 7-chloro-1-methyl-5-(3-pyridylmethyl)benzimidazol-2-amine hydrochloride, Example 2-10 (10.7 mg, 33.8 μmol, 33% yield) as a yellow solid. LCMS [M+3]+: 273.2. ¹H NMR (400 MHZ, DMSO-d₆) δ=8.89 (s, 2H), 8.87 (s, 1H), 8.35 (d, J=8.0 Hz, 1H), 7.89 (dd, J=5.6, 8.0 Hz, 1H), 7.35 (s, 1H), 7.33 (s, 1H), 4.23 (s, 2H), 3.83 (s, 3H).

### EXAMPLE 2-11

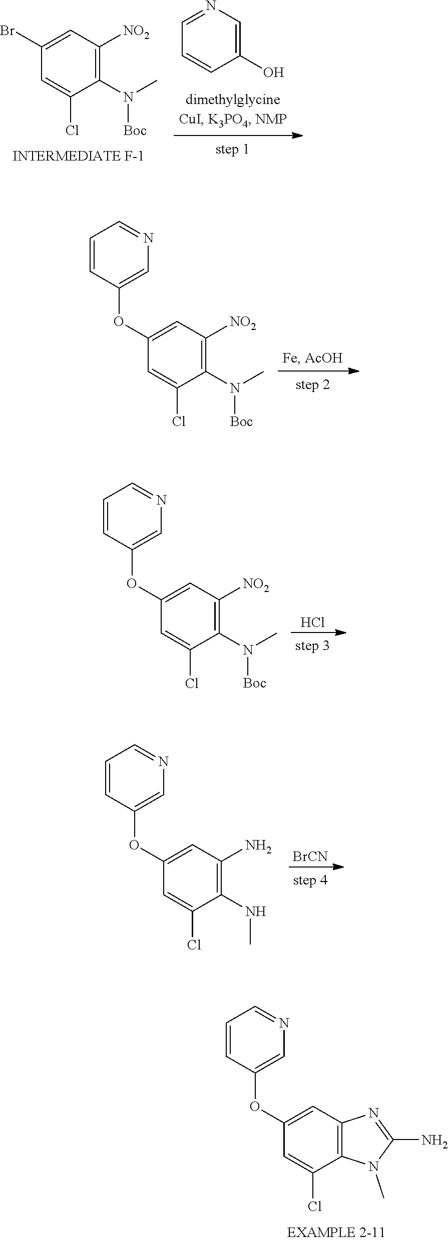

Step 1: A mixture of Intermediate F-1 (600 mg, 1.64 mmol, 1.00 eq.), pyridin-3-ol (312.14 mg, 3.28 mmol, 2.00 eq.), copper iodide (31.3 mg, 164 μmol, 0.10 eq.), potassium phosphate (1.05 g, 4.92 mmol, 3.00 eq.) and 2-(dimethylamino) acetic acid (16.9 mg, 164 μmol, 0.10 eq.) in N-methyl pyrrolidone (10.0 mL) was degassed and stirred at 120° C. for 4 hours under nitrogen atmosphere. After being cooled to room temperature, the mixture was concentrated under vacuum. The residue was diluted with ethyl acetate (20.0 mL) and water (20.0 mL). The layers were separated and the aqueous phase was extracted with ethyl acetate (3×20.0 mL). The organic layer was dried over anhydrous sodium sulfate, filtered and concentrated under vacuum. The residue was purified by column chromatography on silica gel (ethyl acetate in petroleum ether 30-50%) to to get tert-butyl N-[2-chloro-6-nitro-4-(3-pyridyloxy)phenyl]-N-methyl-carbamate (100 mg, 263.3 μmol, 16% yield) as a yellow oil. 1H NMR (400 MHZ, CDCl₃-d)=8.57-8.46 (m, 2H), 7.48-7.36 (m, 3H), 7.32-7.28 (m, 1H), 3.18 (s, 3H), 1.32 (s, 9H).

Step 2: To a solution of tert-butyl N-[2-chloro-6-nitro-4-(3-pyridyloxy)phenyl]-N-methyl-carbamate (80.0 mg, 211 μmol, 1.00 eq.) in ethyl acetate (5.00 mL) was added iron powder (70.0 mg, 1.25 mmol, 5.95 eq.), acetic acid (1.00 mL) and water (0.10 mL). The mixture was stirred at 60° C. for 5 hours. The resulting mixture was filtered over celite, and the filtrate was concentrated under reduced pressure. The filtrate was partitioned between ethyl acetate (30.0 mL) and 1 N aqueous sodium hydroxide (30.0 mL). The layers were separated and the aqueous phase was extracted with ethyl acetate (3×20.0 mL). The combined organic layers were dried over anhydrous sodium sulfate, filtered and concentrated under vacuum to obtain tert-butyl N-[2-amino-6-chloro-4-(3-pyridyloxy)phenyl]-N-methyl-carbamate (100 mg, crude) as a yellow oil.

¹H NMR (400 MHZ, CDCl₃-d) δ=8.41 (s, 2H), 7.37-7.29 (m, 2H), 6.43 (d, J=2.4 Hz, 1H), 6.28 (d, J=2.8 Hz, 1H), 4.06-3.86 (m, 2H), 3.09 (s, 3H), 1.38 (s, 9H).

Step 3: To a solution of tert-butyl N-[2-amino-6-chloro-4-(3-pyridyloxy)phenyl]-N-methyl-carbamate (100 mg, 286 μmol, 1.00 eq.) in dioxane (2.00 mL) was added HCl in dioxane (4 M, 2.00 mL, 27.9 eq.). The mixture was stirred at 15° C. for 1 hour. The mixture was concentrated under vacuum to get 3-chloro-N2-methyl-5-(3-pyridyloxy)benzene-1,2-diamine (100 mg, crude) as a yellow oil. LCMS [M+1]⁺=250.0.

Step 4: To a solution of 3-chloro-N2-methyl-5-(3-pyridyloxy)benzene-1,2-diamine (50.0 mg, 200 μmol, 1.00 eq.) in ethanol (2.00 mL) was added cyanogen bromide (29.5 μL, 400 μmol, 2.00 eq.) and triethylamine (40.5 mg, 400 μmol, 55.7 μL, 2.00 eq.). The mixture was stirred at 15° C. for 2 hours. After being cooled to room temperature, the mixture was concentrated under vacuum. The residue was purified by prep-HPLC (HCl condition). The desired fraction was collected, lyophilized and further re-purified by prep-HPLC (ammonium bicarbonate condition) to produce 7-chloro-1-methyl-5-(3-pyridyloxy)benzimidazol-2-amine, Example 2-11 (6 mg, 21.4 μmol, 11% yield) as a white solid. LCMS [M+1]⁺=275.0. ¹H NMR (400 MHZ, MeOD-d₄) δ=8.30-8.22 (m, 2H), 7.44-7.36 (m, 2H), 6.85 (d, J=2.0 Hz, 1H), 6.73 (d, J=2.0 Hz, 1H), 3.86 (s, 3H).

### EXAMPLE 2-12

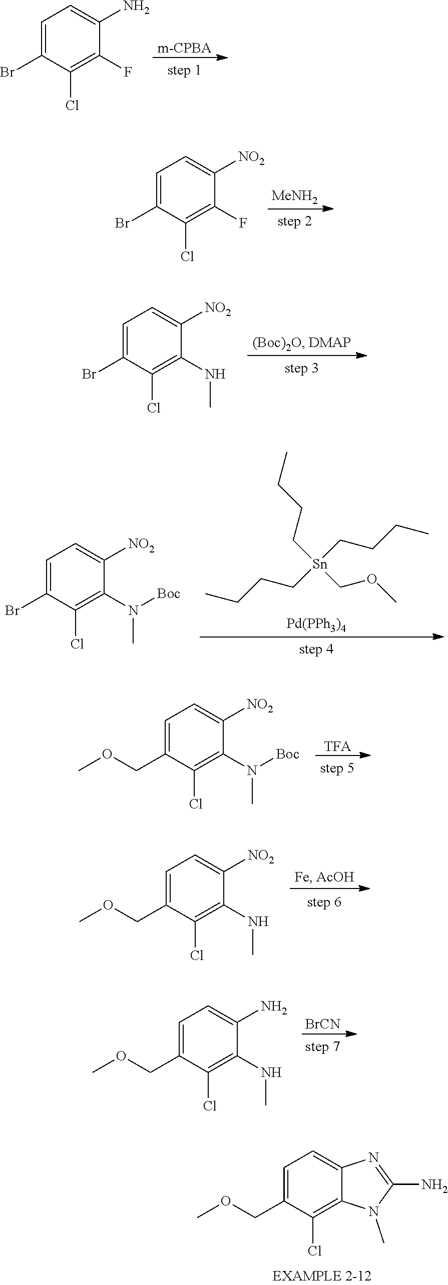

Step 1: To a solution of m-chloroperoxybenzoic acid (36.2 g, 178 mmol, 85.0% purity, 4.00 eq.) in dichloromethane (300 mL) was added a solution of 4-bromo-3-chloro-2-fluoro-aniline (10.0 g, 44.6 mmol, 1.00 eq.) in dichloromethane (100 mL). The reaction was stirred at 85° C. for 10 hours. The mixture was quenched by addition a solution of saturated sodium sulfite (200 mL). The aqueous phase was extracted with dichloromethane (150 mL×3). The combined organic phase was washed with brine (150 mL), dried with anhydrous sodium sulfate, filtered and concentrated in vacuum to produce 1-bromo-2-chloro-3-fluoro-4-nitro-benzene (10.5 g, crude) as a yellow solid. ¹H NMR (400 MHZ, DMSO-d₆) δ=8.10 (dd, J=7.8, 8.8 Hz, 1H), 7.95-7.86 (m, 1H).

Step 2: To a solution of 1-bromo-2-chloro-3-fluoro-4-nitro-benzene (5.00 g, 19.7 mmol, 1.00 eq.) in ethanol (50.0 mL) was added methanamine in ethanol (33.0%, 41.5 mL, 333 mmol, 17.0 eq.). The reaction was stirred at 20° C. for 12 hours. The reaction mixture was diluted with water (30.0 mL) and stirred for 5 min. The aqueous phase was extracted with ethyl acetate (40.0 mL×3). The combined organic phase was dried over anhydrous sodium sulfate, filtered and concentrated in vacuum. The residue was purified by silica gel column chromatography (100% dichloromethane) to get 3-bromo-2-chloro-N-methyl-6-nitro-aniline (5.00 g, 18.8 mmol, 96% yield) as a yellow solid. ¹H NMR (400 MHZ, DMSO-d₆) δ=7.67 (d, J=8.8 Hz, 1H), 7.15-7.08 (m, 1H), 6.93 (br s, 1H), 2.71 (d, J=5.2 Hz, 3H).

Step 3: To a solution of 3-bromo-2-chloro-N-methyl-6-nitro-aniline (1.50 g, 5.65 mmol, 1.00 eq.) in tetrahydrofuran (30.0 mL) was added di-tert-butyl dicarbonate (2.47 g, 11.3 mmol, 2.60 mL, 2.00 eq.), N,N-dimethylpyridin-4-amine (69.0 mg, 565 μmol, 0.10 eq.) and triethylamine (1.14 g, 11.3 mmol, 1.57 mL, 2.00 eq.). The reaction was stirred at 50° C. for 2 hours. The reaction mixture was concentrated under reduced pressure. The crude was purified by silica gel column chromatography (ethyl acetate in petroleum ether 1-2%). The residue was re-purified by prep-HPLC (formic acid condition) to get tert-butyl N-(3-bromo-2-chloro-6-nitro-phenyl)-N-methyl-carbamate (1.60 g, crude) as a yellow solid. ¹H NMR (400 MHZ, CDCl₃) δ=7.76-7.73 (m, 1H), 7.72-7.68 (m, 1H), 3.20 (s, 3H), 1.31 (s, 9H).

Step 4: To a solution of tributyl(methoxymethyl) stannane (289 mg, 862 μmol, 1.50 eq.) in dioxane (4.00 mL) was added tert-butyl N-(3-bromo-2-chloro-6-nitro-phenyl)-N-methyl-carbamate (210 mg, 574 μmol, 1.00 eq.), lithium chloride (73.1 mg, 1.72 mmol, 3.00 eq.) and tetrakis(triphenylphosphine) palladium (0) (66.4 mg, 57.4 μmol, 0.10 eq.). The mixture was purged with nitrogen and stirred at 100° C. for 12 hours under nitrogen atmosphere. The reaction mixture was quenched by addition a solution of potassium fluoride (30.0 mL), and then diluted with ethyl acetate (20.0 mL). The aqueous phase was extracted with ethyl acetate (20.0 mL×3). The combined organic phase was washed with brine (30.0 mL), dried with anhydrous sodium sulfate, filtered and concentrated in vacuum. The residue was purified by prep-TLC (SiO₂, petroleum ether/ethyl acetate 10:1) to get tert-butyl N-[2-chloro-3-(methoxymethyl)-6-nitro-phenyl]-N-methyl-carbamate (157 mg, 456 μmol, 79% yield) as a yellow oil. ¹H NMR (400 MHZ, CDCl₃) δ=7.94-7.83 (m, 1H), 7.63 (d, J=8.4 Hz, 1H), 4.66-4.53 (m, 2H), 3.57-3.51 (m, 3H), 3.21-3.17 (m, 3H), 1.30 (s, 9H).

Step 5: To a solution of tert-butyl N-[2-chloro-3-(methoxymethyl)-6-nitro-phenyl]-N-methyl-carbamate (50.0 mg, 151 μmol, 1.00 eq.) in dichloromethane (1.00 mL) was added trifluoroacetic acid (770 mg, 6.75 mmol, 0.50 mL, 44.7 eq.). The reaction was stirred at 18° C. for 1 hour before being concentrated under reduced pressure. Then water (20.0 mL) was added and the mixture was neutralized to pH=7˜8 with aqueous sodium bicarbonate. The aqueous phase was extracted with ethyl acetate (20.0 mL×3). The combined organic phase was washed with brine (20.0 mL), dried with anhydrous sodium sulfate, filtered and concentrated in vacuum to obtains 2-chloro-3-(methoxymethyl)-N-methyl-6-nitro-aniline (47.0 mg, crude) as a yellow oil. ¹H NMR (400 MHZ, CDCl₃) δ=7.93 (d, J=8.8 Hz, 1H), 7.00 (d, J=8.8 Hz, 1H), 6.58 (br s, 1H), 4.55 (s, 2H), 3.53-3.48 (m, 3H), 3.11-3.03 (m, 3H).

Step 6: To a solution of 2-chloro-3-(methoxymethyl)-N-methyl-6-nitro-aniline (47.0 mg, 204 μmol, 1.00 eq.) in ethyl acetate (1.50 mL) was added iron powder (68.3 mg, 1.22 mmol, 6.00 eq.), water (0.10 mL) and acetic acid (0.50 mL). The reaction was stirred at 70° C. for 2 hours. The reaction mixture was diluted with ethyl acetate (20.0 mL) and filtered. The filtrate was extracted with ethyl acetate (30.0 mL×3). The combined organic phase was washed with brine (40.0 mL), dried with anhydrous sodium sulfate, filtered and concentrated in vacuum to get 3-chloro-4-(methoxymethyl)-N2-methyl-benzene-1,2-diamine (48.0 mg, crude) as a yellow oil.

¹H NMR (400 MHZ, CDCl₃) δ=6.95 (d, J=8.4 Hz, 1H), 6.62 (d, J=8.4 Hz, 1H), 6.65-6.59 (m, 1H), 4.5 (s, 2H), 3.93 (br s, 2H), 3.40 (s, 3H), 2.70 (s, 3H).

Step 7: To a solution of 3-chloro-4-(methoxymethyl)-N2-methyl-benzene-1, 2-diamine (40.0 mg, 127 μmol, 1.00 eq.) in ethyl alcohol (2.00 mL) was added cyanogen bromide (26.9 mg, 254 μmol, 2.00 eq.). The reaction was stirred at 20° C. for 6 hours. The reaction mixture was concentrated under reduced pressure and the crude product was purified by prep-HPLC (HCl condition) to produce 7-chloro-6-(methoxymethyl)-1-methyl-benzimidazol-2-amine hydrochloride, Example 2-12 (6.38 mg, 27.1 μmol, 21% yield) as a white solid.

LCMS [M+1]⁺: 226.3. ¹H NMR (400 MHZ, DMSO-d₆) δ=8.77 (s, 2H), 7.35 (s, 2H), 4.54 (s, 2H), 3.87 (s, 3H), 3.33-3.33 (m, 3H).

### EXAMPLE 3-1

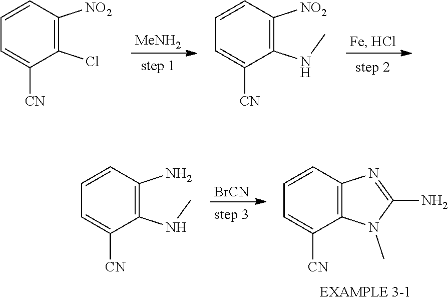

Step 1: To a solution of 2-chloro-3-nitro-benzonitrile (250 mg, 1.37 mmol, 1.00 eq.) in ethanol (2.50 mL) was added methylamine in ethanol (42.5 mg, 1.37 mmol, 2.5 M, 10.0 eq.). The reaction mixture was stirred at 15° C. for 12 hours. The mixture was concentrated under vacuum. The residue was diluted with water (50.0 mL). The layers were separated and the aqueous phase was extracted with ethyl acetate (60.0 mL). The organic layer was dried over sodium sulfate, filtered and concentrated in vacuum to produce 2-(methylamino)-3-nitro-benzonitrile (240 mg, crude) as a yellow solid. ¹H NMR (400 MHZ, CDCl₃) δ=8.51 (s, 1H), 8.39 (d, J=8.8 Hz, 1H), 7.76 (d, J=7.6 Hz, 1H), 6.72 (t, J=8.0 Hz, 1H), 3.49 (d, J=5.2 Hz, 3H).

Step 2: To a solution of 2-(methylamino)-3-nitro-benzonitrile (240 mg, 1.35 mmol, 1.00 eq.) in water (3.00 mL) was added iron powder (378 mg, 6.77 mmol, 5.00 eq.) and hydrochloric acid (6.00 M, 903 μL, 4.00 eq.). The reaction mixture was stirred at 15° C. for 12 hours. The mixture was concentrated under vacuum. The residue was diluted with water (50.0 mL) and extracted with ethyl acetate (100 mL). The organic layer was dried over sodium sulfate, filtered and concentrated in vacuum to get 3-amino-2-(methylamino)benzonitrile (140 mg, 951 μmol, 70% yield) as a black solid. ¹H NMR (400 MHZ, CDCl₃) δ=6.99 (dd, J=1.6, 7.6 Hz, 1H), 6.91-6.85 (m, 1H), 6.84-6.77 (m, 1H), 4.06-3.42 (m, 2H), 3.32-3.08 (m, 1H), 3.01 (s, 3H).

Step 3: To a solution of 3-amino-2-(methylamino)benzonitrile (140 mg, 951 μmol, 1.00 eq.) in ethanol (3.00 mL) was added cyanogen bromide (202 mg, 1.90 mmol, 2.00 eq.). The reaction mixture was stirred at 15° C. for 2 hours. The mixture was concentrated under vacuum and the residue was diluted with water (50.0 mL) and extracted with ethyl acetate (100 mL). The organic layer was dried over sodium sulfate, filtered and concentrated in vacuum. The residue was purified by prep-HPLC (HCl condition) to afford 2-amino-3-methyl-1,2-dihydrobenzimidazole-4-carbonitrile hydrochloride, Example 3-1 (30.0 mg, 171 μmol, 18% yield) as a white solid.

LCMS [M+3]+: 173.0. ¹H NMR (400 MHZ, DMSO-d₆) δ=13.26 (s, 1H), 9.07 (s, 2H), 7.78-7.60 (m, 2H), 7.39 (t, J=8.0 Hz, 1H), 3.85 (s, 3H).

### GENERAL COUPLING METHODS FOR THE PREPARATION OF EXAMPLES 3-2 to 3-10

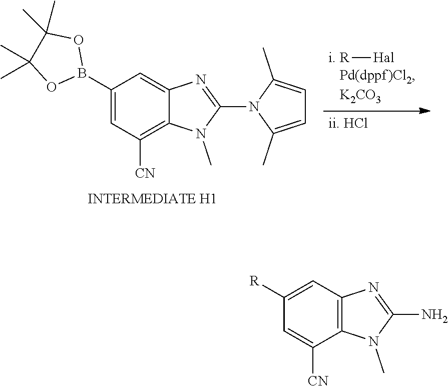

Coupling Method 3-A (CM3-A): To a solution of Intermediate H-1 (120 mg, 319 μmol, 1.00 eq.) and R-Hal (Hal=Cl or I) (478 μmol, 1.50 eq.) in N,N-dimethylformamide (2.00 mL) was added potassium carbonate (132 mg, 957 μmol, 3.00 eq.), followed by [1,1′-bis(diphenylphosphino) ferrocene]dichloropalladium (II) (35.0 mg, 47.8 μmol, 0.15 eq.) at 15° C. The mixture was stirred at 100° C. for 16 hours. The mixture was concentrated in vacuum. The residue was purified by flash silica gel chromatography to afford an intermediate product. To a solution of this intermediate product (174 μmol, 1.00 eq.) in ethanol (1.00 mL) was added hydrochloric acid (12 M, 0.10 mL, 6.89 eq.). The mixture was stirred at 120° C. for 1.5 hours in a microwave reactor. The mixture was basified with ammonium hydroxide until pH 8 and concentrated in vacuum. The residue was purified by Prep-HPLC to give the desired product.

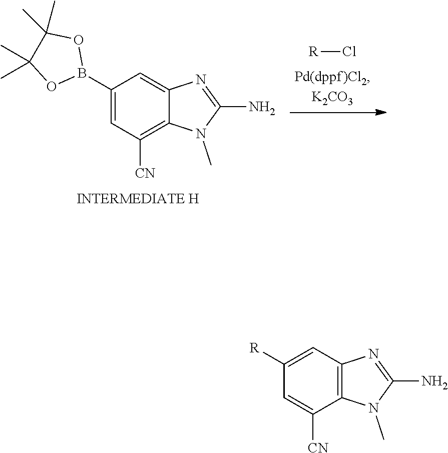

Coupling Method 3-B (CM3-B): A mixture of Intermediate H (100 mg, 335 μmol, 1.00 eq.), R-Cl (402 μmol, 1.20 eq.), potassium carbonate (139 mg, 1.01 mmol, 3.00 eq.), [1,1-bis(diphenylphosphino) ferrocene]dichloropalladium (II) (24.5 mg, 33.5 μmol, 0.10 eq.) and water (0.2 mL) in dioxane (2.00 mL) was degassed stirred at 100° C. for 4 hours under nitrogen atmosphere. After being cooled to room temperature, the mixture was concentrated in vacuum. The residue was purified by prep-HPLC and/or prep-TLC (dichloromethane/methanol 8:1) to give the product.

Following the teachings of the herein provided reaction schemes, the coupling methods CM3-A and CM3-B and the Intermediates disclosed herein, Examples 3-2 to 3-10 were prepared as shown in Table 2, or using similar methods:

TABLE 2

| | | Coupling | Yield, | Compound Name and |
|---|---|---|---|---|
| Example | Structure | Method | % | Characterization |
| 3-2 | 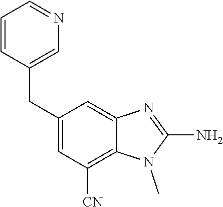 [SMILES: Cn1c(N)nc2cc(Cc3cccnc3)cc(C#N)c21] | CM3-B | 9 | 2-amino-3-methyl-6-(3- pyridylmethyl)benzimidazole-4- carbonitrile. LCMS [M + 1] ⁺: = 264.2. ¹H NMR (400 MHz, MeOD-d₄) δ = 8.47 (d, J = 2.0 Hz, 1H), 8.43 - 8.37 (m, 1H), 7.72 (d, J = 7.6 Hz, 1H), 7.38 (dd, J = 4.8, 7.6 Hz, 1H), 7.33 (s, 1H), 7.20 (s, 1H), 4.10 (s, 2H), 3.84 (s, 3H). |
| 3-3 | 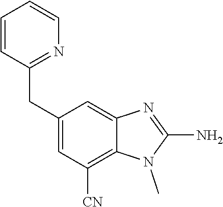 [SMILES: Cn1c(N)nc2cc(Cc3ccccn3)cc(C#N)c21] | CM3-B | 2 | 2-amino-3-methyl-6-(2- pyridylmethyl)benzimidazole-4- carbonitrile. LCMS [M + 1] ⁺ = 264.3. ¹H NMR (400 MHz, DMSO-d₆). δ = 8.46 (d, J = 4.8 Hz, 1H), 7.77 (dt, J = 1.6, 7.6 Hz, 1H), 7.35 − 7.31 (m, 2H), 7.27 (dd, J = 5.2, 7.6 Hz, 1H), 7.20 (s, 1H), 4.18 (s, 2H), 3.82 (s, 3H). |
| 3-4 | 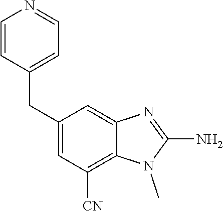 [SMILES: Cn1c(N)nc2cc(Cc3ccncc3)cc(C#N)c21] | CM3-A | 7 | 2-amino-3-methyl-6-(4- pyridylmethyl)benzimidazole-4- carbonitrile. LCMS [M + 1] ⁺: 264.2. ¹H NMR (400 MHz, MeOD-d₄) δ = 8.77 (d, J = 6.8 Hz, 2H), 7.97 (d, J = 6.4 Hz, 2H), 7.77 − 7.52 (m, 2H), 4.49 (s, 2H), 3.94 (s, 3H). |
| 3-5 | 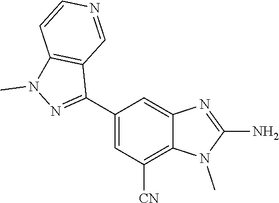 [SMILES: Cn1nc(-c2cc(C#N)c3c(c2)nc(N)n3C)c2cnccc21] | CM3-A | 9 | 2-amino-3-methyl-6-(1- methylpyrazolo[4,3-c]pyridin-3- yl)benzimidazole-4-carbonitrile. LCMS [ESI, M + 1] ⁺: 304.1. ¹H NMR (400 MHz, DMSO-d₆) δ = 9.40 (s, 1H), 8.43 (d, J = 6.0 Hz, 1H), 7.97 (d, J = 1.6 Hz, 1H), 7.85 (d, J = 1.2 Hz, 1H), 7.70 (d, J = 6.0 Hz, 1H), 7.04 (s, 2H), 4.11 (s, 3H), 3.81 (s, 3H). |
| 3-6 | 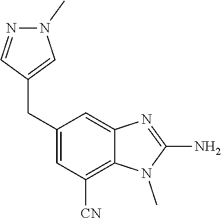 [SMILES: Cn1cc(Cc2cc(C#N)c3c(c2)nc(N)n3C)cn1] | CM3-A | 11 | 2-amino-3-methyl-6-[ (1- methylpyrazol-4- yl)methyl]benzimidazole-4- carbonitrile. LCMS [ESI, M + 1] ⁺: 267.1; ¹H NMR (400 MHz, MeOD-d₄) δ = 7.38 (s, 1H), 7.33 − 7.26 (m, 2H), 7.11 (d, J = 1.56 Hz, 1H), 3.84 (s, 2H), 3.82 (s, 3H), 3.79 (s, 3H). |
| 3-7 | 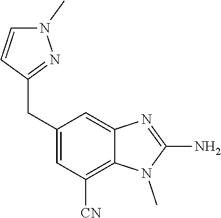 [SMILES: Cn1ccc(Cc2cc(C#N)c3c(c2)nc(N)n3C)n1] | CM3-A | 15 | 2-amino-3-methyl-6-[ (1- methylpyrazol-3- yl)methyl]benzimidazole-4- carbonitrile. LCMS [ESI, M + 1] ⁺: 267.1. ¹H NMR (400 MHz, MeOD-d₄) δ = 7.47 (d, J = 2.0 Hz, 1H), 7.32 (s, 1H), 7.14 (s, 1H), 6.04 (d, J = 2.0 Hz, 1H), 3.97 (s, 2H), 3.84 (s, 3H), 3.81 (s, 3H). |
| 3-8 | 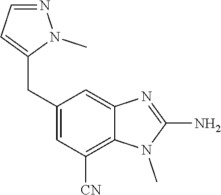 [SMILES: Cn1nccc1Cc1cc(C#N)c2c(c1)nc(N)n2C] | CM3-A | 14 | 2-amino-3-methyl-6-[ (2- methylpyrazol-3- yl)methyl]benzimidazole-4- carbonitrile. LCMS [ESI, M + 1] ⁺: 267.1. ¹H NMR (400 MHz, MeOD-d₄) δ = 7.37 (d, J = 2.0 Hz, 1H), 7.27 (s, 1H), 7.11 (s, 1H), 6.06 (d, J = 1.6 Hz, 1H), 4.10 (s, 2H), 3.82 (S, 3H), 3.71 (s, 3H). |
| 3-9 | 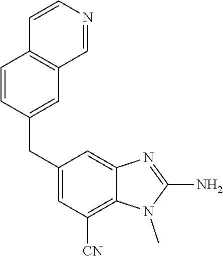 [SMILES: Cn1c(N)nc2cc(Cc3ccc4ccncc4c3)cc(C#N)c21] | CM3-A | 25 | 2-amino-6-(7- isoquinolylmethyl)-3-methyl- benzimidazole-4-carbonitrile. LCMS [M + 1] ⁺ = 314.3 ¹H NMR (400 MHz, MeOH-d₄) δ = 9.73 (s, 1H), 8.56 (d, J = 6.4 Hz, 1H), 8.46 (d, J = 6.4 Hz, 1H), 8.39 (s, 1H), 8.27 (d, J = 8.4 Hz, 1H), 8.14 (d, J = 8.4 Hz, 1H), 7.68 (s, 1H), 7.64 (s, 1H), 4.46 (s, 2H), 3.93 (s, 3H). |
| 3-10 | 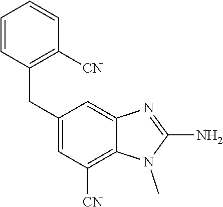 [SMILES: Cn1c(N)nc2cc(Cc3ccccc3C#N)cc(C#N)c21] | CM3-A | 44 | 2-amino-6-[ (2- cyanophenyl)methyl]-3-methyl- benzimidazole-4-carbonitrile. LC-MS [M + 1] ⁺ = 288.2. ¹H NMR (400 MHz, MeOH-d₄) δ = 7.73 (dd, J = 1.1, 7.6 Hz, 1H), 7.69 − 7.62 (m, 1H), 7.57 − 7.49 (m, 3H), 7.45 (dt, J = 1.2, 7.6 Hz, 1H), 4.34 (s, 2H), 3.92 (s, 3H). |

### EXAMPLE 3-11

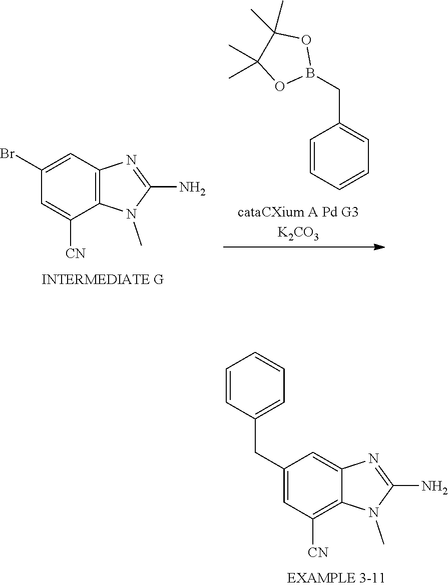 [SMILES: Cn1c(N)nc2cc(Br)cc(C#N)c21 ; CC1(C)OB(Cc2ccccc2)OC1(C)C ; Cn1c(N)nc2cc(Cc3ccccc3)cc(C#N)c21]

A mixture of Intermediate G (100 mg, 398 μmol, 1.00 eq.), 2-benzyl-4,4,5,5-tetramethyl-1,3,2-dioxaborolane (174 mg, 797 μmol, 2.00 eq.), cataCXium A Pd G3 (29.0 mg, 39.8 μmol, 0.10 eq.) and potassium carbonate (165 mg, 1.19 mmol, 3.00 eq.) and in dioxane (3.00 mL) and water (0.60 mL) was degassed and stirred at 90° C. for 12 hours under nitrogen atmosphere. The reaction mixture was diluted with water (20.0 mL) and extracted with ethyl acetate (30.0 mL). The combined organic layers were washed with brine (20.0 mL), dried over anhydrous sodium sulfate, filtered and concentrated under reduced pressure to get a residue. The residue was purified by prep-HPLC (HCl condition) to get 2-amino-5-benzyl-1-methyl-1H-benzo[d]imidazole-7-carbonitrile hydrochloride, Example 3-11 (11.2 mg, 37.5 μmol, 9% yield) as a white solid. LCMS [M+1]⁺=263.1. ¹H NMR (400 MHZ, DMSO-d₆) δ=13.12 (br s, 1H), 8.92 (br s, 2H), 7.61 (s, 1H), 7.50 (s, 1H), 7.34-7.23 (m, 4H), 7.23-7.16 (m, 1H), 4.06 (s, 2H), 3.80 (s, 3H).

### EXAMPLE 3-12

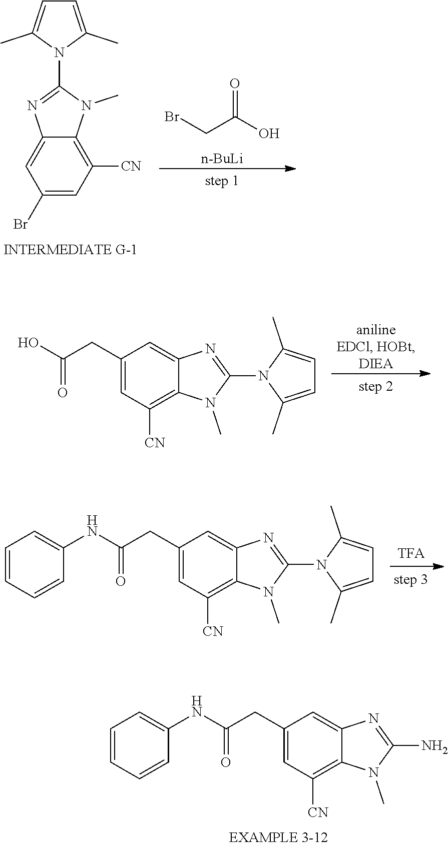

Step 1: To a mixture of Intermediate G1 (300 mg, 911 μmol, 1.00 eq.) in tetrahydrofuran (4.00 mL) at −60° C. under nitrogen atmosphere was added n-butyllithium (2.50 M, 729 μL, 2.00 eq.). The mixture was stirred at −60° C. for 0.5 hour. Then 2-bromoacetic acid (506 mg, 3.65 mmol, 262 μL, 4.00 eq.) in tetrahydrofuran (1.00 mL) was added and the resulting mixture was warmed up to 30° C. and stirred for an additional 0.5 hour. The reaction mixture was quenched with water (1.00 mL) and purified by Prep-HPLC (formic acid condition) to afford 2-[7-cyano-2-(2,5-dimethylpyrrol-1-yl)-1-methyl-benzimidazol-5-yl]acetic acid (35.0 mg, 106 μmol, 12% yield) as a white solid. LCMS [ESI, M+1]: 309.2. ¹H NMR (400 MHZ, MeOD-d₄) δ=7.95 (d, J=1.2 Hz, 1H), 7.75 (d, J=1.2 Hz, 1H), 5.98 (s, 2H), 3.83 (s, 2H), 3.77 (s, 3H), 2.04 (s, 6H).

Step 2: To a mixture of 2-[7-cyano-2-(2,5-dimethylpyrrol-1-yl)-1-methyl-benzimidazol-5-yl]acetic acid (35.0 mg, 114 μmol, 1.00 eq.) and aniline (31.1 μL, 340 μmol, 3.00 eq.) in dimethyl formamide (1.00 mL) was added N-(3-dimethylaminopropyl)-N-ethylcarbodiimide hydrochloride (32.6 mg, 170 μmol, 1.50 eq.), 1-hydroxybenzotriazole (23.0 mg, 170 μmol, 1.50 eq.) and N,N-diisopropylethylamine (44.0 mg, 340 μmol, 59.3 μL, 3.00 eq.). The mixture was stirred at 30° C. for 16 hours. The reaction mixture was concentrated and purified by Prep-TLC (SiO₂, petroleum ether/ethyl acetate 3:1) to afford 2-[7-cyano-2-(2,5-dimethylpyrrol-1-yl)-1-methyl-benzimidazol-5-yl]-N-phenyl-acetamide (40.0 mg, 100 μmol, 89% yield) as a yellow oil. LCMS [ESI, M+1]: 384.3. ¹H NMR (400 MHZ, CDCl₃) δ=8.01 (d, J=1.2 Hz, 1H), 7.71 (d, J=1.2 Hz, 1H), 7.48 (d, J=7.6 Hz, 2H), 7.33-7.29 (m, 3H), 7.17-7.09 (m, 1H), 5.98 (s, 2H), 3.87 (s, 2H), 3.74 (s, 3H), 2.05 (s, 6H).

Step 3: A mixture of 2-[7-cyano-2-(2,5-dimethylpyrrol-1-yl)-1-methyl-benzimidazol-5-yl]-N-phenyl-acetamide (30.0 mg, 78.2 μmol, 1.00 eq.) in trifluoroacetic acid (2.00 mL) was stirred at 80° C. for 16 hours. The mixture was concentrated. The crude was purified by Prep-HPLC (HCl condition) to afford 2-(2-amino-7-cyano-1-methyl-benzimidazol-5-yl)-N-phenyl-acetamide hydrochloride, Example 3-12 (7.52 mg, 21.4 μmol, 27% yield) as a light green solid. LCMS [ESI, M+1]: 306.2. ¹H NMR (400 MHZ, MeOD) δ=7.66 (dd, J=1.2, 14.8 Hz, 2H), 7.55 (dd, J=1.2, 8.8 Hz, 2H), 7.35-7.25 (m, 2H), 7.13-7.05 (m, 1H), 3.93 (s, 3H), 3.84 (s, 2H).

### EXAMPLE 3-13

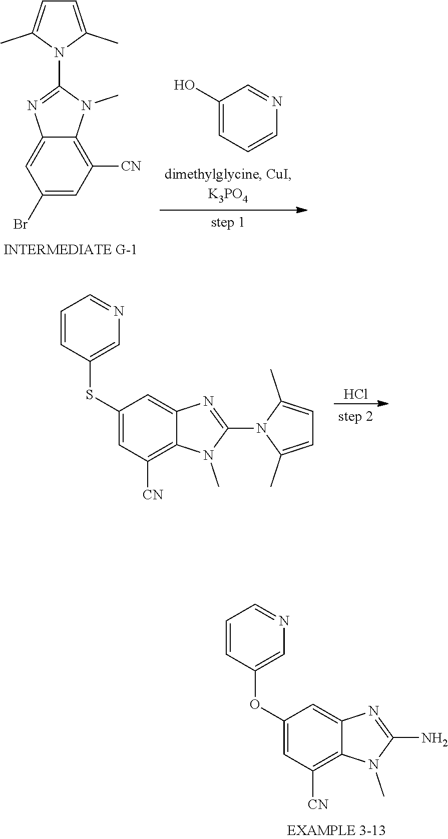 [SMILES: Cc1ccc(C)n1-c1nc2cc(Br)cc(C#N)c2n1C ; Oc1cccnc1 ; Cc1ccc(C)n1-c1nc2cc(Sc3cccnc3)cc(C#N)c2n1C ; Cl ; Cn1c(N)nc2cc(Oc3cccnc3)cc(C#N)c21]

Step 1: To a solution of Intermediate G-1 (150 mg, 455 μmol, 1.00 eq.)pyridin-3-ol (130 mg, 1.37 mmol, 3.00 eq.) and pyridin-3-ol (130 mg, 1.37 mmol, 3.00 eq.) and 2-(dimethylamino) acetic acid (18.8 mg, 182 μmol, 0.40 eq.) in dimethylsulfoxide (3.00 mL) was added copper iodide (17.4 mg, 91.1 μmol, 0.20 eq.) and potassium phosphate (290 mg, 1.37 mmol, 3.00 eq.). The mixture was stirred at 120° C. for 16 hrs. The reaction was poured into water (20.0 mL) and extracted with ethyl acetate (50.0 mL×3), the combined organic layers were concentrated under vacuum. The residue was purified by silica gel chromatography (ethyl acetate in petroleum ether gradient 0-100%) to give 2-(2,5-dimethylpyrrol-1-yl)-3-methyl-6-(3-pyridyloxy)benzimidazole-4-carbonitrile (100 mg, 291 μmol, 64% yield) as a yellow solid. LCMS [ESI, M+1]: 344.2

Step 2: To a solution of 2-(2,5-dimethylpyrrol-1-yl)-3-methyl-6-(3-pyridyloxy)benzimidazole-4-carbonitrile (50.0 mg, 146 μmol, 1.00 eq.) in ethanol (1.50 mL) was added hydrochloric acid (12.0 M, 375 μL, 30.9 eq.). The mixture was stirred at 120° C. for 2 hrs in a microwave reactor. The solution was treated with ammonium hydroxide (0.10 mL) to adjust pH to 7. The mixture was concentrated under vacuum. The crude product was purified by prep-HPLC (NH₄HCO₃ condition) to give 2-amino-3-methyl-6-(3-pyridyloxy)benzimidazole-4-carbonitrile, Example 3-13 (8.28 mg, 30.6 μmol, 21% yield) as a white solid. LCMS [ESI, M+1]: 266.0. ¹H NMR (400 MHZ, MeOD-d₄) δ=8.32-8.24 (m, 2H), 7.41 (d, J=1.6, 3.2 Hz, 2H), 7.16 (d, J=2.4 Hz, 1H), 7.05 (d, J=2.4 Hz, 1H), 3.85 (s, 3H).

### EXAMPLE 3-14

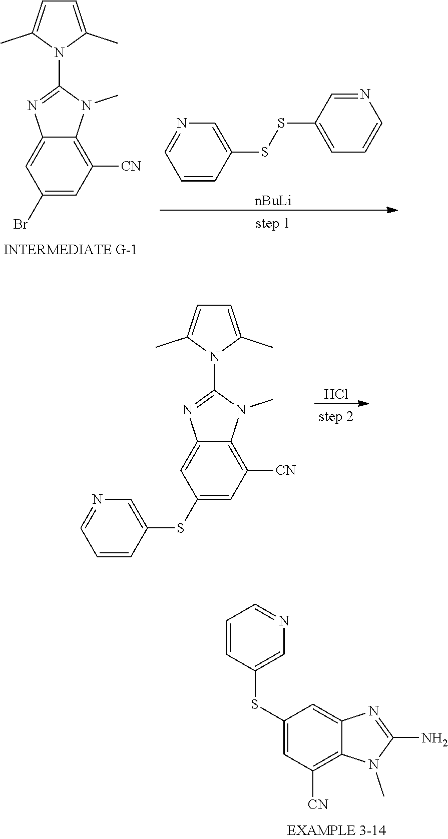

Step 1: To a solution of Intermediate G-1 (100 mg, 303 μmol, 1.00 eq.) in tetrahydrofuran (2.00 mL) was added n-butyllithium (2.50 M, 145 μL, 1.20 eq.) at −65° C. The mixture was stirred at −65° C. for 0.5 h. A solution of 3,3′-dithiodipridine (80.3 mg, 364 μmol, 1.20 eq.) in tetrahydrofuran (0.50 mL) was added at −65° C. The mixture was stirred at −65° C. for 0.5 h, then warmed up slowly to 25° C. and stirred for an additional 0.5 h. The mixture was diluted with saturated ammonium chloride (50.0 mL), extracted with ethyl acetate (50.0 mL), dried over sodium sulfate, filtered and concentrated. The residue was purified by silica gel chromatography with (ethyl acetate in petroleum ether 0-50%) to afford 2-(2,5-dimethylpyrrol-1-yl)-3-methyl-6-(3-pyridylsulfanyl)benzimidazole-4-carbonitrile (37.0 mg, 85.4 μmol, 28% yield) as a yellow gum. LCMS [ESI, M+1]⁺=360.

Step 2: To a solution of 2-(2,5-dimethylpyrrol-1-yl)-3-methyl-6-(3-pyridylsulfanyl)benzimidazole-4-carbonitrile (32.0 mg, 89.0 μmol, 1.00 eq.) in ethyl alcohol (0.50 mL) was added 12M hydrochloric acid (0.05 mL, 503 μmol, 5.60 eq.). The mixture was stirred at 120° C. in a microwave reactor for 1 h and then concentrated. The residue was purified by prep-HPLC (HCl condition) to afford 2-amino-3-methyl-6-(3-pyridylsulfanyl)benzimidazole-4-carbonitrile hydrochloride, Example 3-14 (13.9 mg, 43.1 μmol, 48% yield) as a white solid. LCMS [ESI, M+1]⁺=282. ¹H NMR (400 MHZ, MeOD) δ=8.72 (s, 1H), 8.69 (br d, J=5.2 Hz, 1H), 8.37 (br d, J=8.0 Hz, 1H), 7.98 (s, 2H), 7.94 (s, 1H), 3.99 (s, 3H).

### EXAMPLE 3-15

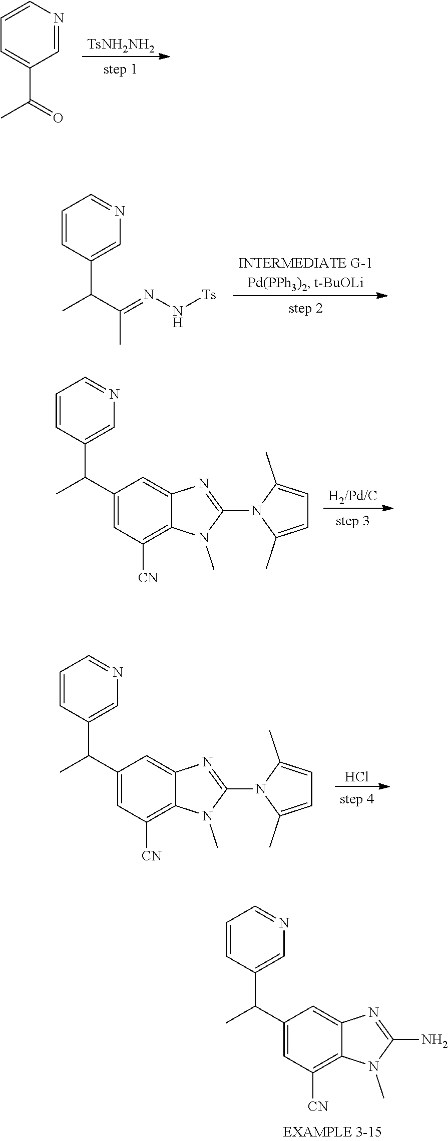

Step 1: A mixture of 1-(3-pyridyl) ethanone (1.30 g, 10.7 mmol, 1.18 mL, 1.00 eq.) and 4-methylbenzenesulfonohydrazide (2.00 g, 10.7 mmol, 1.00 eq.) in methanol (20.0 mL) was heated to 60° C. for 14 hours. The reaction mixture was filtered and the solid was washed with methanol (20.0 mL). The precipitate was dried in vacuum to produce 4-methyl-N—[(E)-1-(3-pyridyl)ethylideneamino]benzenesulfonamide (2.00 g, 6.91 mmol, 64% yield) as a white solid.

¹H NMR (400 MHZ, DMSO-d₆) δ=10.69 (s, 1H), 8.77 (d, J=1.6 Hz, 1H), 8.58-8.51 (m, 1H), 8.02-7.94 (m, 1H), 7.81 (d, J=8.0 Hz, 2H), 7.48-7.35 (m, 3H), 2.37 (s, 3H), 2.20 (s, 3H).

Step 2: To a mixture of 4-methyl-N-[(E)-1-(3-pyridyl)ethylideneamino]benzene-sulfonamide (65.9 mg, 228 μmol, 1.50 eq.), Intermediate G-1 (50.0 mg, 152 μmol, 1.00 eq.) in dioxane (3.00 mL) was added bis(triphenylphosphine) palladium (II) chloride (10.7 mg, 15.2 μmol, 0.10 eq.). The reaction mixture was degassed and heated to 100° C. Then tert-butyl alcohol lithium (48.6 mg, 606 μmol, 54.8 μL, 4.00 eq.) was added and the reaction mixture was heated to 100° C. for 2 hours. Water (30.0 mL) was added and the mixture was extracted with ethyl acetate (20.0 mL×2). The combined organic layers were dried over sodium sulfate and then concentrated under vacuum. The residue was purified by silica gel column chromatography (petroleum ether/ethyl acetate 2:1) to give 2-(2,5-dimethylpyrrol-1-yl)-3-methyl-6-[1-(3-pyridyl) vinyl]benzimid-azole-4-carbonitrile (35.0 mg, 99.0 μmol, 65% yield) as a brown solid. ¹H NMR (400 MHZ, CDCl₃-d) δ=8.68 (s, 2H), 8.12-8.02 (m, 1H), 7.95 (d, J=1.6 Hz, 1H), 7.74-7.63 (m, 1H), 7.62-7.59 (m, 1H), 6.01 (s, 2H), 5.79 (d, J=7.6 Hz, 2H), 3.78 (s, 3H), 2.09 (s, 6H).

Step 3: To a solution of 2-(2,5-dimethylpyrrol-1-yl)-3-methyl-6-[1-(3-pyridyl) vinyl]benzimidazole-4-carbonitrile (35.0 mg, 99.0 μmol, 1.00 eq.) in ethyl acetate (5.00 mL) was added palladium on carbon (10%, 35.0 mg) at 20° C. Then the mixture was degassed and charged with hydrogen for three times and then stirred under hydrogen 1 atm) at 20° C. for 2 hours. The mixture was filtered and the filtrate was concentrated in vacuum to give 2-(2,5-dimethylpyrrol-1-yl)-3-methyl-6-[1-(3-pyridyl)ethyl]benzimidazole-4-carbonitrile (34.0 mg, crude) as a white solid. LCMS [M+1]⁺: 356.3.

Step 4: To a solution of 2-(2,5-dimethylpyrrol-1-yl)-3-methyl-6-[1-(3-pyridyl)ethyl]benzimidazole-4-carbonitrile (30.0 mg, 84.4 μmol, 1.00 eq.) in ethanol (1.50 mL) was added hydrochloric acid (12.0 M, 0.15 mL, 21.3 eq.) at 20° C. The sealed tube was heated at 120° C. for 1.5 hours in a microwave reactor. The mixture was basified with sodium bicarbonate to pH 8. The resulting mixture was concentrated in vacuum to remove ethanol. The residue was purified by prep-HPLC (HCl condition) to give 2-amino-3-methyl-6-[1-(3-pyridyl)ethyl]benzimidazole-4-carbonitrile hydrochloride, Example 3-15 (14.1 mg, 44.2 μmol, 52% yield) as an off-white solid. LCMS [M+1]⁺: 278.2. ¹H NMR (400 MHZ, MeOD-d₄) δ=8.88 (s, 1H), 8.76 (d, J=6.0 Hz, 1H), 8.59 (d, J=8.0 Hz, 1H), 8.06 (dd, J=6.0, 8.0 Hz, 1H), 7.70 (d, J=1.2 Hz, 1H), 7.66 (d, J=1.2 Hz, 1H), 4.68 (d, J=7.2 Hz, 1H), 3.93 (s, 3H), 1.81 (d, J=7.2 Hz, 3H).

### EXAMPLES 3-16 and 3-17

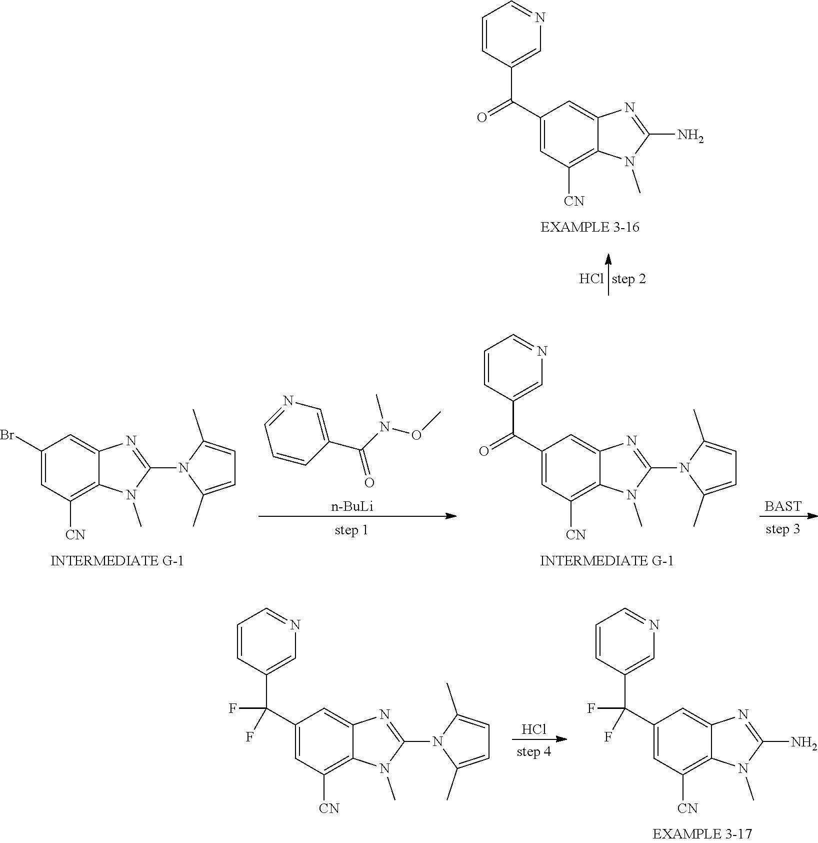

Step 1: To a solution of Intermediate G-1 (75.0 mg, 228 μmol, 1.00 eq.) and N-methoxy-N-methyl-pyridine-3-carboxamide (41.7 mg, 251 μmol, 1.10 eq.) in tetrahydrofuran (1.00 mL) was added n-butyllithium (2.5 M, 182 μL, 2.00 eq.) slowly at −70° C. under nitrogen atmosphere. The mixture was stirred at −70° C. for 1 hour. The mixture was slowly poured into saturated ammonium chloride solution (5.00 mL) and extracted with ethyl acetate (3.00 mL×3). The combined organic layers were washed with brine (5.00 mL), dried over anhydrous sodium sulfate, filtered and concentrated in vacuum. The residue was purified by flash silica gel chromatography (Ethyl acetate/Petroleum ether 4:3) to afford 2-(2,5-dimethylpyrrol-1-yl)-3-methyl-6-(pyridine-3-carbonyl)benzimidazole-4-carbonitrile (20.0 mg, 50.5 μmol, 22% yield) as a yellow oil. LCMS [ESI, M+1]⁻: 356.1

Step 2: To a solution of 2-(2,5-dimethylpyrrol-1-yl)-3-methyl-6-(pyridine-3-carbonyl)benzimidazole-4-carbonitrile (20.0 mg, 50.5 μmol, 1.00 eq.) in ethanol (1.00 mL) was added hydrochloric acid (12 M, 0.10 mL, 23.8 eq.) at 10° C. The mixture was stirred at 120° C. for 1.5 hours in a microwave reactor. The mixture was basified with ammonium hydroxide until pH 8. The resulting mixture was concentrated in vacuum to remove ethanol. The residue was purified by prep-HPLC (ammonium bicarbonate condition) to afford 2-amino-3-methyl-6-(pyridine-3-carbonyl)benzimidazole-4-carbonitrile, Example 3-16 (6.08 mg, 21.8 μmol, 43% yield) as a yellow solid. LCMS [ESI, M+1]⁺: 278.0. ¹H NMR (400 MHZ, DMSO-d₆) δ=8.86 (d, J=2.0 Hz, 1H), 8.83 (dd, J=1.2, 4.8 Hz, 1H), 8.11 (td, J=1.6, 8.0 Hz, 1H), 7.73 (d, J=1.2 Hz, 1H), 7.66 (d, J=1.2 Hz, 1H), 7.60 (dd, J=4.8, 7.6 Hz, 1H), 7.20 (s, 2H), 3.82 (s, 3H).

Step 3: To a solution of 2-(2,5-dimethylpyrrol-1-yl)-3-methyl-6-(pyridine-3-carbonyl)benzimidazole-4-carbonitrile (130 mg, 366 μmol, 1.00 eq.) in dichloromethane (2.00 mL) was added bis(2-methoxyethyl)aminosulfur trifluoride (809 mg, 3.66 mmol, 801 μL, 10.0 eq.) at 0° C. under nitrogen atmosphere. The mixture was stirred at 40° C. for 3 hours and then stirred at 15° C. for another 16 hours. The mixture was poured into ice water (10.0 mL) and extracted with dichloromethane (10.0 mL×3). The combined organic layers were washed with brine (10.0 mL), dried over anhydrous sodium sulfate, filtered and concentrated in vacuum. The residue was purified by silica gel chromatography (Ethyl acetate/Petroleum ether 1:2) to afford 6-[difluoro (3-pyridyl)methyl]-2-(2,5-dimethylpyrrol-1-yl)-3-methyl-benzimidazole-4-carbonitrile (23.0 mg, 60.0 μmol, 16% yield) as a yellow oil. LCMS [ESI, M+1]⁻: 378.1. ¹H NMR (400 MHZ, CDCl₃) δ=8.82-8.69 (m, 2H), 8.16 (d, J=0.8 Hz, 1H), 7.91 (br d, J=8.0 Hz, 1H), 7.85 (d, J=0.8 Hz, 1H), 7.44 (dd, J=4.8, 8.0 Hz, 1H), 5.99 (s, 2H), 3.77 (s, 3H), 2.10-1.99 (m, 6H). 1° F. NMR (400 MHZ, CDCl₃) δ=−88.04 (s, 2F).

Step 4: To a solution of 6-[difluoro (3-pyridyl)methyl]-2-(2,5-dimethylpyrrol-1-yl)-3-methyl-benzimidazole-4-carbonitrile (11.0 mg, 29.2 μmol, 1.00 eq.) in ethanol (0.50 mL) was added hydrochloric acid (12 M, 0.05 mL, 20.6 eq.). The mixture was stirred at 120° C. in a microwave reactor for 1 hour. The resulting mixture was basified with ammonium hydroxide solution until pH 8. The mixture was purified by Prep-HPLC (ammonium bicarbonate condition) to afford 2-amino-6-[difluoro (3-pyridyl)methyl]-3-methyl-benzimidazole-4-carbonitrile, Example 3-17 (5.43 mg, 17.8 μmol, 31% yield) as an off-white solid. LCMS [ESI, M+1]⁺: 300.0. ¹H NMR (400 MHZ, MeOD-d₄) δ=8.70 (br d, J=19.6 Hz, 2H), 8.01 (br d, J=8.0 Hz, 1H), 7.58-7.51 (m, 2H), 7.45 (s, 1H), 3.87 (s, 3H). 19F NMR (400 MHZ, MeOD-d₄) δ=−89.55 (s, 2F).

### EXAMPLE 3-18

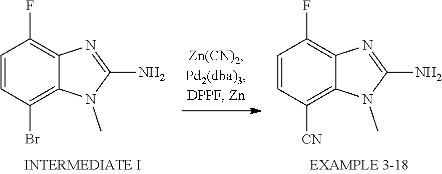

To a mixture of Intermediate I (100 mg, 410 μmol, 1.00 eq.) in dimethyl formamide (2.00 mL) under nitrogen atmosphere was added tris(dibenzylideneacetone) dipalladium (0) (75.0 mg, 82.0 μmol, 0.20 eq.), zinc cyanide (385 mg, 3.27 mmol, 8.00 eq.), 1,1′-bis(diphenylphosphino) ferrocene (90.8 mg, 164 μmol, 0.40 eq.) and zinc powder (21.4 mg, 246 μmol, 1.20 eq.). The reaction mixture was stirred at 100° C. for 48 hours under nitrogen atmosphere. The reaction mixture was filtered and purified by HPLC (formic acid condition) and lyophilizated. Then the product was re-purified by reversed-phase-HPLC (ammonia hydroxide coditions) to afford 2-amino-7-fluoro-3-methyl-benzimidazole-4-carbonitrile, Example 3-18 (11.4 mg, 58.5 μmol, 14% yield) as an off-white solid. LCMS [ESI, M+1]: 191.0. ¹H NMR (400 MHZ, MeOD) δ=7.30 (dd, J=4.4, 8.4 Hz, 1H), 6.93 (dd, J=8.4, 10.2 Hz, 1H), 3.85 (s, 3H).

### EXAMPLE 3-19

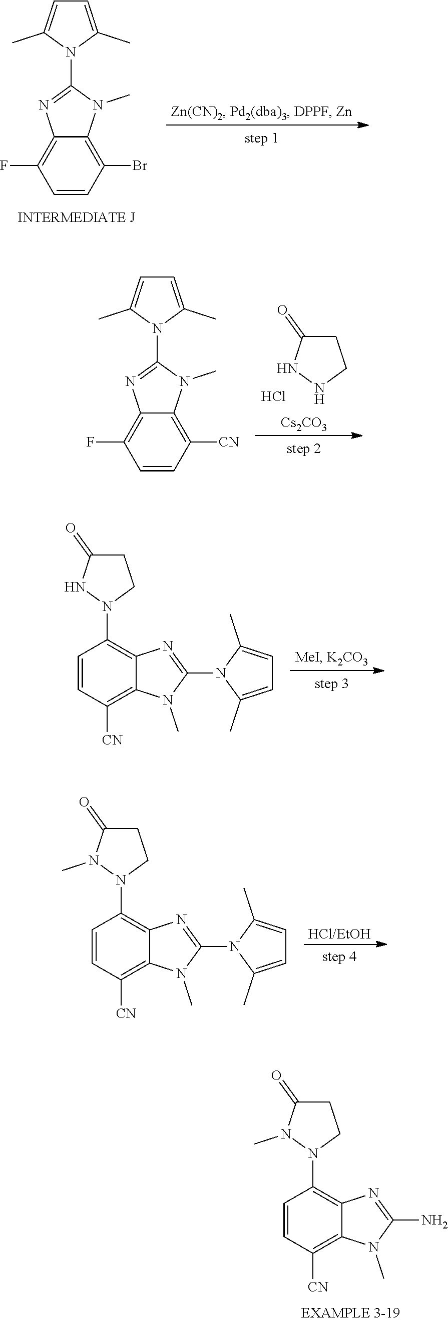

Step 1: To Intermediate J (300 mg, 931 μmol, 1.00 eq.) in dimethyl formamide (5.00 mL) at 20° C. under nitrogen atmosphere was added tris(dibenzylideneacetone) dipalladium (0) (85.3 mg, 93.1 μmol, 0.10 eq.), zinc cyanide (328 mg, 2.79 mmol, 3.00 eq.), 1,1′-bis(diphenylphosphino) ferrocene (103 mg, 186 μmol, 0.20 eq.) and zinc powder (60.9 mg, 931 μmol, 1.00 eq.). The reaction mixture was stirred at 100° C. for 16 hours under nitrogen atmosphere. The mixture was washed with water (30.0 mL), extracted with ethyl acetate (15 mL×4). The combined organic layers were dried over anhydrous sodium sulfate, filtered and concentrated. The residue was purified by silica gel chromatography (ethyl acetate in petroleum ether 0-30%) to afford 2-(2,5-dimethylpyrrol-1-yl)-7-fluoro-3-methyl-benzimidazole-4-carbonitrile (290 mg, 999 μmol, 99% yield) as a yellow solid. LCMS [ESI, M+1]: 269.2. ¹H NMR (400 MHZ, CDCl₃) δ=7.67 (dd, J=4.4, 8.4 Hz, 1H), 7.13-7.09 (m, 1H), 6.00-5.94 (m, 2H), 3.75 (s, 3H), 2.07-2.04 (m, 6H).

Step 2: To a mixture of 2-(2,5-dimethylpyrrol-1-yl)-7-fluoro-3-methyl-benzimidazole-4-carbonitrile (130 mg, 484 μmol, 1.00 eq.) and cesium carbonate (474 mg, 1.45 mmol, 3.00 eq.) in dimethylsulfoxide (2.00 mL) was added pyrazolidin-3-one hydrochloride (89.1 mg, 727 μmol, 1.50 eq.) and the mixture was stirred at 100° C. for 16 hours. The reaction mixture was filtered. The crude was purified by reversed-phase HPLC (formic acidcondition) and lyophilizated to afford 2-(2,5-dimethylpyrrol-1-yl)-3-methyl-7-(3-oxopyrazolidin-1-yl) benzimidazole-4-carbonitrile (110 mg, 329 μmol, 68% yield) as a brown solid. ¹H NMR (400 MHz, CDCl₃) δ=11.08-10.54 (m, 1H), 7.56 (d, J=8.4 Hz, 1H), 6.45 (d, J=8.4 Hz, 1H), 5.99 (s, 2H), 4.06-4.02 (m, 2H), 3.72 (s, 3H), 2.84-2.80 (m, 2H), 2.04 (s, 6H).

Step 3: To a mixture of 2-(2,5-dimethylpyrrol-1-yl)-3-methyl-7-(3-oxopyrazolidin-1-yl)benzimidazole-4-carbonitrile (30.0 mg, 89.7 μmol, 1.00 eq.) and potassium carbonate (16.1 mg, 116 μmol, 1.30 eq.) in dimethyl formamide (0.50 mL) was added methyl iodide (50.9 mg, 359 μmol, 22.3 μL, 4.00 eq.) under nitrogen atmosphere. The reaction mixture was stirred at 30° C. for 2 hours. The mixture was washed with water (3.00 mL), extracted with ethyl acetate (1.00 mL×3). The combined organic layers were dried over anhydrous sodium sulfate, filtered and concentrated. The residue was purified by silica gel chromatography (ethyl acetate in petroleum ether gradient 0-60%) to afford 2-(2,5-dimethylpyrrol-1-yl)-3-methyl-7-(2-methyl-3-oxo-pyrazolidin-1-yl)benzimidazole-4-carbonitrile (20.0 mg, 57.4 μmol, 64% yield) as a colorless oil. ¹H NMR (400 MHZ, CDCl₃) δ=7.61 (d, J=8.4 Hz, 1H), 6.72 (d, J=8.4 Hz, 1H), 5.97 (s, 2H), 4.36-4.32 (m, 2H), 3.73 (s, 3H), 3.18 (s, 3H), 2.59-2.55 (m, 2H), 2.06 (s, 6H).

Step 4: To a mixture of 2-(2,5-dimethylpyrrol-1-yl)-3-methyl-7-(2-methyl-3-oxo-pyrazolidin-1-yl)benzimidazole-4-carbonitrile (15.0 mg, 43.0 μmol, 1.00 eq.) in ethyl alcohol (1.00 mL) was added hydrochloric acid (12M, 0.10 mL). The mixture was stirred at 120° C. for 1 hour in a microwave reactor. The reaction mixture was concentrated and then adjusted to pH 8 with ammonium hydroxide. The crude material was purified by Prep-HPLC (0.1% ammonia hydroxide condition) and lyophilizated to afford 2-amino-3-methyl-7-(2-methyl-3-oxo-pyrazolidin-1-yl)benzimidazole-4-carbonitrile, Example 3-19 (3.41 mg, 12.3 μmol, 29% yield) as a white solid. LCMS [ESI, M+1]: 271.2. ¹H NMR (400 MHZ, MeOD) δ=7.42 (d, J=8.4 Hz, 1H), 6.72 (d, J=8.4 Hz, 1H), 4.01-3.97 (m, 2H), 3.89 (s, 3H), 3.07 (s, 3H), 2.57-2.53 (m, 2H).

### EXAMPLE 3-20

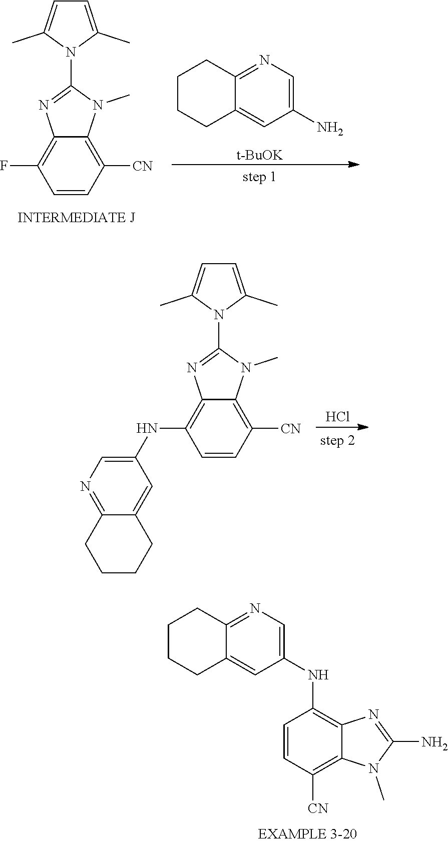 [SMILES: "t-BuOK" ; Cc1ccc(C)n1-c1nc2c(F)ccc(C#N)c2n1C ; Nc1cnc2c(c1)CCCC2 ; Cc1ccc(C)n1-c1nc2c(Nc3cnc4c(c3)CCCC4)ccc(C#N)c2n1C ; Cl ; Cn1c(N)nc2c(Nc3cnc4c(c3)CCCC4)ccc(C#N)c21]

Step 1: A mixture of 5,6,7,8-tetrahydroquinolin-3-amine (66.3 mg, 447 μmol, 1.50 eq.) and potassium tert-butoxide (60.2 mg, 537 μmol, 1.80 eq.) in dimethylsulfoxide (2.00 mL) was stirred at 20° C. for 0.5 hour. Then a solution of Intermediate J (80.0 mg, 298 μmol, 1.00 eq.) in dimethylsulfoxide (2.00 mL) was added to the reaction and the resulting was stirred at 60° C. for 3 hours. The mixture was diluted with water (30.0 mL) and pH was adjusted to 7 with 1M aqueous hydrochloric acid. The mixture was extracted with ethyl acetate (10 mL×3). The organic layer was washed with brine (20.0 mL), dried over anhydrous sodium sulfate, filtered and concentrated. The residue was purified by flash silica gel chromatography (ethyl acetate/petroleum ether 0-30%) to afford 2-(2,5-dimethyl-1H-pyrrol-1-yl)-1-methyl-4-((5,6,7,8-tetrahydroquinolin-3-yl)amino)-1H-benzo[d]imidazole-7-carbonitrile (65.0 mg, 164 μmol, 55% yield) as a light yellow solid. LCMS [ESI, M+1]: 397.1. ¹H NMR (400 MHZ, CDCl₃) δ=8.44 (s, 1H), 7.55 (d, J=8.4 Hz, 1H), 7.46 (br s, 1H), 7.35 (br s, 1H), 6.99 (d, J=8.4 Hz, 1H), 6.00 (s, 2H), 3.74 (s, 3H), 3.08-2.97 (m, 2H), 2.85-2.80 (m, 2H), 2.11-2.05 (m, 6H), 1.99-1.91 (m, 2H), 1.90-1.83 (m, 2H).

Step 2: To a solution of 2-(2,5-dimethyl-1H-pyrrol-1-yl)-1-methyl-4-((5,6,7,8-tetrahydroquinolin-3-yl)amino)-1/-benzo[d]imidazole-7-carbonitrile (50.0 mg, 126 μmol, 1.00 eq.) in ethyl alcohol (2.00 mL) was added hydrochloric acid (12.0 M, 0.20 mL, 19.0 eq.). The mixture was stirred at 120° C. for 1 hour in a microwave reactor. The reaction mixture was concentrated. The residue was purified by prep-HPLC (HCl condition) and lyophilized to afford 2-amino-1-methyl-4-((5,6,7,8-tetrahydroquinolin-3-yl)amino)-1/-benzo[d]imidazole-7-carbonitrile hydrochloride, Example 3-20 (27.4 mg, 75.9 μmol, 60% yield) as a yellow solid. LCMS [ESI, M+1]: 319.2. ¹H NMR (400 MHZ, DMSO-d₆) δ=10.05 (br s, 1H), 8.91-8.55 (m, 2H), 8.34 (d, J=2.4 Hz, 1H), 7.91 (s, 1H), 7.58 (d, J=8.4 Hz, 1H), 7.19 (d, J=8.4 Hz, 1H), 3.83 (s, 3H), 2.97 (br t, J=6.0 Hz, 2H), 2.86 (br t, J=6.0 Hz, 2H), 1.90-1.81 (m, 2H), 1.81-1.73 (m, 2H).

### EXAMPLE 3-21

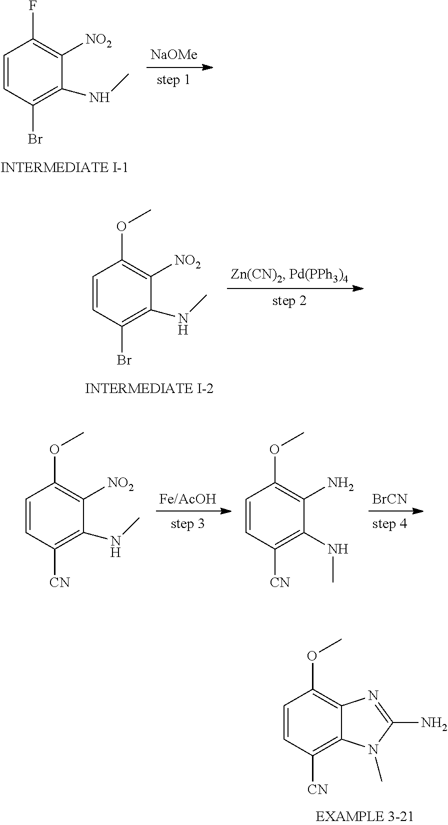

Step 1: To a solution of Intermediate I-1 (690 mg, 2.77 mmol, 1.00 eq) in methanol (5.00 mL) was added to sodium methanolate (5.40 M, 5.13 mL, 10.0 eq.) at 20° C. and the resulting mixture was stirred at 20° C. for 16 hours. The mixture was diluted with water (60.0 mL) and extracted with ethyl acetate (40.0 mL×3). The combined organic extracts were washed brine (60.0 mL) and then dried over anhydrous sodium sulfate, filtered and concentrated in vacuum. The residue was purified by silica gel column chromatography (ethyl acetate in petroleum ether 0-20%) to get 6-bromo-3-methoxy-N-methyl-2-nitro-aniline, Intermediate I-2 (700 mg, 2.68 mmol, 97% yield) as a yellow solid. ¹H NMR (400 MHZ, CDCl₃) δ=7.41 (d, J=8.8 Hz, 1H), 6.24 (d, J=8.8 Hz, 1H), 3.83 (s, 3H), 2.86 (s, 3H).

Step 2: To a solution of 6-bromo-3-methoxy-N-methyl-2-nitro-aniline (700 mg, 2.68 mmol, 1.00 eq.) in dimethyl formamide (5.00 mL) were added to zinc cyanide (630 mg, 5.36 mmol, 340 μL, 2.00 eq.) and tetrakis(triphenylphosphine) palladium (0) (310 mg, 268 μmol, 0.10 eq.) at 20° C. Then the mixture was degassed and stirred at 95° C. for 16 hours. The reaction mixture was quenched by addition sodium hypochlorite solution (5.00 mL) at 20° C., diluted with water (50.0 mL) and extracted with ethyl acetate (30.0 mL×3). The combined organic layers were washed with brine (60.0 mL), dried over anhydrous sodium sulfate, filtered and concentrated under reduced pressure. The residue was purified by silica gel column chromatography (ethyl acetate in petroleum ether 0-20%) to get 4-methoxy-2-(methylamino)-3-nitro-benzonitrile (550 mg, 2.58 mmol, 96% yield) as a yellow solid. ¹H NMR (400 MHZ, CDCl₃) δ=7.49 (d, J=8.8 Hz, 1H), 6.37 (d, J=8.8 Hz, 1H), 3.92 (s, 3H), 3.16 (s, 3H)

Step 3: To a solution of 4-methoxy-2-(methylamino)-3-nitro-benzonitrile (550 mg, 2.65 mmol, 1.00 eq.) in ethyl acetate (10.0 mL) was added iron powder (741 mg, 13.3 mmol, 5.00 eq.), acetic acid (3.20 mL) and water (0.32 mL) at 20° C. Then the mixture was degassed and charged with nitrogen for three times and then stirred at 70° C. for 16 hours. The mixture was neutralized to pH 9 with 1M sodium hydroxide solution. The resulting mixture was filtered over celite, and the filtrate was extracted with ethyl acetate (30.0 mL×3). The combined organic layers were dried over sodium sulfate, filtered and concentrated in vacuum to get 3-amino-4-methoxy-2-(methylamino)benzonitrile (376 mg, crude) as a yellow oil. ¹H NMR (400 MHZ, DMSO-d₆) δ=6.85 (d, J=8.4 Hz, 1H), 6.50 (d, J=8.4 Hz, 1H), 5.04-4.96 (m, 1H), 4.49 (s, 2H), 3.80 (s, 3H), 3.01 (d, J=5.2 Hz, 3H).

Step 4: To a solution of 3-amino-4-methoxy-2-(methylamino)benzonitrile (376 mg, 2.12 mmol, 1.00 eq.) in ethanol (4.00 mL) was added cyanogen bromide (450 mg, 4.24 mmol, 312 μL, 2.00 eq.). The mixture was stirred at 20° C. for 2 hours. The mixture was concentrated under vacuum. The residue was diluted with water (50.0 mL) and extracted with ethyl acetate (100 mL). The organic layer was dried over anhydrous sodium sulfate, filtered and concentrated in vacuum. The residue was purified by prep-HPLC (HCl condition) to produce 2-amino-4-methoxy-1-methyl-1H-benzo[d]imidazole-7-carbonitrile hydrochloride, Example 3-21 (10.8 mg, 44.7 μmol, 2% yield) as an off-white solid. LCMS [M+1]⁺: 203.1. ¹H NMR (400 MHZ, DMSO-d₆) δ=8.60 (s, 2H), 7.71 (d, J=8.8 Hz, 1H), 7.10 (d, J=8.8 Hz, 1H), 4.02 (s, 3H), 3.81 (s, 3H).

### EXAMPLE 3-22

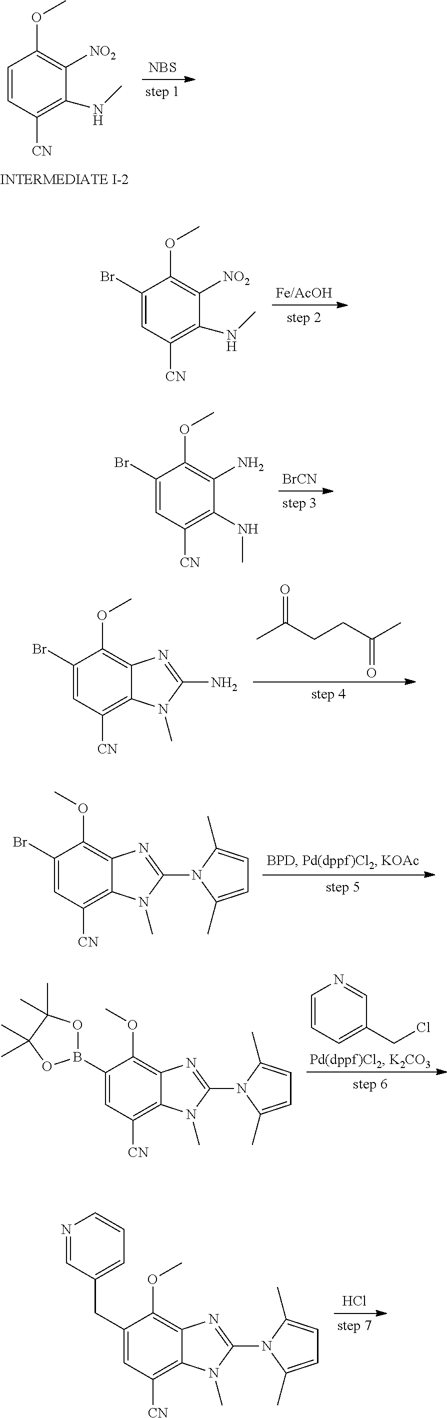

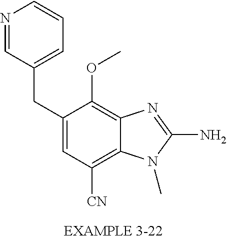 [SMILES: COc1c(Cc2cccnc2)cc(C#N)c2c1nc(N)n2C]

Step 1: To a solution of Intermediate I-2 (1.36 g, 6.56 mmol, 1.00 eq.) in dimethylformamide (10.0 mL) were added to N-Bromosuccinimide (1.17 g, 6.56 mmol, 1.00 eq.) and acetic acid (1.00 mL) at 20° C. Then the mixture was stirred at 20° C. for 4 hours and then concentrated in vacuum. The residue was partitioned between ethyl acetate (30.0 mL) and water (30.0 mL). The aqueos layer was extracted with ethyl acetate (60.0 mL×3). The combined organic extracts were washed with brine (20.0 mL), dried over anhydrous sodium sulfate, filtered and concentrated in vacuum. The residue was purified by flash silica gel chromatography (ethyl acetate in petroleum 0-20%) to give 5-bromo-4-methoxy-2-(methylamino)-3-nitro-benzonitrile (1.65 g, 5.77 mmol, 88% yield) as a yellow solid. LCMS [M+1]⁺: 288.0. ¹HNMR (400 MHZ, MeOD-d₄) δ=7.84 (s, 1H), 3.96 (s, 3H), 3.06 (s, 3H).

Step 2: To a solution of 5-bromo-4-methoxy-2-(methylamino)-3-nitro-benzonitrile (800 mg, 2.80 mmol, 1.00 eq.) in ethyl acetate (15.0 mL) were added to iron powder (781 mg, 14.0 mmol, 5.00 eq.), acetic acid (3.20 mL) and water (0.32 mL) at 20° C. Then the mixture was degassed and stirred at 70° C. for 12 hours. The pH was adjusted to 7-8 with aqueous sodium bicarbonate. The resulting mixture was filtered over celite, and the filtrate was concentrated under reduced pressure. The mixture was diluted with water (50.0 mL), extracted with ethyl acetate (20.0 mL×3). The combined organic layers were dried over sodium sulfate, filtered and concentrated in vacuum to give 3-amino-5-bromo-4-methoxy-2-(methylamino)benzonitrile (700 mg, crude) as a yellow solid. LCMS [M+1]⁺: 258.0

Step 3: To a solution of 3-amino-5-bromo-4-methoxy-2-(methylamino)benzonitrile (700 mg, 2.73 mmol, 1.00 eq.) in ethanol (10.0 mL) was added to cyanogen bromide (579 mg, 5.47 mmol, 402 μL, 2.00 eq.) at 20° C. Then the mixture was stirred at 20° C. for 12 hours. The reaction mixture was concentrated under reduced pressure. The residue was purified by flash silica gel chromatography (ethyl acetate in petroleum 0-100%) to give 2-amino-6-bromo-7-methoxy-3-methyl-benzimidazole-4-carbonitrile (731 mg, 2.60 mmol, 95% yield) as an off-white solid.

LCMS [M+3]⁺: 283.0. ¹H NMR (400 MHZ, MeOD-d₄) δ=7.78 (s, 1H), 4.10 (s, 3H), 3.89 (s, 3H).

Step 4: To a solution of 2-amino-6-bromo-7-methoxy-3-methyl-benzimidazole-4-carbonitrile (200 mg, 711 μmol, 1.00 eq.) in dimethylformamide (1.00 mL) and toluene (4.00 mL) were added hexane-2,5-dione (122 mg, 1.07 mmol, 125 μL, 1.50 eq.) and 4-methylbenzenesulfonic acid (1.23 mg, 7.11 μmol, 0.01 eq.) at 20° C. Then the mixture was stirred at 120° C. for 5 hours. The reaction was diluted with water (30.0 mL) and extracted with ethyl acetate (20.0 mL×3). The combined organic extracts were washed with brine (10.0 mL×3), dried over anhydrous sodium sulfate, filtered and concentrated in vacuum. The residue was purified by flash silica gel chromatography (ethyl acetate in petroleum 0-50%) to produce 6-bromo-2-(2,5-dimethylpyrrol-1-yl)-7-methoxy-3-methyl-benzimidazole-4-carbonitrile (120 mg, 334 μmol, 47% yield) as an off-white solid. LCMS [M+1]⁺: 359.1. ¹H NMR (400 MHZ, CDCl₃-d) δ=7.82 (s, 1H), 5.99 (s, 2H), 4.60 (s, 3H), 3.71 (s, 3H), 2.06 (s, 6H).

Step 5: To a solution of 6-bromo-2-(2,5-dimethylpyrrol-1-yl)-7-methoxy-3-methyl-benzimidazole-4-carbonitrile (120 mg, 334 μmol, 1.00 eq.), potassium acetate (98.4 mg, 1.00 mmol, 3.00 eq.), 4,4,5,5-tetramethyl-2-(4,4,5,5-tetramethyl-1,3,2-dioxaborolan-2-yl)-1,3,2-dioxaborolane (127 mg, 501 1.50 μmol, eq.), [1,1-bis(diphenylphosphino) ferrocene]dichloropalladium (II) (24.4 mg, 33.4 μmol, 0.10 eq.) in dioxane (3.00 mL) was degassed and stirred at 95° C. for 4 hours. The residue was diluted with water (40.0 mL), extracted with ethyl acetate (30.0 mL×3). The combined organic extracts were washed with brine (10.0 mL×3) and then dried over anhydrous sodium sulfate, filtered and concentrated in vacuum to give a residue. The residue was purified by silica gel chromatography (ethyl acetate in petroleum 0-30%) to give 2-(2,5-dimethylpyrrol-1-yl)-7-methoxy-3-methyl-6-(4,4,5,5-tetramethyl-1,3,2-dioxaborolan-2-yl)benzimidazole-4-carbonitrile (140 mg, crude) as a brown solid. LCMS [M+1]⁺: 407.1. ¹H NMR (400 MHZ, CDCl₃-d) δ=7.99 (s, 1H), 5.97 (s, 2H), 4.51 (s, 3H), 3.70 (s, 3H), 2.04 (s, 6H), 1.38 (s, 12H).

Step 6: To a solution of 2-(2,5-dimethylpyrrol-1-yl)-7-methoxy-3-methyl-6-(4,4,5,5-tetramethyl-1,3,2-dioxaborolan-2-yl)benzimidazole-4-carbonitrile (140 mg, 345 μmol, 1.00 eq.) and 3-(chloromethyl)pyridine (65.9 mg, 517 μmol, 1.50 eq.) in dioxane (3.00 mL) and water (0.10 mL) were added potassium carbonate (143 mg, 1.03 mmol, 3.00 eq.) and [1,1-bis(diphenylphosphino) ferrocene]dichloropalladium (II) (25.2 mg, 34.5 μmol, 0.10 eq.). The mixture was degassed and stirred at 90° C. for 2 hours before being concentrated. The residue was partitioned between ethyl acetate (20.0 mL) and water (20.0 mL). The aqueos layer was extracted with ethyl acetate (20.0 mL×3). The combined organic extracts were washed brine (10.0 mL), dried over anhydrous sodium sulfate, filtered and concentrated in. The residue was purified by prep-TLC (SiO₂, petroleum ether/ethyl acetate 3:1) to give 2-(2,5-dimethylpyrrol-1-yl)-7-methoxy-3-methyl-6-(3-pyridylmethyl)benzimidazole-4-carbonitrile (63.0 mg, 170 μmol, 49% yield) as a brown solid. ¹H NMR (400 MHZ, CDCl₃-d) δ=8.63 (s, 2H), 7.49 (s, 1H), 7.41 (s, 2H), 5.97 (s, 2H), 4.60 (s, 2H), 4.49 (s, 3H), 3.69 (s, 3H), 2.05 (s, 6H).

Step 7: To a solution of 2-(2,5-dimethylpyrrol-1-yl)-7-methoxy-3-methyl-6-(3-pyridylmethyl)benzimidazole-4-carbonitrile (40.0 mg, 108 μmol, 1.00 eq.) in ethanol (2.00 mL) was added hydrochloric acid (12 M, 0.2 mL, 22.3 eq.) at 20° C. The sealed tube was heated at 120° C. for 40 minutes in a microwave reactor. The mixture was basified with aqueous sodium bicarbonate until pH 8. The resulting mixture was concentrated in and the residue was purified by prep-HPLC (HCl condition) to give 2-amino-7-methoxy-3-methyl-6-(3-pyridylmethyl)benzimidazole-4-carbonitrile hydrochloride, Example 3-22 (2.27 mg, 6.84 μmol, 6% yield) as a white solid. LCMS [M+1]⁺: 294.2. ¹H NMR (400 MHZ, MeOD-d₄) δ=8.81 (s, 1H), 8.74 (d, J=6.0 Hz, 1H), 8.50 (d, J=8.4 Hz, 1H), 8.02 (dd, J=5.6, 8.0 Hz, 1H), 7.69 (s, 1H), 4.35 (s, 2H), 4.04 (s, 3H), 3.94 (s, 3H).

### EXAMPLE 3-23

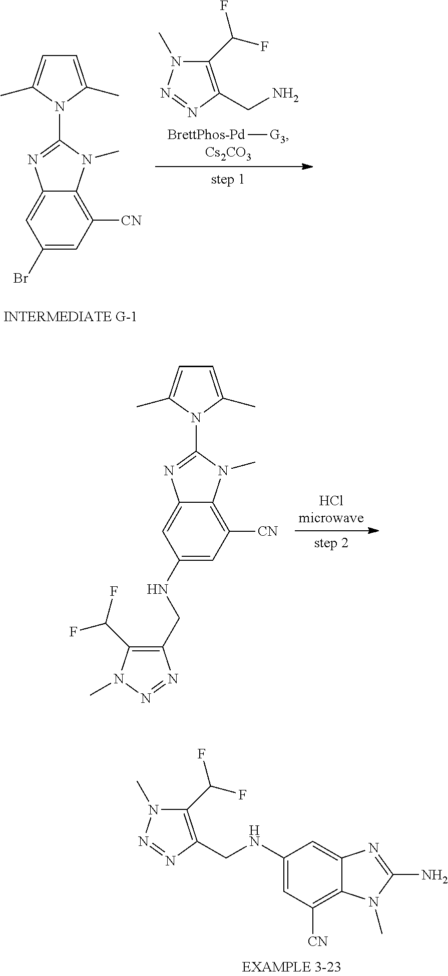

Step 1: To a solution of [5-(difluoromethyl)-1-methyl-triazol-4-yl]methanamine (37.0 mg, 186 μmol, 1.00 eq., hydrochloride) and Intermediate G-1 (49.1 mg, 149 μmol, 0.80 eq.) in dioxane (1.00 mL) was added cesium carbonate (182 mg, 559 μmol, 3.00 eq.) and methanesulfonato 2-dicyclohexylphosphino-3,6-dimethoxy-2-4-6-tri-i-propyl-1,1-biphenyl) (2-amino-1,1-biphenyl-2-yl) palladium (II) (16.9 mg, 18.6 μmol, 0.10 eq.) under nitrogen atmosphere. The mixture was stirred at 100° C. for 12 hours. The mixture was filtered to remove the insoluble materials. The filtrate was concentrated and the residue was purified by prep-HPLC (TFA condition) and lyophilized to afford 5-(((5-(difluoromethyl)-1-methyl-1H-1,2,3-triazol-4-yl)methyl)amino)-2-(2,5-dimethyl-1H-pyrrol-1-yl)-1-methyl-1H-benzo[d]imidazole-7-carbonitrile (50.0 mg, 122 μmol, 65.4% yield, 99.9% purity) as a white solid. LCMS [ESI, M+1]⁺=411.1.

Step 2: To a solution of 5-(((5-(difluoromethyl)-1-methyl-1H-1,2,3-triazol-4-yl)methyl)amino)-2-(2,5-dimethyl-1H-pyrrol-1-yl)-1-methyl-1H-benzo[d]imidazole-7-carbonitrile (50.0 mg, 122 μmol, 1.00 eq.) in ethanol (2.00 mL) was added hydrochloric acid (12 M, 0.20 mL). The mixture was stirred at 120° C. for 1 hour under microwave irradiation. The pH of the reaction solution was adjusted to ˜7 with diisopropylethylamine and concentrated. The crude was purified by prep-HPLC (formic acid) and lyophilized to afford 2-amino-5-(((5-(difluoromethyl)-1-methyl-1H-1,2,3-triazol-4-yl)methyl)amino)-1-methyl-1H-benzo[d]imidazole-7-carbonitrile, Example 3-23 (11.9 mg, 31.2 μmol, 25.7% yield, 99.7% purity, formate) as an off-white solid. LCMS [ESI, M+1]⁺=333.0. ¹H NMR (400 MHZ, DMSO-d₆) δ=8.14 (s, 1H), 7.50 (t, J=52.1 Hz, 1H), 6.80 (d, J=1.8 Hz, 1H), 6.68 (br s, 2H), 6.56 (d, J=1.8 Hz, 1H), 5.93 (br s, 1H), 4.39 (br s, 2H), 4.11 (s, 3H), 3.63 (s, 3H)

### EXAMPLE 3-24

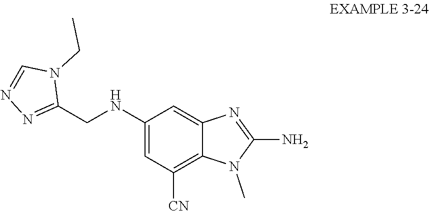 [SMILES: CCn1cnnc1CNc1cc(C#N)c2c(c1)nc(N)n2C]

Example 3-24 was synthesized from Intermediate G-1 and (4-ethyl-4H-1,2,4-triazol-3-yl) methanamine following the two-step procedure described for Example 3-23 to afford 2-amino-5-(((4-ethyl-4H-1,2,4-triazol-3-yl)methyl)amino)-1-methyl-1H-benzo[d]imidazole-7-carbonitrile, Example 3-24 (4.08 mg, 13.5 μmol, 14% yield over 2 steps) as a white solid. LCMS [ESI, M+1]⁺=297.1. ¹H NMR (400 MHZ, DMSO-d₆) δ=8.50 (s, 1H), 6.93 (br s, 2H), 6.88 (d, J=2.0 Hz, 1H), 6.65 (d, J=1.8 Hz, 1H), 6.13 (br s, 1H), 4.43 (br s, 2H), 4.05 (d, J=7.2 Hz, 2H), 3.65 (s, 3H), 1.31 (s, 3H)

### EXAMPLE 3-25

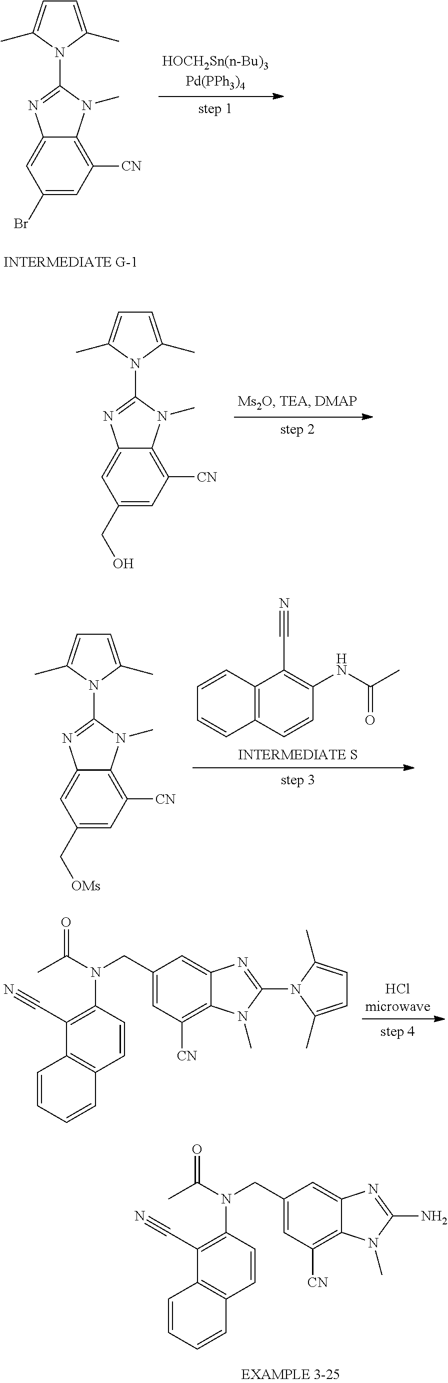

Step 1: To a solution of Intermediate G-1 (700 mg, 2.13 mmol, 1.00 eq.) and tributylstannylmethanol (1.02 g, 3.19 mmol, 1.50 eq.) in dioxane (14.0 mL) was added tetrakis [triphenylphosphine]palladium (0) (246 mg, 213 μmol, 0.10 eq.). The mixture was stirred at 80° C. for 12 hours under nitrogen atmosphere. The mixture was concentrated under vacuum. The residue was purified by column chromatography (SiO₂, petroleum ether/ethyl acetate 1:0 to 1:1) to afford 2-(2,5-dimethylpyrrol-1-yl)-6-(hydroxymethyl)-3-methyl-benzimidazole-4-carbonitrile (280 mg, 999 μmol, 47.0% yield) as an off-white solid.

Step 2: To a solution of 2-(2,5-dimethylpyrrol-1-yl)-6-(hydroxymethyl)-3-methyl-benzimidazole-4-carbonitrile (120 mg, 428 μmol, 1.00 eq.), N,N-4-dimethylaminopyridine (5.23 mg, 42.8 μmol, 0.10 eq.) and methylsulfonyl methanesulfonate (89.5 mg, 514 μmol, 1.20 eq.) in dichloromethane (4.00 mL) was added triethylamine (108 mg, 1.07 mmol, 149 μL, 2.50 eq.) at 0° C. The resulting mixture was stirred at 0° C. for 0.5 hour. The mixture was diluted with water (20.0 mL) and extracted with dichloromethane (10.0 mL). The combined organic layers were washed with brine, dried over sodium sulfate, filtered and concentrated. The residue was purified quickly by flash silica gel chromatography (50% ethyl acetate in petroleum ether) to afford [7-cyano-2-(2,5-dimethylpyrrol-1-yl)-1-methyl-benzimidazol-5-yl]methyl methanesulfonate (120 mg, 335 μmol, 78.2% yield) as a colorless oil.

Step 3: To a solution of N-(1-cyano-2-naphthyl) acetamide (50.0 mg, 238 μmol, 1.00 eq.) in tetrahydrofuran (2.00 mL) was added sodium hydride (9.51 mg, 238 μmol, 60% purity, 1.00 eq.) at 0° C. and the mixture was stirred at 0° C. for 1 hour. Then a solution of [7-cyano-2-(2,5-dimethylpyrrol-1-yl)-1-methyl-benzimidazol-5-yl]methyl methanesulfonate (111 mg, 309 μmol, 1.30 eq.) in tetrahydrofuran (1.00 mL) was added. The resulting mixture was stirred at 0° C. for 1 hour and then warmed up to 20° C. and stirred at 10-20° C. for 16 hours. The mixture was quenched with saturated ammonium chloride solution (20.0 mL) and extracted with ethyl acetate (10.0 mL×3). The combined organic layers were washed with brine, dried over sodium sulfate, filtered and concentrated. The residue was purified by flash silica gel chromatography (50-70% ethyl acetate in petroleum ether) to afford N-[[7-cyano-2-(2,5-dimethylpyrrol-1-yl)-1-methyl-benzimidazol-5-yl]methyl]-N-(1-cyano-2-naphthyl) acetamide (103 mg, 207 μmol, 87.0% yield) as a colorless oil. LCMS [ESI, M+1]⁺=473.3.

Step 4: N-[[7-cyano-2-(2,5-dimethylpyrrol-1-yl)-1-methyl-benzimidazol-5-yl]methyl]-N-(1-cyano-2-naphthyl) acetamide (50.0 mg, 106 μmol, 1.00 eq.) was taken up into a microwave tube with ethanol (1.00 mL) and hydrochloric acid (0.10 mL). The sealed tube was heated at 120° C. for 1 hour under microwave irradiation. The pH of the mixture was adjusted to 8 with sodium hydroxide (4 M) and the resulting was filtered. The filtrate was concentrated. The residue was purified by prep-HPLC (formic acid condition) and lyophilized to afford N-[(2-amino-7-cyano-1-methyl-benzimidazol-5-yl)methyl]-N-(1-cyano-2-naphthyl) acetamide, Example 3-25 (21.1 mg, 53.6 μmol, 50.6% yield) as a white solid. LCMS [ESI, M+1]⁺=395.2. 1H NMR (400 MHZ, DMSO-d₆) δ=8.32 (br d, J=8.8 Hz, 1H), 8.18-8.10 (m, 1H), 8.06 (br d, J=8.2 Hz, 1H), 7.82 (br t, J=7.4 Hz, 1H), 7.78-7.69 (m, 1H), 7.45 (br d, J=8.8 Hz, 1H), 7.23 (s, 1H), 7.11 (s, 1H), 6.83 (br s, 2H), 5.13 (br d, J=14.8 Hz, 1H), 4.99-4.87 (m, 1H), 3.70 (s, 3H), 1.86 (s, 3H)

### EXAMPLE 3-30

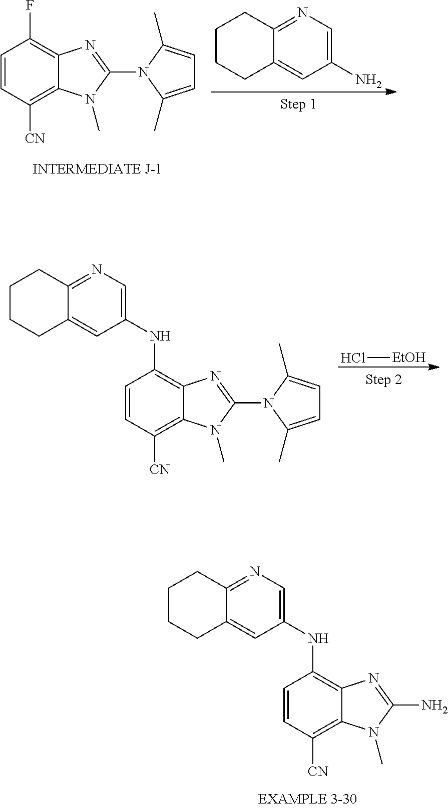

Step 1: A mixture of 5,6,7,8-tetrahydroquinolin-3-amine (66.3 mg, 447 μmol, 1.5 eq.) and potassium tert-butoxide (60 mg, 537 μmol, 1.8 eq.) in DMSO (2.0 mL) was stirred at 20° C. for 0.5 hour. Then a solution of Intermediate J-1 (80 mg, 298 μmol, 1.0 eq.) in DMSO (2.0 mL) was added and stirred at 60° C. for 3 hours. The mixture was then diluted with water (30 mL) and adjusted to pH=7 with aqueous of hydrochloric acid (1 M). The mixture was extracted with ethyl acetate (10 mL×3). The organic layer was washed with brine (20 mL), dried over anhydrous sodium sulfate, filtered and concentrated. The residue was purified by flash silica gel chromatography (20 g silica, eluent of 10 ˜ 30% ethyl acetate/petroleum ether gradient) to afford 2-(2,5-dimethyl-1H-pyrrol-1-yl)-1-methyl-4-((5,6,7,8-tetrahydroquinolin-3-yl)amino)-1H-benzo[d]imidazole-7-carbonitrile (65 mg, 164 μmol, 55% yield) as light yellow solid. LCMS [ESI, M+1]: 397.1. ¹H NMR (400 MHZ, CDCl₃) δ=8.44 (s, 1H), 7.55 (d, J=8.4 Hz, 1H), 7.46 (br s, 1H), 7.35 (br s, 1H), 6.99 (d, J=8.4 Hz, 1H), 6.00 (s, 2H), 3.74 (s, 3H), 3.08-2.97 (m, 2H), 2.85-2.80 (m, 2H), 2.11-2.05 (m, 6H), 1.99-1.91 (m, 2H), 1.90-1.83 (m, 2H).

Step 2: To a solution of 2-(2,5-dimethyl-1H-pyrrol-1-yl)-1-methyl-4-((5,6,7,8-tetrahydroquinolin-3-yl)amino)-1H-benzo[d]imidazole-7-carbonitrile (50 mg, 126 μmol, 1.0 eq.) in ethyl alcohol (2.0 mL) was added hydrochloric acid (12.0 M, 0.2 mL, 19 eq.). The mixture was stirred at 120° C. for 1 hour under microwave. The mixture was concentrated. The residue was purified by prep-HPLC (column: YMC Triart 30×150 mm×7 μm; mobile phase: [water (hydrochloric acid)-acetonitrile]; B %: 5%-25%, 7 min) and lyophilized to afford 2-amino-1-methyl-4-((5,6,7,8-tetrahydroquinolin-3-yl)amino)-1H-benzo[d]imidazole-7-carbonitrile, EXAMPLE 3-30 (27 mg, 75.9 μmol, 60% yield, hydrochloric acid salt) as yellow solid. LCMS [ESI, M+1]: 319.2. ¹H NMR (400 MHZ, DMSO-d₆) δ=10.05 (br s, 1H), 8.91-8.55 (m, 2H), 8.34 (d, J=2.4 Hz, 1H), 7.91 (s, 1H), 7.58 (d, J=8.4 Hz, 1H), 7.19 (d, J=8.4 Hz, 1H), 3.83 (s, 3H), 2.97 (br t, J=6.0 Hz, 2H), 2.86 (br t, J=6.0 Hz, 2H), 1.90-1.81 (m, 2H), 1.81-1.73 (m, 2H).

### EXAMPLE 4-1

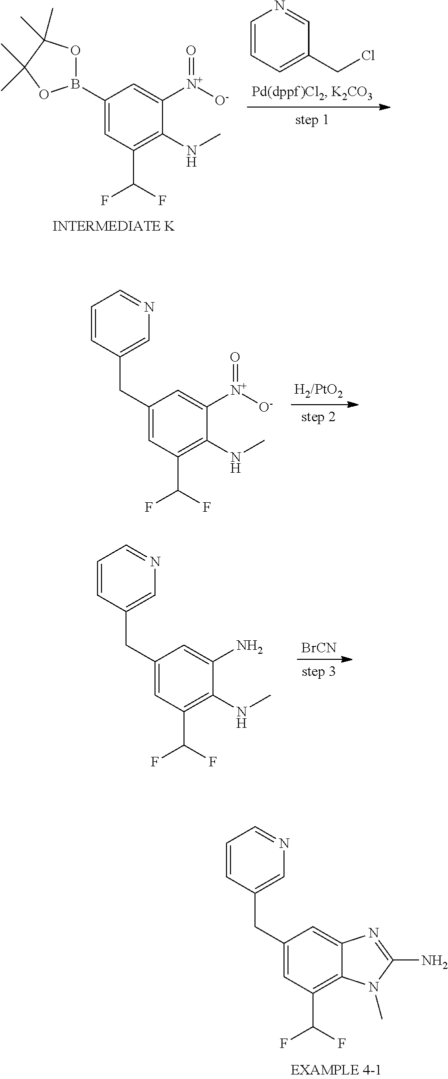

Step 1: To a solution of Intermediate K (180 mg, 549 μmol, 1.00 eq.) and potassium carbonate (228 mg, 1.65 mmol, 3.00 eq.) in dioxane (6.00 mL) and water (2.00 mL) were added 3-(chloromethyl)pyridine (84.0 mg, 658 μmol, 1.20 eq.) and Pd(dppf)Cl₂ (40.0 mg, 55.0 μmol, 0.10 eq.). The mixture was stirred at 90° C. for 4 hours under nitrogen atmosphere. The reaction mixture was cooled down and filtered, then diluted with water (10.0 mL) and extracted with ethyl acetate (10.0 mL×3). The combined organic layers were washed with brine (10.0 mL×2), dried over sodium sulfate, filtered and concentrated under reduced pressure. The residue was purified by prep-TLC (SiO₂, petroleum ether/ethyl acetate 5:1) to give 2-(difluoromethyl)-N-methyl-6-nitro-4-(3-pyridylmethyl) aniline (130 mg, 443 μmol, 81% yield) as a yellow oil.

¹H NMR (400 MHZ, CDCl₃) ¿=8.51-8.37 (m, 2H), 8.01 (s, 1H), 7.76-7.64 (m, 2H), 7.40 (dd, J=4.8, 8.0 Hz, 1H), 7.10 (t, J=54.4 Hz, 1H), 4.04 (s, 2H), 3.01 (s, 3H).

Step 2: To a solution of 2-(difluoromethyl)-N-methyl-6-nitro-4-(3-pyridylmethyl) aniline (60.0 mg, 205 μmol, 1.00 eq.) in methanol (2.00 mL) was added platinum dioxide (46.0 mg, 205 μmol, 1.00 eq.) under nitrogen. The suspension was degassed under vacuum and purged with hydrogen three times. The mixture was stirred under hydrogen (1 atm) at 25° C. for 1 hour. The reaction mixture was used in the next step directly.

Step 3: To a solution of 3-(difluoromethyl)-nitrogen-methyl-5-(3-pyridylmethyl)benzene-1,2-diamine (36.0 mg, 137 μmol, 1.00 eq.) in methanol (2.00 mL) was added cyanogen bromide (0.32 g, 3.02 mmol, 222 μL, 22.1 eq.). The mixture was stirred at 25° C. for 1 hour under nitrogen atmosphere. The reaction mixture was filtered and concentrated under reduced. The residue was purified by prep-HPLC (NH₃·H₂O condition) to give 7-(difluoromethyl)-1-methyl-5-(3-pyridylmethyl)benzimidazol-2-amine, Example 4-1 (2.86 mg, 9.48 μmol, 7% yield) as a yellow solid. LCMS [M+1]⁺=289.2. ¹H NMR (400 MHZ, CDCl₃) δ=8.44 (s, 1H), 8.37 (d, J=4.8 Hz, 1H), 7.69 (d, J=8.0 Hz, 1H), 7.35 (dd, J=4.8, 8.0 Hz, 1H), 7.24 (s, 1H), 7.17-6.88 (m, 2H), 4.09 (s, 2H), 3.71 (s, 3H).

### EXAMPLE 4-2

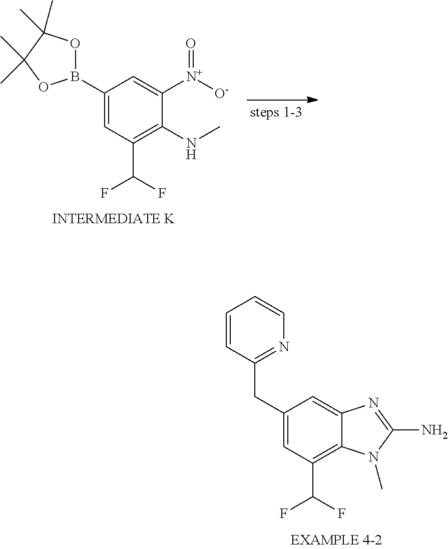

7-(difluoromethyl)-1-methyl-5-(2-pyridylmethyl)benzimidazol-2-amine, Example 4-1, was synthesized from Intermediate K (150 mg, 457 μmol, 1.00 eq.) and 2-(chloromethyl)pyridine (64.0 mg, 503 μmol, 1.10 eq.) following the same 3-step procedure as for Example 4-1 to produce the desired product (3.20 mg, 10.79 μmol, 2% yield over 3 steps) as a yellow solid. LCMS [ESI, M+1]: 289.2. ¹H NMR (400 MHZ, MeOD-d₄) δ=8.50-8.43 (m, 1H), 7.77 (dt, J=1.6, 7.6 Hz, 1H), 7.36-7.23 (m, 3H), 7.20-6.88 (m, 2H), 4.21 (s, 2H), 3.72 (t, J=1.6 Hz, 3H).

### EXAMPLE 5-1

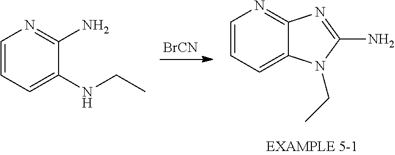

To a solution of N3-ethylpyridine-2,3-diamine (70.0 mg, 510 μmol, 1.00 eq.) in ethanol (1.00 mL) and water (1.00 mL) was added cyanogen bromide (162 mg, 1.53 mmol, 113 μL, 3.00 eq.). The mixture was stirred at 80° C. for 3 hours. The reaction mixture pH was adjusted to 12 with 2 M sodium hydroxide solution and extracted with trichloromethane/isopropyl alcohol=3/1 (5.00 mL×4). The combined organic layers were dried over sodium sulfate, filtered and concentrated. The crude product was purified by prep-HPLC (ammonia hydroxide condition) and then further re-purified by prep-HPLC (HCl condition) to afford 1-ethyl-1H-imidazo[4,5-b]pyridin-2-amine hydrochloride, Example 5-1 (36.6 mg, 226 μmol, 44% yield) as a yellow solid. LCMS [ESI, M+1]: 163.2. ¹H NMR (400 MHz, MeOD) δ=8.06 (d, J=6.0 Hz, 1H), 7.98 (d, J=7.6 Hz, 1H), 7.30 (d, J=6.0, 7.6 Hz, 1H), 4.22 (q, J=7.2 Hz, 2H), 1.39 (t, J=7.2 Hz, 3H).

### EXAMPLE 5-2

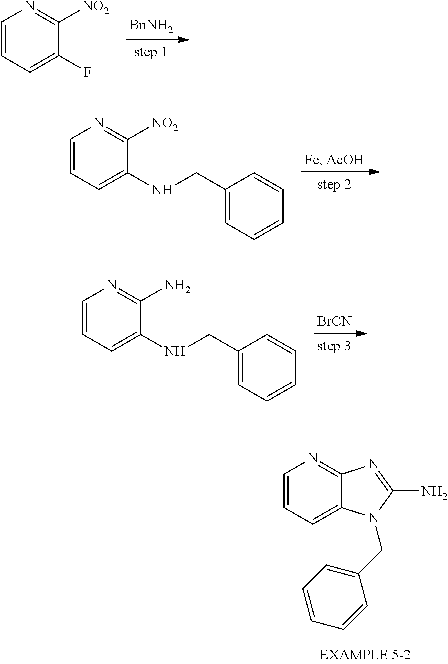

Step 1: To a mixture of 3-fluoro-2-nitro-pyridine (2.00 g, 14.1 mmol, 1.00 eq.) in ethyl alcohol (50.0 mL) was added benzylamine (7.54 g, 70.4 mmol, 7.67 mL, 5.00 eq.). The reaction mixture was stirred at 30° C. for 16 hours. The resulting suspension was filtered and the filter cake was air dried to afford N-benzyl-2-nitro-pyridin-3-amine (2.80 g, 12.2 mmol, 87% yield) as a yellow solid. LCMS [ESI, M+1]: 230.0. ¹H NMR (400 MHZ, CDCl₃) δ=8.14 (s, 1H), 7.89 (dd, J=1.2, 4.0 Hz, 1H), 7.26 (m, 7H), 4.55 (d, J=5.6 Hz, 2H).

Step 2: To a mixture of N-benzyl-2-nitro-pyridin-3-amine (1.00 g, 4.36 mmol, 1.00 eq.) in ethyl acetate (10.0 mL) and water (0.36 mL) was added acetic acid (5.24 g, 87.2 mmol, 4.99 mL, 20.0 eq.) at 25° C. The mixture was warmed to 50° C. and iron powder (974 mg, 17.4 mmol, 4.00 eq.) was added. Then the reaction mixture was stirred at 80° C. for 2 hours. The mixture was filtered and the filtrate was adjusted to pH 8 by saturated sodium bicarbonate solution and extracted with trichloromethane/isopropyl alcohol=3/1 (10.0 mL×4). The combined organic layers were dried over sodium sulfate, filtered and concentrated to afford N3-benzylpyridine-2,3-diamine (800 mg, 3.47 mmol, 80% yield) as a green oil. LCMS [ESI, M+1]: 200.2. ¹H NMR (400 MHZ, CDCl₃) δ=7.53 (d, J=4.4 Hz, 1H), 7.43-7.27 (m, 5H), 6.79 (d, J=7.2 Hz, 1H), 6.65 (dd, J=5.2, 7.6 Hz, 1H), 5.26-4.39 (m br, 2H), 4.31 (s, 2H).

Step 3: To a mixture of N3-benzylpyridine-2,3-diamine (400 mg, 1.73 mmol, 1.00 eq.) in ethyl alcohol (5.00 mL) and water (5.00 mL) was added cyanogen bromide (551 mg, 5.20 mmol, 383 μL, 3.00 eq.). The reaction mixture was stirred at 80° C. for 18 hours. The reaction mixture was adjusted to pH 12 with 2M sodium hydroxide and extracted with trichloromethane/isopropyl alcohol=3/1 (10.0 mL×4). The combined organic layers were dried over sodium sulfate, filtered and concentrated. The crude was purified by Prep-HPLC (HCl condition) to afford 1-benzylimidazo[4,5-b]pyridin-2-amine hydrochloride, Example 5-2 (39.1 mg, 165 μmol, 9% yield) as a yellow solid. LCMS [ESI, M+1]: 225.2. ¹H NMR (400 MHZ, MeOD) δ=8.03-8.02 (m, 1H), 7.78-7.75 (m, 1H), 7.44-7.30 (m, 3H), 7.30-7.25 (m, 2H), 7.22-7.20 (m, 1H), 5.42 (s, 2H).

### EXAMPLE 5-3

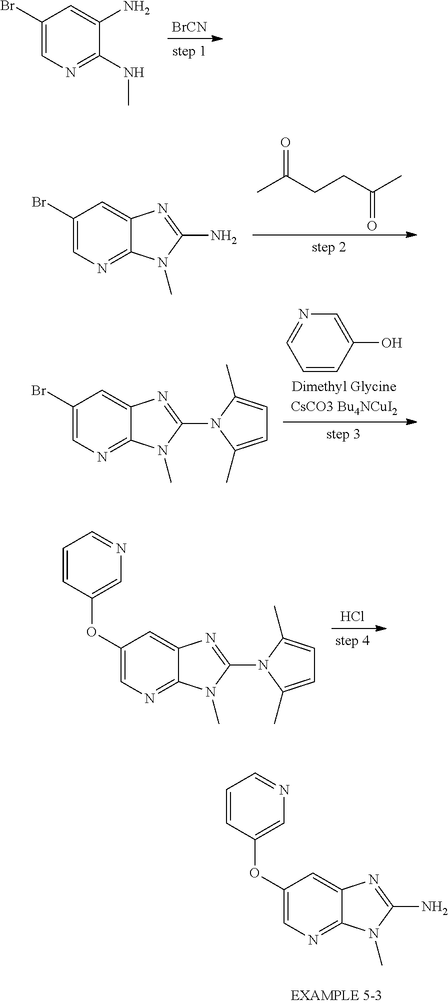

Step 1: To a solution of 5-bromo-N2-methyl-pyridine-2,3-diamine (1.00 g, 4.95 mmol, 1.00 eq.) in Water (20.0 mL) and tetrahydrofuran (2.00 mL) was added cyanogen bromide (1.82 mL, 24.7 mmol, 5.00 eq.). The mixture was stirred at 100° C. for 16 h. The resulting solution was diluted with sat.sodium bicarbonate (300 mL), extracted with ethyl acetate (300 mL), dried over sodium sulfate, filtered and concentrated. The residue was purified by silica gel chromatography (ethyl acetate in petroleum ether 0-100%) to afford 6-bromo-3-methyl-imidazo[4,5-b]pyridin-2-amine (1.80 g, 53% yield) as a pink solid. LCMS [ESI, M+1]+=229. ¹H NMR (400 MHZ, CDCl₃) δ=8.05 (s, 1H), 7.64 (s, 1H), 3.57 (s, 3H).

Step 2: To a solution of 6-bromo-3-methyl-imidazo[4,5-b]pyridin-2-amine (800 mg, 3.52 mmol, 1.00 eq.) and hexane-2,5-dione (4.02 g, 35.2 mmol, 4.13 mL, 10.0 eq.) in toluene (10.0 mL) and dimethyl formamide (2.00 mL) was added p-Toluenesulfonic acid monohydrate (67.0 mg, 352 μmol, 0.10 eq.). The mixture was stirred at 140° C. for 16 h with dean-Stark trap. The mixture was diluted with water (200 mL), extracted with ethyl acetate (200 mL). The organic layer was dried over sodium sulfate, filtered and concentrated. The residue was purified by reverse phase chromatography (formic acid condition) to afford 6-bromo-2-(2, 5-dimethylpyrrol-1-yl)-3-methyl-imidazo[4,5-b]pyridine (800 mg, 2.46 mmol, 70% yield) as a brown oil. LCMS [ESI, M+1]⁺=307.1. ¹H NMR (400 MHZ, CDCl₃) δ=8.52 (d, J=2.0 Hz, 1H), 8.24 (d, J=2.0 Hz, 1H), 5.98 (s, 2H), 3.56 (s, 3H), 2.07-2.04 (m, 6H).

Step 3: To a solution of 6-bromo-2-(2,5-dimethylpyrrol-1-yl)-3-methyl-imidazo[4,5-b]pyridine (300 mg, 983 μmol, 1.00 eq.) and pyridin-3-ol (140 mg, 1.47 mmol, 1.50 eq.) in dimethylacetamide (8.00 mL) was added 2-(dimethylamino) acetic acid (40.5 mg, 393 μmol, 0.40 eq.), cesium carbonate (960 mg, 2.95 mmol, 3.00 eq.) and Bis [(tetrabutylammonium iodide) copper (I) iodide](220 mg, 196 μmol, 0.20 eq.). The mixture was stirred at 120° C. for 16 h before being diluted with water (50.0 mL), extracted with ethyl acetate (50.0 mL), dried over sodium sulfate, filtered and concentrated. The residue was purified by silica gel chromatography (ethyl acetate in petroleum ether 0-80%) to afford 2-(2,5-dimethylpyrrol-1-yl)-3-methyl-6-(3-pyridyloxy) imidazo[4,5-b]pyridine (100 mg, 288 μmol, 29% yield) as a brown solid. LCMS [ESI, M+1]⁺=320.1. ¹H NMR (400 MHZ, CDCl₃) δ=8.46 (d, J=1.6 Hz, 1H), 8.40 (br d, J=4.0 Hz, 1H), 8.36 (d, J=2.4 Hz, 1H), 7.78 (d, J=2.4 Hz, 1H), 7.35-7.29 (m, 2H), 5.98 (s, 2H), 3.59 (s, 3H), 2.09-2.08 (m, 6H).

Step 4: To a solution of 2-(2,5-dimethylpyrrol-1-yl)-3-methyl-6-(3-pyridyloxy) imidazo[4,5-b]pyridine (80.0 mg, 250 μmol, 1.00 eq.) in ethyl alcohol (2.00 mL) was added hydrochloric acid (12 M, 0.20 mL, 2.01 mmol, 8.04 eq.). The mixture was stirred at 120° C. in a sealed tube for 1.5 h in a microwave reactor. The mixture was concentrated. The residue was purified by prep-HPLC (ammonia hydroxide condition) to afford 3-methyl-6-(3-pyridyloxy) imidazo[4,5-b]pyridin-2-amine, Example 5-3 (15.5 mg, 63.0 μmol, 25% yield) as a white solid. LCMS [ESI, M+1]⁺=242.1. ¹H NMR (400 MHZ, MeOD) δ=8.29 (s, 1H), 8.26 (t, J=2.8 Hz, 1H), 7.81 (d, J=2.4 Hz, 1H), 7.46-7.35 (m, 2H), 7.26 (d, J=2.4 Hz, 1H), 3.61 (s, 3H).

### EXAMPLE 5-4

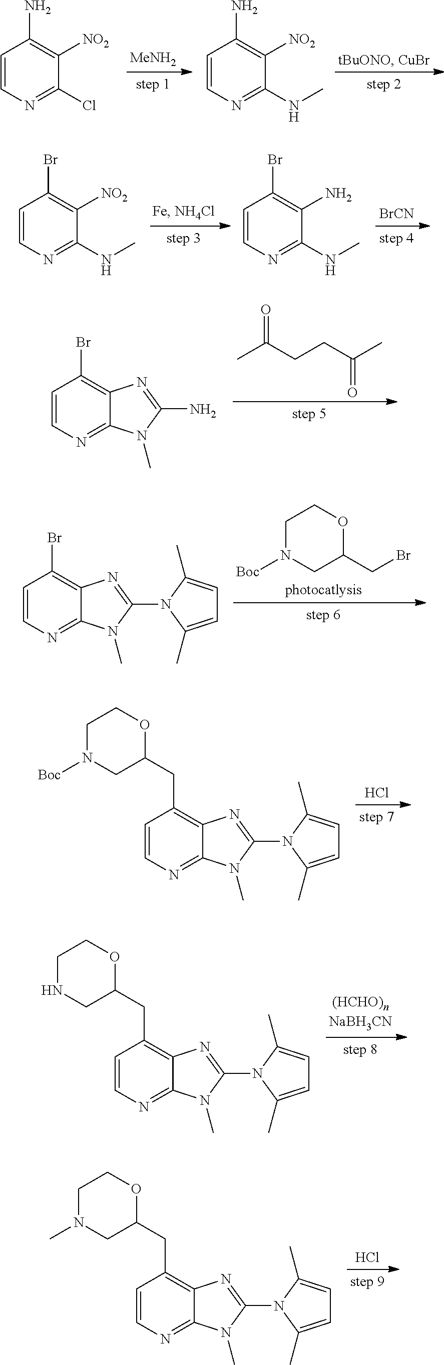

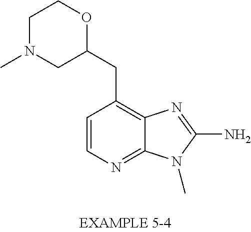 [SMILES: CN1CCOC(Cc2ccnc3c2nc(N)n3C)C1]

Step 1: To a solution of methanamine in ethanol (30%, 30.0 mL, 17.3 mmol, 1.00 eq.) was added 2-chloro-3-nitro-pyridin-4-amine (3.00 g, 17.3 mmol, 1.00 eq.) at 0° C. The mixture was stirred at 30° C. for 16 hours. The mixture was concentrated in vacuum. The residue was triturated with tert-butyl methyl ether (15.0 mL) at 25° C. for 10 minutes and filtered. The filter cake was washed with tert-butyl methyl ether (3.00 mL×3) then the filter cake was dried in vacuum to afford N2-methyl-3-nitro-pyridine-2,4-diamine (3.45 g, crude) as a yellow solid. LCMS [ESI, M+1]⁺: 169.1. ¹H NMR (400 MHZ, DMSO-d₆) δ-8.75 (br d, J=4.0 Hz, 1H), 8.15 (br s, 2H), 7.66 (d, J=6.0 Hz, 1H), 6.11 (d, J=5.6 Hz, 1H), 2.92 (d, J=4.8 Hz, 3H).

Step 2: To a solution of N2-methyl-3-nitro-pyridine-2,4-diamine (2.45 g, 14.6 mmol, 1.00 eq.) and copper (I) bromide (4.18 g, 29.1 mmol, 888 μL, 2.00 eq.) in acetonitrile (70.0 mL) was added tert-butyl nitrite (3.76 g, 36.4 mmol, 4.33 mL, 2.50 eq.) at 0° C. under nitrogen atmosphere. The mixture was stirred at 25° C. for 16 hours. The mixture was filtered. The filter cake was washed with dichloromethane (10.0 mL×3). The filtrate was concentrated in vacuum. The residue was purified by reverse phase (Formic acid condition) and the collected eluent was concentrated in vacuum to remove acetonitrile. The resulting mixture was extracted with ethyl acetate (300 mL×3). The combined organic layers were dried over anhydrous sodium sulfate, filtered and concentrated in vacuum to afford 4-bromo-N-methyl-3-nitro-pyridin-2-amine (1.40 g, 5.85 mmol, 40% yield) as a yellow oil. LCMS [ESI, M+1]⁻: 232.1/234.1.

Step 3: To a solution of 4-bromo-N-methyl-3-nitro-pyridin-2-amine (1.40 g, 6.03 mmol, 1.00 eq.) in ethanol (25.0 mL) and water (5.00 mL) was added iron powder (1.68 g, 30.2 mmol, 5.00 eq.) and saturated ammonium chloride (1.61 g, 30.2 mmol, 5.00 eq.). The mixture was stirred at 80° C. for 1 hour before being filtered. The filtrate was concentrated in vacuum. The residue was diluted with ethyl acetate (10.0 mL) and then filtered. The filter cake was washed with methanol (5.00 mL×2). The combined filtrate was concentrated in vacuum. The residue was purified by reverse phase (formic acid condition) to afford-bromo-N2-methyl-pyridine-2,3-diamine (300 mg, 1.41 mmol, 28% yield) as a yellow solid. LCMS [ESI, M+1]⁻: 201.9/203.9.

Step 4: To a solution of 4-bromo-N2-methyl-pyridine-2,3-diamine (300 mg, 1.48 mmol, 1.00 eq.) in water (5.00 mL) and tetrahydrofuran (1.00 mL) was added cyanogen bromide (328 μL, 4.45 mmol, 3.00 eq.) at 25° C. The mixture was stirred at 100° C. for 16 hours. The mixture was cooled to ambient temperature, basified with sodium hydroxide solution (2N) slowly until pH 10. The resulting mixture was extracted with ethyl acetate (5.00 mL×3). The combined organic layers were washed with water (10.0 mL) and brine (10.0 mL), dried over anhydrous sodium sulfate, filtered and concentrated in vacuum. The residue was purified by flash silica gel chromatography (methanol/ethyl acetate 4:1). The collected mixture was concentrated in vacuum to afford 7-bromo-3-methyl-imidazo[4,5-b]pyridin-2-amine (160 mg, 510 μmol, 34% yield) as a yellow solid. LCMS [ESI, M+1]⁻: 227.0/229.0. ¹H NMR (400 MHZ, MeOD-d₄) δ=7.76 (d, J=5.4 Hz, 1H), 7.22 (d, J=5.4 Hz, 1H), 3.59 (s, 3H).

Step 5: To a solution of 7-bromo-3-methyl-imidazo[4,5-b]pyridin-2-amine (250 mg, 1.10 mmol, 1.00 eq.) in toluene (20.0 mL) and N,N-dimethylformamide (6.00 mL) was added 4-methylbenzenesulfonic acid hydrate (12.6 mg, 66.1 μmol, 0.06 eq.) and hexane-2,5-dione (628 mg, 5.51 mmol, 646 μL, 5.00 eq.) at 25° C. The mixture was stirred at 140° C. (reflux) with dean-Stark trap for 16 hours. The mixture was concentrated in vacuum to remove toluene. The resulting mixture was diluted with water (20.0 mL) and extracted with ethyl acetate (15.0 mL×3). The combined organic layers were washed with water (20.0 mL). The separated organic phase was dried over anhydrous sodium sulfate, filtered and concentrated in vacuum. The residue was purified by flash silica gel chromatography (ethyl acetate in petroleum ether 18%). The collected mixture was concentrated in vacuum to afford 7-bromo-2-(2,5-dimethylpyrrol-1-yl)-3-methyl-imidazo[4,5-b]pyridine (145 mg, 471 μmol, 43% yield) as a yellow oil. LCMS [ESI, M+1]⁻: 305.0/307.0. ¹H NMR (400 MHZ, CDCl₃) δ=8.33-8.19 (m, 1H), 7.63-7.42 (m, 1H), 6.01-5.85 (m, 2H), 3.60-3.52 (m, 3H), 2.10-2.02 (m, 6H).

Step 6: To a 15 mL vial equipped with a stir bar was added 7-bromo-2-(2,5-dimethyl-1H-pyrrol-1-yl)-3-methyl-3H-imidazo[4,5-b]pyridine (145 mg, 475 μmol, 1.00 eq.), tert-butyl 2-(bromomethyl) morpholine-4-carboxylate (173 mg, 618 μmol, 1.30 eq.), (4,4-Di-tert-butyl-2,2-bipyridine)bis [3,5-difluoro-2-[5-trifluoromethyl-2-pyridinyl-kappaN)phenyl-kappaC]iridium (III) hexafluorophosphate (5.33 mg, 4.75 μmol, 0.01 eq.), [4,4′-Bis(1,1-dimethylethyl)-2,2′-bipyridine]nickel (II) dichloride (946 μg, 2.38 μmol, 0.005 eq.), 1,1,1,3,3,3-hexamethyl-2-(trimethylsilyl)trisilane (118 mg, 475 μmol, 147 μL, 1.00 eq.), sodium carbonate (101 mg, 950 μmol, 2.00 eq.) and 1,2-dimethoxyethane (2.00 mL). The vial was sealed under nitrogen atmosphere. The reaction was stirred and irradiated with a 34 W blue LED lamp (7 cm away), with a cooling fan to keep the reaction temperature at 25° C. for 14 hours. The mixture was concentrated in vacuum. The residue was purified by flash silica gel chromatography (ethyl acetate in petroleum ether 57%). The collected mixture was concentrated in vacuum to afford tert-butyl 2-((2-(2,5-dimethyl-1H-pyrrol-1-yl)-3-methyl-3H-imidazo[4,5-b]pyridin-7-yl)methyl) morpholine-4-carboxylate (58.0 mg, 112 μmol, 24% yield) as a yellow oil. LCMS [ESI, M+1]⁻: 426.2. ¹H NMR (400 MHZ, CDCl₃) δ=8.38 (d, J=5.2 Hz, 1H), 7.22 (br d, J=4.4 Hz, 1H), 6.01-5.93 (m, 2H), 5.19-5.07 (m, 1H), 3.96 (br d, J=12.4 Hz, 2H), 3.85 (m, 3H), 3.74 (br t, J=4.8 Hz, 1H), 3.58-3.54 (m, 4H), 2.07 (s, 6H), 1.47 (s, 6H), 1.44 (s, 9H).

Step 7: To a solution of tert-butyl 2-((2-(2,5-dimethyl-1H-pyrrol-1-yl)-3-methyl-3H-imidazo[4,5-b]pyridin-7-yl)methyl) morpholine-4-carboxylate (58.0 mg, 112 μmol, 82.3% purity, 1.00 eq.) in metnanol (1.00 mL) was added HCl in methanol (4 M, 1.00 mL, 35.7 eq.) at 25° C. The mixture was stirred at 25° C. for 1 hour. The mixture was concentrated in vacuum. The residue was diluted with dichloromethane (3.00 mL) and basified with sodium bicarbonate solid until pH 8. The resulting mixture was filtered. The filter cake was washed with dichloromethane (3.00 mL×2). The combined filtrate was concentrated in vacuum to afford 2-[[2-(2,5-dimethylpyrrol-1-yl)-3-methyl-imidazo[4,5-b]pyridin-7-yl]methyl]morpholine (50.0 mg, crude) as a yellow oil. LCMS [ESI, M+1]⁻: 326.1.

Step 8: To a solution of 2-[[2-(2,5-dimethylpyrrol-1-yl)-3-methyl-imidazo[4,5-b]pyridin-7-yl]methyl]morpholine (30.0 mg, 65.0 μmol, ˜70% purity, 1.00 eq.) in methanol (1.00 mL) was added paraformaldehyde (100 mg, 65.0 μmol, 1.00 eq.), sodium cyanoborohydride (6.13 mg, 97.5 μmol, 1.50 eq.) and acetic acid (9.76 mg, 162 μmol, 9.29 μL, 2.50 eq.) at 25° C. The mixture was stirred at 25° C. for 17 hours. The mixture was concentrated in vacuum. The residue was purified by silica gel chromatography (methanol/ethyl acetate 8:1) to afford 2-[[2-(2,5-dimethylpyrrol-1-yl)-3-methyl-imidazo[4,5-b]pyridin-7-yl]methyl]-4-methyl-morpholine (20.0 mg, 58.4 μmol, 54% yield) as a white solid. LCMS [ESI, M+1]⁺: 340.2

Step 9: To a solution of 2-[[2-(2,5-dimethylpyrrol-1-yl)-3-methyl-imidazo[4,5-b]pyridin-7-yl]methyl]-4-methyl-morpholine (15.0 mg, 44.2 μmol, 1.00 eq.) in ethanol (1.50 mL) was added hydrochloric acid (12 M, 0.10 mL, 27.2 eq.). The mixture was stirred at 120° C. for 1.5 hours in a microwave reactor. The mixture was concentrated in vacuum. The residue was diluted with methanol (2.00 mL) and then basified with ammonium hydroxide solution until pH 8. The resulting mixture was purified by prep-HPLC (ammonium bicarbonate condition) to afford 3-methyl-7-[(4-methylmorpholin-2-yl)methyl]imidazo[4,5-b]pyridin-2-amine (3.57 mg, 13.5 μmol, 23% yield) as a yellow oil. LCMS [ESI, M+1]⁺: 262.1. ¹H NMR (400 MHZ, MeOD-d₄) δ=7.85 (d, J=5.2 Hz, 1H), 6.97 (d, J=5.2 Hz, 1H), 3.95-3.87 (m, 1H), 3.87-3.80 (m, 1H), 3.66-3.53 (m, 4H), 3.00 (dq, J=6.8, 14.0 Hz, 2H), 2.78-2.61 (m, 2H), 2.24 (s, 3H), 2.14 (dt, J=3.2, 11.6 Hz, 1H), 1.93 (dd, J=10.4, 11.6 Hz, 1H).

### EXAMPLE 6-1

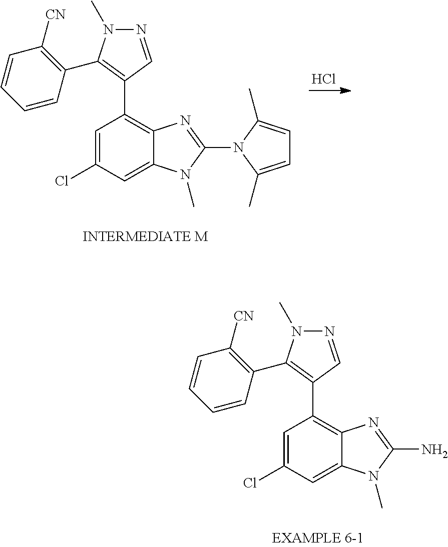 [SMILES: Cc1ccc(C)n1-c1nc2c(-c3cnn(C)c3-c3ccccc3C#N)cc(Cl)cc2n1C ; Cl ; Cn1ncc(-c2cc(Cl)cc3c2nc(N)n3C)c1-c1ccccc1C#N]

Step 1: To a solution of Intermediate M (35.0 mg, 79.4 μmol, 1.00 eq.) in ethanol (0.70 mL) was added hydrochloric acid (12 M, 233 μL, 35.3 eq.). The mixture was stirred at 120° C. for 1.5 hours in a microwave reactor. The mixture was concentrated in vacuum and then basified with ammonium hydroxide solution until pH 8. The residue was purified by prep-HPLC (ammonium bicarbonate condition) to afford 2-[4-(2-amino-6-chloro-1-methyl-benzimidazol-4-yl)-2-methyl-pyrazol-3-yl]benzonitrile (10.4 mg, 27.0 μmol, 24% yield) as an orange solid. LCMS [ESI, M+1]⁺: 363.0. ¹H NMR (400 MHZ, MeOD-d₄) δ=8.11 (s, 1H), 7.88 (d, J=8.0 Hz, 1H), 7.78 (dt, J=1.2, 7.6 Hz, 1H), 7.70-7.63 (m, 1H), 7.60 (d, J=7.6 Hz, 1H), 7.03 (d, J=2.0 Hz, 1H), 6.43 (d, J=2.0 Hz, 1H), 3.79 (s, 3H), 3.51 (s, 3H).

### EXAMPLE 6-2

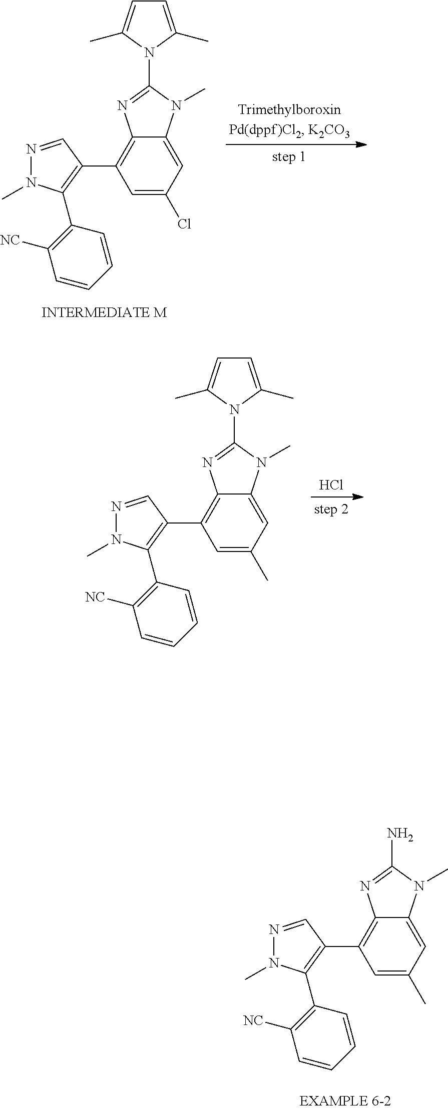

Step 1: To a solution of Intermediate M (100 mg, 227 μmol, 1.00 eq.) and 2,4,6-trimethyl-1,3,5,2,4,6-trioxatriborinane (285 mg, 1.13 mmol, 317 μL, 50% purity, 5.00 eq.) in dioxane (1.20 mL) was added potassium carbonate (94.0 mg, 680 μmol, 3.00 eq.), followed by [1,1′-bis(diphenylphosphino) ferrocene]dichloropalladium (II) (33.2 mg, 45.4 μmol, 0.20 eq.) at 25° C. under nitrogen atmosphere. The mixture was stirred at 100° C. for 16 hours. The resulting mixture was poured into water (10.0 mL) and then extracted with ethyl acetate (5.00 mL×3). The combined organic layers were washed with water (5.00 mL). The organic layer was separated, dried over anhydrous sodium sulfate, filtered and concentrated in vacuum. The residue was purified by silica gel chromatography (ethyl acetate in petroleum ether 65%). The collected mixture was concentrated in vacuum to afford 2-[4-[2-(2,5-dimethylpyrrol-1-yl)-1,6-dimethyl-benzimidazol-4-yl]-2-methyl-pyrazol-3-yl]benzonitrile (120 mg, 267 μmol, 59% yield) as a yellow oil. LCMS [ESI, M+1]⁺: 421.2.

Step 2: To a solution of 2-[4-[2-(2,5-dimethylpyrrol-1-yl)-1,6-dimethyl-benzimidazol-4-yl]-2-methyl-pyrazol-3-yl]benzonitrile (120 mg, 285 μmol, 1.00 eq.) in ethanol (1.00 mL) was added hydrochloric acid (12 M, 0.10 mL, 4.21 eq.) at 25° C. The mixture was stirred at 120° C. for 1.5 hours under microwave. The mixture was basified with ammonium hydroxide solution till pH 8 and then concentrated in vacuum. The residue was purified by prep-HPLC (HCl condition) to afford 2-[4-(2-amino-1,6-dimethyl-benzimidazol-4-yl)-2-methyl-pyrazol-3-yl]benzonitrile hydrochloride, Example 6-2 (27.7 mg, 72.5 μmol, 25% yield) as an off-white solid. LCMS [ESI, M+1]⁺: 343.2. ¹H NMR (400 MHz, MeOD-d₄) δ=7.89-7.82 (m, 2H), 7.82-7.72 (m, 2H), 7.70-7.63 (m, 1H), 7.17 (s, 1H), 6.54 (s, 1H), 3.86 (s, 3H), 3.65 (s, 3H), 2.23 (s, 3H).

### EXAMPLE 6-3

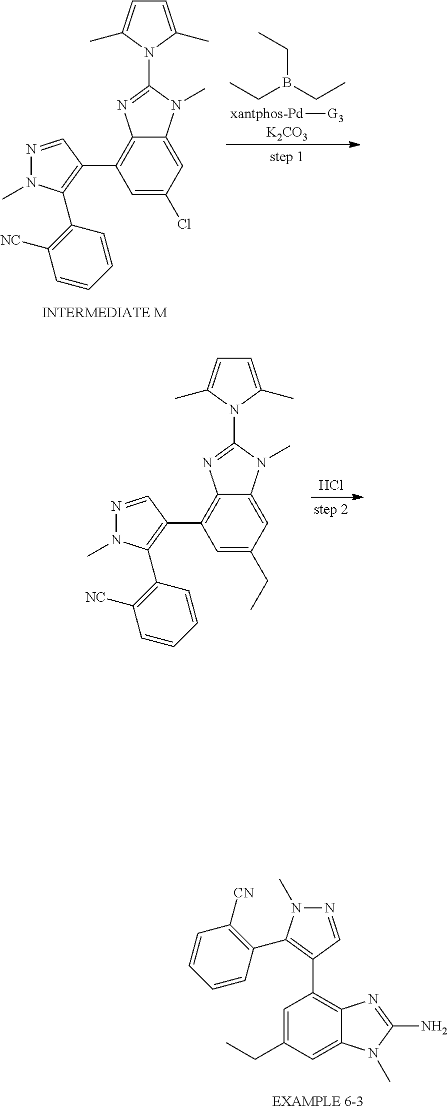

Step 1: To a solution of Intermediate M (100 mg, 226 μmol, 1.00 eq.) and triethylborane (1.00 M, 1.13 mL, 5.00 eq) in dioxane (2.00 mL) and water (0.40 mL) was added [1,1′-bis(diphenylphosphino) ferrocene]dichloropalladium (II) (18.4 mg, 22.6 μmol, 0.10 eq.) and potassium carbonate (94.0 mg, 680 μmol, 3.00 eq.) at 25° C. The mixture was stirred at 90° C. for 16 h. The resulting solution was diluted with water (50.0 mL), extracted with ethyl acetate (50.0 mL), dried over anhydrous sodium sulfate, filtered and concentrated. The residue was purified by silica gel chromatography (ethyl acetate in petroleum ether 0-70%) to affor d₂— [4-[2-(2,5-dimethylpyrrol-1-yl)-6-ethyl-1-methyl-benzimidazol-4-yl]-2-methyl-pyrazol-3-yl]benzonitrile (70.0 mg, 146 μmol, 65% yield) as a colorless gum. LCMS [ESI, M+1]⁺=435.3.

Step 2: To a solution of 2-[4-[2-(2,5-dimethylpyrrol-1-yl)-6-ethyl-1-methyl-benzimidazol-4-yl]-2-methyl-pyrazol-3-yl]benzonitrile (65.0 mg, 149 μmol, 1.00 eq.) in ethyl alcohol (2.00 mL) was added hydrochloric acid (12M, 0.20 mL, 1.96 mmol, 13.0 eq.). The mixture was stirred at 120° C. a microwave reactor for 1.5 h. The mixture was concentrated. The residue was purified by Prep-HPLC (HCl condition) to afford 2-[4-(2-amino-6-ethyl-1-methyl-benzimidazol-4-yl)-2-methyl-pyrazol-3-yl]benzonitrile hydrochloride, Example 6-3 (17.6 mg, 42.1 μmol, 28% yield) as a white solid. LCMS [ESI, M+1]⁺=357.2. ¹H NMR (400 MHZ, MeOD) δ=7.89 (s, 1H), 7.86 (dt, J=1.2, 7.6 Hz, 1H), 7.81-7.73 (m, 2H), 7.70-7.63 (m, 1H), 7.19 (s, 1H), 6.54 (d, J=1.2 Hz, 1H), 3.87 (s, 3H), 3.67 (s, 3H), 2.53 (q, J=7.6 Hz, 2H), 1.00 (t, J=7.6 Hz, 3H).

### EXAMPLE 7-1

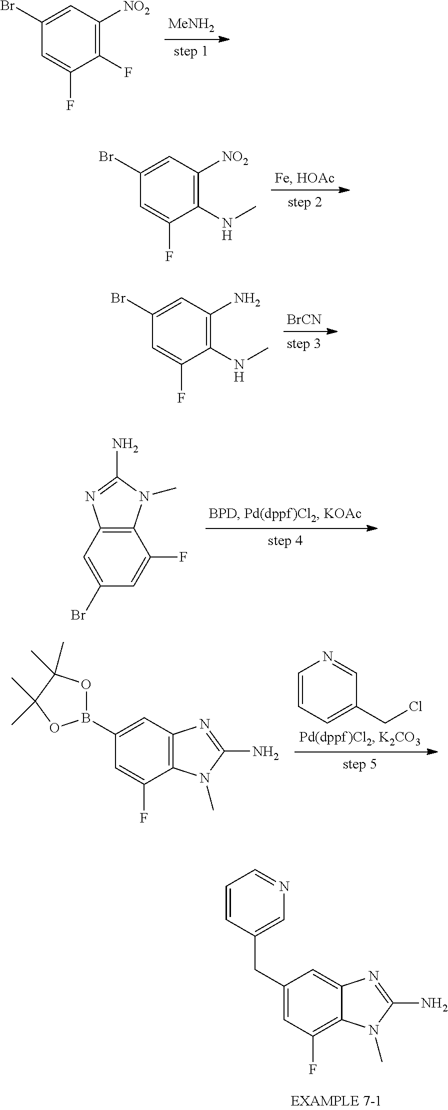

Step 1: A mixture of 5-bromo-1,2-difluoro-3-nitro-benzene (5.00 g, 21.0 mmol, 1.00 eq.) and methanamine ethyl alcohol (33%, 50.0 mL, 21.0 mmol, 1.00 eq.) was stirred at 20° C. for 0.5 hours. The reaction mixture was filtered and the filtrate was concentrated under reduced pressure to obtain 4-bromo-2-fluoro-N-methyl-6-nitro-aniline (5.00 g, 19.9 mmol, 95% yield) as a yellow solid. LCMS [M+1]⁺=250.9. ¹H NMR (400 MHZ, DMSO-d₆) δ=7.98 (t, J=2.0 Hz, 1H), 7.84 (br s, 1H), 7.74 (d, J=2.4, 13.6 Hz, 1H), 3.33 (br s, 3H).

Step 2: To a solution of 4-bromo-2-fluoro-N-methyl-6-nitro-aniline (2.30 g, 9.24 mmol, 1.00 eq.) in ethyl acetate (15.0 mL) and water (1.00 mL) was added iron powder (3.09 g, 55.4 mmol, 6.00 eq.) and acetic acid (5.00 mL). The reaction was stirred at 50° C. for 1 hour. The reaction mixture was filtered and the filtrate was neutralized to pH 7 with aqueous sodium bicarbonate solution, the aqueous phase was extracted with ethyl acetate (30.0 mL×3). The combined organic layers were washed with brine (40.0 mL), dried over anhydrous sodium sulfate, filtered and concentrated in vacuum to produce 5-bromo-3-fluoro-N2-methyl-benzene-1,2-diamine (1.38 g, crude) as a yellow oil. ¹H NMR (400 MHZ, DMSO-d₆) δ=6.57 (dd, J=1.6, 2.0 Hz, 1H), 6.51 (dd, J=2.4, 11.2 Hz, 1H), 5.24 (s, 2H), 4.06 (d, J=2.0 Hz, 1H), 3.31 (s, 3H).

Step 3: To a solution of 5-bromo-3-fluoro-N2-methyl-benzene-1,2-diamine (1.38 g, 6.30 mmol, 1.00 eq.) in ethanol (20.0 mL) was added cyanogen bromide (1.33 g, 12.6 mmol, 2.00 eq.). The reaction was stirred at 20° C. for 2 hours. The mixture was filtered and the filtrate was concentrated under reduced pressure to get 5-bromo-7-fluoro-1-methyl-benzimidazol-2-amine (916 mg, 3.57 mmol, 56% yield) as a white solid. LCMS [M+1]⁺=244.2. ¹H NMR (400 MHz, DMSO-d₆) δ=8.74 (s, 2H), 7.48 (dd, J=1.2, 10.8 Hz, 1H), 7.40 (d, J=1.2 Hz, 1H), 3.69 (s, 3H).

Step 4: To a mixture of 5-bromo-7-fluoro-1-methyl-benzimidazol-2-amine (500 mg, 2.05 mmol, 1.00 eq.), 4,4,5,5-tetramethyl-2-(4,4,5,5-tetramethyl-1,3,2-dioxaborolan-2-yl)-1,3,2-dioxaborolane (624 mg, 2.46 mmol, 1.20 eq.) in dioxane (10.0 mL) were added Pd(dppf)Cl₂ (150 mg, 205 μmol, 0.10 eq.) and potassium acetate (603 mg, 6.15 mmol, 3.00 eq.). The mixture was degassed and stirred at 90° C. for 14 hours. The mixture was concentrated under vacuum. The residue was diluted with water (20.0 mL) and extracted with ethyl acetate (20.0×3 mL). The organic layer was dried over anhydrous sodium sulfate, filtered and concentrated in vacuum. The residue was purified by silica gel column chromatography (ethyl acetate in petroleum ether 50-100%) to get 7-fluoro-1-methyl-5-(4,4,5,5-tetramethyl-1,3,2-dioxaborolan-2-yl)benzimidazol-2-amine (400 mg, 1.37 mmol, 67% yield) as a dark brown gum. ¹H NMR (400 MHZ, DMSO-d₆) δ=7.26 (s, 1H), 6.92 (d, J=11.6 Hz, 1H), 6.70 (s br, 2H), 3.66 (s, 3H), 1.28 (s, 12H).

Step 5: A mixture of 3-(chloromethyl)pyridine (92.0 mg, 721 μmol, 1.05 eq.), 7-fluoro-1-methyl-5-(4,4,5,5-tetramethyl-1,3,2-dioxaborolan-2-yl)benzimidazol-2-amine (200 mg, 687 μmol, 1.00 eq.), Pd(dppf)Cl₂ (50.3 mg, 68.7 μmol, 0.10 eq.), potassium carbonate (285 mg, 2.06 mmol, 3.00 eq.), dioxane (5.00 mL) and water (1.00 mL was degassed and stirred at 100° C. for 4 hours. The mixture was cooled to room temperature and separated between ethyl acetate (30.0 mL) and water (30.0 mL). The organic layer was dried over sodium sulfate and then concentrated in vacuum. The residue was purified by silica gel column chromatography (methanol in dichloromethane 10-20%) and then further purified by prep-HPLC (HCl condition) to obtain 7-fluoro-1-methyl-5-(3-pyridylmethyl)benzimidazol-2-amine hydrochloride, Example 7-1 (25.6 mg, 85.6 μmol, 15% yield) as a white solid. LCMS [M+1]*=257.0. ¹H NMR (400 MHZ, CD₃OD) δ=8.83 (s, 1H), 8.76 (d, J=5.6 Hz, 1H), 8.53 (d, J=8.0 Hz, 1H), 8.05 (dd, J=6.0, 8.4 Hz, 1H), 7.23 (s, 1H), 7.11 (dd, J=0.8, 12.0 Hz, 1H), 4.35 (s, 2H), 3.81 (s, 3H).

### EXAMPLE A

This Example illustrates that compounds of Formula (I) in the present invention are capable of binding to PRMT5 in presence of MTA.

The PRMT5 binding activity of compounds of the present invention was determined using an HTRF binding assay or SPR assay.

### HTRF Binding Assay

A recombinant human dual expressed Avi PRMT5/His-MEP50) protein (corresponding to amino acids for PRMT5 2-637, and 2-342 for MEP50 expressed in baculovirus) was incubated with target fragments in final buffer (25 mM ADA pH 7.2, 30 μM MTA, 1 mM TCEP, 50 mM NaCl, 0.002% Tween, 5 nM proprietary Tracer binding compound prepared in-house), overnight at 2-8° C. After overnight incubation the binding is monitored after the addition of 0.5 nM Anti-His-Tb (Cisbio) after 1 hr incubation at RT (˜20-24 hrs total binding time). The HTRF signal was measured using a Clariostar reader (BMG) excitation filter (Ex Tr), dichroic filter (LP TP) and emission filters (F 665-10 and F 620-10) manufacturer's instructions. The HTRF ratio was calculated using the formula: [emission 665/emission 620]*10000. IC50's were fit using Xlfit software (IDBS) with the Hill equation fixed to 1 (fit Background+Bmax/(1+((x/IC50)\hat{ }Hill))) and results for representative compounds of Formula (I) are shown in Table A1.

TABLE A1
IC₅₀ Values for PRMT5 Binding Activity by Representative Compounds
of Formula (I) in the Presence of MTA in the HTRF Assay

| Example | IC₅₀ (nM) | Example | IC₅₀ (nM) |
|---|---|---|---|
| 1-1 | 25140 | 1-7 | >100000 |
| 1-10 | >100000 | 1-70 | >100000 |
| 1-11 | 36416 | 1-71 | >100000 |
| 1-12 | 7274 | 1-72 | >100000 |
| 1-13 | >100000 | 1-73 | >100000 |
| 1-14 | 8018 | 1-74 | >100000 |
| 1-15 | 45585 | 1-75 | >100000 |
| 1-16 | 46832 | 1-76 | >100000 |
| 1-17 | 11054 | 1-77 | 546 |
| 1-18 | 90160 | 1-78 | 72786 |
| 1-19 | 13535 | 1-79 | 9790 |
| 1-2 | 40459 | 1-8 | >100000 |
| 1-20 | 29061 | 1-80 | 15418 |
| 1-21 | 27827 | 1-81 | 13146 |
| 1-22 | >100000 | 1-82 | >100000 |
| 1-23 | 61631 | 1-83 | 13674 |
| 1-24 | >100000 | 1-84 | >100000 |
| 1-25 | 23760 | 1-85 | 20680 |
| 1-26 | 5101 | 1-86 | 29 |
| 1-27 | 86560 | 1-87 | 875 |
| 1-28 | 18559 | 1-88 | 23543 |
| 1-29 | 37670 | 1-9 | 1527 |
| 1-3 | 29984 | 2-1 | 956759 |
| 1-30 | 30501 | 2-10 | 942 |
| 1-31 | >100000 | 2-11 | 13766 |
| 1-32 | >100000 | 2-3 | 4209 |
| 1-33 | >100000 | 2-4 | 17155 |
| 1-34 | 86372 | 2-5 | >100000 |
| 1-35 | >100000 | 2-6 | >100000 |
| 1-36 | 20003 | 2-7 | >100000 |
| 1-37 | 10089 | 2-8 | 33763 |
| 1-38 | >100000 | 2-9 | 58326 |
| 1-39 | >100000 | 3-1 | 2732 |
| 1-4 | >100000 | 3-10 | 17 |
| 1-40 | 18177 | 3-11 | 691 |
| 1-41 | 29037 | 3-12 | 92.5 |
| 1-42 | 42708 | 3-13 | 1142 |
| 1-43 | 5382 | 3-14 | 1762 |
| 1-44 | >100000 | 3-15 | 875 |
| 1-45 | >100000 | 3-16 | 13831 |
| 1-46 | 2248 | 3-17 | 493 |
| 1-47 | >100000 | 3-18 | 23973 |
| 1-48 | 22593 | 3-19 | 11401 |
| 1-49 | >100000 | 3-2 | 711 |
| 1-5 | 87362 | 3-21 | 9426 |
| 1-50 | >100000 | 3-22 | 12900 |
| 1-51 | >100000 | 3-3 | 260 |
| 1-52 | >100000 | 3-30 | 15637 |
| 1-53 | 96776 | 3-4 | 2078 |
| 1-54 | >100000 | 3-5 | 2187 |
| 1-55 | >100000 | 3-6 | 260 |
| 1-56 | 47912 | 3-7 | 249 |
| 1-57 | 21469 | 3-8 | 4.38E+3 |
| 1-58 | >100000 | 3-9 | 524 |
| 1-59 | 40406 | 3-23 | 35 |
| 1-6 | 4394 | 3-24 | 53 |
| 1-60 | >100000 | 3-25 | 2 |
| 1-61 | 3678 | 4-1 | 14391 |
| 1-62 | >100000 | 4-2 | 4995 |
| 1-63 | 2.23E+4 | 5-1 | 2.72E+5 |
| 1-64 | >100000 | 5-2 | 2.46E+5 |
| 1-65 | >100000 | 5-3 | 82669 |
| 1-66 | >100000 | 5-4 | >100000 |
| 1-67 | >100000 | 6-1 | 288 |
| 1-68 | >100000 | 6-2 | 224 |
| 1-69 | 26026 | 6-3 | 215 |
| 2-12 | 7058 | 7-1 | 3749 |
| 2-2 | 25 | 1.7 | >100000 |
| 1-70 | >10000 | 1-71 | >10000 |
| 1-72 | >100000 | 1-73 | >100000 |
| 1-74 | >100000 | 1-75 | >100000 |
| 1-76 | >100000 | 1-77 | 545 |
| 1-78 | 72785 | 1-79 | 9789 |
| 1-8 | >100000 | 1-80 | 15417 |
| 1-81 | 13146 | 1-82 | >100000 |
| 1-83 | 13673 | 1-84 | >100000 |
| 1-85 | 20680 | 1-86 | 24 |
| 1-87 | 875 | | |

### SPR Binding Assay

In vivo biotinylated PRMT5-MEP50 was diluted to 4.5 M in 25 mM Bicine pH 7.6, 100 mM NaCl, 1 mM TCEP, and 0.05% Tween-20 and injected at 5 μl/min flow rate into flow cell 2 (FC2) of a Series S Sensor Chip SA (Cytiva) in a Biacore T200 or in a Biacore 8K plus (Cytiva). SPR screening was performed in MTA running buffer (25 mM Bicine pH 7.6, 100 mM NaCl, 1 mM TCEP, 20 UM MTA, 0.05% Tween-20 and 2% DMSO). The biotinylated PRMT5-MEP50 surface was equilibrated with MTA running buffer for 12 hours prior to the start. The test compound affinity was determined using multi-cycle injection of each fragment from 0.001 to 500 μM over the PRMT5.MTA at a flow rate of 30 μl/min and with association and dissociation times of 20 and 60 seconds respectively. PRMT5.MTA surface activity was confirmed at the initiation, and the end of the run by titration of EPZ015666 (K_{D}=11 and 13 mM respectively). Subsequently, compound titration was repeated in SAM-running buffer (25 mM Bicine pH 7.6, 100 mM NaCl, 1 mM TCEP, 20 μM SAM, 0.05% Tween-20, and 2% DMSO). The PRMT5·SAM surface was equilibrated for at least 5 hours prior to compound titration and the PRMT5·SAM surface activity was confirmed at the end of the fragment titration run by titration of EPZ015666 (K_{D}<1 nM). After double referencing, the steady-state response was extracted for each fragment concentration and was fit to the Langmuir isotherm equation to determine the equilibrium dissociation constant (K_{D}).

The data in Table A2 was generated using a surface plasmon resonance (“SPR”) binding assay.

TABLE A2
K_{D} Values for PRMT5 Binding Activity by Representative
Compounds of Formula (I) in the Presence
of MTA and SAM in the SPR Assay

K_{D} μM

| Example | MTA | SAM |
|---|---|---|
| 1-12 | 2.447 | 19.7 |
| 1-16 | 152.4 | 165 |
| 1-19 | 74.2 | 203 |
| 1-2 | 49.0 | 65.8 |
| 1-20 | 55.2 | 423 |
| 1-23 | 33.6 | 104 |
| 1-37 | 13.951 | 94.7 |
| 1-21 | 17.02 | 56.5 |
| 1-27 | 52.57 | 62.4 |
| 1-36 | 7.35 | 55.2 |
| 1-40 | 12.011 | 62.3 |
| 1-42 | 20.766 | — |
| 1-48 | 39.078 | 108 |
| 1-53 | 44.616 | 187 |
| 1-56 | 24.467 | 51.9 |
| 1-57 | 14.607 | 37.8 |
| 1-59 | 53.3 | 68.7 |
| 1-63 | 13.562 | 57 |
| 1-69 | 16.005 | 58.5 |
| 1-80 | 15.4 | 60.2 |
| 1-83 | 0.089 | 41.8 |
| 1-87 | 0.512 | 1.67 |
| 1-88 | 10.776 | 48 |
| 2-10 | 1.524 | 48.4 |
| 2-11 | 15.2 | 65.6 |
| 2-12 | 3.638 | 58.032 |
| 2-2 | 0.151 | 0.679 |
| 2-3 | 13.198 | 95 |
| 2-4 | 26.76 | 40.9 |
| 2-8 | 24.74 | — |
| 2-9 | 65.7 | 175 |
| 2-10 | 1.524 | 48.4 |
| 3-1 | 2.204 | 11.58 |
| 3-10 | 0.008 | 0.519 |
| 3-11 | 1.191 | 18.436 |
| 3-12 | 0.057 | 0.128 |
| 3-13 | 1.483 | 1.657 |
| 3-14 | 1.018 | 3.36 |
| 3-15 | 0.519 | 7.72 |
| 3-16 | 7.512 | 23.6 |
| 3-18 | 11.242 | 57.4 |
| 3-2 | 0.513 | 35.587 |
| 3-21 | 7.965 | 9.674 |
| 3-22 | 16.28 | 79.6 |
| 3-3 | 0.187 | 2.747 |
| 3-30 | 16.915 | 32.5 |
| 3-5 | 1.14 | 2.77 |
| 3-6 | 0.179 | 1.94 |
| 3-7 | 0.208 | 1.017 |
| 3-8 | 4.43 | 10.226 |
| 3-9 | 0.879 | 3.67 |
| 5-2 | — | 176 |
| 6-1 | 0.322 | 0.816 |
| 6-2 | 0.047 | — |
| 7-1 | 0.443 | 6.004 |

### EXAMPLE B

This Example illustrates that compounds of Formula (I) of the present invention cooperatively inhibit PRMT5 enzymatic activity in the presence of MTA.

The PRMT5 inhibitory activity of compounds of the present invention was determined using a PRMT5: MEP50 FlashPlate Assay and a PRMT5: MEP50 HotSpot Assay (Reaction Biology Corporation).

### PRMT5: MEP50 FlashPlate Assay

The assay uses purified human, PRMT5 enzyme to convert S-adenosyl-L-[methyl-3H]methionine plus histone H4 L-arginine to S-adenosyl-L-homocysteine plus histone H4 [methyl-3H]-L-arginine. The assay was carried out using Streptavidin-coated FlashPlates (Perkin Elmer), which contained a scintillant embedded in the plastic of the plate. The histone H4 peptide substrate was conjugated with biotin, which binds to the streptavidin-coated well of the plate, placing the H4 peptide in close proximity to the side well and the scintillant. The transfer of the tritiated methyl group from S-adenosyl-L-[methyl-3H]methionine to the bound histone H4 peptide generated a radiolabeled histone H4, which was quantitated by measuring in a scintillation counter to determine the activity of PRMT5 enzyme in the presence and absence of compound. The assay reactions also were conducted in the presence and absence of MTA to determine whether the compounds exhibit MTA-cooperative activity. Briefly, compounds of the present invention were solubilized in 100% DMSO at a highest concentration of 10 mM. For IC₅₀ determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity, 3 nM PRMT5/MEP50 complex (Reaction Biology Corporation) was preincubated with test compound in assay buffer containing 40 nM histone H4 peptide (amino acids 1-15)-Biotin conjugate for 20 min at room temperature. The enzymatic reaction was initiated by adding 1 μM tritiated S-adenosyl methionine (final concentration) and the reaction is allowed to proceed for 20 min. The reaction was stopped and the amount of bound, tritiated H4 peptide in each sample was determined using a scintillation counter. The IC₅₀ value for each compound was calculated from each 10-point dose-response curve for samples plus and minus MTA using GraphPad Prism software and the results for representative compounds of Formula (I) is shown in Table B1.

TABLE B1
IC₅₀ Values for PRMT5-mediated Enzymatic Activity by Representative Compounds
of Formula (I) in the Presence and Absence of MTA in the FlashPlate Assay

| | IC₅₀ + 2 μM | | | IC₅₀ + 2 μM | |
|---|---|---|---|---|---|
| Example | MTA (μM) | IC₅₀ (μM) | Example | MTA (μM) | IC₅₀ (μM) |
| 1-87 | 17.9 | >300 | 3-18 | 251 | >300 |
| 1-88 | >300 | >300 | 3-2 | 110 | >300 |
| 2-1 | >300 | >300 | 3-21 | >300 | >300 |
| 2-10 | 97.6 | >300 | 3-3 | 17.6 | 134 |
| 2-12 | >300 | >300 | 3-4 | >300 | >300 |
| 2-2 | 0.527 | 10.7 | 3-5 | >300 | >300 |
| 2-4 | >300 | >300 | 3-6 | 44.4 | >300 |
| 2-5 | >300 | >300 | 3-7 | 27.9 | 222 |
| 2-6 | >300 | >300 | 3-8 | >300 | >300 |
| 2-7 | >300 | >300 | 3-9 | 16.9 | 219 |
| 2-8 | >300 | >300 | 3-23 | 0.43 | 155 |
| 2-9 | >300 | >300 | 3-24 | 0.73 | 280 |
| 3-1 | 248 | >300 | 3-25 | 0.006 | 2.48 |
| 3-10 | 0.069 | >300 | 4-2 | 122 | >300 |
| 3-11 | >300 | >300 | 5-1 | >300 | >300 |
| 3-12 | 10.9 | 114 | 5-2 | 76.2 | >300 |
| 3-13 | 165 | >300 | 5-3 | >300 | >300 |
| 3-14 | >300 | >300 | 6-1 | 14 | >300 |
| 3-15 | 198 | >300 | 6-2 | 29.3 | >300 |
| 3-16 | >300 | >300 | 6-3 | 17.8 | 198 |
| 3-17 | >300 | >300 | 7-1 | 34.7 | >300 |
| 1-12 | >300 | — | 1-19 | >300 | — |
| 1-13 | >300 | — | 1-2 | >300 | — |
| 1-16 | >300 | — | 1-20 | >300 | — |
| 1-21 | >300 | — | 1-32 | >300 | — |
| 1-23 | >300 | — | 1-36 | >300 | — |
| 1-27 | 188 | — | 1-37 | >300 | — |
| 1-31 | >300 | — | 1-40 | >300 | — |
| 1-42 | >300 | — | 1-50 | >300 | — |
| 1-51 | >300 | — | 1-52 | >300 | — |
| 1-53 | >300 | — | 1-54 | >300 | — |
| 1-56 | >300 | — | 1-57 | >300 | — |
| 1-58 | >300 | — | 1-59 | 226 | — |
| 1-60 | >300 | — | 1.61 | 34.5 | — |
| 1-62 | >300 | — | 1-63 | >300 | — |
| 1-64 | >300 | — | 1-65 | >300 | — |
| 1-66 | >300 | — | 1-67 | >300 | — |
| 1-68 | >300 | — | 1-69 | >300 | — |
| 1-7 | >300 | — | 1-70 | >300 | — |
| 1-71 | >300 | — | 1-72 | >300 | — |
| 1-73 | >300 | — | 1-74 | >300 | — |
| 1-75 | 180 | — | 1-76 | >300 | — |
| 1-78 | >300 | — | 1-8 | >300 | — |
| 1-82 | 50.7 | — | 1-83 | >300 | — |

### PRMT5:MEP50 HotSpot Assay

The assay uses recombinant full-length histone H2A as the PRMT5 substrate. Enzymatic transfer of the tritiated methyl group from S-adenosyl-L-[methyl-3H]methionine to the histone H2A protein generated a radiolabeled histone H2A4 by measuring in a scintillation counter to determine the activity of PRMT5 enzyme in the presence and absence of compound. The assay reactions also were conducted in the presence of MTA to determine whether the compounds exhibit MTA-cooperative activity. Briefly, compounds of the present invention were solubilized in 100% DMSO at a highest concentration of 10 mM. For IC₅₀ determinations, the initial starting concentration for the serial dilutions of each compound was 50 μM. Control samples lacking compound, PRMT5/MEP50 complex or various reaction components also were prepared and processed in parallel with compound test samples. SAH was used as a positive control for assay validation. To measure PRMT5 inhibitory activity, 1 nM PRMT5/MEP50 complex (Reaction Biology Corporation) was preincubated with test compound in assay buffer containing 5 μM full-length histone H2A for 20 min at room temperature. The enzymatic reaction was initiated by adding 1 μM tritiated S-adenosyl methionine (final concentration) and the reaction was allowed to proceed for 60 min. The reaction was stopped and transferred to filter paper for detection. The amount of tritiated H2A in each sample was determined using a scintillation counter. The IC₅₀ value for each compound was calculated from each 10-point dose-response curve using GraphPad Prism software and the results for representative compounds of Formula (I) is shown in Table B2.

TABLE B2
IC₅₀ Values for PRMT5-mediated Enzymatic Activity
by Representative Compounds of Formula (I) in
the Presence of MTA in the HotSpot Assay

| | IC₅₀ + 2 μM |
|---|---|
| Example | MTA (μM) |
| 1-89 | 7.58 |
| 1-90 | 12 |
| 1-91 | 0.12 |
| 1-92 | 0.09 |
| 3-10 | >10 |
| 3-23 | >10 |
| 3-25 | 4.03 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A compound of Formula I: 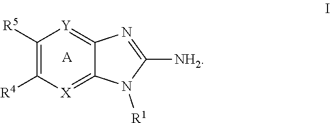
or a pharmaceutically acceptable salt thereof, wherein:
X is CR² or N
Y is CR³ or N;
R¹ is —C₁-C₂ alkyl or —CH₂-phenyl;
R² is hydrogen, cyano, —C₁-C₂ haloalkyl, or halogen;
R³ is hydrogen, halogen, or -L-R³⁰, wherein
-L is absent or is selected from the group consisting of —(CH₂)₀₋₂—NH—(CH₂)₀₋₂—, —N(CH₃)—(CH₂)₀₋₂—, —N(C(O)CH₃)—(CH₂)₀₋₂—, (—CH₂—)₁₋₂, —C(halo)₂—, —C(CH₃)H—, —O—, —S—, —C(O)—, —NH—(CH₂)₀₋₁—CH(CH₃)—(CH₂) ₀₋₁—, —NH—C(O)—(CH₂)₀₋₁— and —NH—CH(phenyl)-(CH₂)₀₋₁—;
R³⁰ is phenyl, isoxazole, oxazole, pyrrolidinone, morpholine, imidazopyridine, piperidinone, pyridinone, naphthalene, pyrazine, pyrazolidinone, —N(C₁-C₂ alkyl)₂, thiazole, thiadiazole, pyrimidine, imidazole optionally substituted with naphthyl where said naphthyl is further optionally substituted with one or more substituents selected from of cyano, halogen and —C(O)NH₂, pyrrolidine optionally fused to cyclopropane, pyrazole optionally substituted with —CH₂—CH₂-pyrrolidinone or naphthalene, pyridine optionally fused to cyclohexane, triazole optionally fused to a ring that forms pyrrolidine or morpholine, or tetrahydrofuran optionally spiro-bound to cyclobutane,
wherein each R³⁰ is optionally substituted, or is optionally further substituted, with one or more substituents independently selected from —C₁-C₃ alkyl, —C₁-C₂ haloalkyl, halogen, hydroxy, —C(O)NH₂, —C₁-C₂ alkoxy, phenyl, cyano-phenyl and cyano;
R⁴ is hydrogen, —C₁-C₂ alkyl, —CH₂—O—CH₃ or halogen;
R⁵ is
hydrogen,
-L-R⁵⁰, wherein L is defined as above and R⁵⁰ is CH(OH)—CH₃, pyridine, pyrazine, isoxazole, pyridazine, pyrimidine, pyrazole, triazole optionally fused to a ring that forms pyrrolidine or morpholine, tetrahydro-thiopyran-dioxide, thiazole, furan, isoquinoline, naphthalene, pyrrolidine, phenyl where L is not absent, imidazole optionally fused with piperidine, tetrahydrofuran optionally substituted with imidazole, tetrahydropyran optionally spiro-bound to cyclobutane, or cyclobutane fused to tetrahydropyran,
wherein each R⁵⁰ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl;
(C₀-C₃)alkyl-(CO)N(R¹⁰)(R¹¹) where
R¹⁰ is pyridyl(C₁-C₆ alkyl) where the pyridyl is optionally substituted with halogen or trifluoromethyl and
R¹¹ is pyridyl(C₁-C₆ alkyl), pyrimidinyl(C₁-C₆ alkyl), naphthyl optionally substituted in the 1, 3 or 4 position, where position 2 is the point of attachment to N, independently with one, two or three of cyano, C₁-C₃ alkyl, cyclopropylmethyl, cyclopropyl, halogen or C₁-C₂ alkoxy, or 5,6,7,8-tetrahydroquinoxalinyl, or
—(C₀-C₃)alkyl-NH—C(O)(C₁-C₂ alkyl)(R¹²) where R¹² is hydrogen, C₁-C₂ alkyl, or naphthyl optionally substituted with cyano, chloro, fluoro, cyclopropyloxy, cyclopropylmethyl, or C₁-C₂ alkyl;
wherein at least one of R³, R⁴ and R⁵ are present and are not (i) hydrogen, (ii) C₁ alkyl, (iii) C₁ alkoxy where R² is not cyano, (iv) halogen, (v) cyano or (vi) unsubstituted phenyl at R⁵, wherein Y is nitrogen, or wherein R² is cyano and R¹ is C₁ alkyl; and wherein R¹ is not C₁ alkyl if Y is nitrogen.

2. The compound or salt of claim 1, wherein ring A is selected from:

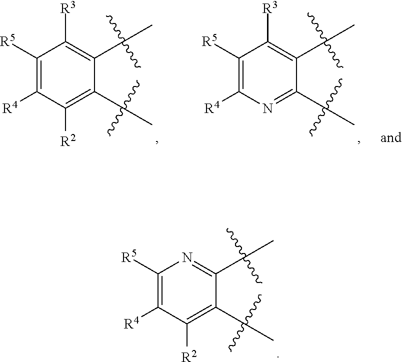

3. The compound or salt of claim 2, wherein A is:

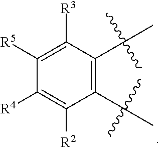

4. The compound or salt of claim 2, wherein ring A is:

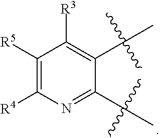

5. The compound or salt of claim 2, wherein ring A is:

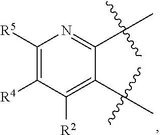

6. A compound of Formula IA: 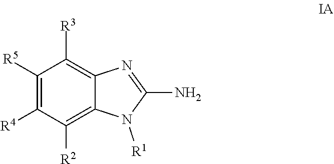
wherein:
R¹ is —C₁-C₂ alkyl or —CH₂-phenyl;
R² is hydrogen, cyano, —C₁-C₂ haloalkyl, or halogen;
R³ is hydrogen, halogen, or -L-R³⁰, wherein
-L is absent or is selected from the group consisting of —(CH₂)₀₋₂—NH—(CH₂)₀₋₂—, —N(CH₃)—(CH₂)₀₋₂—, —N(C(O)CH₃)—(CH₂)₀₋₂—, (—CH₂—)₁₋₂, —C(halo)₂—, —C(CH₃)H—, —O—, —S—, —C(O)—, —NH—(CH₂)₀₋₁—CH(CH₃)—(CH₂)₀₋₁—, —NH—C(O)—(CH₂)₀₋₁— and —NH—CH(phenyl)-(CH₂)₀₋₁—;
R³⁰ is phenyl, isoxazole, oxazole, pyrrolidinone, morpholine, imidazopyridine, piperidinone, pyridinone, naphthalene, pyrazine, pyrazolidinone, —N(C₁-C₂ alkyl)₂, thiazole, thiadiazole, pyrimidine, imidazole optionally substituted with naphthyl where said naphthyl is further optionally substituted with one or more substituents selected from of cyano, halogen and —C(O)NH₂, pyrrolidine optionally fused to cyclopropane, pyrazole optionally substituted with —CH₂—CH₂-pyrrolidinone or naphthalene, pyridine optionally fused to cyclohexane, triazole optionally fused to a ring that forms pyrrolidine or morpholine, or tetrahydrofuran optionally spiro-bound to cyclobutane,
wherein each R³⁰ is optionally substituted, or is optionally further substituted, with one or more substituents independently selected from —C₁-C₃ alkyl, —C₁-C₂ haloalkyl, halogen, hydroxy, —C(O)NH₂, —C₁-C₂ alkoxy, phenyl, cyano-phenyl and cyano;
R⁴ is hydrogen, —C₁-C₂ alkyl, —CH₂—O—CH₃ or halogen;
R⁵ is
hydrogen,
-L-R⁵⁰, wherein L is defined as above and R⁵⁰ is
—CH(OH)—CH₃, pyridine, pyrazine, isoxazole, pyridazine, pyrimidine, pyrazole, triazole optionally fused to a ring that forms pyrrolidine or morpholine, tetrahydro-thiopyran-dioxide, thiazole, furan, isoquinoline, naphthalene, pyrrolidine, phenyl where L is not absent, imidazole optionally fused with piperidine, tetrahydrofuran optionally substituted with imidazole, tetrahydropyran optionally spiro-bound to cyclobutane, or cyclobutane fused to tetrahydropyran,
wherein each R⁵⁰ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl;
—(C₀-C₃)alkyl-(CO)N(R¹⁰)(R¹¹) where
R¹⁰ is pyridyl(C₁-C₆ alkyl) where the pyridyl is optionally substituted with halogen or trifluoromethyl and
R¹¹ is pyridyl(C₁-C₆ alkyl), pyrimidinyl(C₁-C₆ alkyl), naphthyl optionally substituted in the 1, 3 or 4 position, where position 2 is the point of attachment to N, independently with one, two or three of cyano, C₁-C₃ alkyl, cyclopropylmethyl, cyclopropyl, halogen or C₁-C₂ alkoxy, or 5,6,7,8-tetrahydroquinoxalinyl, or
—(C₀-C₃)alkyl-NH—C(O)(C₁-C₂ alkyl)(R¹²) where R¹² is hydrogen, C₁-C₂ alkyl, or naphthyl optionally substituted with cyano;
wherein at least one of R³, R⁴ and R⁵ are present and are not (i) hydrogen, (ii) C₁ alkyl, (iii) C₁ alkoxy where R² is not cyano, (iv) halogen, (v) cyano or (vi) unsubstituted phenyl at R⁵, or wherein R² is cyano and R¹ is C₁ alkyl,
or a pharmaceutically acceptable salt thereof.

7. The compound or salt of any of claims 1-6, wherein L is absent.

8. The compound or salt of any of claims 1-6, wherein L is —NH—.

9. The compound or salt of any of claims 1-6, wherein L is —(CH₂)₀₋₂—NH—(CH₂)₀₋₂.

10. The compound or salt of any of claims 1-9, wherein R³ is -L-isoxazole, -L-oxazole, -L-imidazopyridine, -L-pyrazine, -L-pyrazolidinone, -L-thiazole, -L-thiadiazole, -L-pyrimidine, —L-imidazole optionally substituted with naphthyl where said naphthyl is further optionally substituted with one or more substituents selected from of cyano, halogen and —C(O)NH₂, -L-pyrazole optionally substituted with —CH₂—CH₂-pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted.

11. The compound or salt of any of claims 1-9, wherein R³ is -L-pyrrolidinone, -L-morpholine, -L-piperidinone, -L-pyridinone, -L-pyrrolidine optionally fused to cyclopropane, or -L-tetrahydrofuran optionally spiro-bound to cyclobutane, wherein R³ is optionally substituted, or is optionally further substituted.

12. The compound or salt of any of claims 1-11, wherein R⁵ is -L-pyridine, -L-pyrazine, —L-isoxazole, -L-pyridazine, -L-pyrimidine, -L-pyrazole, -L-triazole optionally fused to a ring that forms pyrrolidine or morpholine, -L-thiazole, -L-furan, -L-isoquinoline, or -L-imidazole optionally fused with piperidine, wherein R⁵ is optionally substituted, or is optionally further substituted.

13. The compound or salt of any of claims 1-11, wherein R⁵ is -L-tetrahydro-thiopyran-dioxide, -L-pyrrolidine, -L-tetrahydrofuran optionally substituted with imidazole, -L-tetrahydropyran optionally spiro-bound to cyclobutane, wherein R⁵ is optionally substituted, or is optionally further substituted.

14. The compound or salt of any of claims 1-9, wherein
(a) R³ is -L-isoxazole, -L-oxazole, -L-imidazopyridine, -L-pyrazine, -L-pyrazolidinone, -L-thiazole, -L-thiadiazole, -L-pyrimidine, -L-imidazole optionally substituted with naphthyl where said naphthyl is further optionally substituted with one or more substituents selected from of cyano, halogen and —C(O)NH₂, -L-pyrazole optionally substituted with —CH₂—CH₂—pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted, and
R⁵ is hydrogen, -L-CH(OH)—CH₃, -L-tetrahydro-thiopyran-dioxide, -L-naphthalene, -L-pyrrolidine, -L-phenyl where L is not absent, -L-tetrahydrofuran optionally substituted with imidazole, -L-tetrahydropyran optionally spiro-bound to cyclobutane, or -L-cyclobutane fused to tetrahydropyran, wherein R⁵ is optionally substituted, or is optionally further substituted;
or
(b) R³ is hydrogen, halogen, -L-phenyl, -L-pyrrolidinone, -L-morpholine, -L-piperidinone, -L-pyridinone, -L-naphthalene, -L-N(C₁-C₂ alkyl)₂, -L-pyrrolidine optionally fused to cyclopropane, or -L-tetrahydrofuran optionally spiro-bound to cyclobutane, wherein R³ is optionally substituted, or is optionally further substituted, with one or more substituents, and R⁵ is -L-pyridine, -L-pyrazine, -L-isoxazole, -L-pyridazine, -L-pyrimidine, -L-pyrazole, -L-triazole optionally fused to a ring that forms pyrrolidine or morpholine, -L-thiazole, -L-furan, —L-isoquinoline, or -L-imidazole optionally fused with piperidine, wherein R⁵ is optionally substituted, or is optionally further substituted.

15. The compound or salt of any of claims 1-9, wherein
(a) R³ is -L-pyrrolidinone, -L-morpholine, -L-piperidinone, -L-pyridinone, -L-pyrrolidine optionally fused to cyclopropane, or -L-tetrahydrofuran optionally spiro-bound to cyclobutane, wherein R³ is optionally substituted, or is optionally further substituted, and R⁵ is hydrogen, -L-CH(OH)—CH₃, -L-pyridine, -L-pyrazine, -L-isoxazole, -L-pyridazine, -L-pyrimidine, -L-pyrazole, -L-triazole optionally fused to a ring that forms pyrrolidine or morpholine, -L-thiazole, -L-furan, -L-isoquinoline, -L-naphthalene, -L-phenyl where L is not absent, -L-imidazole optionally fused with piperidine, or -L-cyclobutane fused to tetrahydropyran, wherein R⁵ is optionally substituted, or is optionally further substituted, with one or more substituents independently selected from halogen, hydroxy, —C₁-C₄ alkyl, —C₁-C₄ alkoxy, —C₁-C₄ haloalkyl, cyano and phenyl or
(b) R³ is hydrogen, halogen, -L-phenyl, -L-isoxazole, -L-oxazole, -L-imidazopyridine, -L-naphthalene, -L-pyrazine, -L-pyrazolidinone, -L-N(C₁-C₂ alkyl)₂, -L-thiazole, -L-thiadiazole, —L-pyrimidine, -L-imidazole optionally substituted with naphthyl where said naphthyl is further optionally substituted with one or more substituents selected from of cyano, halogen and —C(O)NH₂, -L-pyrazole optionally substituted with —CH₂—CH₂-pyrrolidinone or naphthalene, -L-pyridine optionally fused to cyclohexane, or -L-triazole optionally fused to a ring that forms pyrrolidine or morpholine, wherein R³ is optionally substituted, or is optionally further substituted, and
R⁵ is -L-tetrahydro-thiopyran-dioxide, -L-pyrrolidine, -L-tetrahydrofuran optionally substituted with imidazole, -L-tetrahydropyran optionally spiro-bound to cyclobutane, wherein R⁵ is optionally substituted, or is optionally further substituted.

16. The compound or salt of any of claims 1-7, wherein R⁵ is
(CO)N(R¹⁰)(R¹¹) where
R¹⁰ is pyridyl(C₁-C₆ alkyl) where the pyridyl is optionally substituted with halogen or trifluoromethyl and
R¹¹ is pyridyl(C₁-C₆ alkyl), pyrimidinyl(C₁-C₆ alkyl), naphthyl optionally substituted in the 1, 3 or 4 position, where position 2 is the point of attachment to N, independently with one, two or three of cyano, C₁-C₃ alkyl, cyclopropylmethyl, cyclopropyl, halogen or C₁-C₂ alkoxy, or 5,6,7,8-tetrahydroquinoxalinyl, or
—(C₁-C₂ alkyl)-NH—C(O)(C₁-C₂ alkyl)(R¹²) where R¹² is hydrogen, C₁-C₂ alkyl, or naphthyl optionally substituted with cyano, chloro, fluoro, cyclopropyloxy, cyclopropylmethyl, or C₁-C₂ alkyl.

17. A compound which is: 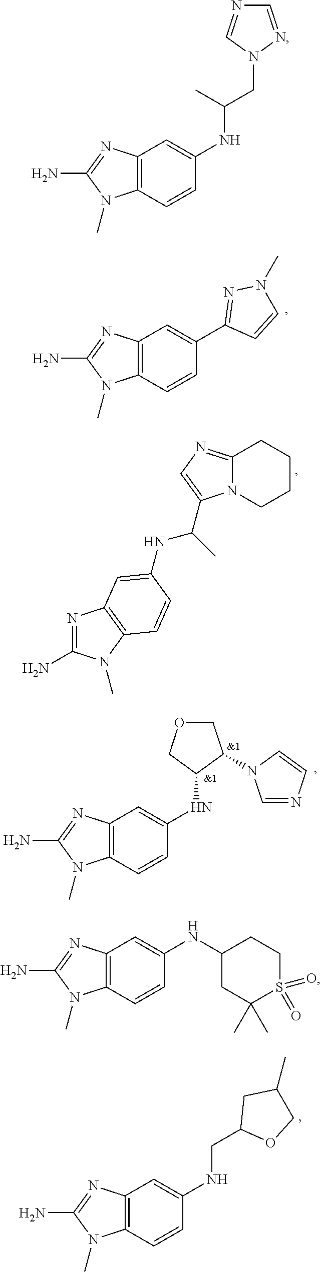 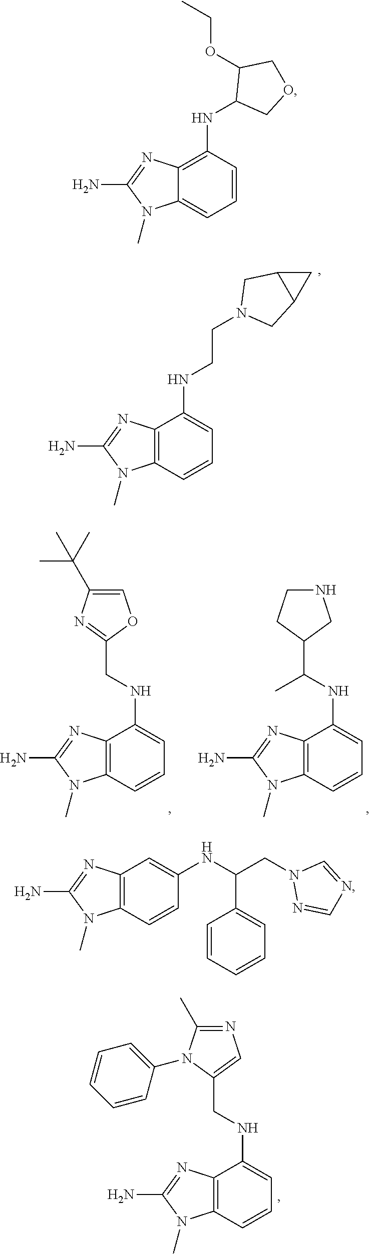 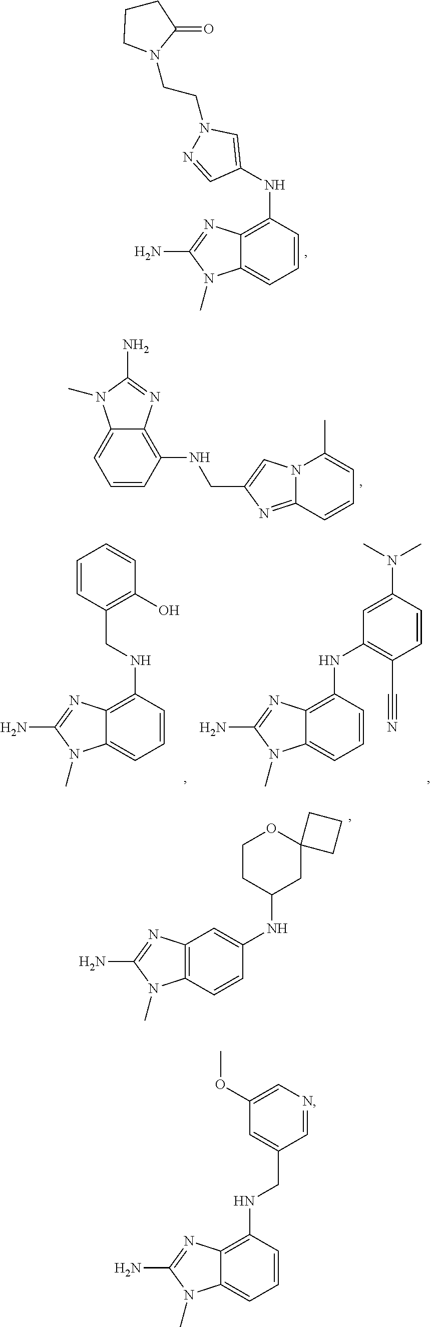 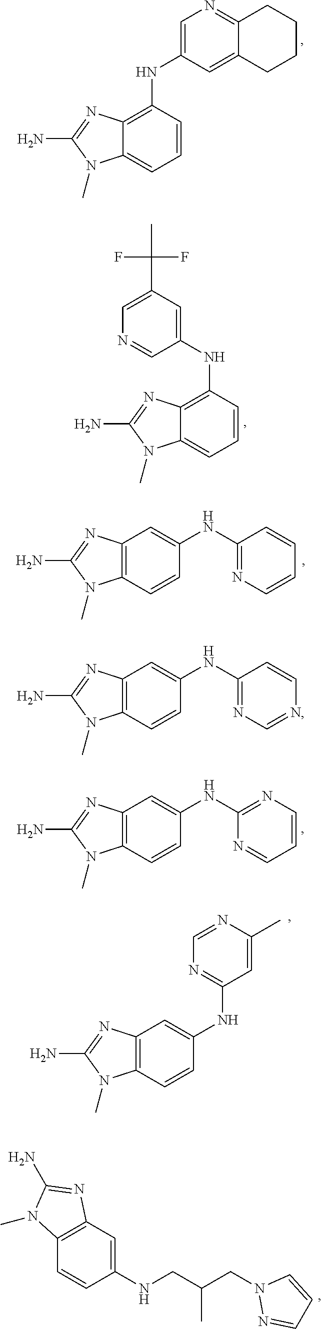 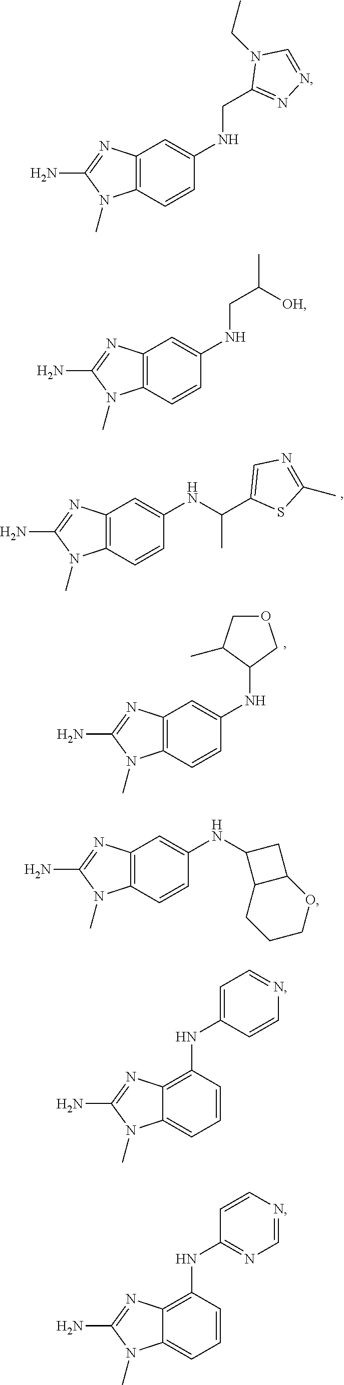 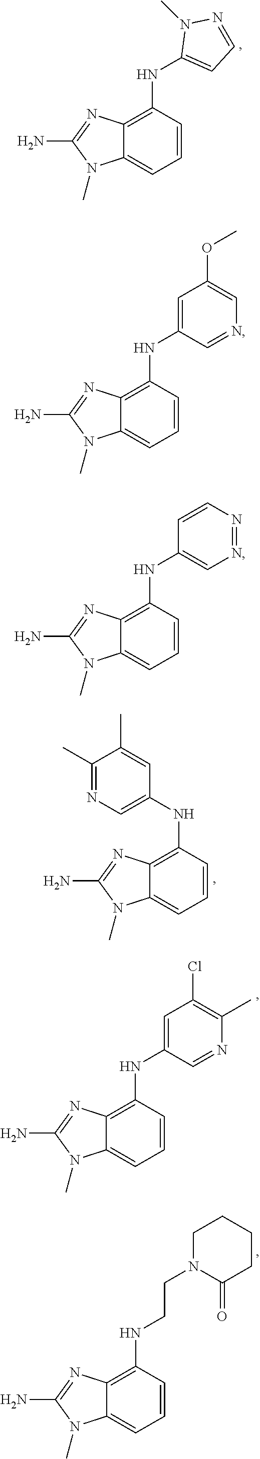 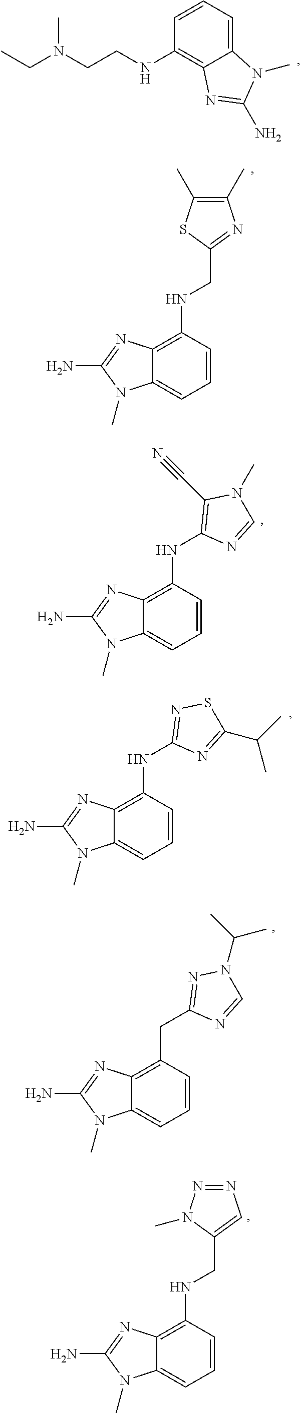 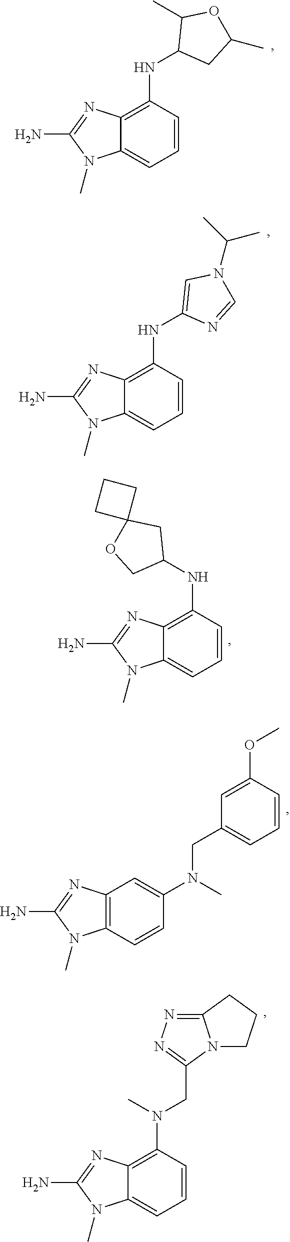 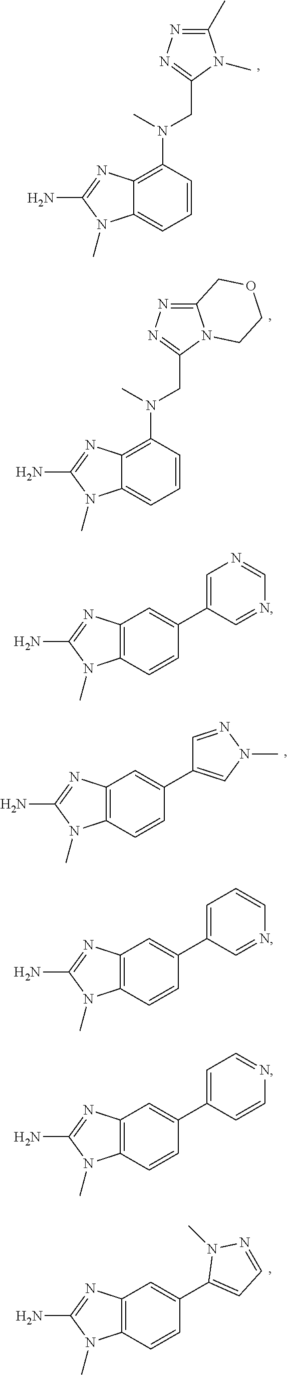 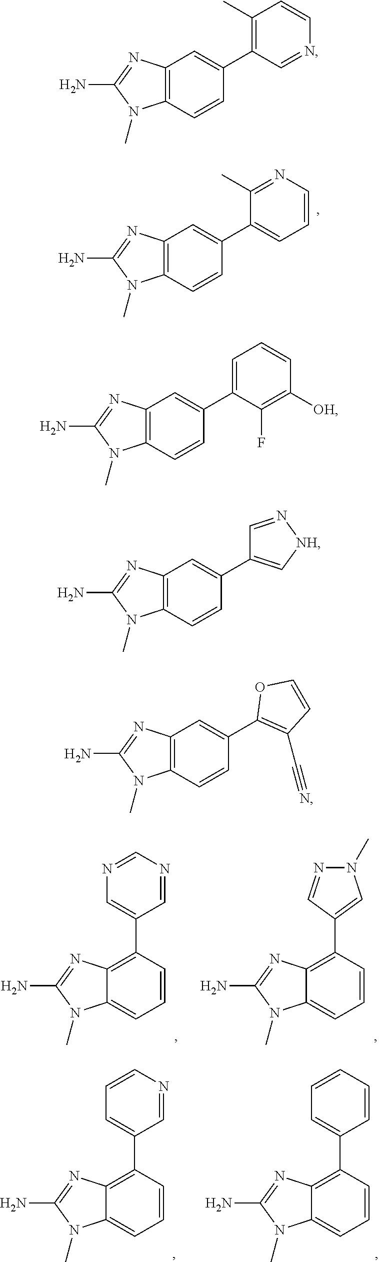 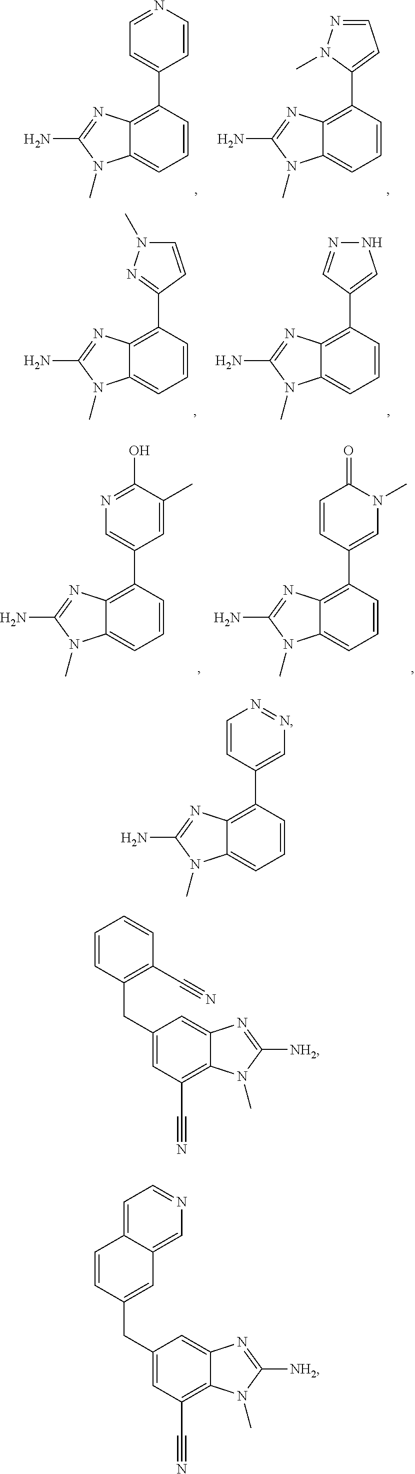 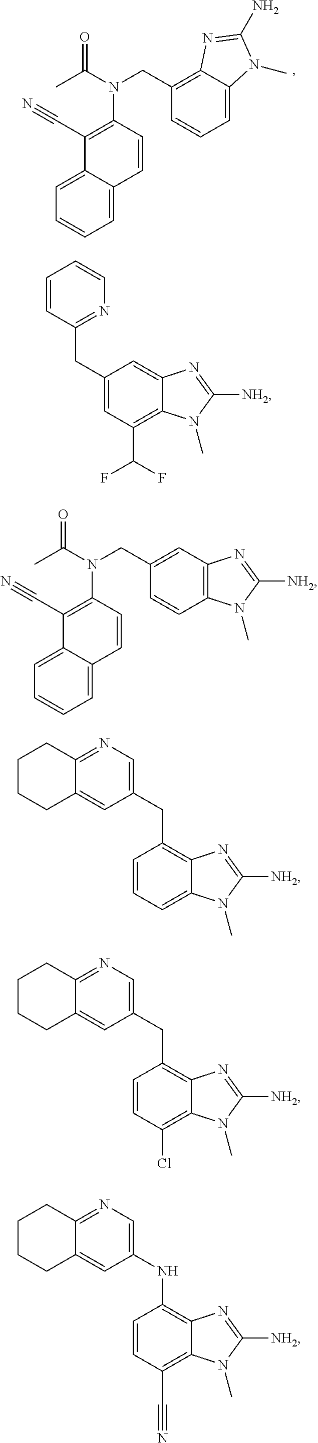 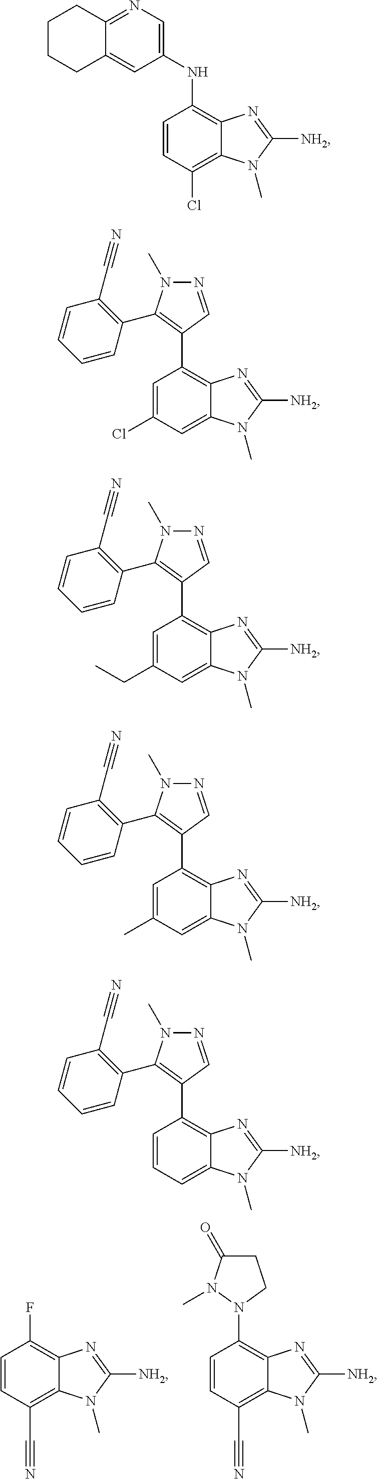 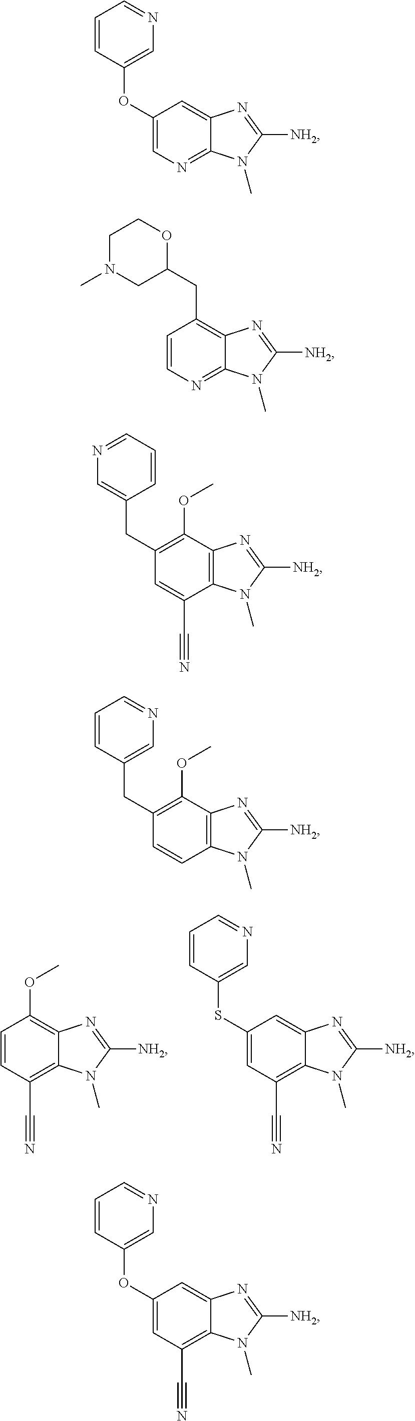 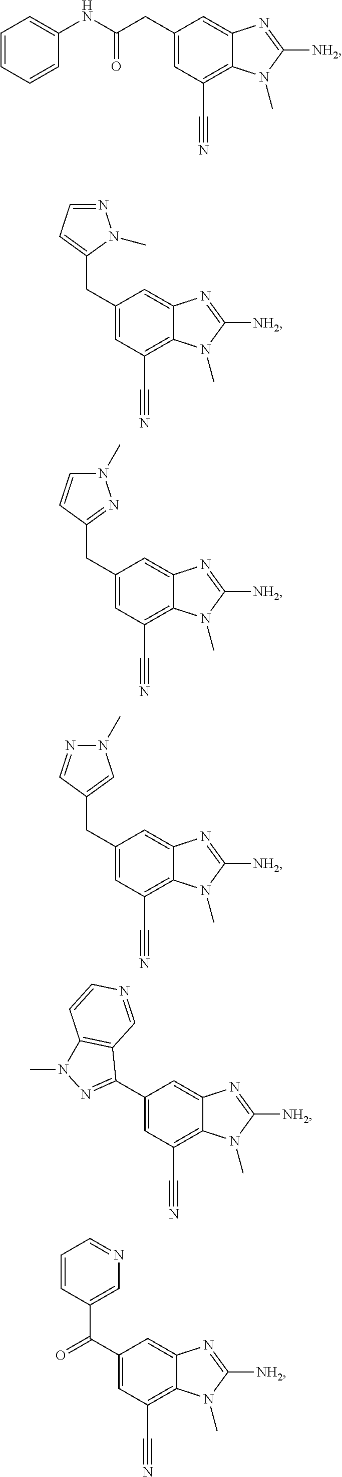 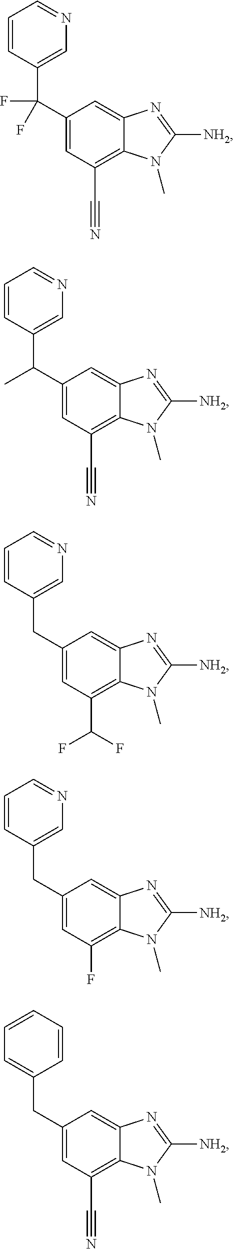 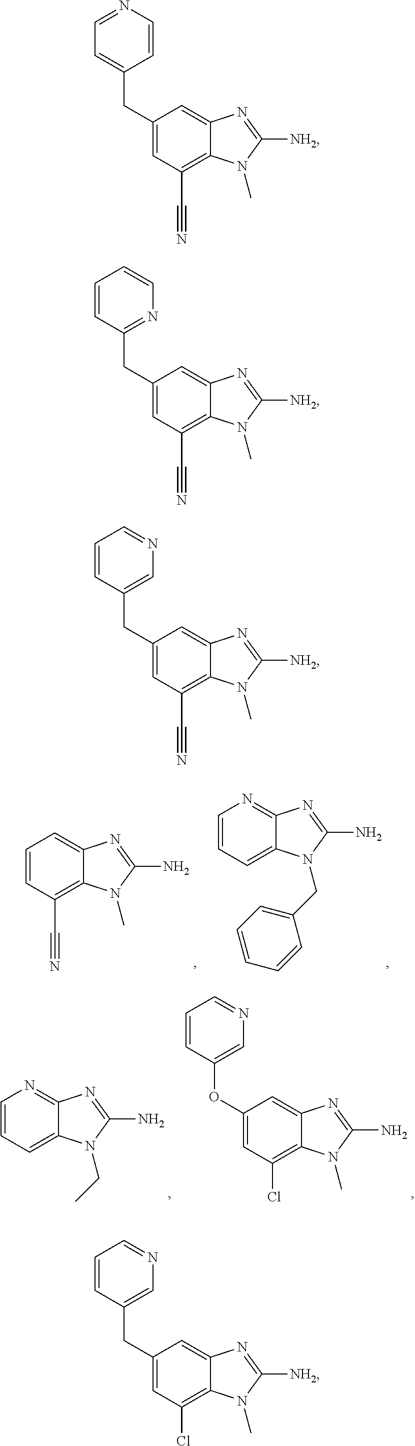 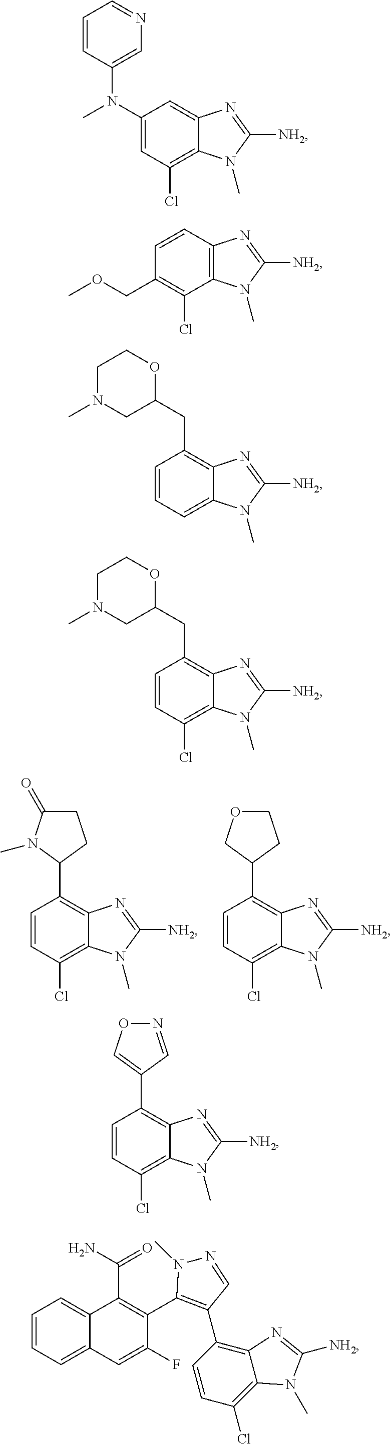 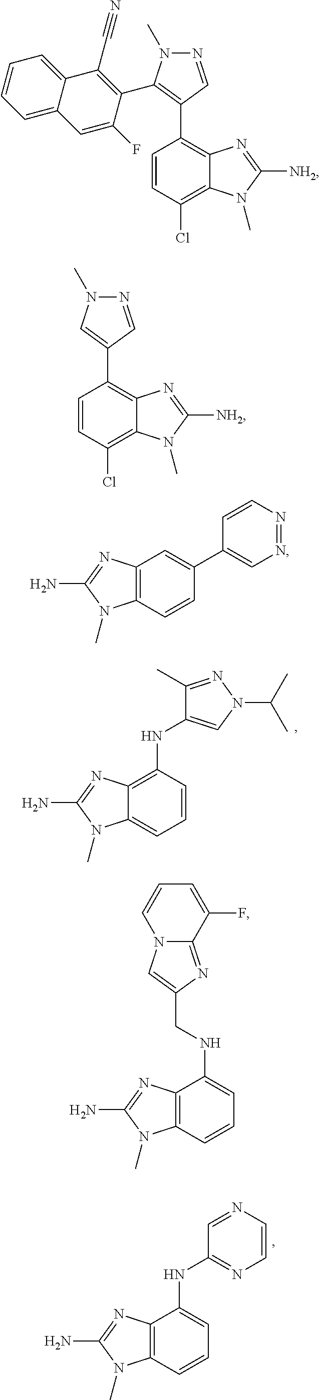 [SMILES: Cn1ncc(-c2ccc(Cl)c3c2nc(N)n3C)c1-c1c(F)cc2ccccc2c1C#N ; Cn1cc(-c2ccc(Cl)c3c2nc(N)n3C)cn1 ; Cn1c(N)nc2cc(-c3ccnnc3)ccc21 ; Cc1nn(C(C)C)cc1Nc1cccc2c1nc(N)n2C ; Cn1c(N)nc2c(NCc3cn4cccc(F)c4n3)cccc21 ; Cn1c(N)nc2c(Nc3cnccn3)cccc21] 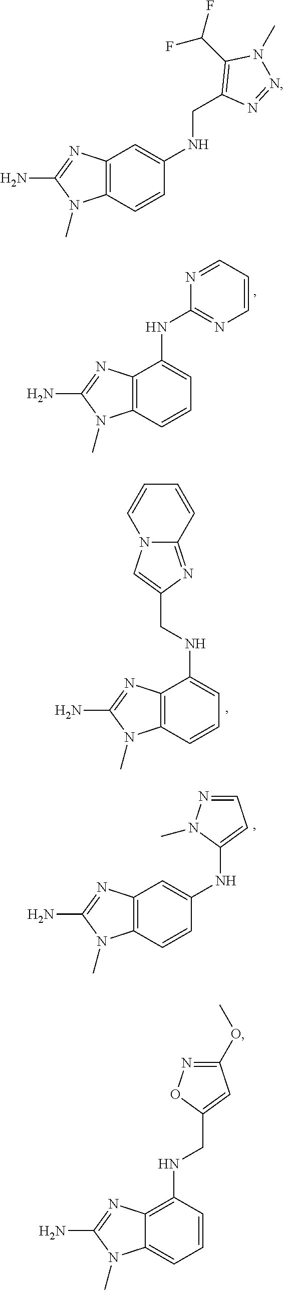 [SMILES: Cn1nnc(CNc2ccc3c(c2)nc(N)n3C)c1C(F)F ; Cn1c(N)nc2c(Nc3ncccn3)cccc21 ; Cn1c(N)nc2c(NCc3cn4ccccc4n3)cccc21 ; Cn1nccc1Nc1ccc2c(c1)nc(N)n2C ; COc1cc(CNc2cccc3c2nc(N)n3C)on1] 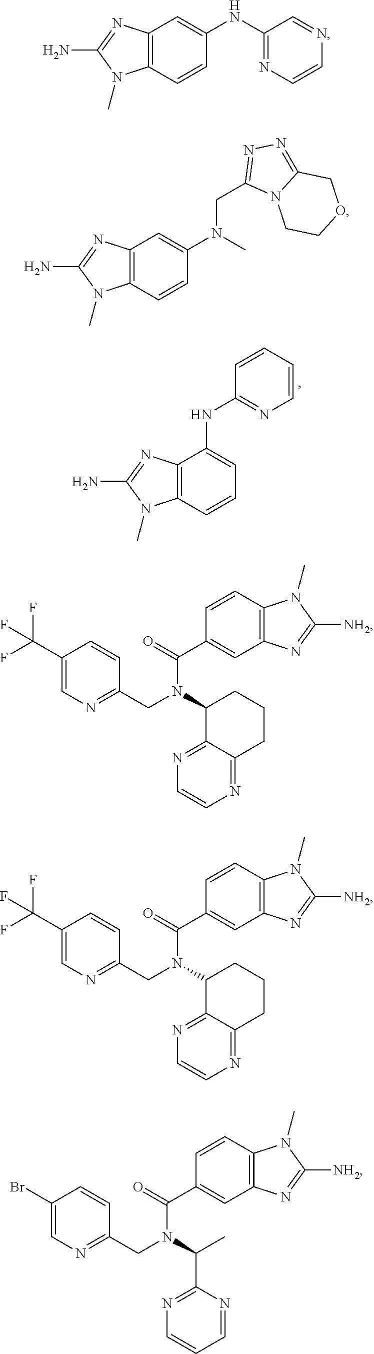 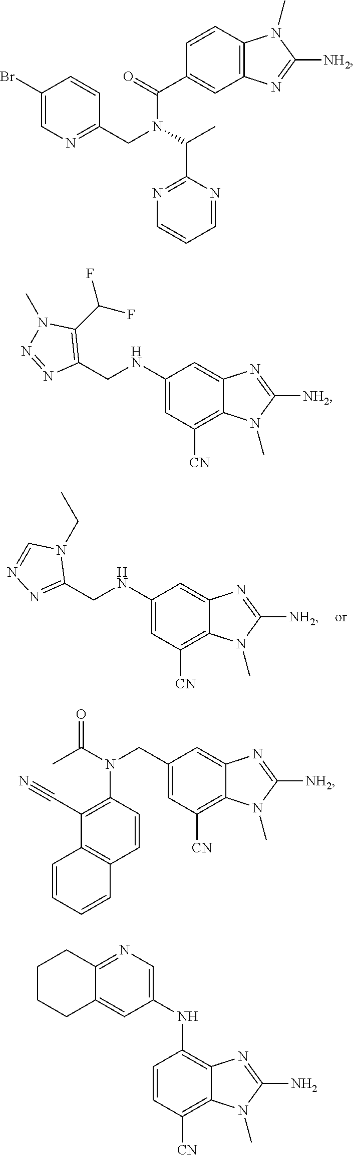
or a pharmaceutically acceptable salt of the foregoing compounds.

18. A compound which is
rac-N5-(1-(1H-1,2,4-triazol-1-yl) propan-2-yl)-1-methyl-1H-benzo[d]imidazole-2,5-diamine;
1-methyl-5-(1-methyl-1H-pyrazol-3-yl)-1H-benzo[d]imidazol-2-amine;
rac-1-methyl-N5-(1-(5,6,7,8-tetrahydroimidazo[1,2-a]pyridin-3-yl)ethyl)-1/-benzo[d]imidazole-2,5-diamine;
rac-4-((2-amino-1-methyl-1/-benzo[d]imidazol-5-yl)amino)-2,2-dimethyltetrahydro-2H-thiopyran 1,1-dioxide;
1-methyl-N5-((4-methyltetrahydrofuran-2-yl)methyl)-1/-benzo[d]imidazole-2,5-diamine;
N4-((4-(tert-butyl) oxazol-2-yl)methyl)-1-methyl-1H-benzo[d]imidazole-2,4-diamine;
rac-(R)-1-methyl-N5-(1-phenyl-2-(1//-1,2,4-triazol-1-yl)ethyl)-1H-benzo[d]imidazole-2,5-diamine;
1-methyl-N4-((2-methyl-1-phenyl-1H-imidazol-5-yl)methyl)-1H-benzo[d]imidazole-2,4-diamine;
1-methyl-N4-((5-methylimidazo[1,2-a]pyridin-2-yl)methyl)-1H-benzo[d]imidazole-2,4-diamine;
2-(((2-amino-1-methyl-1/-benzo[d]imidazol-4-yl)amino)methyl) phenol;
2-((2-amino-1-methyl-1H-benzo[d]imidazol-4-yl)amino)-4-(dimethylamino)benzonitrile;
rac-1-methyl-N5-(5-oxaspiro[3.5]nonan-8-yl)-1H-benzo[d]imidazole-2,5-diamine;
N4-((5-methoxypyridin-3-yl)methyl)-1-methyl-1H-benzo[d]imidazole-2,4-diamine;
1-methyl-N4-(5,6,7,8-tetrahydroquinolin-3-yl)-1H-benzo[d]imidazole-2,4-diamine;
N4-(5-(1,1-difluoroethyl) pyridin-3-yl)-1-methyl-1H-benzo[d]imidazole-2,4-diamine;
1-methyl-N5-(pyridin-2-yl)-1H-benzo[d]imidazole-2,5-diamine;
1-methyl-N5-(pyrimidin-2-yl)-1H-benzo[d]imidazole-2,5-diamine;
rac-(1-methyl-N5-(2-methyl-3-(1H-pyrazol-1-yl) propyl)-1H-benzo[d]imidazole-2,5-diamine;
N5-((4-ethyl-4H-1,2,4-triazol-3-yl)methyl)-1-methyl-1H-benzo[d]imidazole-2,5-diamine;
rac-1-((2-amino-1-methyl-1H-benzo[d]imidazol-5-yl)amino) propan-2-ol;
rac-1-methyl-N5-(1-(2-methylthiazol-5-yl)ethyl)-1H-benzo[d]imidazole-2,5-diamine;
1-methyl-N5-(4-methyltetrahydrofuran-3-yl)-1H-benzo[d]imidazole-2,5-diamine;
N5-(2-oxabicyclo[4.2.0]octan-7-yl)-1-methyl-1H-benzo[d]imidazole-2,5-diamine;
N4-(5-methoxypyridin-3-yl)-1-methyl-1H-benzo[d]imidazole-2,4-diamine;
N4-(5,6-dimethylpyridin-3-yl)-1-methyl-1H-benzo[d]imidazole-2,4-diamine;
N4-(5-chloro-6-methylpyridin-3-yl)-1-methyl-1H-benzo[d]imidazole-2,4-diamine;
N4-((4,5-dimethylthiazol-2-yl)methyl)-1-methyl-1H-benzo[d]imidazole-2,4-diamine;
4-((2-amino-1-methyl-1H-benzo[d]imidazol-4-yl)amino)-1-methyl-1H-imidazole-5-carbonitrile;
N4-(5-isopropyl-1,2,4-thiadiazol-3-yl)-1-methyl-1H-benzo[d]imidazole-2,4-diamine;
N4-(1-isopropyl-1H-1,2,4-triazol-3-yl)-1-methyl-1H-benzo[d]imidazole-2,4-diamine;
N4-(1-isopropyl-1H-imidazol-4-yl)-1-methyl-1H-benzo[d]imidazole-2,4-diamine;
rac-(1-methyl-N4-(5-oxaspiro[3.4]octan-7-yl)-1H-benzo[d]imidazole-2,4-diamine;
N5-(3-methoxybenzyl)-N5,1-dimethyl-1H-benzo[d]imidazole-2,5-diamine;
1-methyl-5-(1-methyl-1H-pyrazol-4-yl)-1H-benzo[d]imidazol-2-amine;
1-methyl-5-(1-methyl-1H-pyrazol-5-yl)-1H-benzo[d]imidazol-2-amine;
1-methyl-5-(4-methylpyridin-3-yl)-1H-benzo[d]imidazol-2-amine;
3-(2-amino-1-methyl-1H-benzo[d]imidazol-5-yl)-2-fluorophenol;
2-(2-amino-1-methyl-1H-benzo[d]imidazol-5-yl) furan-3-carbonitrile;
1-methyl-4-(1-methyl-1H-pyrazol-4-yl)-1H-benzo[d]imidazol-2-amine;
1-methyl-4-(1H-pyrazol-4-yl)-1H-benzo[d]imidazol-2-amine;
N5-((5-(difluoromethyl)-1-methyl-1H-1,2,3-triazol-4-yl)methyl)-1-methyl-1H-benzo[d]imidazole-2,5-diamine;
1-methyl-N4-(pyrimidin-2-yl)-1H-benzo[d]imidazole-2,4-diamine;
N4-(imidazo[1,2-a]pyridin-2-ylmethyl)-1-methyl-1H-benzo[d]imidazole-2,4-diamine;
1-methyl-N5-(1-methyl-1H-pyrazol-5-yl)-1H-benzo[d]imidazole-2,5-diamine;
N5-((5,6-dihydro-8H-[1,2,4]triazolo[3,4-c][1,4]oxazin-3-yl)methyl)-N5,1-dimethyl-1H-benzo[d]imidazole-2,5-diamine;
N-[(2-amino-1-methyl-benzimidazol-5-yl)methyl]-N-(1-cyano-2-naphthyl) acetamide;
7-chloro-1-methyl-4-(1-methylpyrazol-4-yl)benzimidazol-2-amine;
2-[4-(2-amino-7-chloro-1-methyl-benzimidazol-4-yl)-2-methyl-pyrazol-3-yl]-3-fluoro-naphthalene-1-carbonitrile;
7-chloro-1-methyl-4-(5,6,7,8-tetrahydroquinolin-3-ylmethyl)benzimidazol-2-amine;
7-chloro-1-methyl-N4-(5,6,7,8-tetrahydroquinolin-3-yl)-1H-benzo[d]imidazole-2,4-diamine;
7-chloro-1-methyl-4-tetrahydrofuran-3-yl-benzimidazol-2-amine;
7-chloro-N5-1-dimethyl-N5-(3-pyridyl)benzimidazole-2,5-diamine;
7-chloro-1-methyl-5-(3-pyridyloxy)benzimidazol-2-amine
7-chloro-6-(methoxymethyl)-1-methyl-benzimidazol-2-amine;
2-amino-3-methyl-1,2-dihydrobenzimidazole-4-carbonitrile;
2-amino-3-methyl-6-(3-pyridylmethyl)benzimidazole-4-carbonitrile;
2-amino-3-methyl-6-(2-pyridylmethyl)benzimidazole-4-carbonitrile;
2-amino-3-methyl-6-(4-pyridylmethyl)benzimidazole-4-carbonitrile;
2-amino-3-methyl-6-(1-methylpyrazolo[4,3-c]pyridin-3-yl)benzimidazole-4-carbonitrile;
2-amino-3-methyl-6-[(1-methylpyrazol-4-yl)methyl]benzimidazole-4-carbonitrile;
2-amino-3-methyl-6-[(1-methylpyrazol-3-yl)methyl]benzimidazole-4-carbonitrile;
2-amino-3-methyl-6-[(2-methylpyrazol-3-yl)methyl]benzimidazole-4-carbonitrile;
2-amino-6-(7-isoquinolylmethyl)-3-methyl-benzimidazole-4-carbonitrile;
2-amino-6-[(2-cyanophenyl)methyl]-3-methyl-benzimidazole-4-carbonitrile;
2-amino-5-benzyl-1-methyl-1H-benzo[d]imidazole-7-carbonitrile;
2-(2-amino-7-cyano-1-methyl-benzimidazol-5-yl)-N-phenyl-acetamide;
2-amino-3-methyl-6-(3-pyridyloxy)benzimidazole-4-carbonitrile;
2-amino-3-methyl-6-(3-pyridylsulfanyl)benzimidazole-4-carbonitrile;
2-amino-3-methyl-6-[1-(3-pyridyl)ethyl]benzimidazole-4-carbonitrile;
2-amino-3-methyl-6-(pyridine-3-carbonyl)benzimidazole-4-carbonitrile;
2-amino-6-[difluoro (3-pyridyl)methyl]-3-methyl-benzimidazole-4-carbonitrile;
2-amino-7-fluoro-3-methyl-benzimidazole-4-carbonitrile;
2-(2,5-dimethylpyrrol-1-yl)-3-methyl-7-(3-oxopyrazolidin-1-yl)benzimidazole-4-carbonitrile;
2-amino-4-methoxy-1-methyl-1H-benzo[d]imidazole-7-carbonitrile;
2-amino-7-methoxy-3-methyl-6-(3-pyridylmethyl)benzimidazole-4-carbonitrile;
2-amino-5-(((5-(difluoromethyl)-1-methyl-1H-1,2,3-triazol-4-yl)methyl)amino)-1-methyl-1H-benzo[d]imidazole-7-carbonitrile;
2-amino-5-(((4-ethyl-4H-1,2,4-triazol-3-yl)methyl)amino)-1-methyl-1H-benzo[d]imidazole-7-carbonitrile;
N-[(2-amino-7-cyano-1-methyl-benzimidazol-5-yl)methyl]-N-(1-cyano-2-naphthyl) acetamide;
2-amino-1-methyl-4-((5,6,7,8-tetrahydroquinolin-3-yl)amino)-1H-benzo[d]imidazole-7-carbonitrile;
7-(difluoromethyl)-1-methyl-5-(3-pyridylmethyl)benzimidazol-2-amine;
7-(difluoromethyl)-1-methyl-5-(2-pyridylmethyl)benzimidazol-2-amine;
1-ethyl-1H-imidazo[4,5-b]pyridin-2-amine;
1-benzylimidazo[4,5-b]pyridin-2-amine;
3-methyl-6-(3-pyridyloxy) imidazo[4,5-b]pyridin-2-amine;
2-[4-(2-amino-6-chloro-1-methyl-benzimidazol-4-yl)-2-methyl-pyrazol-3-yl]benzonitrile;
2-[4-(2-amino-1,6-dimethyl-benzimidazol-4-yl)-2-methyl-pyrazol-3-yl]benzonitrile;
2-[4-(2-amino-6-ethyl-1-methyl-benzimidazol-4-yl)-2-methyl-pyrazol-3-yl]benzonitrile;
7-fluoro-1-methyl-5-(3-pyridylmethyl)benzimidazol-2-amine; or
a pharmaceutically acceptable salt thereof.

19. A compound which is
1-methyl-5-(1-methyl-1H-pyrazol-3-yl)-1H-benzo[d]imidazol-2-amine;
1-methyl-N4-((2-methyl-1-phenyl-1H-imidazol-5-yl)methyl)-1H-benzo[d]imidazole-2,4-diamine;
1-methyl-N4-((5-methylimidazo[1,2-a]pyridin-2-yl)methyl)-1H-benzo[d]imidazole-2,4-diamine;
2-((2-amino-1-methyl-1H-benzo[d]imidazol-4-yl)amino)-4-(dimethylamino)benzonitrile;
1-methyl-N4-(5,6,7,8-tetrahydroquinolin-3-yl)-1H-benzo[d]imidazole-2,4-diamine;
N4-(5-(1,1-difluoroethyl) pyridin-3-yl)-1-methyl-1H-benzo[d]imidazole-2,4-diamine;
1-methyl-N5-(pyrimidin-2-yl)-1H-benzo[d]imidazole-2,5-diamine;
rac-1-((2-amino-1-methyl-1H-benzo[d]imidazol-5-yl)amino) propan-2-ol;
N4-(5,6-dimethylpyridin-3-yl)-1-methyl-1H-benzo[d]imidazole-2,4-diamine;
N4-(5-chloro-6-methylpyridin-3-yl)-1-methyl-1H-benzo[d]imidazole-2,4-diamine;
N4-((4,5-dimethylthiazol-2-yl)methyl)-1-methyl-1H-benzo[d]imidazole-2,4-diamine;
N4-(5-isopropyl-1,2,4-thiadiazol-3-yl)-1-methyl-1H-benzo[d]imidazole-2,4-diamine;
N5-(3-methoxybenzyl)-N5,1-dimethyl-1H-benzo[d]imidazole-2,5-diamine;
1-methyl-5-(1-methyl-1H-pyrazol-4-yl)-1H-benzo[d]imidazol-2-amine;
1-methyl-5-(1-methyl-1H-pyrazol-5-yl)-1H-benzo[d]imidazol-2-amine;
1-methyl-5-(4-methylpyridin-3-yl)-1H-benzo[d]imidazol-2-amine;
3-(2-amino-1-methyl-1H-benzo[d]imidazol-5-yl)-2-fluorophenol;
1-methyl-4-(1-methyl-1H-pyrazol-4-yl)-1H-benzo[d]imidazol-2-amine;
1-methyl-4-(1H-pyrazol-4-yl)-1H-benzo[d]imidazol-2-amine;
N5-((5,6-dihydro-8H-[1,2,4]triazolo[3,4-c][1,4]oxazin-3-yl)methyl)-N5,1-dimethyl-1H-benzo[d]imidazole-2,5-diamine;
2-(4-(2-amino-1-methyl-1H-benzo[d]imidazol-4-yl)-1-methyl-1H-pyrazol-5-yl)benzonitrile;
4-methoxy-1-methyl-5-(3-pyridylmethyl)benzimidazol-2-amine;
2-[4-(2-amino-7-chloro-1-methyl-benzimidazol-4-yl)-2-methyl-pyrazol-3-yl]-3-fluoro-naphthalene-1-carbonitrile;
7-chloro-1-methyl-4-(5,6,7,8-tetrahydroquinolin-3-ylmethyl)benzimidazol-2-amine;
7-chloro-1-methyl-N4-(5,6,7,8-tetrahydroquinolin-3-yl)-1H-benzo[d]imidazole-2,4-diamine;
7-chloro-1-methyl-4-tetrahydrofuran-3-yl-benzimidazol-2-amine;
7-chloro-N5-1-dimethyl-N5-(3-pyridyl)benzimidazole-2,5-diamine;
7-chloro-1-methyl-5-(3-pyridyloxy)benzimidazol-2-amine
7-chloro-6-(methoxymethyl)-1-methyl-benzimidazol-2-amine;
2-amino-3-methyl-1,2-dihydrobenzimidazole-4-carbonitrile;
2-amino-3-methyl-6-(3-pyridylmethyl)benzimidazole-4-carbonitrile;
2-amino-3-methyl-6-(2-pyridylmethyl)benzimidazole-4-carbonitrile;
2-amino-3-methyl-6-(1-methylpyrazolo[4,3-c]pyridin-3-yl)benzimidazole-4-carbonitrile;
2-amino-3-methyl-6-[(1-methylpyrazol-4-yl)methyl]benzimidazole-4-carbonitrile;
2-amino-3-methyl-6-[(1-methylpyrazol-3-yl)methyl]benzimidazole-4-carbonitrile;
2-amino-3-methyl-6-[(2-methylpyrazol-3-yl)methyl]benzimidazole-4-carbonitrile;
2-amino-6-(7-isoquinolylmethyl)-3-methyl-benzimidazole-4-carbonitrile;
2-amino-6-[(2-cyanophenyl)methyl]-3-methyl-benzimidazole-4-carbonitrile;
2-amino-5-benzyl-1-methyl-1H-benzo[d]imidazole-7-carbonitrile;
2-(2-amino-7-cyano-1-methyl-benzimidazol-5-yl)-N-phenyl-acetamide;
2-amino-3-methyl-6-(3-pyridyloxy)benzimidazole-4-carbonitrile;
2-amino-3-methyl-6-(3-pyridylsulfanyl)benzimidazole-4-carbonitrile;
2-amino-3-methyl-6-[1-(3-pyridyl)ethyl]benzimidazole-4-carbonitrile;
2-amino-3-methyl-6-(pyridine-3-carbonyl)benzimidazole-4-carbonitrile;
2-amino-7-fluoro-3-methyl-benzimidazole-4-carbonitrile;
2-(2,5-dimethylpyrrol-1-yl)-3-methyl-7-(3-oxopyrazolidin-1-yl)benzimidazole-4-carbonitrile;
2-amino-4-methoxy-1-methyl-1H-benzo[d]imidazole-7-carbonitrile;
2-amino-7-methoxy-3-methyl-6-(3-pyridylmethyl)benzimidazole-4-carbonitrile;
2-amino-5-(((5-(difluoromethyl)-1-methyl-1H-1,2,3-triazol-4-yl)methyl)amino)-1-methyl-1H-benzo[d]imidazole-7-carbonitrile;
N-[(2-amino-7-cyano-1-methyl-benzimidazol-5-yl)methyl]-N-(1-cyano-2-naphthyl) acetamide;
2-amino-1-methyl-4-((5,6,7,8-tetrahydroquinolin-3-yl)amino)-1H-benzo[d]imidazole-7-carbonitrile;
1-benzylimidazo[4,5-b]pyridin-2-amine;
2-[4-(2-amino-6-chloro-1-methyl-benzimidazol-4-yl)-2-methyl-pyrazol-3-yl]benzonitrile;
2-[4-(2-amino-1,6-dimethyl-benzimidazol-4-yl)-2-methyl-pyrazol-3-yl]benzonitrile;
2-[4-(2-amino-6-ethyl-1-methyl-benzimidazol-4-yl)-2-methyl-pyrazol-3-yl]benzonitrile;
7-fluoro-1-methyl-5-(3-pyridylmethyl)benzimidazol-2-amine; or
a pharmaceutically acceptable salt thereof.

20. A compound which is
2-(4-(2-amino-1-methyl-1H-benzo[d]imidazol-4-yl)-1-methyl-1H-pyrazol-5-yl)benzonitrile;
7-chloro-1-methyl-5-(3-pyridylmethyl)benzimidazol-2-amine;
2-[4-(2-amino-7-chloro-1-methyl-benzimidazol-4-yl)-2-methyl-pyrazol-3-yl]-3-fluoro-naphthalene-1-carbonitrile;
2-amino-3-methyl-1,2-dihydrobenzimidazole-4-carbonitrile;
2-amino-6-[(2-cyanophenyl)methyl]-3-methyl-benzimidazole-4-carbonitrile;
2-(2-amino-7-cyano-1-methyl-benzimidazol-5-yl)-N-phenyl-acetamide;
2-amino-3-methyl-6-(3-pyridyloxy)benzimidazole-4-carbonitrile;
2-amino-3-methyl-6-[1-(3-pyridyl)ethyl]benzimidazole-4-carbonitrile;
rac-1-((2-amino-1-methyl-1H-benzo[d]imidazol-5-yl)amino) propan-2-ol;
N4-((8-fluoroimidazo[1,2-a]pyridin-2-yl)methyl)-1-methyl-1H-benzo[d]imidazole-2,4-diamine;
1-methyl-N5-(pyrazin-2-yl)-1H-benzo[d]imidazole-2,5-diamine;
2-amino-7-fluoro-3-methyl-benzimidazole-4-carbonitrile;
2-amino-3-methyl-6-(3-pyridylmethyl)benzimidazole-4-carbonitrile;
2-amino-3-methyl-6-(2-pyridylmethyl)benzimidazole-4-carbonitrile;
2-amino-3-methyl-6-[(1-methylpyrazol-4-yl)methyl]benzimidazole-4-carbonitrile;
2-amino-3-methyl-6-[(1-methylpyrazol-3-yl)methyl]benzimidazole-4-carbonitrile;
2-amino-6-(7-isoquinolylmethyl)-3-methyl-benzimidazole-4-carbonitrile;
2-amino-5-(((5-(difluoromethyl)-1-methyl-1H-1,2,3-triazol-4-yl)methyl)amino)-1-methyl-1H-benzo[d]imidazole-7-carbonitrile;
2-amino-5-(((4-ethyl-4H-1,2,4-triazol-3-yl)methyl)amino)-1-methyl-1H-benzo[d]imidazole-7-carbonitrile;
N-[(2-amino-7-cyano-1-methyl-benzimidazol-5-yl)methyl]-N-(1-cyano-2-naphthyl) acetamide;
7-(difluoromethyl)-1-methyl-5-(2-pyridylmethyl)benzimidazol-2-amine;
1-benzylimidazo[4,5-b]pyridin-2-amine;
2-[4-(2-amino-6-chloro-1-methyl-benzimidazol-4-yl)-2-methyl-pyrazol-3-yl]benzonitrile;
2-[4-(2-amino-1,6-dimethyl-benzimidazol-4-yl)-2-methyl-pyrazol-3-yl]benzonitrile;
2-[4-(2-amino-6-ethyl-1-methyl-benzimidazol-4-yl)-2-methyl-pyrazol-3-yl]benzonitrile;
7-fluoro-1-methyl-5-(3-pyridylmethyl)benzimidazol-2-amine;
3-(2-amino-1-methyl-1H-benzo[d]imidazol-5-yl)-2-fluorophenol;
2-(2-amino-1-methyl-1H-benzo[d]imidazol-5-yl) furan-3-carbonitrile; or
a pharmaceutically acceptable salt thereof.

21. A compound which is(S)
(S)-2-amino-1-methyl-N-(5,6,7,8-tetrahydroquinoxalin-5-yl)-N-((5-(trifluoromethyl)pyridin-2-yl)methyl)-1H-benzo[d]imidazole-5-carboxamide;
(R)-2-amino-1-methyl-N-(5,6,7,8-tetrahydroquinoxalin-5-yl)-N-((5-(trifluoromethyl)pyridin-2-yl)methyl)-1H-benzo[d]imidazole-5-carboxamide;
(S)-2-amino-N-((5-bromopyridin-2-yl)methyl)-1-methyl-N-(1-(pyrimidin-2-yl)ethyl)-1H-benzo[d]imidazole-5-carboxamide;
(R)-2-amino-N-((5-bromopyridin-2-yl)methyl)-1-methyl-N-(1-(pyrimidin-2-yl)ethyl)-1H-benzo[d]imidazole-5-carboxamide
2-amino-6-[(2-cyanophenyl)methyl]-3-methyl-benzimidazole-4-carbonitrile;
2-amino-5-(((5-(difluoromethyl)-1-methyl-1H-1,2,3-triazol-4-yl)methyl)amino)-1-methyl-1H-benzo[d]imidazole-7-carbonitrile;
N-[(2-amino-7-cyano-1-methyl-benzimidazol-5-yl)methyl]-N-(1-cyano-2-naphthyl) acetamide; or
a pharmaceutically acceptable salt thereof.

22. A pharmaceutical composition, comprising a therapeutically effective amount of a compound of any one of claims 1-21 or a pharmaceutically acceptable salt thereof, and a pharmaceutically acceptable excipient.

23. A method for inhibiting PRMT5 activity in a cell, comprising contacting the cell in which inhibition of PRMT5 activity is desired with an effective amount of a compound according to any one of claims 1-21 or a pharmaceutically acceptable salt thereof, or a pharmaceutical composition according to claim 22.

24. A method for treating cancer comprising administering to a patient having cancer a therapeutically effective amount of a compound according to any one of claims 1-21 or a pharmaceutically acceptable salt thereof, alone or combined with a pharmaceutically acceptable carrier, excipient or diluents.

25. The method of claim 24, wherein the therapeutically effective amount of the compound is between about 0.01 to 300 mg/kg per day.

26. The method of claim 24, wherein the therapeutically effective amount of the compound is between about 0.1 to 100 g/kg per day.

27. The method according to any one of claims 24-26, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

28. The method according to any one of claims 24-26, wherein the cancer is a MTAP-associated cancer.

29. The method according to any one of claims 24-26, wherein the cancer is hepatocellular carcinoma, breast cancer, skin cancer, bladder cancer, liver cancer, pancreatic cancer, or head and neck cancer.
